(19) United States
(12) Patent Application Publication
(10) Pub. No.: US 2025/0325642 A1
(43) Pub. Date: Oct. 23, 2025

(54) VACCINE COMPOSITION FOR BREAKING SELF-TOLERANCE

(71) Applicant: BAYER ANIMAL HEALTH GMBH, Leverkusen (DE)

(72) Inventor: Thomas ILG, Monheim (DE)

(21) Appl. No.: 18/262,949

(22) PCT Filed: Jan. 29, 2022
(86) PCT No.: PCT/EP2022/052154

§ 371 Date: Jul. 26, 2023

(30) Foreign Application Priority Data
Jan. 29, 2021 (EP) 21154244.4

(51) Int. Cl. A61K 39/00; A61K 39/385; A61P 37/06; C12N 15/85; G01N 33/543; G01N 33/564; G01N 33/68

(52) U.S. Cl. CPC A61K 39/0008; A61K 39/00114; A61K 39/385; A61P 37/06; C12N 15/85; G01N 33/543; G01N 33/564; G01N 33/6854; A61K 2039/552; A61K 2039/55561; A61K 2039/575; A61K 2039/6037

(57) ABSTRACT

The present invention relates to a vaccine composition for breaking self-tolerance against a self-protein of a host, in particular for breaking self-tolerance against endogenous cytokines, in particular against the endogenous IL-4, IL-5, IL-13, IL-31 and IL-33 proteins in an animal host. The vaccine composition of the invention contains a polyprotein, a DNA encoding for the polyprotein and/or an RNA encoding for the polyprotein and one or more immunostimulatory oligonucleotides. The polyprotein comprises at least two self-protein segments of the host and one or more T-cell epitopes of non-host origin in between and/or adjacent to the at least two self-protein segments.

The present invention further concerns the use of the vaccine composition for the prevention and/or treatment of diseases including the prevention and/or treatment of a pruritic condition and/or an allergic condition. In another aspect, the present invention provides a method for detecting the presence of autoantibodies against self-proteins that can be generated with the vaccine composition of the invention.

### FIELD OF THE INVENTION

The present invention relates to a vaccine composition for breaking self-tolerance against a self-protein of a host, in particular for breaking self-tolerance against endogenous cytokines, including cytokines derived from IL-4, IL-5, IL-13, IL-31, IL-33, and TNF-alpha proteins, and in particular combinations of cytokines comprising the endogenous IL-31 protein, in a mammalian host. The present invention further concerns the use of the vaccine composition for the prevention and/or treatment of diseases including the prevention and/or treatment of a pruritic condition and/or an allergic condition. The present invention further concerns a polyprotein, which is derived from the self-protein and which is used as immunogen in the vaccine composition. In another aspect, the present invention provides a method for detecting the presence of autoantibodies against self-proteins that can be generated with the vaccine composition of the invention.

### BACKGROUND OF THE INVENTION

Vaccines are of paramount importance for the prevention and/or treatment of infectious diseases. Vaccine technology, however, also gains more and more importance for the prevention and/or treatment of noninfectious, often chronic diseases such as allergies, autoimmune diseases and cancer. The targets for these diseases are in general not foreign molecules but instead self-proteins or other self-antigens. Since the immune system has evolved to ensure tolerance for all self-proteins and self-antigens, it is very difficult to vaccinate against a self-protein. The research underlying the present invention aimed to find ways to circumvent or break self-tolerance.

Autoreactive B cells may be present in the circulation at low levels, but they do not expand or cause any harm, primarily due to the lack of T-cell help. In contrast, any self-reactive T cells that occur are either clonally deleted in the thymus or anergized in the periphery. It is known, however, that if a self-antigen is covalently coupled to a foreign (non-self) protein or part thereof, meaning that a fusion protein comprising self and non-self proteins or protein parts is provided, T-cells specific for the non-self-protein (part) are recruited and activated.

In parallel, the auto-reactive B cells may selectively take up the fusion protein containing self and non-self proteins/protein parts and therefore present both the self and the foreign peptides on MHC class II molecules. The non-self peptides presented by the autoreactive B cells are then recognized by the activated T-cells, which stimulate the autoreactive B cells to expand and initiate an immune response against the self-protein/self-antigen. If the immune response is strong enough, these self-produced antibodies have the capacity to reduce the level of the target self-protein. If a self-protein is chosen as target, which is responsible for or contributes to a disease, the in vivo generated autoantibodies can act as therapeutic antibodies by neutralizing the target self-protein. Such a robust immune response, however, is difficult to obtain.

In various pathologies including allergy, autoimmunity, cancer and AIDS an abnormal release of cytokines contributes to pathogenesis and/or disease progression. Typically, a number of different cytokines are involved pathologies.

Atopic dermatitis, for example, is a frequent allergic skin disorder that is characterized by aberrant and excessive Th2 cell and ILC2 activation, with robust expression of type 2 cytokines, including interleukin (IL)-4, IL-5, IL-13 and IL-31, and variable activation of other cytokines, in particular IL-22 and IL-33, but also IL-17, IL-9 and IFN-γ (Moyle et al. (2019) Experimental Dermatology, 28:756-768; Renert-Yuval & Guttman-Yassky (2019) Dermatol Clin 37:205-213). Atopic dermatitis is not only a frequent disorder in humans, but also in animals, in particular dogs. In fact, atopic dermatitis is the most common allergy in dogs and affects approximately 10% of the dog population, resulting in 15 million to 20 million dogs suffering from the disease in Europe and the United States alone (Griffin, et al. (2001), “The ACVD task force on canine atopic dermatitis (XIV): clinical manifestations of canine atopic dermatitis”, Veterinary immunology and immunopathology, 81 (3-4), 255-269). The itching or pruritus which is caused by this allergic skin disease is usually recurrent or chronic. It deeply impacts the quality of life for both the dogs and their owners.

In particular, the endogenous pruritogen, Interleukin-31 (IL-31), appears to play a critical role in atopic itch. Since IL-31 seems to be a key regulator of pruritus in atopic dermatitis in humans and dogs (Sonkoly et al., “IL-31: a new link between T cells and pruritus in atopic skin inflammation”, Journal of Allergy and Clinical Immunology 117.2 (2006): 411-417; Furue et al., “Emerging role of interleukin-31 and interleukin-31 receptor in pruritus in atopic dermatitis”, Allergy 73.1 (2018): 29-36; Gonzales et al., “Interleukin-31: its role in canine pruritus and naturally occurring canine atopic dermatitis.” Veterinary dermatology 24.1 (2013): 48-e12), IL-31 itself and its receptor binding has been a major focus for pharmacologically intervening itch in the context of atopic dermatitis.

Asthma is another highly prevalent condition with a pathophysiology linked to the abnormal release of cytokines, both of the Type 2, but also Type 1 type. Major targets of asthma-related treatment studies include IL-4, IL-5, and IL-13, as well as IL-33.

Different strategies have been pursued to treat conditions linked to aberrant cytokine production. In atopic dermatitis, for example, one of the strategies has focused on inhibiting downstream signal transduction using, e.g. kinase inhibitors. This strategy, however, has the disadvantage that the inhibitor has to be given repeatedly in short time intervals to the human or animal patient. Another strategy that has been widely used is the development of neutralizing monoclonal antibodies against a particular cytokine of interest in order to reduce circulating ligand levels and/or otherwise inhibit their receptor binding and thus biological activity. In atopic dermatitis, for example, anti-4, anti-5, anti-13, anti-17A, anti-17C, anti-22, anti-31, and anti-33 antibodies have been developed to prevent and/or treat this condition (Moyle et al. (2019) Experimental Dermatology, 28:756-768; Renert-Yuval & Guttman-Yassky (2019) Dermatol Clin 37:205-213). This strategy, however, has the disadvantage of high production costs due to the expensive antibody production procedure in cell culture and the need to repeat the antibody treatment in short intervals. Another disadvantage of this strategy is that typically progressive immunization against the monoclonal therapeutic antibody occurs in the patient so that in the long run, the therapeutic antibody treatment is no longer effective.

Bachmann et al. 2018 attempted to vaccinate against the cytokine, IL-31, by administering a vaccine which contains native, full-length IL-31 chemically coupled to virus-like particles (VLPs) derived from cucumber mosaic virus and containing a universal T-cell epitope (Bachmann et al., “Vaccination against IL-31 for the treatment of atopic dermatitis in dogs”, Journal of Allergy and Clinical Immunology 142.1 (2018): 279-281). The chemical coupling of native IL-31 to the VLP was achieved by derivatinzing IL-31 and the VLP coat proteins. IL-31 was derivatized with N-succinimidyl S-acetylthioacetate followed by deacetylation to introduce reactive SH-groups into IL-31. VLP coat proteins were derivatized with succinimidyl-6-((beta-maleimidopropionamido) hexanoate) to introduce SH-reactive chemical moieties. The derivatized preparations of IL-31 and VLPs were reacted with one another and purified. This strategy, however, has the disadvantage that the production of the IL-31-VLP conjugates is highly complex and expensive, requiring purified VLPs and IL-31, multiple chemical steps for derivatization and chemical coupling and subsequent purification. Moreover, a well-defined chemical product is not obtained by this production method. The obtained IL-31-VLP conjugates also contain non-natural components and chemical linkages whose biodegradability can be problematic.

A goal of the research underlying the present invention was to provide human and veterinary medicines in the form of therapeutic vaccines which can stimulate an immune response, in particular against deleterious cytokines.

### SUMMARY OF THE INVENTION

Against the aforementioned background, it is an object of the present invention to provide effective pharmacological means to inhibit or perturb the function of a disease-causing or disease-contributing target self-protein as compared to the means known in the art. It also is an object of the present invention to provide pharmacological means that induce a long lasting effect against the target self-protein in the host so that the pharmacological means need to be readministered only after a long time interval, preferably in the range of weeks, most preferably in the range of months. A further object of the invention is to provide pharmacological means that can be produced in an economical manner and are chemically well defined in their components.

In connection with the above objects, it is another object of the invention to provide a simple and effective method to investigate the effect of the pharmacological means of the invention to inhibit or perturb the function of a disease-involved or disease-causing target self-protein.

These objects are achieved by the vaccine composition according to claim 1, the polyprotein according to claim 17, the uses according to claims 18 and 19 and the method according to claim 23.

The invention provides a polyprotein, a DNA encoding for this polyprotein and/or an RNA encoding for this polyprotein for use in a vaccine composition to break self-tolerance against a self-protein of a host, wherein the polyprotein comprises at least two self-protein segments and one or more T-cell epitopes of non-host origin in between and/or adjacent to the at least two self-protein segments. In particular, it comprises at least two self-protein segments derived from one self-protein of a host and at least two self-protein segments derived from another self-protein of the same host, in addition to the one or more T-cell epitopes of non-host origin in between and/or adjacent to the self-protein segments. In a preferred embodiment of the invention, the polyprotein comprises two or three copies of one self-protein of a host, two or three copies of another self-protein of the same host, and one or two T-cell epitopes of non-host origin in between and/or adjacent to the self-protein segments.

The research underlying the invention surprisingly found that a polyprotein comprising self-protein segments and non-host T-cell epitopes in between and/or adjacent to these self-protein segments is capable to break or circumvent the self-tolerance of a host against the self-protein segments of the polyprotein. The design of the polyprotein of the invention has not only immunological advantages, but also allows the administration of large amounts of the polyprotein of the invention, in particular subcutaneously, without producing significant negative effects caused by the self-protein segments in the polyprotein exerting their normal biological and/or disease-causing functions. This makes the polyprotein according to the invention a particularly suitable antigen for a vaccine composition.

The vaccine composition of the invention comprises the polyprotein, the DNA encoding for the polyprotein and/or the RNA encoding for the polyprotein according to the invention. More precisely, the invention provides a vaccine composition for breaking self-tolerance against a self-protein of a host, wherein the vaccine composition is capable of raising autoantibodies against said self-protein when the vaccine composition is administered to the host. The vaccine composition of the invention comprises:
a) a polyprotein, a DNA encoding for the polyprotein and/or an RNA encoding for the polyprotein, wherein the polyprotein comprises
   at least two self-protein segments derived from a first self-protein of the host;
   at least two self-protein segments derived from a second self-protein of the host;
   optionally at least two self-protein segments derived from a third self-protein of the host; and
   one or more T-cell epitopes of non-host origin in between and/or adjacent to the self-protein segments;
and
b) one or more immunostimulatory oligonucleotides.

The inventors surprisingly found that a vaccine comprising a polyprotein containing self-protein segments and non-host T-cell epitopes in between and/or adjacent to the self-protein segments in combination with one or more immunostimulatory oligonucleotides as adjuvants is capable to induce a potent immune response against the self-protein segments of the polyprotein in the host to which the vaccine composition is administered. This potent immune response in the host includes the production of autoantibodies against the self-protein segments of the polyprotein. Experiments of the inventors showed that the autoantibodies produced after vaccination with the vaccine composition according to the invention also bind to the native self-proteins from which the self-protein segments were derived. The inventors further observed that the produced autoantibodies were present in the host's circulation system for weeks and could perturb or even neutralize the function of the bound self-proteins. This was not only the case when two or more segments from just one type of self-protein was comprised in the polyprotein, but also when two or more segments from more than one different type of self-proteins were comprised in the polyprotein. Thus, the vaccine composition according to the invention allows the induction of a long lasting therapeutic autoantibody response in vivo.

Important is that at least two segments (used interchangeably with the term “self-protein segment”) are present for each self-protein comprised in the polyprotein. These segments typically have a high level of sequence identity, and most preferably are exact copies of each other. However, different splicing events may occur, leading to lower sequence identity, but without effect on the function of the segment or of the polyprotein as a whole. In this sense, the at least two segments derived from the same self-protein may have at least 85%, at least 90%, at least 95%, at least 98%, at least 99%, or at least 99.5% sequence identity with one another. For sequence identities less than 100%, the differences in sequence identity should allow the two segments to still result in similar biological activity when tested individually, i.e. not in the form of a polyprotein.

The present invention also concerns the use of a polyprotein to break self-tolerance against a self-protein of a host, wherein the self-tolerance is broken by the production of autoantibodies when the polyprotein is administered to the host, and wherein the polyprotein comprises at least two self-protein segments, and one or more T-cell epitopes of non-host origin in between and/or adjacent to the at least two self-protein segments; in particular wherein the polyprotein comprises at least two self-protein segments derived from one self-protein of a host, at least two self-protein segments derived from another self-protein of the same host, and one or more T-cell epitopes of non-host origin in between and/or adjacent to the self-protein segments. This use is particularly relevant in prophylactic and therapeutic medical applications, and in particular those where multiple self-proteins are involved in the pathology.

Further the invention concerns a vaccine composition according to the invention for use in a method of preventing or treating a disease in a subject, wherein the method comprises the step of administering the vaccine to the subject.

Lastly, the invention concerns an enzyme-linked immunosorbent assay for detecting neutralizing autoantibodies comprising the steps of
a) Adsorbing an antigen onto a test surface;
b) Blocking of free binding sites on the test surface;
c) Incubating the antigen-coated and blocked test surface with a mixture comprising a labeled neutralizing antibody against the antigen and a to-be-tested neutralizing autoantibody against the antigen; and
d) Detecting the binding of the labeled neutralizing antibody.

The assay according to the invention allows to determine in a robust und unambigious fashion the presence of neutralizing autoantibodies against an antigen of interest, in particular after vaccination of a host with a vaccine composition according to the invention.

### DETAILED DESCRIPTION OF ILLUSTRATIVE EMBODIMENTS

### The Polyprotein

The polyprotein, the DNA encoding for the polyprotein or the RNA encoding for the polyprotein of the invention is designed to break self-tolerance against a self-protein of a host when administered to said host, e.g, in the vaccine composition of the invention. The polyprotein comprises at least two self-protein segments of the host and one or more T-cell epitopes of non-host origin in between and/or adjacent to the at least two self-protein segments of the host. In particular, it comprises at least two self-protein segments derived from one self-protein of a host and at least two self-protein segments derived from another self-protein of the same host, in addition to the one or more T-cell epitopes of non-host origin in between and/or adjacent to the self-protein segments. In a preferred embodiment of the invention, the polyprotein comprises two or three copies of one self-protein of a host, two or three copies of another self-protein of the same host, and one or two T-cell epitopes of non-host origin in between and/or adjacent to the self-protein segments.

Breaking self-tolerance against a self-protein of a host means eliciting an immune response in a host which comprises the production of autoantibodies, preferably neutralizing autoantibodies, against the self-protein of the host. “To break self-tolerance against a self-protein of a host” therefore means “to elicit the production of autoantibodies, preferably neutralizing autoantibodies, against a self-protein of a host”. The term “autoantibody” as used herein refers to an antibody produced by a host which binds to a self-protein of this host. A “neutralizing autoantibody” perturbs and preferably entirely inhibits the biological function of the host's self-protein to which it binds. As an example, a neutralizing autoantibody against IL-31 perturbs and preferably substantially entirely inhibits the biological function of the same in the host. In particular, a neutralizing autoantibody against IL-31 perturbs and preferably entirely inhibits IL-31's role in the induction and onset of pruritus.

The polyprotein of the invention comprises two critical structural elements: self-protein segments of a host and T-cell epitopes of non-host origin.

The polyprotein of the invention comprises at least two segments, preferably two or three segments, of each self-protein comprised in the polyprotein. Most preferably, the polyprotein according to the invention comprises two or three segments derived from a first self-protein with a sequence identity selected from the group consisting of SEQ ID NO: 3, 41, 46, 50, 51, 56, 60, 64, or SEQ ID NO: 68-201, two or three segments derived from a second self-protein with a sequence identity selected from the group consisting of SEQ ID NO: 3, 41, 46, 50, 51, 56, 60, 64, or SEQ ID NO: 68-201, and optionally two or three segments derived from a third self-protein a first self-protein with a sequence identity selected from the group consisting of SEQ ID NO: 3, 41, 46, 50, 51, 56, 60, 64, or SEQ ID NO: 68-201, in particular wherein the first, second, and optionally third self-proteins are different proteins of the same host.

Experiments of the inventors showed that when administered to a host, a polyprotein containing at least two segments of either two or three different self-proteins of a host is very potent in eliciting the production of autoantibodies in the host against each of the individual self-proteins of the polyprotein and thus also against the native self-proteins of the host from which the self-protein segments of the polyprotein were derived from.

The self-protein segments that are derived from the same self-protein can be the same or different. In particular, the self-protein segments can differ in length and/or amino acid sequence. Good results were achieved when the self-protein segments derived from the same self-protein were the same in the polyprotein. In these cases, the polyprotein, when administered to a host, induces an immune response in the host which is focused on the production of autoantibodies against each type of self-protein or even each type of self-protein segment, both of which results in an autoimmune response against the native self-proteins of the host that the protein segments were derived from.

A self-protein segment of the polyprotein according to the invention comprises at least one B-cell epitope. The term B-cell epitope as used herein means a linear or conformational proportion of the self-protein segment to which an autoantibody binds.

“Segments” as used herein means distinguishable and separate protein entities or domains. Thus, a single contiguous self-protein sequence of a host can only be considered as constituting at least two self-protein segments according to the invention if within the self protein sequence, segments have been separated by an intervening sequence (e.g., a T-cell epitope). Multiples of the same protein segment or different protein segments can also be directly fused to one another without any intervening sequences being present. Preferably, the intervening sequence comprises or consists of one or more T-cell epitopes of non-host origin.

The self-protein segment of the host can be
(i) a full-length self-protein; or
(ii) a truncated self-protein containing a B-cell epitope; or
(iii) a derivative of a self-protein which has at least 80% sequence identity, preferably at least 90% sequence identity and most preferably at least 95% sequence identity to the full-length self-protein.

The self-protein segments contained in the polyprotein according to the invention can all be of the same self-protein segment type or of different self-protein segment types wherein the self-protein segment type is selected from the group consisting of
(i) a full-length self-protein; or
(ii) a truncated self-protein containing a B-cell epitope; or
(iii) a derivative of a self-protein which has at least 80% sequence identity, preferably at least 90% sequence identity and most preferably at least 95% sequence identity to the self-protein.

Preferably, the self-protein segments of the polyprotein according to the invention are full-length self-proteins, preferably, multiple copies of the same full-length self-protein, e.g., IL-4, IL-5, IL-13, IL-31, IL-33, or TNF-alpha. For self-proteins where the full-length form is different from the mature form, the term “full-length” for the purpose of this invention, refers to the full-length mature form of said self-protein.

More preferably, the polyprotein according to the invention contains three self-protein segments wherein the self-protein segments are all full-length self-proteins. The use of full-length self-proteins in the polyprotein according to the invention (option (i)) has the advantage that the individual self-protein segments can in principle adapt their native fold. Because of this, the self-protein segment not only provides the same linear epitope but also the same conformational B-cell epitope as the native self-protein from which the self-protein segment of the host is derived from. This makes this type of self-protein segment in the polyprotein according to the invention particularly effective in breaking the self-tolerance of the host against the target self-protein.

The self-protein segment(s) contained in the polyprotein according to the invention can also be a truncated self-protein. In this case the truncation must be performed in a way that the remaining protein segment still contains at least one functioning B-cell epitope. The use of truncated self-proteins containing a B-cell epitope in the polyprotein according to the invention (option (ii)) has the advantage that the self-protein segments can be reduced in their size to primarily contain the relevant B-cell epitope(s). In this way, the polyprotein according to the invention can be reduced in size, which may aid in clonability and delivery, and/or it becomes possible to add even more self-protein segments in a polyprotein according to the invention while not exceeding a certain size limit of the polyprotein.

The self-protein segment(s) contained in the polyprotein according to the invention can be a derivative of a self-protein which has at least 80% sequence identity, preferably at least 90% sequence identity and most preferably at least 95% sequence identity to the full-length self-protein. Even more preferably, the derivative of the self-protein has 96%, 97%, 98% or 99% sequence identity to the full-length self-protein. A self protein segment according to the invention can at the same time fulfill the definition of a truncated self-protein and a derivative of a self-protein according to the invention. Preferably, the polyprotein according to the invention contains only self-proteins and/or derivatives of a self-protein which has at least 80% sequence identity, preferably at least 90% sequence identity and most preferably at least 95%, 97%, 98% or 99% sequence identity to the respective full-length self-protein. More preferably, the polyprotein according to the invention contains two self-protein segments of each self-protein wherein the self-protein segments are full-length self-proteins and/or derivatives of a self-protein which has at least 80% sequence identity, preferably at least 90% sequence identity and most preferably at least 95%, 97%, 98% or 99% sequence identity to the full-length self-protein. The use of derivative self-proteins in the polyprotein according to the invention (option (iii)) can be advantageous for multiple reasons, for example, it could allow the expression of a more stable or more soluble polyprotein according to the invention. It is also advantageous to use derivative self-proteins in the polyprotein according to the invention which carry mutations leading to impaired or entirely inhibited biological functions of these derivate self-proteins. In this regard, it is in particular conceivable to use a self-protein which bears mutations leading to a loss of receptor engagement and/or signal transduction potential. Such a derivative self-protein can, for example, be obtained by site-directed mutagenensis of residues critical for receptor binding.

In one embodiment, at least one, at least two, or at least three of the self-proteins, from which the self-protein segments are derived in the polyprotein according to the invention, are cytokines. Preferably, all of the self-proteins, from which the self-protein segments are derived, are cytokines. Cytokine as defined herein has its normal meaning in the art. Cytokines can for example be grouped by structure into families, for example into the IL-1 family, the hematopoietin superfamily, the interferons, and the tumor necrosis factor family. For the IL-1 family, it is known that most members of this family are produced as inactive proproteins that are cleaved (removing an amino-terminal peptide) to produce the mature cytokine. In such cases, the full-length protein refers to the mature form of said self-protein. The exception to this rule is IL-1-alpha, for which both the proprotein and its cleaved forms are biologically active. The hematopoietin superfamily of cytokines includes non-immune-system growth and differentiation factors such as erythropoietin and growth hormone, as well as interleukins with roles in innate and adaptive immunity. Many of the soluble cytokines made by activated T cells are members of the hematopoietin family. The TNF family, of which TNF-alpha is the prototype, contains more than 17 cytokines with important functions in adaptive and innate immunity. Cytokines also include colony-stimulating factors.

In one embodiment, at least one, at least two, or at least three of the self-proteins from which the self-protein segments are derived in the polyprotein according to the invention, is/are selected from the group of cytokines consisting of interleukin family members, tumor necrosis factor family members, interferon family members, and/or colony-stimulating factor family members. Examples of interleukin family members are interleukins selected from the group consisting of IL-1-alpha, IL-1-beta, IL-1 RA, IL-2, IL-3, IL-4, IL-5, IL-6, IL-7, IL-8, IL-9, IL-10, IL-11, IL-12, IL-13, IL-14, IL-15, IL-16, IL-17A-F, IL-18, IL-19, IL-20, IL-21, IL-22, IL-23, IL-24, IL-25, IL-26, IL-27, IL-28A,B, IL-29, IL-30, IL-31, IL-32, IL-33, IL-34, IL-35, IL-36-alpha, beta, or gamma, IL-36 Ra, IL-37, IL-38, IL-39, IL-40, IL-41, and IL-42, TSLP, leukemia inhibitory factor, and oncostatin, or a family member thereof. Examples of TNF family member self-proteins are proteins selected from the group consting of TNF-alpha, lymphotoxin (LT)-alpha, LT-beta, CD40 ligand, Fas ligand, APRIL, LIGHT, TWEAK, and BAFF. Examples of IFN family member self-proteins are proteins selected from the group consting of IFN-alpha, IFN-beta, and IFN-gamma. Examples of colony-stimulting factor cytokines are granulocyte colony stimulating factor (G-CSF) and granulocyte-macrophage colony-stimulating factor (GM-CSF). Preferably, the self-protein segments are derived from a self-protein, in particular a cytokine, that is monomeric, homodimeric, homotrimeric, or homotetrameric. In a preferred embodiment, the polyprotein according to the invention comprises at least two, in particular two or three, self-protein segments, wherein the self-protein segments are derived from a cytokine selected from the group consisting of IL-1-alpha, IL-1-beta, IL-1 RA, IL-2, IL-3, IL-4, IL-5, IL-6, IL-7, IL-8, IL-9, IL-10, IL-11, IL-13, IL-14, IL-15, IL-16, IL-17A-F, IL-18, IL-19, IL-20, IL-21, IL-22, IL-24, IL-25, IL-26, IL-28A,B, IL-29, IL-30, IL-31, IL-32, IL-33, IL-34, IL-36-alpha, beta, or gamma, IL-36 Ra, IL-37, IL-38, IL-40, IL-41, and IL-42, TSLP, leukemia inhibitory factor, oncostatin, TNF-alpha, IFN-alpha, IFN-beta, and IFN-gamma, G-CSF, and GM-CSF.

In a particularly preferred embodiment, the at least two self-protein segments of the polyprotein according to the invention are derived from IL-4, IL-5, IL-13, IL-31, IL-33, or TNF-alpha, in particular from canine IL-4, canine IL-5, canine IL-13, canine IL-31, canine IL-33, or canine TNF-alpha. Preferably, the polyprotein according to the invention comprises three of the same self-protein segments, all derived from the same self-protein selected from the group consisting of IL-4, IL-5, IL-13, IL-31, IL-33, and TNF-alpha, in particular from the group consisting of canine IL-4, IL-5, IL-13, IL-31, IL-33, and TNF-alpha, in which case the host is a canine species. Self-protein segments derived from the same self-protein are not required to be identical, but typically have a high level of identity with one another. In a preferred embodiment, self-protein segments derived from the same self-protein are at least 95%, at least 98%, at least 99%, or at least 99.5%, or are 100% identical to each other.

In a particularly preferred embodiment, at least one, at least two, or at least three of the self-proteins, from which the self-protein segments are derived, is/are derived from or are selected from the group consisting of IL-4, IL-5, IL-13, IL-31, IL-33, or TNF-alpha in particular canine IL-4, IL-5, IL-13, IL-31, IL-33, or TNF-alpha. Preferably, the polyprotein according to the invention comprises two copies of each of the different self-proteins.

In another particularly preferred embodiment, at least one of the self-proteins, from which the self-protein segments are derived in the polyprotein according to the invention, is/are an IL-31, in particular canine IL-31. Preferably, thus, the polyprotein comprises at least two, preferably two, segments derived from an IL-31 protein, in particular canine IL-31, in which case the host is a canine species. Most preferred is the embodiment wherein the polyprotein of the invention comprises two segments of an IL-31 self-protein, in particular canine IL-31, in which case the host is a canine species.

The term “derived from” means that self-protein segments are selected from (i) full-length protein, (ii) a truncated form of the full-length protein containing a B-cell epitope or (iii) a derivative of the protein which has at least 80% sequence identity, preferably at least 90% sequence identity, preferably at least 95% sequence identity to the full-length protein.

Experiments of the inventors have shown that by administering a polyprotein comprising three self-protein segments derived from IL-31 to a host, in particular three self-protein segments derived from canine IL-31 to a canine host, autoantibodies, against (canine) IL-31 protein can be efficiently raised. This polyprotein was demonstrated to be able to particularly efficiently break the self-tolerance against (canine) IL-31. The same was shown with numerous other self-protein segments, in particular by administering a polyprotein with self-protein segments derived from canine IL-4, canine IL-5, canine IL-13, or canine IL-33-CS, and administered to a canine host, as well as with self-protein segments derived from feline IL-31 and administered to a feline host. Autoantibodies against the respective (canine/feline) IL protein were efficiently raised and self-tolerance against it was efficiently broken. Antibodies were also raised against TNF-alpha when immunizing rabbits with a polyprotein according to the invention comprising three repeats of TNF-alpha, showing that the principle is in no way limited to a particular protein or species.

Furthermore, when the polyprotein construct includes at least two segments each of two or three different self-proteins, autoantibodies against each of the individual self-proteins (e.g. cIL-4, cIL-13, and cIL-31) can be efficiently raised and self-tolerance against each of the individual self-proteins is efficiently broken. Thus, the invention provides a flexible platform to raise autoantibodies efficiently against multiple self-proteins using only one construct. The invention is not limited to a particular group of self-proteins, but is suitable for all self-proteins and provides a flexible platform to target various combinations of self-proteins.

As used herein in connection with amino acid sequences, “percent sequence identity” and like terms are used to describe the sequence relationships between two or more amino acid sequence and are understood in the context of and in conjunction with the terms including: a) reference sequence, b) comparison window, c) sequence identity and d) percentage of sequence identity.
a) A “reference sequence” is a defined sequence used as a basis for sequence comparison. A reference sequence may be a subset of or the entirety of a specified sequence. For example, in the present case, the reference sequence for canine IL-31 is for example SEQ ID NO: 3; the reference sequence for canine IL-4 is for example SEQ ID NO: 56; the reference sequence for canine IL-5 is for example SEQ ID NO: 41; the reference sequence for canine IL-13 is for example SEQ ID NO: 46; the reference sequence for canine IL-33 is for example SEQ ID NO: 50 or SEQ ID NO: 51; the reference sequence for feline IL-31 is for example SEQ ID NO: 60; and the reference sequence for bovine TNF-alpha is for example SEQ ID NO: 64. SEQ ID NO: 50 and 51 differ only in that the 3 cysteine residues of SEQ ID NO: 50 (“IL-33-WT”) have been replaced by serine in SEQ ID NO: 51 (“IL-33-CS”). Replacing the cysteine residues with serine in this fashion was found by the inventors to further improve stability of the gene product.
b) A “comparison window” includes reference to a contiguous and specified part of an amino acid sequence, wherein the amino acid sequence may be compared to a reference sequence and wherein the portion of the amino acid sequence in the comparison window may comprise additions, substitutions, or deletions (i.e., gaps) compared to the reference sequence (which does not comprise additions, substitutions, or deletions) for optimal alignment of the two sequences. Those of skill in the art understand that to avoid a misleadingly high similarity to a reference sequence due to inclusion of gaps in the amino acid sequence a gap penalty is typically introduced and is subtracted from the number of matches.
c) Methods of alignment of sequences for comparison are well known in the art. Many pair-wise sequence alignment (PSA) methods have been developed such as EMBOSS (Rice et al., “EMBOSS: the European molecular biology open software suite”, (2000): 276-277), BLAST (Johnson et al., “NCBI BLAST: a better web interface”, Nucleic acids research 36.suppl_2 (2008): W5-W9.), CD-HIT (Li et al., “Cd-hit: a fast program for clustering and comparing large sets of protein or nucleotide sequences”, Bioinformatics 22.13 (2006): 1658-1659), ESPRIT (Sun et al., “ESPRIT: estimating species richness using large collections of 16S rRNA pyrosequences”, Nucleic acids research 37.10 (2009): e76-e76.), and UCLUST (Edgar, Robert, “Search and clustering orders of magnitude faster than BLAST”, Bioinformatics 26.19 (2010): 2460-2461.), etc.
d) “Percent identity” means the value determined by comparing two optimally aligned sequences over a comparison window, wherein the portion of the amino acid sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical amino acid occurs in both sequences to yield the number of matched positions, dividing the number of matched positions by the total number of positions in the window of comparison and multiplying the result by 100 to yield the percentage of sequence identity.

The polyprotein according to the invention comprises as second structural component one or more T-cell epitopes of non-host origin. The term “T-cell epitope” as used herein refers to short peptides which can bind to and thus be presented by major histocompatibility complex (MHC) molecules. MHC class I molecules can bind short peptides of 8 to 10 amino acids in length and MHC class II peptides of 13 to 17 amino acids in length. It is well known that T-cells recognize MHC molecules that have bound peptide epitopes derived from the intracellular processing of an antigen. The immunogenicity of a given epitope is dependent upon three factors: the generation of the appropriate peptide fragment from the antigen, the presence of MHC molecules that bind this fragment and the presence of T-cells capable of recognizing the complex.

The term “one or more” in connection with T-cell epitopes means that the same or different T-cell epitopes can be present in the polyprotein according to the invention. T-cell epitopes contained in the polyprotein of the invention can thus differ from each other in length and/or sequence.

The one or more T-cell epitopes can be selected from the group consisting of an artificial T-cell epitope peptide sequence and a T-cell epitope peptide sequence derived from a non-self protein, in particular from a pathogenic protein, which often harbor particularly potent T-cell epitopes. Suitable artificial T-cell epitopes or suitable pathogenic proteins from which a T-cell epitope can be derived from are known to the skilled person. Such T-cell epitopes are particularly immunogenic upon administration to a host.

Preferably, the polyprotein according to the invention comprises one or more universal T-cell epitopes of non-host origin. Most preferably, the polyprotein according to the invention comprises one or more T-cell epitopes wherein all the T-cell epitopes are universal. The term “universal T-cell epitope” as used herein refers to a T-cell epitope that is universally immunogenic and can be recognized in association with a large number of class II MHC molecules. Using universal T-cell epitopes in the polyprotein according to the invention has the advantage that the T-cell epitopes are particularly immunogenic independent from the chosen host.

Most preferably, the one or more T-cell epitopes contained in the polyprotein according to the invention are Tetanus Toxin T-cell epitopes, in particular Tetanus Toxin T-cell epitopes (i) comprising at least 95% sequence identity with SEQ ID NO: 1, SEQ ID NO: 2 or SEQ ID NO: 39 or (ii) selected from the group consisting of SEQ ID NO: 1, SEQ ID NO: 2 and SEQ ID NO: 39. Even more preferably, the one or more T-cell epitopes are derived from or are identical to SEQ ID NO: 1 or SEQ ID NO: 2. These T-cell epitopes are particularly immunogenic universal T-cell epitopes (Panina-Bordignon et al., “Universally immunogenic T cell epitopes: promiscuous binding to human MHC class II and promiscuous recognition by T cells”, European journal of immunology 19.12 (1989): 2237-2242).

Even more preferably, the T-cell epitope contained in the polyprotein according to the invention is a Tetanus Toxin T-cell epitope, in particular a Tetanus Toxin T-cell epitope (i) comprising at least 96, more preferably 97, and most preferably 98 or 99% sequence identity with SEQ ID NO: 1, SEQ ID NO: 39 or SEQ ID NO: 2 or (ii) selected from the group consisting of SEQ ID NO: 1, SEQ ID NO: 39 and SEQ ID NO: 2.

In the polyprotein according to the invention, the one or more T-cell epitopes of non-host origin are located in between and/or adjacent to the at least two self-protein segments. Preferably, the one or more T-cell epitopes of non-host origin are located in between and, optionally, additionally also adjacent to the at least two self-protein segments. The term “adjacent” in this context means “upstream of the most N-terminal protein segment and/or downstream of the most C-terminal protein segment”.

The polyprotein according to the invention can additionally comprise further components. One example of such additional components are one or more linkers in between the at least two self-protein segments and the one or more T-cell epitopes of non-host origin. These linkers can in particular be 4 to 50 amino acids in length, preferably 4 to 30 amino acids in length and most preferably 4 to 20 amino acids in length. The use of linkers is advantageous since the flexible linkers can facilitate the independent folding of the individual self-protein segments in the polyprotein according to the invention.

When a DNA and/or an RNA encoding for the polyprotein is used instead of the polyprotein itself, the DNA and/or RNA can also additionally encode one or more ER-import signals. An example of an amino acid sequence for an artificial ER signal is SEQ ID NO: 67. This ensures that upon expression of the polyprotein from DNA or RNA in the host, the polyprotein is imported into the ER and later on secreted.

In one embodiment, the polyprotein according to the invention comprises self-protein segments from self-proteins, wherein at least one, at least two, or all of the self-proteins are self-protein(s) selected from the group consisting of SEQ ID NO: 3, 41, 46, 50, 51, 56, 60, 64, and 68-201, preferably wherein at least one self-protein is selected from the group consisting of SEQ ID NO: 3, 41, 46, 50, 51, 56, 60 and 64. In these embodiments, the polyprotein preferably additionally comprises one or more T-cell epitopes in between and/or adjacent to each of the self-protein segments comprised there; wherein the one or more T-cell epitopes are Tetanus toxin T-cell epitopes (i) comprising at least 95% sequence identity with SEQ ID NO: 1, SEQ ID NO: 39 or SEQ ID NO: 2 or (ii) selected from the group consisting of SEQ ID NO: 1, SEQ ID NO: 39 and SEQ ID NO: 2. Most preferably, the one or more T-cell epitopes are Tetanus toxin T-cell epitopes (i) comprising at least 95% sequence identity with SEQ ID NO: 1 or SEQ ID NO: 2 or (ii) selected from the group consisting of SEQ ID NO: 1 and SEQ ID NO: 2.

Preferred combinations of at least two different self-proteins for the polyprotein of the invention are the combinations of IL-31 and any one or two self-proteins of the group selected from IL-4, IL-5, IL-13, and IL-33, preferably the combination of canine IL-31 and any one or two of the group selected from canine IL-4, canine IL-5, canine IL-13, and canine IL-33-CS. For example, preferred embodiments of the polyprotein of the invention comprising at least two segments each of two different self-proteins include the combinations (canine) IL-31 and (canine) IL-5, (canine) IL-31 and (canine) IL-4, (canine) IL-31 and (canine) IL-13, or (canine) IL-31 and (canine) IL-33-CS. Which protein initiates the polyprotein does not have an effect on the immunogenicity of the polyprotein construct. For expression purposes, it is preferred to have the initiating self-protein the one that expresses well in the expression system of choice. For example, in embodiments comprising cIL-31, the inventors found it advantageous to have the first self-protein segment be derived from cIL-31, because the cIL-31 protein expresses very well on its own and also leads to high expression of the polyprotein.

Another preferred combination for the polyprotein of the invention is the combination of at least two segments derived from an IL-4 protein, and at least two segments derived from any one or two self-proteins selected from the group consisting of IL-5, IL-13, IL-31, and IL-33, preferably the combination of at least two segments derived from canine IL-4 and at least two segments derived from any one or two of the group selected from cIL-5, cIL-13, cIL-31 and cIL-33-CS. For example, preferred embodiments of the invention comprising at least two segments of two different self-proteins are constructs comprising the combination of (canine) IL-4 and (canine) IL-13, (canine) IL-4 and (canine) IL-33-CS, (canine) IL-4 and (canine) and IL-5, and the already mentioned (canine) IL-4 and (canine) IL-31.

Another preferred combination for the polyprotein of the invention is the combination of at least two segments derived from an IL-5 protein, and at least two segments derived from any one or two self-proteins selected from the group consisting of IL-4, IL-13, IL-31, and IL-33, preferably the combination of at least two segments derived from canine IL-5 and at least two segments derived from any one or two of the group selected from cIL-4, cIL-13, cIL-31 and cIL-33-CS. For example, preferred embodiments of the invention comprising at least two segments of two different self-proteins are constructs comprising the combination of (canine) IL-5 and (canine) IL-13, (canine) IL-5 and (canine) IL-33-CS, (canine) IL-5 and (canine) and IL-5, and (canine) IL-4 and (canine) IL-31.

Another preferred combination for the polyprotein of the invention is the combination of at least two segments derived from an IL-13 protein, and at least two segments derived from any one or two self-proteins selected from the group consisting of IL-4, IL-5, IL-31, and IL-33, preferably the combination of at least two segments derived from canine IL-13 and at least two segments derived from any one or two of the group selected from cIL-4, cIL-5, cIL-31 and cIL-33-CS. For example, preferred embodiments of the invention comprising at least two segments of two different self-proteins are constructs comprising the combination of (canine) IL-13 and (canine) IL-4, (canine) IL-13 and (canine) IL-5, (canine) IL-13 and (canine) and IL-31, and (canine) IL-13 and (canine) IL-33 (-CS).

Particularly preferred embodiments comprising at least two segments of three different self-proteins are constructs comprising IL-31, IL-4, and IL-13; IL-31, IL-4, and IL-5; IL-31, IL-4, and IL-33; IL-31, IL-5, and IL-13; IL-31, or IL-13, and IL-33. Preferred embodiments also include a polyprotein comprising IL-4, IL-5, and IL-13; IL-4, IL-5, and IL-33; IL-4, IL-13, and IL-33; or IL-5, IL-13, and IL-33.

The inventors found that both polyprotein construct combinations comprising cIL-4 and cIL-13, as well as those comprising cIL-31, cIL-13, and cIL-4, expressed well and resulted in high autoantibody titers for each of the individual self-proteins comprised in the respective polyprotein.

In a further embodiment of the invention, the polyprotein of the invention comprises at least two segments derived from a TNF-alpha protein, and preferably further comprises at least two segments derived from an IL-6, IL-8, or IL-1-beta protein. In a preferred embodiment, the polyprotein of the invention comprises at least two segments derived from a TNF-alpha protein and at least two segments derived from a IL-8 protein; at least two segments derived from a TNF-alpha protein and at least two segments derived from a IL-1-beta protein; or at least two segments derived from a TNF-alpha protein and at least two segments derived from an IL-6 protein. In one embodiment, the polyprotein of the invention comprises at least two segments derived from a TNF-alpha protein, at least two segments derived from a IL-1-beta protein, and at least two segments derived from a TNF-alpha protein and at least two segments derived from a IL-8 protein.

In a further embodiment, the first and second, and optionally third, self-proteins in the polyprotein for use in a vaccine composition to break self-tolerance according to the invention are selected from the group consisting of IL-4, IL-5, IL-13, IL-31 and IL-33, most preferably from the group consisting of IL-31, IL-4, IL-13, and IL-33; and one or more T-cell epitopes in between and/or adjacent to the self-protein segments, wherein the one or more T-cell epitopes are Tetanus toxin T-cell epitopes (i) comprising at least 95% sequence identity with SEQ ID NO: 1, SEQ ID NO: 39 or SEQ ID NO: 2 or (ii) selected from the group consisting of SEQ ID NO: 1, SEQ ID NO: 39 and SEQ ID NO: 2. Most preferably, the one or more T-cell epitopes are Tetanus toxin T-cell epitopes (i) comprising at least 95% sequence identity with SEQ ID NO: 1 or SEQ ID NO: 2 or (ii) selected from the group consisting of SEQ ID NO: 1 and SEQ ID NO: 2.

Experiments of the inventors have shown that by administering such a polyprotein to a canine host, autoantibodies, in particular neutralizing autoantibodies, against all of the self-proteins comprised in the polyprotein, in particular against canine IL-4, IL-13 and IL-31 proteins, can be raised in a particularly B class efficient manner. Experiments have also shown the same results against individual self-proteins comprised in the repetitive modular polyprotein structure underlying the invention, not only for canine, IL-4, IL-13, and IL-31, but also for IL-5, IL-33 and TNF-alpha. Thus, such a polyprotein according to the invention is very efficient in breaking self-tolerance against self-proteins, including but not limited to examples explicitly presented herein.

In one embodiment, the polyprotein for use in a vaccine composition to break self-tolerance against a self-protein of a host has (i) at least 85% sequence identity with SEQ ID NO: 203 or 205 or (ii) has the sequence of SEQ ID NO: 203 or 205. More preferably, the polyprotein according to the invention has (i) at least 90%, more preferably 95% and most preferably 97%, 98% or 99% sequence identity with SEQ ID NO: 203 or SEQ ID NO: 205 or (ii) has the sequence of SEQ ID NO: 203 or SEQ ID NO: 205. Experiments of the inventors have shown that using a polyprotein encoding for SEQ ID NO: 203 or SEQ ID NO: 205 or for a derivative thereof with the aforementioned sequence identities are capable upon administration to a canine host to induce a highly efficient immune response including the production of autoantibodies, in particular neutralizing antibodies, against the cytokines cIL-4 and cIL-13, and for SEQ ID NO: 205 also against cIL-31.

The skilled person knows how to clone the DNA or RNA sequence encoding the polyprotein of the invention into a suitable expression vector and how to produce the polyprotein of the invention.

In some embodiments, the polyprotein of the invention is produced by expression in cultured cells, e.g. such as HEK293 cells, in particular a fast-growing variant of the HEK293 cell line (HEK293-F), e.g. Expi293F cells. The expression in eukaryotic cells has the advantage that the expressed polyprotein is equipped with a glyosylation pattern similar or identical to that of the host. Preferably the polyprotein comprising (canine) IL-4, (canine) IL-5, (canine) IL-13, and/or (canine or feline) IL-31 is produced using mammalian expression.

In some embodiments, the polyprotein of the invention is produced by expression in prokaryotic cells, e.g. bacterial cells such as Escherichia (E.) coli. Any suitable strain of bacteria may be used. Suitable bacterial strains are well known in the art and the skilled person is capable of selecting one compatible for their system. Suitable strains include, but are in no way limited to, BL21 (DE3), BL21 (DE3)-pLysS, BL21-AI, Tuner, Origami, Rosetta, BL21 CodonPlus, BL21trxB, C41 (DE3), JM109, XL1-Blue, NEBexpress, and M15. A particularly suitable strain for the expression of the polyproteins of the invention, in particular the (canine) IL-33 and bovine TNF-alpha polyproteins of the invention, is BL21 (DE3) and variants thereof. Expression in bacterial cells has the advantages of simple procedure due to the less complex bacteria physiology, relatively low costs, short generation times, and high product yield.

The polyprotein of the invention can be encoded by a nucleic acid. The nucleic acid can be RNA or DNA. The nucleic acids can also comprise one or more nucleotides having a modified nucleobase. This can for example make the employed nucleic acid particularly stable against the attack of nucleases. The DNA or RNA encoding the polyprotein, in particular when encompassed in a suitable vector, can be used directly in the vaccine composition of the invention. The polyprotein is then expressed inside the host as is well-known for other DNA and RNA vaccines.

The nucleic acid encoding the polyprotein of the invention can be codon-optimized for efficient translation of the polyprotein in a eukaryotic cell or a host of interest. For example, codons can be optimized for expression in humans, cows, pigs, cats, dogs, bacteria, and so forth (see Codon Usage Database at www.kazusa.or.jp/codon/). Programs for codon optimization are available as freeware (e.g., OPTIMIZER at genomes.urv.es/OPTIMIZER; OptimumGene™ from GenScript at www.genscript.com/codon_opt.html). Commercial codon optimization programs are also available.

DNA encoding the polyprotein of the invention can be operably linked to at least one promoter control sequence. The DNA coding sequence can be operably linked to a promoter control sequence for expression in the eukaryotic cell or host of interest. The promoter control sequence can be a constitutive promoter control sequence.

Suitable constitutive promoter control sequences for expression in a eukaryotic cell, e.g. in a HEK293 cell include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing.

Suitable promoter control sequences for expression in a bacterial cell, e.g. in an E. coli cell include, but are not limited to, lac promoter, trc and tac promoter, T7 RNA polymerase, phage promoter pL, tetA promoter/operator, PPBAD promoter, PBAD promotor, fragments thereof, or combinations of any of the foregoing.

The DNA encoding the polyprotein of the invention also can be linked to a polyadenylation signal (e.g., SV40 polyA signal, bovine growth hormone (BGH) polyA signal, etc.) and/or at least one transcriptional termination sequence. This is particularly advantageous when using mammalian expression systems.

The DNA encoding the polyprotein of the invention can be present in a vector. Suitable vectors include plasmid vectors. Non-limiting examples of suitable plasmid vectors include pUC, pBR322, pET, pBluescript, pcDNA, pCI, pCMV, and variants thereof, wherein pcDNA-type vectors are particularly suitable. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, NY, 3rd edition, 2001.

The nucleic acid encoding the polyprotein of the invention can also be RNA, in particular mRNA. The mRNA can be 5′ capped and/or 3′ polyadenylated.

Further, the nucleic acid encoding the polyprotein of the invention can be a self-replicating RNA. Self-replicating RNAs suitable for immunization are well-known in the field of RNA vaccines. A self-replicating RNA molecule can, when delivered to a eukaryotic cell, lead to the production of multiple daughter RNAs by transcription of itself. A self-replicating RNA molecule is typically a +-strand molecule which can be directly translated after its delivery to a cell. Translation of the self-replicating RNA molecules provides next to the encoded polyprotein of the invention also an RNA-dependent RNA polymerase which then produces both antisense and sense transcripts from the initially delivered RNA. The overall results of this sequence of transcriptions is an amplification in the number of the introduced self-replicating RNAs. In this way, the encoded polyprotein becomes a major polypeptide product of the cells harbouring the delivered self-replicating RNA. Suitable alphavirus self-replicating RNAs can use, e.g., a replicase from a sindbis virus, a semliki forest virus, an eastern equine encephalitis virus or a venezuelan equine encephalitis virus.

A preferred self-replicating RNA molecule thus encodes (i) an RNA-dependent RNA polymerase which can transcribe RNA from the self-replicating RNA molecule and (ii) the polyprotein of the invention. The polymerase can be an alphavirus RNA-dependent RNA polymerase. Whereas natural alphavirus genomes encode structural virion proteins in addition to the RNA-dependent RNA polymerase, it is preferred for the present invention that the self-replicating RNA molecule does not encode alphavirus structural proteins. Thus, a preferred self-replicating RNA used for the present invention can lead to the cellular production of RNA copies of itself, but not to the production of RNA-containing virions. As is known from the field of RNA vaccines, the alphavirus structural proteins which are necessary to produce infectious virions are absent from the self-replicating RNA used in the present invention and their place is taken by the construct encoding the polyprotein of the invention. The self-replicating RNA suitable for the present invention therefore can have two open reading frames. One open reading frame encodes an RNA-dependent RNA polymerase; the other open reading frame encodes the polyprotein of the invention. The self-replicating RNA may have additional (e.g. downstream) open reading frames, e.g., to encode one or more further polyproteins of the invention.

### The Vaccine Composition

The invention further provides a vaccine composition for breaking self-tolerance against a self-protein of a host. The vaccine composition according to the invention comprises two mandatory components:
a) a polyprotein, a DNA encoding for the polyprotein and/or an RNA encoding for the polyprotein, wherein the polyprotein comprises
   at least two self-protein segments derived from a first self-protein of the host;
   at least two self-protein segments derived from a second self-protein of the host;
   optionally at least two self-protein segments derived from a third self-protein of the host; and
   one or more T-cell epitopes of non-host origin in between and/or adjacent to the self-protein segments;
and
b) one or more immunostimulatory oligonucleotides.

The vaccine composition is capable of raising autoantibodies against the self-protein when the vaccine composition is administered to the host.

The polyprotein is as described above.

The vaccine composition of the invention comprises the one or more immunostimulatory oligonucleotides as adjuvants. The term “one or more” used in connection with the immunostimulatory oligonucleotides of the invention means that chemically different oligonucleotides may be part of the vaccine composition of the invention. For example, oligonucleotides of different length, base sequences or differences in the sugar phosphate backbones can be used. The “one” in “one or more” is not meant to refer to single oligonucleotides molecules.

An “immunostimulatory oligonucleotide” as used herein is an oligonucleotide that elicits an immune response in a vertebrate by being detected as foreign by the vertebrate's innate immune system and thereby activating innate immune response pathways.

Typically, the immunostimulatory oligonucleotides of the invention are of synthetic origin. Thus, they can be synthesized with any desired sequence and/or with modifications in the sugar phosphate backbone.

The one or more immunostimulatory oligonucleotides are linear, at least partially single-stranded DNA molecules. The single-stranded DNA immunostimulatory oligonucleotides may, however, interact with themselves or each other inter alia by Watson-Crick base pairing to form secondary structures and agglomerates. Single-stranded stretches will, however, always be present in the immunostimulatory oligonucleotides.

Preferably, the one or more immunostimulatory oligonucleotides are CpG oligodesoxynucleotides (CpG ODN). These are short single-stranded synthetic DNA molecules that contain a cytosine triphosphate deoxynucleotide (“C”) followed by a guanine triphosphate deoxynucleotide (“G”). The “p” refers to the phosphodiester link between consecutive nucleotides, although an ODN according to the invention may have a modified phosphorothioate backbone instead. Using CpG ODNs as immunostimulatory oligonucleotides has the advantage that CpG dinucleotides represent pathogen-associated molecular patterns (PAMPs) sensed by the Toll-like receptors (TLR) 9.

Activation of TLR9 leads to the activation of different proinflammatory signaling pathways dependent on, e.g., nuclear factor ‘kappa-light-chain-enhancer’ of activated B-cells (NF-κB). Activation of NF-κB typically leads to the expression of proinflammatory cytokines. Accordingly, CpG ODNs are particularly potent vaccine adjuvants in the vaccine composition according to the invention.

Preferably, the immunostimulatory oligonucleotides are selected from the group consisting of A-class, B-class and C-class immunostimulatory oligonucleotides. The classification of immunostimulatory oligonucleotides into “A-class”, “B-class” and “C-class” is well-known to the skilled person and described, e.g., in Vollmer, Jörg. “CpG motifs to modulate innate and adaptive immune responses”, International reviews of immunology 25.3-4 (2006): 125-134.

A-class immunostimulatory oligonucleotides are typically characterized by a central phosphodiester CpG-containing palindromic motif and a partially phosphorothioate-modified backbone, in particular a phosphorothioate 3′ poly-G stretch.

B-class immunostimulatory oligonucleotides are typically characterized by a full phosphorothioate backbone with one or more CpG dinucleotides.

C-class immunostimulatory oligonucleotides exhibit properties of class A and class B immunostimulatory oligonucleotides. They contain a full phosphorothioate backbone and one or more palindromic CpG-containing motif(s).

The immunostimulatory oligonucleotides according to the invention typically have a length of from 14 to 500 nucleotides, preferably from 14 to 400 nucleotides, more preferably from 14 to 300 nucleotides, still more preferably from 14 to 200 nucleotides, even more preferably from 16 to 40 and most preferably from 18 to 30 nucleotides.

Immunostimulatory oligonucleotides of this size can be easily synthesized in vitro and were found to be effective as vaccine adjuvants.

Preferably, the one or more immunostimulatory oligonucleotides are selected from the group consisting of B-class immunostimulatory oligonucleotides. The research underlying the invention demonstrated that B-class immunostimulatory oligonucleotides in the vaccine composition according to the invention, are particularly efficient to enhance the immunogenicity of the polyprotein used in the vaccine composition according to the invention.

The one or more immunostimulatory oligonucleotides can also preferably comprise at least 75% sequence identity, more preferably at least 80% sequence identity, even more preferably 85% sequence identity, still more preferably 90%, 95% or 97% sequence identity with SEQ ID NO: 5 or SEQ ID NO: 6.

As used herein in connection with nucleic acid sequences, “percent sequence identity” and like terms are used to describe the sequence relationships between two or more nucleic acids and are understood in the context of and in conjunction with the terms including: a) reference sequence, b) comparison window, c) sequence identity and d) percentage of sequence identity.
a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. In the present case, the reference sequence is SEQ ID NO: 5 or SEQ ID NO: 6.
b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Those of skill in the art understand that to avoid a misleadingly high similarity to a reference sequence due to inclusion of gaps in the polynucleotide sequence a gap penalty is typically introduced and is subtracted from the number of matches.
c) Methods of alignment of sequences for comparison are well known in the art. Optimal alignment of sequences for comparison may be conducted by the local homology algorithm of Smith and Waterman, Adv. Appl. Math., 2:482, 1981; by the homology alignment algorithm of Needleman and Wunsch, J. Mol. Biol., 48:443, 1970; by the search for similarity method of Pearson and Lipman, Proc. Natl. Acad. Sci. USA, 8:2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73:237-244, 1988; Corpet, et al., Nucleic Acids Research, 16:881-90, 1988; Huang, et al., Computer Applications in the Biosciences, 8:1-6, 1992; and Pearson, et al., Methods in Molecular Biology, 24:7-331, 1994. The BLAST family of programs which may be used for database similarity searches includes: BLASTN for nucleotide query sequences against nucleotide database sequences; BLASTX for nucleotide query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and may be used with the present disclosure.
d) “Percent identity” means the value determined by comparing two optimally aligned sequences over a comparison window, wherein the Portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

Preferably, the one or more immunostimulatory oligonucleotides are selected from the group consisting of SEQ ID NO: 5 and SEQ ID NO: 6. Experimental studies have shown that the use of immunostimulatory oligonucleotides encoding for SEQ ID NO: 5 or SEQ ID NO: 6 in the vaccine composition according to the invention are particularly efficient in activating NF-κB proinflammatory response pathways in canine cells and thus strongly enhance the immunogenicity of the polyprotein in the vaccine composition according to the invention.

Preferably, the one or more immunostimulatory oligonucleotides comprise a phosphorothioate in the sugar-phosphate backbone, i.e. a partially phosphorothioate-modified backbone. More preferably, the one or more immunostimulatory oligonucleotides comprise a full phosphorothioate backbone. Phosphorothioate modification has the advantage that the immunostimulatory oligonucleotides are protected from degradation by nucleases. As a consequence, the immunostimulatory activity of these immunostimulatory oligonucleotides is increased.

The vaccine composition of the invention can comprise additional components. Preferably, the vaccine composition of the invention additionally comprises an adjuvant c) conferring a depot effect for the polyprotein and/or the immunostimuluatory oligonucleotides contained in the vaccine composition of the invention. The term “depot effect” refers to the sustained release of the polyprotein and/or the immunostimulatory oligonucleotides from the site of injection. Using such an adjuvant in the vaccine composition according to the invention has the advantage that the immunogenicity of the polyprotein and of the immunostimulatory oligonucleotides in the vaccine composition is further increased so that the self-tolerance against the self-proteins in the polyprotein is broken particularly efficiently.

Most preferably, the adjuvant conferring a depot effect is a copolymer adjuvant capable to form a cross-linked high molecular weight gel in solution. An example of such an adjuvant is Polygen™. A suitable adjuvant conferring a depot effect could also be a mineral or metabolisable oil combined with a surfactant system. Experiments of the inventors have shown that a copolymer adjuvant capable of forming a cross-linked high molecular weight gel in solution is highly compatible with and a suitable carrier for the other components of the vaccine composition according to the invention and provides at the same time a particularly high degree of safety in companion and farm animals.

The vaccine composition according to the invention, in particular when containing DNA or RNA encoding for the polyprotein, can contain liposomes, cationic proteins, cationic polymers or cationic cell penetrating peptides and/or other chemical means which enhance half-life, cellular uptake and translatability of the introduced nucleic acids.

Thus, preferably, the vaccine composition according to the invention for breaking self-tolerance against a self-protein of a host, wherein the vaccine composition is capable of raising autoantibodies against said self-protein when the vaccine composition is administered to the host, and wherein the vaccine composition comprises:
a) a polyprotein, a DNA encoding for the polyprotein and/or an RNA encoding for the polyprotein, wherein the polyprotein comprises
   at least two self-protein segments derived from a first self-protein of the host;
   at least two self-protein segments derived from a second self-protein of the host;
   optionally at least two self-protein segments derived from a third self-protein of the host; and
   one or more T-cell epitopes of non-host origin in between and/or adjacent to the self-protein segments;
and
b) one or more immunostimulatory oligonucleotides and
c) an adjuvant conferring a depot effect.

Even more preferably, the vaccine composition according to the invention for breaking self-tolerance against a self-protein of a host, wherein the vaccine composition is capable of raising autoantibodies against said self-protein when the vaccine composition is administered to the host, and wherein the vaccine composition comprises:
a) a polyprotein, a DNA encoding for the polyprotein and/or an RNA encoding for the polyprotein, wherein the polyprotein comprises at least two self-protein segments derived from a first self-protein of the host, wherein the first self-protein is a cytokine, preferably IL-4, IL-5, IL-13, IL-31, IL-33, or TNF-alpha, most preferably IL-31;
   at least two self-protein segments derived from a second self-protein of the host, wherein the second self-protein is a cytokine, preferably IL-4, IL-5, IL-13, IL-31, IL-33, or TNF-alpha;
   optionally at least two self-protein segments derived from a third self-protein of the host; wherein the third self-protein is a cytokine, preferably IL-4, IL-5, IL-13, IL-31, IL-33, or TNF-alpha; and
   one or more T-cell epitopes of non-host origin in between and/or adjacent to the at least two self-protein segments wherein the one or more T-cell epitopes are Tetanus toxin T-cell epitopes (i) comprising at least 95% sequence identity with SEQ ID NO: 1, SEQ ID NO: 39 or SEQ ID NO: 2 or (ii) selected from the group consisting of SEQ ID NO: 1, SEQ ID NO: 39 and SEQ ID NO: 2; preferably the one or more T-cell epitopes are Tetanus toxin T-cell epitopes (i) comprising at least 95% sequence identity with SEQ ID NO: 1 or SEQ ID NO: 2 or (ii) selected from the group consisting of SEQ ID NO: 1 and SEQ ID NO: 2;
and
b) one or more immunostimulatory oligonucleotides,, wherein the one or more immunostimulatory oligonucleotides comprise at least 75% sequence identity, more preferably at least 80% sequence identity, even more preferably 85% sequence identity, still more preferably 90%, 95% or 97% sequence identity with SEQ ID NO: 5 or SEQ ID NO: 6 or are selected from the group consisting of SEQ ID NO: 5 and SEQ ID NO: 6 and
c) an adjuvant conferring a depot effect wherein the adjuvant conferring a depot effect is a copolymer adjuvant capable to form a cross-linked high molecular weight gel in solution.

Experiments of the inventors have shown that by administering such a polyprotein to a canine host, autoantibodies, in particular neutralizing autoantibodies, against all of the self-proteins comprised in the polyprotein, in particular against canine IL-4, IL-13 and IL-31 proteins, can be raised in a particularly B class efficient manner. Experiments have also shown that a vaccine composition comprising three segments derived from a single self-protein is particularly effective in breaking self-tolerance against IL-4, IL-5, IL-13, IL-31, IL-33, and TNF-alpha, in particular canine IL-5, IL-13, IL-31, and IL-33, as well as feline IL-31 and bovine TNF-alpha. Thus, such a polyprotein according to the invention is very efficient in breaking self-tolerance against self-proteins, including but not limited to examples explicitly presented herein.

The skilled person knows how to identify and analyze individual components of a vaccine composition. To analyze the claimed vaccine composition, the skilled person typically first performs an extraction and/or separation procedure to separate the individual components from each other, e.g., by using liquid chromatography, in particular HPLC. The nucleic acids used in the claimed vaccine composition can be analyzed, e.g., by mass spectrometry and/or sequencing analysis. Proteins in the vaccine composition can also be analyzed, e.g., by mass spectrometry. The immunostimulatory properties of the oligonucleotides contained in the vaccine composition of the invention can be assessed by using a reporter cell line, e.g., a dog monocyte cell line (DH82) allowing to evaluate the NFkB-stimulating potential of these oligonucleotides.

### Use of the Polyprotein

The invention also concerns the use of the polyprotein, DNA encoding for the polyprotein and/or RNA encoding for the polyprotein as described herein in a vaccine composition to break self-tolerance against a self-protein of a host, wherein the polyprotein comprises at least two self-protein segments of the host and one or more T-cell epitopes of non-host origin in between and/or adjacent to the at least two self-protein segments. In the research that led up to the invention, it was found that the use of polyprotein according to the invention makes it possible to efficiently induce the production of autoantibodies, in particular neutralizing autoantibodies, against the self-protein segments of the polyprotein and thus the native self-protein of the host from which the self-protein segment of the polyprotein were derived.

It seems that the presence of T-cell epitopes of non-host origin in the polyprotein according to the invention, in particular the presence of Tetanus toxin T-cell epitopes, allows to efficiently break the self-tolerance against the self-protein segments of the host.

In preferred uses, the polyprotein has one or more of the characteristics defined for the polyprotein according to the invention above.

### Use of the Vaccine Composition

The vaccine compositions described herein are suitable for use in a method of preventing or treating a disease in a subject, wherein the method comprises the step of administering the vaccine composition to the subject. This method comprises administering to a subject an effective amount of the immunostimulatory vaccine to elicit an immune response in the subject. The immune response comprises the induction of autoantibodies, preferably neutralizing antibodies, against the targeted self-protein of the subject. The terms “subject” and “host” are used interchangeably herein.

Upon using the vaccine compositions described herein, in particular with a polyprotein containing self-protein segments derived from (canine) IL-4, L-5, IL-13, IL-31, IL-33, and/or TNF-alpha, it is possible to break self-tolerance against all of the respective self-proteins, in a subject of interest.

It is believed that the breaking of the subject's self-tolerance towards a disease-causing self-protein allows to prevent or treat the disease caused or influenced by this self-protein since the autoantibodies neutralize the function of the self-protein and/or help to reduce the available levels of the self-protein in the subject. The self-protein from which the self-protein segments in the polyprotein of the invention are derived from is also referred to herein as the targeted self-protein in the host.

Subject as used herein may in particular mean a mammal species such as humans and non-human animals. Thus, the subject is a mammal including humans and non-human animals. Preferably, the subject is a non-human animal, in particular a non-human animal selected from the group consisting of cattle, poultry, swine, and companion animals such as cats and dogs. Most preferably, the subject is an animal, in particular an animal selected from the group consisting of cattle, poultry, swine, and companion animals such as a cats and a dogs. Even more preferably, the subject is a dog.

Preferably, the vaccine compositions described herein are used to the prevent or treat
a chronic diseases selected from the group consisting of an autoimmune disease, AIDS and cancer; or
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

The term “allergic condition” is defined herein as a disease or disorder caused by an interaction between the immune system and a substance foreign to the body.

The term “pruritic condition” is defined herein as a disease or disorder characterized by an intense itching sensation that produces the urge to rub or scratch the skin to obtain relief.

More preferably, the vaccine compositions described herein are used to the prevent or treat
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

Most preferably, the vaccine compositions described herein are used to prevent or treat atopic dermatitis.

In a preferred embodiment, the vaccine compositions described herein comprising at least two self-protein segments derived from (canine) IL-31, and in particular those comprising at least two self-protein segments derived from (canine) IL-31 and at least two self-protein segments derived from (canine) IL-4, (canine) IL-13, and/or (canine) IL-33, are used to the prevent or treat
a chronic diseases selected from the group consisting of an autoimmune disease, AIDS and cancer; or
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

Most preferably, the vaccine compositions described herein comprise at least two self-protein segments derived from (canine) IL-31, preferably in addition to at least two self-protein segments derived from (canine) IL-4, (canine) IL-13, and/or (canine) IL-33, and are used to prevent or treat atopic dermatitis.

In another embodiment, the vaccine compositions described herein comprise at least two self-protein segments derived from canine IL-5 and are used to the prevent and/or treat
a pruritic condition, in particular selected from the group consisting of atopic dermatitis, eczema, psoriasis, scleroderma and pruritis; and/or
an eosinophilic disorders, in particular eosinophilic asthma, eosinophilic esophagitis, hypereosinophilic syndromes, and chronic rhinosinusitis, in particular chronic rhinosinusitis with nasal polyps.

More preferably, the vaccine compositions described herein comprise at least two self-protein segments derived from canine IL-5 and are used to prevent and/or treat
atopic dermatitis; or
eosinophilic asthma or chronic rhinosinusitis with nasal polyps.

In another embodiment, the vaccine compositions described herein comprise at least two self-protein segments derived from canine IL-4 and are used to the prevent or treat
a chronic diseases selected from the group consisting of an autoimmune disease, AIDS and cancer; or
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

More preferably, the vaccine compositions comprising IL-4 described herein comprise at least two self-protein segments derived from canine IL-4 and are used to prevent or treat
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

Most preferably, the vaccine compositions described herein comprise at least two self-protein segments derived from canine IL-4 and are used to prevent or treat atopic dermatitis.

In a further embodiment, the vaccine compositions described herein comprise at least two self-protein segments derived from canine IL-13 and are used to the prevent or treat
a chronic diseases selected from the group consisting of an autoimmune disease, AIDS and cancer; or
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

More preferably, the vaccine compositions comprising IL-13 described herein comprise at least two self-protein segments derived from canine IL-13 and are used to prevent or treat
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

Most preferably, the vaccine compositions described herein comprise at least two self-protein segments derived from canine IL-13 and are used to prevent or treat atopic dermatitis.

In another embodiment, the vaccine compositions described herein comprise at least two self-protein segments derived from canine IL-33 and are used to the prevent or treat
a chronic diseases selected from the group consisting of an autoimmune disease, AIDS and cancer; or
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity.

More preferably, the vaccine compositions comprising IL-33 described herein comprise at least two self-protein segments derived from canine IL-33 and are used to prevent or treat
a pruritic condition, in particular selected from the group consisting of atopic dermatitis, eczema, psoriasis, scleroderma and pruritis; or
an allergic condition, in particular selected from the group consisting of allergic dermatitis, summer eczema, urticaria, heaves, inflammatory airway disease, recurrent airway obstruction, airway hyper-responsivness, allergic asthma, eosinophilic asthma, and neutrophilic asthma, rhinitis, chronic obstruction pulmonary disease and inflammatory process resulting from autoimmunity.

Most preferably, the vaccine compositions described herein comprise at least two self-protein segments derived from canine IL-33 and are used to prevent or treat atopic dermatitis and/or allergic rhinitis.

“Treatment”, “treating”, and the like refer to therapeutic treatment. The terms “prevention” and “preventing” refer to prophylactic or preventative measures. Animals in need of treatment or prevention include those already with the disorder or disease condition as well as those in which the disorder or disease condition is to be prevented.

The terms “treating” or “preventing” of a disease or disorder includes preventing or protecting against the disease or disorder (that is, causing the clinical symptoms not to develop), inhibiting the disease or disorder (i.e., arresting or suppressing the development of clinical symptoms), and/or relieving the disease or disorder (i.e., causing the regression of clinical symptoms). It is not always possible to distinguish between “preventing” and “suppressing” a disease or disorder since the ultimate inductive event or events may be unknown or latent. Accordingly, the term “prophylaxis” can also be understood to constitute a type of “treatment” that encompasses both “preventing” and “suppressing.” The term “treatment” can thus include “prophylaxis “.

A variety of administration routes are available for administering the vaccine compositions of the invention. The particular mode selected will depend upon the particular subject group selected, the age and general health status of the subject, the particular condition being treated and the dosage required for therapeutic and/or prophylactic efficacy. The methods of this invention may be practiced using any mode of administration that produces effective levels of an immune response without causing clinically unacceptable adverse effects.

The treatment comprises administering an effective amount of the vaccine composition described herein may to a subject in need thereof. The effective amount is sufficient to elicit an immune response characterized by the production of autoantibodies against the targeted self-protein of the recipient subject. Such effective amount is any amount that causes an immune response comprising the production of autoantibodies in the recipient subject. A method of measuring the strength and quality of the immune response elicited by the vaccine composition of the invention, including the production of autoantibodies, is also part of the invention (see below). A skilled person knows that the effective amount depends on host factors such as the animal species, age, weight, stage of disease, as well as other factors known in the art. The term “suitable effective amount of the vaccine composition” refers to the sum in μg of the polyprotein in the form of protein, DNA or RNA, the immunostimulatory oligonucleotides and optionally the adjuvant conferring a depot effect contained in the vaccine composition of the invention.

Suitable effective amounts may range from about 0.1 μg to 5000 μg per subject. In some embodiments, the effective amount may range from about 0.5 μg to about 4500 μg, from about from about 0.5 μg to about 4500 μg, from about 0.5 μg to about 4500 μg, from about 1 μg to about 4000 μg, from about 1 μg to about 3500 μg, from about 1 μg to about 3000 μg, from about 1 μg to about 2500 μg, from about 1 μg to about 2000 μg, from about 1 μg to about 1500 μg, from about 1 μg to about 1000 μg, from about 1 μg to about 900 μg, from about 1 μg to about 800 μg, from about 1 μg to about 700 μg, from about 1 μg to about 600 μg, from about 1 μg to about 500 μg, from about 1 μg to about 400 μg, from about 1 μg to about 300 μg.

In some embodiments, an immune response can be elicited in a human by administering an effective amount of any of the vaccine compositions described herein to the human subject. The effective amount is sufficient to elicit an immune response comprising the production of autoantibodies against the targeted self-protein in the recipient subject. For example, the effective amount of the vaccine composition for a human can be from about 0.1 μg to about 5000 μg per subject, from about 0.5 μg to about 5000 μg per subject, from about 1 μg to about 4500 μg per subject, from about 1 μg to about 4000 μg per subject, or from about 1 μg to about 3500 μg per subject. By way of example, suitable effective amounts for a human subject may be about 5000 μg, about 4750 μg, about 4500 μg, about 4250 μg, about 4000 μg, about 3750 μg, about 3500 μg, about 3250 μg, about 3000 μg, about 2750 μg, about 2500 μg, about 2250 μg, about 2000 μg, about 1750 μg, about 1500 μg, 1250 μg, about 1000 μg, about 500 μg, about 100 μg, about 75 μg, about 50 μg, about 25 μg, about 10 μg, about 1 μg or about 0.1 μg.

In some embodiments, an immune response can be elicited in a non-human animal, in particular in a dog, by administering an effective amount of any of the vaccine compositions described herein to the non-human subject. The effective amount is sufficient to elicit an immune response comprising the production of autoantibodies in the recipient subject, in particular a dog. For example, the effective amount of the vaccine composition for a non-human animal, in particular a dog, can be from about 0.1 μg to about 5000 μg per subject, from about 0.5 μg to about 5000 μg per subject, from about 1 μg to about 4500 μg per subject, from about 1 μg to about 4000 μg per subject, or from about 1 μg to about 3500 μg per subject. By way of example, suitable effective amounts for a non-human subject may be about 5000 μg, about 4750 μg, about 4500 μg, about 4250 μg, about 4000 μg, about 3750 μg, about 3500 μg, about 3250 μg, about 3000 μg, about 2750 μg, about 2500 μg, about 2250 μg, about 2000 μg, about 1750 μg, about 1500 μg, 1250 μg, about 1000 μg, about 500 μg, about 100 μg, about 75 μg, about 50 μg, about 25 μg, about 10 μg, about 1 μg, 0.5 μg or about 0.1 μg.

The use of the term “about” in connection with numerical values herein indicates that the numerical values may be affected by measurement errors which typically change the numerical values by not more than +5%. Numerical values disclosed herein together with the term “about” are also meant to be disclosed as such, i.e. without the term “about”. Further, numerical ranges as stated herein are meant to include and disclose each and every value within that range. All upper and lower end points of ranges for the same parameter disclosed herein are combinable with each other. All ranges for different parameters disclosed herein are combinable with each other. In particular, the ranges of different or the same “preference level” are particularly compatible with each other.

The vaccine composition may be administered intravenously, intramuscularly, intradermally, intraperitoneally, subcutaneously, by spray, in ovo by feather follicle method, orally, intraocularly, intratracheally, intranasally, or by other methods known in the art. The vaccine composition can be administered subcutaneously. The vaccine composition can also be administered intramuscularly. The vaccine composition may also be administered orally.

The methods of the invention elicit an immune response in a subject such that a disease in a subject is prevented or treated.

Administration can be achieved in various ways. For instance, injection via a needle (e.g. a hypodermic needle) can be used, particularly for intramuscular, subcutaneous, intraocular, intraperitoneal or intravenous administration. Needle-free injection can be used as an alternative.

### Enzyme-Linked Immunosorbent Assay

The vaccine composition, polyprotein and uses of the invention are supplemented by an assay that the inventors developed to specifically detect the autoantibodies produced upon using the polyprotein or the vaccine composition of the invention in a host. This assay method is an enzyme-linked immunosorbent assay (ELISA) and comprises the steps of
a) Adsorbing an antigen onto a test surface;
b) Blocking of free binding sites on the test surface;
c) Incubating the antigen-coated and blocked test surface with a mixture comprising a labeled antibody against the antigen and a to-be-tested autoantibody against the antigen; and
d) Detecting the binding of the labeled antibody.

Detection of autoantibodies, in particular autoantibodies to cytokines, is typically fraught with difficulties, which are, however, overcome by the assay of the invention.

For example, prior art assays often delivered false positive test results because of the nonspecific and low-affinity binding occurring between intact IgG molecules and (recombinant) antigens attached to plastic or nitrocellulose membranes. The use of glycosylated antigens such as cytokines produced in eukaryotic cells for detection may also lead to false positive results because of antipolysaccharide antibodies in the tested serum. Surprisingly, the assay of the invention seems to overcome these difficulties because of its reliance on the competition of a labeled known antibody against the targeted host protein of interest with the to-be-tested autoantibody. This competition principle of the assay minimizes false-positive results.

In step a) of the assay according to the invention, an antigen is adsorbed to a test surface. The term “adsorbing” as used herein means “incubating a test surface with an antigen so that the antigen is adhered to the test surface”. “Adhered to” in this context is meant in the sense of “bound to” or “attached to”. The test surface can be any surface typically used for ELISA formats such as the surface of a well plate, in particular the surface of a plastic well plate, preferably a polystyrene well plate.

The “antigen” as used herein refers a molecule or a portion of a molecule capable of being bound by an antibody which is additionally capable of inducing a host to produce an antibody capable of binding to an epitope of that antigen. An antigen may have one or more than one epitope. Preferably, the antigen used in step a) is or comprises the polyprotein of the invention, a single protein segment thereof or the targeted self-protein that the protein segments in the polyprotein of the invention are derived from.

In step b) of the assay according to the invention, free binding sites on the test surface are blocked. This prevents unspecific binding of the labeled antibody and the to-be-tested autoantibody to the test surface. Suitable solutions, so-called blocking solutions, to achieve step b) are known to the skilled person from other typical ELISA formats. A blocking solution always contains a blocking agent. The blocking agent can be a protein or a mixture of proteins. In particular, the blocking agent can be bovine serum albumin (BSA), newborn calf serum (NBCS), casein, non-fat dry milk or gelatin. Preferably, the blocking agent is gelatin. When using gelatin in the assay of the invention as blocking agent, experiments of the inventors have shown that the background signal is particularly low.

Step c) of the assay according to the invention reflects the assay's competition principle. Step c) involves the competition of a labeled antibody with a to-be-tested autoantibody for binding to the test-surface-adsorbed antigen of step a). Preferably, the labeled antibody is a labeled neutralizing antibody. Since for the assay a labeled antibody against the antigen of interest is used, this antibody can only be displaced or outperformed if the to-be-tested autoantibody has at least a similarly high binding affinity to the employed antigen as the labeled antibody. The mixture used in step c) comprising the labeled antibody and the to-be-tested autoantibody contains a defined amount of labeled antibody. The term “defined amount” in this context means, that the skilled person knows the amount or concentration of the labeled antibody that was employed in the assay. Preferably, 25 to 200 ng/ml of labeled antibody, more preferably 50 to 150 ng/ml labeled antibody and most preferably 75 to 125 ng/ml labeled antibody are used in the mixture of step c). Experiments of the inventors have shown that upon using such concentrations of the labeled antibody in the assay of the invention, the competition with the to-be-tested autoantibody can be particularly well detected.

Preferably, the competition between the two antibodies of step c) is tested by providing a series of mixtures wherein the mixtures of the series differ in the dilution of the to-be-tested autoantibody. The to-be-tested autoantibody can be used in dilutions of 1:1 to 1:20.000, preferably of 1:1 to 1:15.000 and most preferably of 1:1 to 1:10.000.

The term “labeled antibody” is meant to include both intact immunoglobulin molecules as well as portions, fragments, peptides and derivatives thereof such as, for example, Fab, Fab′, F(ab′)2, Fv, Fse, CDR regions, paratopes, or any portion or peptide sequence of the antibody that is capable of binding the antigen of step a). A labeled antibody is said to be “capable of binding” an antigen of step a) if it is capable of specifically reacting with the antigen molecule to thereby bind the antigen molecule to the antibody.

Labeled antibodies also include chimeric antibodies or heterochimeric antibodies as well as fragments, portions, regions, peptides or derivatives thereof, provided by any known technique, such as, but not limited to, enzymatic cleavage, peptide synthesis, or recombinant techniques.

Step d) of the assay of the invention concerns the detection of the antigen-bound labeled antibody. The detection of the labeled antibody is based on its label. The amount of antigen-bound labeled antibody ultimately depends on how effective the to-be-tested autoantibody outperformed the labeled antibody in the binding to the test surface-bound antigen.

Suitable labels of an antibody are known to the skilled person. For example, the skilled person could use as label radioactive isotopes such as 14C or a tag such as biotin.

Preferably, biotin is used as the label. Biotin as label has the advantage that it can be attached directly to an existing protein.

A radiolabeled antibody can be directly detected by measuring the radioactivity, e.g., with a radiometric detector or using a scintillation cocktail and a scintillation counter.

Labels of the antibody in form of a tag can be detected with a suitable label-binding moiety coupled to a reporter. A suitable label-binding moiety for biotin can be for example avidin or streptavidin. The matching system of biotin-strepavidin or biotin-avidin has the advantage that the binding of the two matching partners is particularly specific and strong.

A suitable reporter coupled to the label-binding moiety could be an enzyme such as alkaline phosphatase or horse-radish peroxidase or a fluorescent tag such as GFP. While fluorescent tags can be directly detected by measuring their fluorescence, reporter enzymes can be used to catalyze reactions that lead to a measurable colored product.

A suitable colorimetric substrate for alkaline phosphatase is for example 4-nitrophenyl phosphate disodium salt hexahydrate (pNPP). Upon dephosphorylation of pNPP, a water soluble yellow product is obtained which has a strong absorption at 405 nm. Absorption at 405 nm can be measure with an ELISA reader, for example the Epoch Reader (150115E) or the Synergy H1 Reder (180427C). Other suitable colorimetric substrates for alkaline phosphatase are 5-bromo-4-chloro-3-indolyl phosphate (BCIP) and nitroblue tetrazolium (NBT) produce a purple colored precipitate.

Suitable colorimetric substrate for horse-radish peroxidase are for example 3,3′,5,5′ tetramethylbenzidine (TMB) and 2,2′-azino-di [3-ethylbenzthiazoline] sulfonate (ABTS).

The assay of the invention can contain additional steps in between steps a) to d) such as washing steps to remove any antibody material that has not bound to the test-surface adsorbed antigen.

Experiments of the inventors showed that the assay of the invention is particularly well-suited to detect autoantibodies against IL-31, in particular canine IL-31. In this case, a polyprotein as described above containing (canine) IL-31 or a (canine) IL-31 protein segment of this polyprotein is used as antigen and as labeled antibody a labeled antibody which perturbs or even neutralizies the function of (canine) IL-31 is used.

Preferably, a labeled antibody against canine IL-31 comprising at least one of the groups consisting of
a variable heavy (VH) chain complementary determining region (CDR) 1 having the amino acid sequence YYDIN (SEQ ID NO: 8), SYDMS (SEQ ID NO: 9), or NYGMS (SEQ ID NO: 10);
a variable heavy chain CDR2 having the amino acid sequence WIFPGDGGTKYNETFKG (SEQ ID NO: 11), TITSGGGYTYSADSVKG (SEQ ID NO: 12), or TISYGGSYTYYPDNIKG (SEQ ID NO: 13); and
a variable heavy chain CDR3 having the amino acid sequence ARGGTSVIRDAMDY (SEQ ID NO: 14), ARQNWVVGLAY (SEQ ID NO: 15), or VRGYGYDTMDY (SEQ ID NO: 16)
   is used in the assay of the invention.

Still preferably, a labeled antibody against canine IL-31 comprising at least one of the groups consisting of
a variable light (VL) chain comprising a complementary determining region (CDR) 1 having the amino acid sequence RASESVDNYGISFMH (SEQ ID NO: 17),
KSSQSLLNSGNQKNYLA (SEQ ID NO: 18), or KASQSVSFAGTGLMH (SEQ ID NO: 19);
a variable light chain CDR2 having the amino acid sequence RASNLES (SEQ ID NO: 20), GASTRES (SEQ ID NO: 21), or RASNLEA (SEQ ID NO: 22); and
a variable light chain CDR3 having the amino acid sequence QQSNKDPLT (SEQ ID NO: 23), QNDYSYPYT (SEQ ID NO: 24), or QQSREYPWT (SEQ ID NO: 25).
   is used in the assay of the invention.

More preferably, a labeled antibody against caine IL-31 comprising at least one of the groups consisting of
a) a variable light chain comprising (SEQ ID NO: 26), (SEQ ID NO: 27), (SEQ ID NO: 28), (SEQ ID NO: 29), (SEQ ID NO: 30), (SEQ ID NO: 31), or (SEQ ID NO: 32);
b) a variable heavy chain comprising (SEQ ID NO: 33), (SEQ ID NO: 34), (SEQ ID NO: 35), (SEQ ID NO: 36), (SEQ ID NO: 37), or (SEQ ID NO: 38) is used in the assay of the invention.

Such labeled neutralizing antibodies bind very efficiently to canine IL-31 (see WO2013/011407 A1). The commercial antibody lokivetmab is particularly suitable for use in the assay method according to the invention to detect neutralizing autoantibodies against canine IL-31.

### Sequence Listing

This application contains a Sequence Listing which has been submitted electronically and is hereby incorporated by reference in its entirety. Said Sequence Listing file is named 220057WO_Sequence listing_FINAL.txt and 343 KB in size.

SEQ ID NO: 1 is the amino acid sequence of the Tetanus Toxin T-cell epitope p2.

SEQ ID NO: 39 is the amino acid sequence of the Tetanus Toxin T-cell epitope p4.

SEQ ID NO: 2 is the amino acid sequence of the Tetanus Toxin T-cell epitope p30.

SEQ ID NO: 3 is the amino acid sequence of canine IL-31.

SEQ ID NO: 4 is one version of the amino acid sequence of the cIL-31 polyprotein used for the vaccine of Example 13a.

SEQ ID NO: 40 is another version of the amino acid sequence of the cIL-31 polyprotein used for the vaccine of Example 13a.

SEQ ID NO: 40 differs from SEQ ID NO: 4 only in its N-terminus. In comparison to SEQ ID NO: 4 SEQ ID NO: 40 has three additional amino acids (“SHM”) at the N-terminus. The different N-termini of the polyproteins encoded by SEQ ID NO: 4 and SEQ ID NO: 40 result from different cleavage events of the N-terminal ER-signal sequence.

SEQ ID NO: 5 is the nucleic acid sequence of the immunostimulatory oligonucleotide 1668-PTO.

SEQ ID NO: 6 is the nucleic acid sequence of the immunostimulatory oligonucleotide 2006-PTO:

SEQ ID NO: 7 is the nucleic acid sequence of the plasmid pcDNA3.4-cIL31-poly encoding the cIL-31 polyprotein construct of Example 1a.

The following sequences concern the amino acid sequences related to the anti-canine IL-31 labeled neutralizing antibody suitable for the assay method according to the invention: SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38.

SEQ ID NO: 41 is the amino acid sequence of canine IL-5.

SEQ ID NO: 42 is the amino acid sequence of the cIL-5 polyprotein used for the vaccine construct of Example 13b.

SEQ ID NO: 43 is an alternative amino acid sequence of the cIL-5 polyprotein that can be used for the vaccine construct of Example 13b.

SEQ ID NO: 44 is the nucleic acid sequence of the plasmid pcDNA3.4-cIL-5-poly encoding the cIL-5 polyprotein construct of Example 1b.

SEQ ID NO: 45 is the nucleic acid sequence of the plasmid pcDNA3.4-cIL-5 encoding the cIL-5 protein construct of Example 3b.

SEQ ID NO: 46 is the amino acid sequence of canine IL-13.

SEQ ID NO: 47 is the amino acid sequence of the cIL-13 polyprotein used for the vaccine construct of Example 13c.

SEQ ID NO: 48 is the nucleic acid sequence of the plasmid pcDNA3.4-cIL-13-poly encoding the cIL-13 polyprotein construct of Example 1c.

SEQ ID NO: 49 is the nucleic acid sequence of the bacterial expression plasmid pET30a-cIL-13 encoding the cIL-13 protein construct of Example 3c.

SEQ ID NO: 50 is the amino acid sequence of amino acids 110-263 of the full length dog IL-33 protein (Uniprot 097863) canine IL-33-WT

SEQ ID NO: 51 is the altered amino acid sequence of canine IL-33-CS, which is amino acids 110-263 of the full length dog IL-33 protein (Uniprot 097863), where the 3 cysteine residues are replaced by serine (IL-33-CS) to improve stability of the gene product.

SEQ ID NO: 52 is the nucleic acid sequence of the plasmid pET30a(+)-canIL33-WT encoding the cIL-33_WT protein construct of Example 3d.

SEQ ID NO: 53 is the nucleic acid sequence of the plasmid pET30a(+)-canIL33-CS encoding the cIL-33_CS protein construct construct of Example 3e.

SEQ ID NO: 54 is the amino acid sequence of the cIL-33-CS polyprotein used for the vaccine construct of Example 13d.

SEQ ID NO: 55 is the nucleic acid sequence of the plasmid pET30a-cIL33-(CS-) poly encoding the cIL-33-CS polyprotein construct of Example 1d.

SEQ ID NO: 56 is the amino acid sequence of canine IL-4

SEQ ID NO: 57 is the amino acid sequence of the cIL-4 polyprotein used for the vaccine construct of Example 13e.

SEQ ID NO: 58 is the nucleic acid sequence of the plasmid pcDNA3.4-cIL-5-poly encoding the cIL-4 polyprotein construct of Example 1e.

SEQ ID NO: 59 is the nucleic acid sequence of the bacterial expression plasmid pET30a-cIL-4 encoding the cIL-4 protein construct of Example 3f.

SEQ ID NO: 60 is the amino acid sequence of feline IL-31.

SEQ ID NO: 61 is the amino acid sequence of the feline IL-31 polyprotein used for the vaccine construct of Example 13f.

SEQ ID NO: 62 is the nucleic acid sequence of the plasmid pcDNA3.4-felIL31-poly encoding the feline IL-31 polyprotein construct of Example 1g.

SEQ ID NO: 63 is the nucleic acid sequence of the plasmid pcDNA3.4-fel-IL31 encoding the feline IL-31 protein construct of Example 3g.

SEQ ID NO: 64 is the amino acid sequence of bovine TNF-alpha.

SEQ ID NO: 65 is the amino acid sequence of the bovine TNF-alpha polyprotein used for the immunization of Example 6h.

SEQ ID NO: 66 is the nucleic acid sequence of the bacterial expression plasmid pET30a-bov-TNF-alpha-poly encoding the bovine TNF-alpha polyprotein used for the immunization of Example 6h.

SEQ ID NO: 67 is the amino acid sequence of the artificial ER import signal used for mammalian expression in the given examples.

SEQ ID NOs: 68 to 201 are the amino acid sequences of the protein described in the

### SEQUENCE LISTING

SEQ ID NO: 202 is the nucleic acid sequence of the plasmid pcDNA3.4-cIL-13-cIL-4-poly encoding the cIL-13-cIL-4 polyprotein construct of Example 1h. This sequence is human codon-optimized and derived from the cIL-4-poly-His₆ polypeptide construct.

SEQ ID NO: 203 is the amino acid sequence of the cIL-13-cIL-4 polyprotein used for the vaccine construct of Example 13h.

SEQ ID NO: 204 is the nucleic acid sequence of the plasmid pcDNA3.4-cIL31-cIL-13-cIL-4-poly encoding the cIL-31-cIL-13-cIL-4-poly-His6 construct of Example 1i.

SEQ ID NO: 205 is the amino acid sequence of the cIL-31-cIL-13-cIL-4 polyprotein used for the vaccine construct of Example 13i.

### EXAMPLES

### Example 1a: Design of an IL-31 Polyprotein Comprising Three Segments of cIL-31

A DNA construct was designed encoding for a polyprotein comprising three copies of mature canine IL-31 proteins wherein the mature canine IL-31 (cIL-31) proteins are separated from each other by a Tetanus toxin T-cell epitope and wherein the C-terminal mature cIL-31 is followed by two additional Tetanus toxin T-cell epitopes (see FIG. 1). The encoded polyprotein further contains an artificial ER import signal at the N-terminus and a His₆-tag at the C-terminus (see FIG. 1). This DNA construct was further designed to contain a Kozak sequence upstream of the start codon of the polyprotein to improve expression in mammalian cells and to contain flanking unique restriction enzyme sites for straightforward cloning. After synthesizing this DNA construct, the DNA construct was cloned into the pcDNA3.4 mammalian expression vector, resulting in the vector pcDNA3.4-cIL31-poly with 7637 bp in size (SEQ ID NO: 7 and plasmid map in FIG. 2). Large amounts of transfection grade plasmid was prepared for Expi293F cell expression.

### Example 1b: Design of an IL-5 Polyprotein Comprising Three Segments of cIL-5

A DNA construct was designed encoding for a polyprotein comprising three copies of mature canine IL-5 proteins (see FIG. 39) in the same manner as for the canine IL-31 polyprotein in Example 1a. The DNA construct encoding for cIL-5-polyprotein was sub-cloned into the pcDNA3.4 mammalian expression vector, resulting in the vector pcDNA3.4-cIL-5-poly with 7430 bp in size (SEQ ID NO: 43 and plasmid map in FIG. 40). Large amounts of transfection grade plasmid was prepared for Expi293F cell expression.

### Example 1c: Design of an IL-13 Polyprotein Comprising Three Segments of cIL-13

A DNA construct was designed encoding for a polyprotein comprising three copies of mature canine IL-13 proteins (see FIG. 47) in the same manner as for the canine IL-31 polyprotein in Example 1a. The DNA construct encoding for cIL-13-polyprotein was sub-cloned into the pcDNA3.4 mammalian expression vector, resulting in the vector pcDNA3.4-cIL-13-poly with 7418 bp in size (SEQ ID NO: 48 and plasmid map in FIG. 48). Large amounts of transfection grade plasmid was prepared for Expi293F cell expression.

### Example 1d: Design of an IL-33-CS Polyprotein Comprising Three Segments of cIL-33-CS

A DNA construct was designed encoding for a cIL-33-CS polyprotein (polyprotein-SEQ ID NO: 54) comprising three copies of mature canine IL-33-CS proteins wherein the mature canine IL-33-CS (cIL-33) proteins are separated from each other by a Tetanus toxin T-cell epitope and wherein the C-terminal mature cIL-33-CS is followed by two additional Tetanus toxin T-cell epitopes (see FIG. 63). The polyprotein further contains an artificial start methionine and the His₆ tag, as shown in FIG. 63.

Based on this polypeptide sequence, the DNA encoding for Hi6-cIL33-CS-poly (SEQ ID NO: 55) was designed and synthesized, including a start ATG codon to improve expression in Escherichia coli, and flanking unique restriction enzyme sites (NdeI and HindIII) for straightforward subcloning into the bacterial expression vector pET30a.

The DNA construct was sub-cloned into the pET30a bacterial expression vector, resulting in the vector pET30a-cIL-33-poly (i.e. pET30a-cIL-33-CS poly) with 6954 bp in size (plasmid map in FIG. 64).

The inventors constructed the cIL-33 poly-form with cIL-33-CS as the base protein instead of cIL-33-WT. The inventors found that while human IL-33-WT is known to be sensitively recognized by HEK-Blue™ IL-33 cells, canine IL-33-WT was sensitively recognized only after the free cysteines were mutated to serines, as in cIL-33-CS (see Example 12c below). Without being bound to a theory, it seems that the mutation overcame a potential structural liability of the free cysteines that could be disadvantageous in a cIL-33-poly construct. While this mutation of the free cysteines is not required for all IL-33 constructs, it is advantageous and is a preferred embodiment of the invention.

### Example 1e: Design of an IL-4 Polyprotein Comprising Three Segments of cIL-4

A DNA construct was designed encoding for a polyprotein comprising three copies of mature canine IL-4 protein (see FIG. 68) in the same manner as for the canine IL-31 polyprotein in Example 1a. The DNA construct encoding for cIL-4-polyprotein was sub-cloned into the pcDNA3.4 mammalian expression vector, resulting in the vector pcDNA3.4-cIL-4-poly with 7373 bp in size (SEQ ID NO: 58 and plasmid map in FIG. 69). Large amounts of transfection grade plasmid was prepared for Expi293F cell expression.

### Example 1f: Design of a Feline IL-31 Polyprotein Comprising Three Segments of Fel-IL-31

A DNA construct (SEQ ID NO: 62) was designed encoding for a polyprotein (SEQ ID NO: 61) comprising three copies of mature feline (Felis catus; Felis silvestris catus) IL-31 proteins (SEQ ID NO: 60) wherein the mature feline IL-31 (fel-IL-31) proteins are separated from each other by a Tetanus toxin T-cell epitope and wherein the C-terminal mature fel-IL-31 is followed by two additional Tetanus toxin T-cell epitopes (see FIG. 74). The encoded polyprotein further contains an artificial ER import signal at the N-terminus and a His₆-tag at the C-terminus (see FIG. 74). This DNA construct was further designed to contain a Kozak sequence upstream of the start codon of the polyprotein to improve expression in mammalian cells and to contain flanking unique restriction enzyme sites for straightforward cloning. After synthesizing this DNA construct, the DNA construct was sub-cloned into the pcDNA3.4 mammalian expression vector resulting in the expression plasmid pcDNA3.4-felIL31-poly (7597 bp). Large amounts of transfection grade plasmid was prepared for Expi293F cell expression.

### Example 1g: Design of Polyprotein Embodiments According to the Invention

DNA constructs can be designed to encode for a polyprotein comprising two copies of two or three different self-proteins in the same manner as for the canine IL-31 polyprotein in Example 1a. Namely, the DNA construct encoding for the polyprotein is sub-cloned into a pcDNA3.4 mammalian expression vector, and transfection grade plasmid is prepared for Expi293F cell expression.

The design of a polyprotein comprising two copies of two different self-proteins could be achieved as follows:

A polyprotein is designed to comprise two copies of a first self-protein having one of the sequences SEQ ID NOs: 3, 41, 46, 50, 51, 56, 60, 64, or SEQ ID NO: 68-201, and two copies of a second self-protein having one of the sequences SEQ ID NOs: 3, 41, 46, 50, 51, 56, 60, 64, or SEQ ID NO: 68-201, wherein the first and second self-proteins are not the same, but are from the same host organism.

These examples foresee a polyprotein construct according to the invention that comprises a first copy of the first self-protein, followed by a tetanus toxin T cell epitope (e.g. p2, p4, or p30), followed by a first copy of the second self-protein, followed by a tetanus toxin T cell epitope (e.g. p2, p4, or p30), followed by a second copy of the first self-protein, followed by tetanus toxin T cell epitope (e.g. p2, p4, or p30), followed by a second copy of the second self-protein, followed by one or two tetanus toxin T cell epitopes (p2, p4, or p30). It is advantageous to alternate the tetanus toxin T cell epitopes (e.g. first p2, then p30, then p2, then p30).

For example, the polyprotein construct according to the invention could be designed to comprise a first copy of a first self-protein, followed by tetanus toxin T cell epitope p30 (SEQ ID NO: 2), followed by the first copy of the second self-protein, followed by tetanus toxin T cell epitope p2 (SEQ ID NO: 1), followed by the second copy of the first self-protein, followed by tetanus toxin T cell epitope p30 (SEQ ID NO: 2), followed by the second copy of the second self-protein, followed by one or two tetanus toxin T cell epitopes p30 and/or p2 (SEQ ID NO: 2 and 1, respectively). The same principles regarding the linkers, tags, and ER import signals duly apply to these examples.

Alternatively, the second copy of the first and the second copy of the second self-proteins can be fused, only separated by a tetraglycine spacer, but then followed by an extra tetanus toxin T cell epitope (e.g. p2, p4, or p30), with all other construct details remaining the same.

The design of a polyprotein comprising two copies of three different self-proteins is also foreseen in these examples, and can be achieved as follows:

A polyprotein construct comprising at least two segments from three different self-proteins can be designed by additionally encoding a first and a second copy of a third (different) self-protein, selected from SEQ ID NOs: 3, 41, 46, 50, 51, 56, 60, 64, or SEQ ID NO: 68-201 that are of the same host as the first two self-proteins, and including one tetanus toxin T cell epitope (e.g. p2, p4, p30) per added self-protein copy as well as the necessary linkers.

These examples foresee a polyprotein construct according to the invention that comprises a first copy of the first self-protein, followed by a tetanus toxin T cell epitope (e.g. p2, p4, or p30), followed by a first copy of the second self-protein, followed by a tetanus toxin T cell epitope (e.g. p2, p4, or p30), followed by a first copy of the third self-protein, followed by a tetanus toxin T cell epitope (e.g. p2, p4, or p30), followed by a second copy of the first self-protein, followed by tetanus toxin T cell epitope (e.g. p2, p4, or p30), followed by a second copy of the second self-protein, followed by a tetanus toxin T cell epitope (e.g. p2, p4, or p30), followed by a second copy of the third self-protein, followed by one or two tetanus toxin T cell epitopes (p2, p4, or p30). It is advantageous to alternate the tetanus toxin T cell epitopes (e.g. first p2, then p30, then p2, then p30, and so on).

As a particular example, a polyprotein construct according to the invention comprises a first copy of a first self-protein selected from SEQ ID NOs: 3, 41, 46, 50, 51, 56, 60, 64, or SEQ ID NO: 68-201, followed by tetanus toxin T cell epitope p30 (SEQ ID NO: 2), followed by the first copy of a second self-protein selected from SEQ ID NOs: 3, 41, 46, 50, 51, 56, 60, 64, or SEQ ID NO: 68-201, followed by tetanus toxin T cell epitope p2 (SEQ ID NO: 1), followed by a first copy of a third self-protein selected from SEQ ID NOs: 3, 41, 46, 50, 51, 56, 60, 64, or SEQ ID NO: 68-201, followed by tetanus toxin T cell epitope p30 (SEQ ID NO: 2), followed by a second copy of the first self-protein, followed by tetanus toxin T cell epitope p2 (SEQ ID NO: 1), followed by a second copy of the second self-protein, followed by tetanus toxin T cell epitope p30 (SEQ ID NO: 2), followed by a second copy of the third self-protein, followed by tetanus toxin T cell epitope p30 (SEQ ID NO: 2), followed by tetanus toxin T cell epitope p2 (SEQ ID NO: 1). The same principles of linker, tag, and signal sequences apply.

### Example 1h: Design of a IL-13-IL-4-Polyprotein Embodiment According to the Invention

A consecutive arrangement of cIL-13 (SEQ ID NO: 46) and cIL-4 (SEQ ID NO: 56) mature polypeptides was designed, wherein the first cIL-13 and cIL-4 segments are separated by tetanus toxin T cell epitope p2 (SEQ ID NO: 1). This is followed by tetanus toxin T cell epitope p30 (SEQ ID NO: 2) (FIG. 77). The second cIL-13 (SEQ ID NO: 46) and second cIL-4 (SEQ ID NO: 56) segments attached to this first arrangement were fused, only separated by a tetraglycine spacer, but then followed by two copies of tetanus toxin T cell epitope p30 (SEQ ID NO: 2) and one tetanus toxin T cell epitope p2 (SEQ ID NO: 1). All the individual elements were separated by G/S/A-containing tetrapeptide bridges. N-terminally, a signal sequence for import into the endoplasmic reticulum and the secretory pathway was attached (SEQ ID NO: 63), and C-terminally a tag (His₆) for straightforward purification was added. Of note, while the order should not matter in practice, the order used in these experiments was cIL-13-cIL-4. Any difference in labeling or reference hereto is an error and was meant to refer to this correct order.

### Example 1i: Design of a Triple (IL-31-IL4-IL-13) Polyprotein Embodiment According to the Invention

A repetitive modular arrangement of mature polypeptides cIL-31 (SEQ ID NOs: 3), cIL-13 (SEQ ID NO: 46), and cIL-4 (SEQ ID NO: 56) was designed, where the first cIL-31 and cIL-13 segments are separated by tetanus toxin T cell epitope p30 (SEQ ID NO: 2). This is followed by tetanus toxin T cell epitope p2 (SEQ ID NO: 2) and a first cIL-4 segment (see FIG. 84). The second copy of the [cIL-31]-[p2]-[cIL-13]-[p30]-[cIL-4] module is separated from the first module by a p30 tetanus toxin T cell epitope (SEQ ID NO: 2). The C-terminus is formed by an arrangement of a p30 tetanus toxin T cell epitope (SEQ ID NO: 2) followed by a p2 tetanus toxin T cell epitope (SEQ ID NO: 1). All the individual elements are separated by G/S/A-containing tetrapeptide bridges. N-terminally, a signal sequence for import into the endoplasmic reticulum and the secretory pathway is attached, and C-terminally a tag (His₆) for straightforward purification is added.

Of note, while the order should not matter in practice, the order used in these experiments was cIL-31-cIL-13-cIL-4. Any difference in labeling or reference hereto is an error and was meant to refer to this correct order.

### Example 1j: Design of a Bovine TNF-Alpha Polyprotein Comprising Three Segments of Bovine TNF-Alpha

A DNA construct (SEQ ID NO: 66) was designed encoding for a bovine TNF-alpha polyprotein (polyprotein-SEQ ID NO: 65) comprising three copies of TNF-alpha (SEQ ID NO: 64) proteins wherein the TNF-alpha proteins are separated from each other by a Tetanus toxin T-cell epitope and wherein the C-terminal TNF-alpha is followed by two additional Tetanus toxin T-cell epitopes (see FIG. 92). The polyprotein further contains an artificial start methionine and the His₆ tag, as shown in FIG. 92. Hexa-G/S-linkers were used in this construct.

Based on this polypeptide sequence, the DNA encoding for Hi6-bov-TNF-alpha-poly (SEQ ID NO: 66) was designed and synthesized, including a start ATG codon to improve expression in Escherichia coli, and flanking unique restriction enzyme sites (NdeI and HindIII) for straightforward subcloning into the bacterial expression vector pET30a.

The DNA construct was sub-cloned into the pET30a bacterial expression vector, resulting in the vector pET30a(+)-bov-TNF-alpha-poly with 7017 bp in size.

### Example 2a: Expression of a cIL-31 Polyprotein, Comprising Three Segments of cIL-31, in Expi293F Cells and Purification of the Expressed Polyprotein

Expi293F cells were grown in serum-free Expi293™ expression Medium (Thermo Fisher Scientific). The Expi293F cells were maintained in Erlenmeyer Flasks (Corning Inc.) at 37° C. with 8% CO2 on an orbital shaker (VWR Scientific). One day before transfection, the cells were seeded at an appropriate density in Corning Erlenmeyer Flasks. On the day of transfection, the plasmid pcDNA3.4-cIL31-poly and transfection reagent were mixed at an optimal ratio and then added into the flask with cells ready for transfection. The cell culture supernatants collected on day 6 were used for purification of the polyprotein expressed from pcDNA3.4-cIL31-poly (SEQ ID NO: 7 and plasmid map in FIG. 2). The produced mature polyprotein is expected to no longer include the artificial ER import signal at the N-terminus and then has the sequence of SEQ ID NO: 4 or SEQ ID NO: 40. SEQ ID NO: 4 and SEQ ID NO: 40 differ from each other in their N-terminus due to different cleavage events of the ER import signal.

The purification and analysis of the expressed polyprotein were performed as follows:

The cell culture broth was centrifuged. Thereafter, the cell culture supernatant was loaded onto an Ni²⁺-NTA affinity purification column at an appropriate flowrate. After washing and elution with appropriate buffers, the eluted fractions were pooled and the buffer was exchanged to the final formulation buffer which was PBS, pH 7.2.

The purified polyprotein was analyzed by SDS-PAGE and Coomassie Blue staining to determine its molecular weight and purity. To to do so, the concentration of the purified polyprotein was determined by the Bradford assay with BSA as a standard for the calibration curve. Approximately 16 mg of (in phosphate-buffered saline, PBS) soluble (cIL-31)-p4-(cIL-31)-p30-(cIL-31)-p30-p4-His₆ polyprotein, referred to in the following Examples as cIL-31 polyprotein or cIL-31 poly, were obtained from 100 ml crude cell culture supernatant.

For the SDS-PAGE analysis the following loading buffers were used:
Reducing Loading buffer: 300 mM Tris-HCl, 10% SDS, 30% Glycerol, 0.5% bromophenol blue, 250 mM DTT, pH 6.8.
Non-Reducing Loading buffer: 300 mM Tris-HCl, 10% SDS, 30% Glycerol, 0.5% bromophenol blue, pH 6.8.

Reducing and non-reducing loading buffer were added to the polyprotein samples, respectively. The polyprotein samples with reducing or non-reducing loading buffer had a concentration close to 0.5 mg/ml.

After mixing the polyprotein samples with reducing loading buffer, heating at 100° C. for 5-10 min was performed.

The polyprotein samples with reducing or non-reducing loading buffer were centrifuged at 10000 rpm for 1 min, and then loaded in a gel chamber of a precast gel (Genscript, Cat.No. M42012). SDS-PAGE with these gels was performed as outlined by the manufacturer (140 V for approximately 60 min). Thereafter, the gel was stained with Coomassie Blue. The stained gel is shown in FIG. 3.

The dominant band in lane 1 of the Coomassie Blue-stained gel in FIG. 3 is slightly larger than expected from the protein sequence (56865.04 Da, calculated from the mature sequence using https://web.expasy.org/cgi-bin/protparam/protparam). This difference likely arises from extensive N-glycosylation since the cIL-31 protein sequence contains 8 N-glycosylation sites, of which 7, based on NetNGlyc analysis (http://www.cbs.dtu.dk/services/NetNGlyc/), are likely to be modified.

Under non-reducing conditions (lane 2 of the Coomassie Blue-stained gel in FIG. 3), only approximately half of the loaded protein migrated in a band which is indicative of a monomer. A large proportion appears to be in dimeric and multimeric forms.

### Example 2b: Expression of a cIL-5 Polyprotein, Comprising Three Segments of cIL-5, in Expi293F Cells and Purification of the Expressed Polyprotein

Expression of a cIL-5 polyprotein was conducted the same as for the expression of the cIL-31 polyprotein of Example 2a, except that plasmid pcDNA3.4-cIL-5-poly (SEQ ID NO: 43; see plasmid map in FIG. 40) instead of pcDNA3.4-cIL31-poly (SEQ ID NO: 7) was used.

As in Example 2a, the produced mature polyprotein is expected to no longer include the artificial ER import signal at the N-terminus and then has the sequence of SEQ ID NO: 42.

Approximately 0.23 mg of (in phosphate-buffered saline, PBS) soluble (cIL-5)-p2-(cIL-5)-p30-(cIL-5)-p30-p2-His₆ polyprotein, referred to in the following Examples as cIL-5 polyprotein or cIL-5 poly, were obtained from 100 ml crude cell culture supernatant.

The stained gel is shown in FIG. 41.

The dominant band in lane 1 of the SDS-PAGE/Western Blot in FIG. 41 is much larger than expected from the protein sequence (50477.67 Da, calculated from the mature sequence using https://web.expasy.org/cgi-bin/protparam/protparam). This difference likely arises from extensive N-glycosylation, since the protein sequence contains 8 N-glycosylation sites, of which 5, based on NetNGlyc analysis (http://www.cbs.dtu.dk/services/NetNGlyc/), are likely to be modified.

Under non-reducing conditions (lane 2 of the SDS-PAGE in FIG. 41), the appearance of the loaded protein is changed compared to that under reducing conditions, which is indicative of a disulfide linked oligomeric or polymeric forms.

### Example 2c: Expression of a cIL-13 Polyprotein, Comprising Three Segments of cIL-13, in Expi293F Cells and Purification of the Expressed Polyprotein

Expression of a cIL-13 polyprotein was conducted the same as for the expression of the cIL-31 polyprotein of Example 2a, except that plasmid pcDNA3.4-cIL-13-poly (SEQ ID NO: 48; see plasmid map in FIG. 48) instead of pcDNA3.4-cIL31-poly (SEQ ID NO: 7) was used.

As in Example 2a, the produced mature polyprotein is expected to no longer include the artificial ER import signal at the N-terminus and then has the sequence of SEQ ID NO: 47.

Approximately 0.6 mg of (in phosphate-buffered saline, PBS) soluble (cIL-13)-p2-(cIL-13)-p30-(cIL-13)-p30-p2-His₆ polyprotein, referred to in the following Examples as cIL-13 polyprotein or cIL-13 poly, were obtained from 100 ml crude cell culture supernatant.

The stained gel is shown in FIG. 49.

The dominant band in lane 1 of the SDS-PAGE/Western Blot in FIG. 49 is much larger than expected from the protein sequence (47496.39 Da, calculated from the mature sequence using https://web.expasy.org/cgi-bin/protparam/protparam). This difference likely arises from extensive N-glycosylation, since the protein sequence contains 14 N-glycosylation sites, of which 13, based on NetNGlyc analysis (http://www.cbs.dtu.dk/services/NetNGlyc/), are likely to be modified.

Under non-reducing conditions (lane 2 of the SDS-PAGE in FIG. 49), the appearance of the loaded protein is very similar to that under reducing conditions, which is indicative of a largely monomeric form.

### Example 2d: Expression of a cIL-33 Polyprotein, Comprising Three Segments of cIL-33, in E. Coli Cells and Purification of the Expressed Polyprotein

E. coli strain BL21 Star™ (DE3) was transformed with recombinant plasmid. A single colony was inoculated into Lysogeny Broth (LS) medium containing related antibiotic. The culture was incubated at 37° C. at 200 rpm and then induced with Isopropyl β-D-1-thiogalactopyranoside (IPTG). SDS-PAGE was used to monitor the expression.

Expression was scaled up as follows: Recombinant BL21 (DE3) stored in glycerol was inoculated into Terrific Broth (TB) medium containing related antibiotic and cultured at 37° C. When the OD600 reached about 1.2, the cell culture was induced with IPTG at 15° C. for 16h. Bacteria were harvested by centrifugation.

The purification of the expressed protein was performed as follows: Bacterial pellets were resuspended with lysis buffer followed by sonication. The precipitate after centrifugation was dissolved using denaturing agent. Target protein was obtained by one-step purification using a Ni column. Target protein was sterilized by 0.22 μm filter before stored in aliquots. The concentration was determined by Bradford protein assay with BSA as standard. The protein purity and molecular weight were determined by standard SDS-PAGE.

The SDS-PAGE analysis of the expressed protein was performed as described in Example 2a. The stained gel is shown in FIG. 65.

Approximately 0.9 mg of soluble (in phosphate-buffered saline, PBS) cIL-13-His₆ was obtained from the bacterial pellet of 1 L culture. The size of the dominant band in lane 2 of the SDS-PAGE depicted in FIG. 65 is in good agreement with the prediction expected from the protein sequence (63604.11 Da, calculated from the mature sequence using https://web.expasy.org/cgi-bin/protparam/protparam).

### Example 2e: Expression of a cIL-4 Polyprotein, Comprising Three Segments of cIL-4, in Expi293F Cells and Purification of the Expressed Polyprotein

Expression of a cIL-4 polyprotein was conducted the same as for the expression of the cIL-31 polyprotein of Example 2a, except that plasmid pcDNA3.4-cIL-4-poly (SEQ ID NO: 58; see plasmid map in FIG. 69) instead of pcDNA3.4-cIL31-poly (SEQ ID NO: 7) was used.

The Western blot analysis revealed the recombinant product as a broad band between 100 kDa and 120 kDa in reduced samples, while in non-reduced samples two fuzzy band zones at 100-120 kDa and >>120 kDa were visible.

As in Example 2a, the produced mature polyprotein is expected to no longer include the artificial ER import signal at the N-terminus and then has the sequence of SEQ ID NO: 47.

Approximately 4.5 mg of (in phosphate-buffered saline, PBS) soluble (cIL-4)-p2-(cIL-4)-p30-(cIL-4)-p30-p2-His₆ polyprotein, referred to in the following Examples as cIL-4 polyprotein or cIL-4 poly, were obtained from 100 ml crude cell culture supernatant.

The stained gel is shown in FIG. 49.

The dominant band under reducing conditions is much larger than expected from the protein sequence (51039.30 Da), calculated from the mature sequence using https://web.expasy.org/cgi-bin/protparam/protparam). This difference likely arises from extensive N-glycosylation, since the protein sequence contains 20 N-glycosylation sites, of which 17, based on NetNGlyc analysis (http://www.cbs.dtu.dk/services/NetNGlyc/), are likely to be modified.

The potentially and likely to be heterogeneous, massive glycosylation may explain the broad band appearance of the protein in Western blots, and the larger size than predicted by the amino acid composition.

### Example 2f: Expression of a Fel-IL-31 Polyprotein, Comprising Three Segments of Fel-IL-31, in Expi293F Cells and Purification of the Expressed Polyprotein

Expi293F cells were grown in serum-free Expi293™ expression Medium (Thermo Fisher Scientific). The Expi293F cells were maintained in Erlenmeyer Flasks (Corning Inc.) at 37° C. with 8% CO2 on an orbital shaker (VWR Scientific). One day before transfection, the cells were seeded at an appropriate density in Corning Erlenmeyer Flasks. On the day of transfection, the plasmid pcDNA3.4-fel-IL31-poly and transfection reagent were mixed at an optimal ratio and then added into the flask with cells ready for transfection. The cell culture supernatants collected on day 6 were used for purification of the polyprotein expressed from pcDNA3.4-fel-IL31-poly. The produced mature polyprotein is expected to no longer include the artificial ER import signal at the N-terminus and then has the sequence of SEQ ID NO: 61.

The purification and analysis of the expressed polyprotein were performed as follows:

The cell culture broth was centrifuged. Thereafter, the cell culture supernatant was loaded onto an Ni²⁺-NTA affinity purification column at an appropriate flowrate. After washing and elution with appropriate buffers, the eluted fractions were pooled and the buffer was exchanged to the final formulation buffer which was PBS, pH 7.2.

The purified polyprotein was analyzed by SDS-PAGE and Coomassie Blue staining to determine its molecular weight and purity. To to do so, the concentration of the purified polyprotein was determined by the Bradford assay with BSA as a standard for the calibration curve. Approximately 7.29 mg of (in phosphate-buffered saline, PBS) soluble fel-IL-31-poly-His₆ were obtained from 100 ml crude cell culture supernatant.

For the SDS-PAGE analysis the following loading buffers were used:
Reducing Loading buffer: 300 mM Tris-HCl, 10% SDS, 30% Glycerol, 0.5% bromophenol blue, 250 mM DTT, pH 6.8.
Non-Reducing Loading buffer: 300 mM Tris-HCl, 10% SDS, 30% Glycerol, 0.5% bromophenol blue, pH 6.8.

Reducing and non-reducing loading buffer were added to the polyprotein samples, respectively. The polyprotein samples with reducing or non-reducing loading buffer had a concentration close to 0.5 mg/ml. After mixing the polyprotein samples with reducing loading buffer, heating at 100° C. for 5-10 min was performed. The polyprotein samples with reducing or non-reducing loading buffer were centrifuged at 10000 rpm for 1 min, and then loaded in a gel chamber of a precast gel (Genscript, Cat.No. M42012). SDS-PAGE with these gels was performed as outlined by the manufacturer (140 V for approximately 60 min). Thereafter, the gel was stained with Coomassie Blue.

In a Coomassie Blue stained SDS-gel, fel-IL-31-poly is represented by a dominant band around 80 kDa in reduced gels. This is larger than expected (calculated molecular weight of the mature polypeptide: 55615.65 Da), which may be caused by N-glycosylation. Based on NetNGlyc analysis (http://www.cbs.dtu.dk/services/NetNGlyc/), up to 6 positions are likely to be modified.

In the non-reduced gel lane, mobility of the fel-IL-31-poly band is somewhat faster than in the reduced one, which is typical for intra-chain disulfide bonds leading to more compact structure. Some smear on top of the dominant band indicates aggregated material. However, overall the SDS-PAGE analysis suggest that fel-IL-31-poly is largely monomeric.

### Example 2g: Expression and Purification of a Further Polyproteins Comprising Three Segments of a Single Self-Protein

Expression of a polyprotein designed in Example 1g can be conducted the same as for the expression of the cIL-31 polyprotein of Example 2a or of Example 2d. The produced mature polyprotein is expected to no longer include an artificial ER import signal at the N-terminus, if included in the construct, and expected to have the corresponding amino acid sequence SEQ ID NO: 68 to 201.

### Example 2h: Expression of a cIL-13-cIL-4 Polyprotein According to the Invention in Expi293F Cells and Purification of the Expressed Polyprotein

A DNA (SEQ ID NO: 202) encoding for the cIL-13-cIL-4-poly-His₆ polyprotein of Example 1h was designed and synthesized, including a Kozak sequence upstream the start ATG to improve expression in mammalian cells, and flanking unique restriction enzyme sites (EcoRI and HindIII) for straightforward subcloning. The complete cIL-13-cIL-4-poly DNA sequence was sub-cloned into the pcDNA3.4 mammalian expression vector (FIG. 78) and large amounts of transfection grade plasmid was prepared for Expi293F cell expression.

Expression of a cIL-13-cIL-4 polyprotein was conducted the same as for the expression of the cIL-31 polyprotein of Example 2a, except that plasmid pcDNA3.4-cIL-13-cIL-4-poly (SEQ ID NO: 202; see plasmid map in FIG. 78) instead of pcDNA3.4-cIL31-poly (SEQ ID NO: 7) was used.

As in Example 2a, the produced mature polyprotein is expected to no longer include the artificial ER import signal at the N-terminus and then has the sequence of SEQ ID NO: 203.

Approximately 5.4 mg of soluble (in phosphate-buffered saline, PBS) cIL-13-cIL-4-poly-His₆ protein was obtained from 100 ml crude cell culture supernatant.

The Western blot analysis revealed the recombinant product as a broad band close to 120 kDa in reduced samples, while in non-reduced samples a fuzzy band at >120 kDa were visible. The dominant band in lane R of the SDS-PAGE/Western Blot is much larger than expected from the protein sequence (66094.30 Da, calculated from the mature sequence using https://web.expasy.org/cgi-bin/protparam/protparam). It is likely that the difference is accounted for by extensive N-glycosylation, since the protein sequence contains 23 N-glycosylation sites, of which 20, based on NetNGlyc analysis (http://www.cbs.dtu.dk/services/NetNGlyc/), are likely to be modified. This potentially massive, and most likely heterogeneous, glycosylation explains the broad band appearance of the protein in Western blots, and the larger size than predicted by the amino acid composition.

### Example 2i: Expression of a cIL-31-cIL-13-cIL-4 Polyprotein According to the Invention in Expi293F Cells and Purificiation of the Expressed Polyprotein

A DNA (SEQ ID NO: 204) encoding for the cIL-31-cIL-13-cIL-4-poly-His₆ polyprotein of Example 1i was designed and synthesized, including a Kozak sequence upstream the start ATG to improve expression in mammalian cells, and flanking unique restriction enzyme sites (EcoRI and HindIII) for straightforward subcloning. The DNA sequence was sub-cloned into the pcDNA3.4 mammalian expression vector (FIG. 85) and large amounts of transfection grade plasmid was prepared for Expi293F cell expression.

Expression of a cIL-31-cIL-13-cIL-4 polyprotein was conducted the same as for the expression of the cIL-31 polyprotein of Example 2a, except that the corresponding plasmid was used. As in Example 2a, the produced mature polyprotein is expected to no longer include the artificial ER import signal at the N-terminus and then has the sequence of SEQ ID NO: 205. Approximately 2.73 mg of soluble (in phosphate-buffered saline, PBS) cIL-31-cIL-13-cIL-4-poly-His₆ was obtained from 100 ml crude cell culture supernatant.

In Coomassie Blue-stained gels (data not shown), a dominant band above the 150 kDa marker is visible in reduced samples. Non-reduced samples led to a smear above >150 kDa extending to the top of the gel. The Western blot analysis revealed the recombinant product as a broad band close to 120 kDa in reduced samples, while in non-reduced samples a smear at >120 kDa extending to the top of the gel was visible. The dominant band in lane R of the SDS-PAGE/Coomassie Blue and SDS-PAGE/Western Blot is much larger than expected from the protein sequence (99987.98 Da, calculated from the mature sequence using https://web.expasy.org/cgi-bin/protparam/protparam). It is likely that the difference is accounted for by extensive N-glycosylation, since the protein sequence contains 28 N-glycosylation sites, of which 21, based on NetNGlyc analysis (http://www.cbs.dtu.dk/services/NetNGlyc/), are likely to be modified. This potentially massive, and most likely heterogeneous, glycosylation explains the broad band appearance of the protein in Western blots, and the larger size than predicted by the amino acid composition.

### Example 3a: Expression of a Properly Folded Native cIL-31 in Mammalian Cells

A DNA construct encoding for cIL-31 with an artificial ER import signal at the N-terminus and a His₆-tag at the C-terminus was designed. This cIL-31-DNA construct was further designed to contain a Kozak sequence upstream of the start codon of the protein to improve expression in mammalian cells and to contain flanking unique restriction enzyme sites for straightforward cloning. After synthesizing this DNA construct, the DNA construct was cloned into the pcDNA3.4 mammalian expression vector, resulting in the vector pcDNA3.4-cIL31 with 6521 bp in size. Large amounts of transfection grade plasmid were prepared for Expi293F cell expression.

Expression of the cIL-31 construct from pcDNA3.4-cIL31 was achieved as follows:

Expi293F cells were grown in serum-free Expi293™ expression Medium (Thermo Fisher Scientific). The cells were maintained in Erlenmeyer Flasks (Corning Inc.) at 37° C. with 8% CO₂ on an orbital shaker (VWR Scientific). One day before transfection, the cells were seeded at an appropriate density in Corning Erlenmeyer Flasks. On the day of transfection, the plasmid pcDNA3.4-cIL31 and transfection reagent were mixed at an optimal ratio and then added into the flask with cells ready for transfection. The cell culture supernatants collected on day 6 were used for purification.

The purification and analysis of the expressed protein was performed as follows:

The cell culture broth was centrifuged. The cell culture supernatant was loaded onto an Ni²⁺-NTA affinity purification column at an appropriate flowrate. After washing and elution with appropriate buffers, the eluted fractions were pooled and buffer exchanged to the final formulation buffer, which was PBS pH 7.2.

The purified protein was analyzed by SDS-PAGE and Coomassie Blue staining to determine its molecular weight and purity. To to do so, the concentration of the purified polyprotein was determined by the Bradford assay with BSA as a standard for the calibration curve. Approximately 2.61 mg of (in phosphate-buffered saline, PBS) soluble cIL31-His₆, referred to in the following examples as cIL-31, were obtained from 100 ml crude cell culture supernatant.

For the SDS-PAGE analysis the following loading buffers were used:
Reducing Loading buffer: 300 mM Tris-HCl, 10% SDS, 30% Glycerol, 0.5% bromophenol blue, 250 mM DTT, pH 6.8.
Non-Reducing Loading buffer: 300 mM Tris-HCl, 10% SDS, 30% Glycerol, 0.5% bromophenol blue, pH 6.8.

Reducing and non-reducing loading buffer were added to the protein samples, respectively. The protein samples with reducing or non-reducing loading buffer had a concentration close to 0.5 mg/ml.

After mixing the protein samples with reducing loading buffer, heating at 100° C. for 5-10 min was performed.

The protein samples with reducing or non-reducing loading buffer were centrifuged at 10000 rpm for 1 min, and then loaded in a gel chamber of a precast gel (Genscript, Cat.No. M42012). SDS-PAGE with these gels was performed as outlined by the manufacturer (140 V for approximately 60 min). Thereafter, the gel was stained with Coomassie Blue. The stained gel is shown in FIG. 4.

The dominant band in lane 1 of the SDS-PAGE is considerably larger than expected from the protein sequence (16196.54 Da, calculated from the mature sequence using https://web.expasy.org/cgi-bin/protparam/protparam). It is likely that the difference is accounted for by extensive N-glycosylation, since the protein sequence contains two N-glycosylation sites, both of which, based on NetNGlyc analysis (http://www.cbs.dtu.dk/services/NetNGlyc/), are likely to be modified.

Under non-reducing conditions (lane 2 in SDS-PAGE), the bulk of the loaded protein migrated in a band which is indicative of a monomer. Only a minor proportion appears to be present in dimeric and multimeric forms.

### Example 3b: Expression of a Properly Folded Native cIL-5 in Mammalian Cells

Design, synthesis and subcloning of a DNA construct encoding for cIL-5 was performed as described for cIL-31 in Example 3a. The vector pcDNA3.4-cIL-5 (SEQ ID NO: 45) with 6452 bp in size is depicted in FIG. 42. Large amounts of transfection grade plasmid were prepared for Expi293F cell expression.

Expression of the cIL-5 construct from pcDNA3.4-cIL-5, as well as purification and analysis of the expressed protein was achieved as for the cIL-31 construct in Example 3a.

Approximately 7.28 mg of (in phosphate-buffered saline, PBS) soluble cIL-5-His₆, referred to in the following examples as cIL-5, were obtained from 100 ml crude cell culture supernatant.

The observed apparent molecular size in reducing SDS-PAGE (FIG. 43, Lane “1”) is slightly higher than expected from prediction (13921.91 Da, calculated from the mature sequence using https://web.expasy.org/cgi-bin/protparam/protparam), which may be accounted for by N-glycosylation at one position. Non-reducing SDS-PAGE (Lane “2” of FIG. 43) suggests that the protein is expressed as a dimer, which is in agreement with literature knowledge on IL-5 from other species.

### Example 3c: Expression of a Properly Folded Native cIL-13 in Bacteria (E. Coli) Cells

A DNA construct encoding for cIL-13 of SEQ ID NO: 46 with a start methionine (M) at the N-terminus and a His₆-tag at the C-terminus was designed. This cIL-13-DNA construct was further designed to contain an artificial start codon ATG, flanking unique restriction enzyme sites (NdeI and HindIII) for straightforward subcloning, and a stop codon TGA.

After synthesizing this DNA construct, the DNA construct was subcloned into the pET30(+) E. coli expression vector via the restriction sites NdeI and HindIII, resulting in the vector pET30a-cIL-13 with 5619 bp in size (SEQ ID NO: 49; plasmid map shown in FIG. 50). Transfection grade plasmid was prepared for E. coli expression.

Expression of the cIL-13 construct from pET30a-cIL-13 was achieved as follows: E. coli strain BL21 Star™ (DE3) was transformed with recombinant plasmid. A single colony was inoculated into Lysogeny Broth (LS) medium containing related antibiotic. The culture was incubated at 37° C. at 200 rpm and then induced with Isopropyl β-D-1-thiogalactopyranoside (IPTG). SDS-PAGE was used to monitor the expression.

Expression was scaled up as follows: Recombinant BL21 (DE3) stored in glycerol was inoculated into Terrific Broth (TB) medium containing related antibiotic and cultured at 37° C. When the OD600 reached about 1.2, the cell culture was induced with IPTG at 15° C. for 16h. Bacteria were harvested by centrifugation.

The purification of the expressed protein was performed as follows: Bacterial pellets were resuspended with lysis buffer followed by sonication. The precipitate after centrifugation was dissolved using denaturing agent. Target protein was obtained by one-step purification using a Ni column. Target protein was sterilized by 0.22 μm filter before stored in aliquots.

The concentration was determined by Bradford protein assay with BSA as standard. Approximately 10.5 mg of (in phosphate-buffered saline, PBS) soluble cIL-13-His₆, referred to in the following examples as cIL-13, was obtained from a bacterial pellet of a 1L E. coli culture.

The protein purity and molecular weight of the expressed protein was determined by standard SDS-PAGE using a reducing loading buffer. BSA was used as a control. Reducing loading buffer (300 mM Tris-HCl, 10% SDS, 30% Glycerol, 0.5% bromophenol blue, 250 mM DTT, pH 6.8) was added to the protein samples, such that the protein samples had a concentration close to 0.5 mg/ml.

After mixing the protein samples with reducing loading buffer, they were heated at 100° C. for 5-10 min, centrifuged at 10000 rpm for 1 min, and then loaded (BSA 2 μg; cIL-13 1.86 μg) in a gel chamber of a precast gel (Genscript, Cat.No. M42012). SDS-PAGE with these gels was performed as outlined by the manufacturer (140 V for approximately 60 min). Thereafter, the gel was stained with Coomassie Blue.

The stained gel is shown in FIG. 51. Lane 1 depicts the size of BSA. Lane 2 depicts the size of the cIL-13 protein.

The band in lane 1 of the SDS-PAGE of FIG. 51 is in good agreement with the expected 66 kDa molecular weight of BSA. The size of the dominant band in lane 2 of the SDS-PAGE of FIG. 51 is in good agreement with the prediction expected from the protein sequence (13394.39 Da, calculated from the mature sequence using https://web.expasy.org/cgi-bin/protparam/protparam).

### Example 3d: Expression of a Properly Folded Native cIL-33-WT in Bacteria (E. Coli) Cells

The cIL-33-WT protein sequence (SEQ ID NO: 50) corresponds to amino acids 110-263 of the full length dog IL-33 protein (Uniprot 097863), in analogy to the well-described amino acid 109-266 form of mouse IL-33 (Uniprot Q8BVZ5).

A DNA construct (SEQ ID NO: 52) encoding for cIL-33-WT (SEQ ID NO: 50) was designed to also encode for a start methionine (M) and a His₆-tag at the N-terminus. This construct was designed to contain an artificial start codon ATG, flanking unique restriction enzyme sites (NdeI and HindIII) for straightforward subcloning, and a stop codon TGA.

After synthesizing this DNA construct, it was subcloned into an pET30 (+) E. coli expression vector via the restriction sites NdeI and HindIII, resulting in the vector pET30a-canIL33-CS with 5742 bp in size (plasmid map shown in FIG. 57).

Expression of the His₆-cIL-33-WT construct from pET30a(+)-canIL33-WT was achieved as follows: pET30a(+)-canIL-33-WT-transformed BL21 (DE3) E. coli were inoculated into Terrific Broth (TB) medium containing kanamycin and cultured at 37° C. When the OD600 reached about 1.2, cell culture was induced with IPTG at 15° C. for 16 hours. Cells were harvested by centrifugation.

The purification of cIL-33-WT protein followed a typical His-tag protein purification scheme using a Ni²⁺column. Cell pellets were resuspended with lysis buffer followed by sonication. The supernatant (soluble expression) after centrifugation was used for column chromatography. Target protein was dialyzed and sterilized by 0.22 μm filter before stored in aliquots. The concentration was determined by Bradford protein assay with BSA as a standard.

The concentration was determined by Bradford protein assay with BSA as standard. Approximately 10.01 mg of soluble (in 50 mM Tris-HCl, 10% Glycerol, 150 mM NaCl, pH 8.0) His₆-cIL-33-WT, referred to in the following examples as cIL-33-WT, was obtained from bacterial pellet of 1 L culture.

The protein purity and molecular weight of the expressed protein was determined by standard SDS-PAGE using a reducing loading buffer and BSA as a control as described for cIL-13 in Example 3c (2.00 μg of each loaded into gel chamber). The stained gel is shown in FIG. 58. Lane 1 depicts the size of BSA and is in good agreement with its expected 66 kDa molecular weight. Lane 2 depicts the size of the cIL-33-WT protein and is in good agreement with the prediction expected from the protein sequence (18578.54 Da, calculated from the mature sequence using https://web.expasy.org/cgi-bin/protparam/protparam).

### Example 3e: Expression of a Properly Folded Native cIL-33-CS in Bacteria (E. Coli) Cells

A DNA construct encoding for cIL-33-CS was designed starting from cIL-33-WT. Each of the three 3 cysteine residues present in a IL-33-WT (SEQ ID NO: 50) were replaced by serine residues to improve stability of the gene product, resulting in IL-33-CS (SEQ ID NO: 51).

The DNA construct (SEQ ID NO: 53) encoding for cIL-33-CS was designed with a start methionine (M) and a His₆-tag at the N-terminus. This construct was designed to contain an artificial start codon ATG, flanking unique restriction enzyme sites (NdeI and HindIII) for straightforward subcloning, and a stop codon TGA.

After synthesizing this DNA construct, it was subcloned into an pET30 (+) E. coli expression vector via the restriction sites NdeI and HindIII, resulting in the vector pET30a-canIL33-CS with 5742 bp in size (plasmid map shown in FIG. 60).

Expression of the cIL-33-CS construct from pET30a-cIL33-CS was achieved as follows: pET30a(+)-canIL-33-CS-transformed BL21 (DE3) (E. coli) were inoculated into Terrific Broth (TB) medium containing kanamycin and cultured at 37° C. When the OD600 reached about 1.2, cell culture was induced with IPTG at 15° C. for 16 hours. Cells were harvested by centrifugation. The purification of His₆-canIL33-CS protein followed a typical His-tag protein purification scheme using a Ni²⁺column. Cell pellets were resuspended with lysis buffer followed by sonication. The precipitate after centrifugation (inclusion bodies) was dissolved using denaturing agent and then subjected to column chromatography. Target protein was sterilized by 0.22 μm filter before stored in aliquots.

The concentration was determined by Bradford protein assay with BSA as standard. By the above procedure, approximately 10.4 mg of soluble (in phosphate buffered saline [PBS], 10% Glycerol, 0.5 M L-Arginine, pH 7.4) His₆-cIL-33-CS were obtained from bacterial pellet of 1 L culture.

The protein purity and molecular weight of the expressed protein was determined by standard SDS-PAGE using a reducing loading buffer and BSA as a control as described for cIL-13 in Example 3c (2.00 μg of each loaded into gel chamber). The stained gel is shown in FIG. 61. Lane 1 depicts the size of BSA and is in good agreement with its expected 66 kDa molecular weight. Lane 2 depicts the size of the cIL-33-CS protein, where the dominant band is in good agreement with the prediction expected from the protein sequence of cIL-33-CS (18530.36 Da, calculated from the mature sequence using https://web.expasy.org/cgi-bin/protparam/protparam).

### Example 3f: Expression of a Properly Folded Native cIL-4 in Bacteria (E. Coli) Cells

A DNA construct (SEQ ID NO: 59) encoding for cIL-4 (SEQ ID NO: 56) was designed to also encode for a start methionine (M) and a His₆-tag at the N-terminus. This construct was designed to contain an artificial start codon ATG, flanking unique restriction enzyme sites (NdeI and HindIII) for straightforward subcloning, and a stop codon TGA.

After synthesizing this DNA construct, it was subcloned into an pET30 (+) E. coli expression vector via the restriction sites NdeI and HindIII, resulting in the vector pET30a-cIL-4 with 5601 bp in size (plasmid map shown in FIG. 70).

Expression of the cIL-4 construct from pET30a-cIL-4 was achieved as follows: E. coli strain BL21 Star™ (DE3) was transformed with recombinant plasmid. A single colony was inoculated into Lysogeny Broth (LS) medium containing related antibiotic. The culture was incubated at 37° C. at 200 rpm and then induced with Isopropyl β-D-1-thiogalactopyranoside (IPTG). SDS-PAGE was used to monitor the expression.

Expression was scaled up as follows: Recombinant BL21 (DE3) stored in glycerol was inoculated into Terrific Broth (TB) medium containing related antibiotic and cultured at 37° C. When the OD600 reached about 1.2, the cell culture was induced with IPTG at 15° C. for 16h. Bacteria were harvested by centrifugation.

The purification of the expressed protein was performed as follows: Bacterial pellets were resuspended with lysis buffer followed by sonication. The precipitate after centrifugation was dissolved using denaturing agent. Target protein was obtained by one-step purification using a Ni column. Target protein was sterilized by 0.22 μm filter before stored in aliquots.

The concentration was determined by Bradford protein assay with BSA as standard. Approximately 10.78 mg of (in phosphate-buffered saline, PBS) soluble cIL-4-His₆, referred to in the following examples as cIL-4, was obtained from from a bacterial pellet of a 1L E. coli culture . . .

The protein purity and molecular weight of the expressed protein was determined by standard SDS-PAGE using a reducing loading buffer. BSA was used as a control. Reducing loading buffer (300 mM Tris-HCl, 10% SDS, 30% Glycerol, 0.5% bromophenol blue, 250 mM DTT, pH 6.8) and non-reducing loading buffer (300 mM Tris-HCl, 10% SDS, 30% Glycerol, 0.5% bromophenol blue, pH 6.8) were added to the protein samples respectively, such that the protein samples had a concentration close to 0.5 mg/ml.

After mixing, the protein sample with reducing loading buffer was heated at 100° C. for 5-10 min. The protein samples were centrifuged at 10000 rpm for 1 min, and then loaded (BSA 2 μg; cIL-4 2 μg) in a gel chamber of a precast gel (Genscript, Cat.No. M42012) and the appropriate running buffer. Electrophoresis was performed at 140 V for approximately 60 min. The Coomassie Blue-stained gel showed a band in Lane 1 that is in good agreement with the expected 66 kDa molecular weight of BSA. The size of the dominant band in lane 2 of the SDS-PAGE is in good agreement with the prediction expected from the cIL-4 protein sequence (13585.88 Da, calculated from the mature sequence using https://web.expasy.org/cgi-bin/protparam/protparam).

### Example 3g: Expression of a Properly Folded Native Fel-IL-31 in Mammalian Cells

A DNA construct (SEQ ID NO: 63) encoding for fel-IL-31 with an artificial ER import signal at the N-terminus and a His₆-tag at the C-terminus was designed. This fel-IL-31-DNA construct was further designed to contain a Kozak sequence upstream of the start codon of the protein to improve expression in mammalian cells and to contain flanking unique restriction enzyme sites for straightforward cloning. After synthesizing this DNA construct, the DNA construct was cloned into the pcDNA3.4 mammalian expression vector, resulting in the vector pcDNA3.4-fel-IL31. Large amounts of transfection grade plasmid were prepared for Expi293F cell expression.

Expression of the cIL-31 construct from pcDNA3.4-fel-IL31 was achieved as follows:

Expi293F cells were grown in serum-free Expi293™ expression Medium (Thermo Fisher Scientific). The cells were maintained in Erlenmeyer Flasks (Corning Inc.) at 37° C. with 8% CO₂ on an orbital shaker (VWR Scientific). One day before transfection, the cells were seeded at an appropriate density in Corning Erlenmeyer Flasks. On the day of transfection, the plasmid pcDNA3.4-fel-IL31 and transfection reagent were mixed at an optimal ratio and then added into the flask with cells ready for transfection. The cell culture supernatants collected on day 6 were used for purification.

The purification and analysis of the expressed protein was performed as follows:

The cell culture broth was centrifuged. The cell culture supernatant was loaded onto an Ni²⁺-NTA affinity purification column at an appropriate flowrate. After washing and elution with appropriate buffers, the eluted fractions were pooled and buffer exchanged to the final formulation buffer, which was PBS pH 7.2.

The purified protein was analyzed by SDS-PAGE and Coomassie Blue staining to determine its molecular weight and purity. To to do so, the concentration of the purified polyprotein was determined by the Bradford assay with BSA as a standard for the calibration curve. Approximately 7.29 mg of (in phosphate-buffered saline, PBS) soluble fel-IL31-His₆, referred to in the following examples as fIL-31, were obtained from 100 ml crude cell culture supernatant.

For the SDS-PAGE analysis the following loading buffers were used:
Reducing Loading buffer: 300 mM Tris-HCl, 10% SDS, 30% Glycerol, 0.5% bromophenol blue, 250 mM DTT, pH 6.8.
Non-Reducing Loading buffer: 300 mM Tris-HCl, 10% SDS, 30% Glycerol, 0.5% bromophenol blue, pH 6.8.

Reducing and non-reducing loading buffer were added to the protein samples, respectively. The protein samples with reducing or non-reducing loading buffer had a concentration close to 0.5 mg/ml. After mixing the protein samples with reducing loading buffer, heating at 100° C. for 5-10 min was performed. The protein samples with reducing or non-reducing loading buffer were centrifuged at 10000 rpm for 1 min, and then loaded in a gel chamber of a precast gel (Genscript, Cat.No. M42012). SDS-PAGE with these gels was performed as outlined by the manufacturer (140 V for approximately 60 min). Thereafter, the gel was stained with Coomassie Blue.

The dominant band in lane 1 of the Coomassie Blue-stained is considerably larger than expected from the protein sequence (16196.54 Da, calculated from the mature sequence using https://web.expasy.org/cgi-bin/protparam/protparam). This difference likely arises from extensive N-glycosylation since the fel-IL-31 protein sequence contains 2 N-glycosylation sites, both of which, based on NetNGlyc analysis (http://www.cbs.dtu.dk/services/NetNGlyc/), are likely to be modified.

Under non-reducing conditions, the bulk of the loaded protein migrated in a band indicative of a monomer. Only a minor proportion appears to be dimeric and multimeric or aggregated forms.

### Example 4: Performance of an In Vivo cIL-31 Activity Test (Pruritus/Itch Induction in Dogs)

To 16 dogs cIL-31 purified as described above in Example 3 was administered intravenously at a single dose of 1.75 μg/kg body weight. To this end, cIL-31 was prepared as liquid formulation in a sterile NaCl solution. The administered dose volume was 1 ml per dog.

The pruritic behavior was video recorded approximately 20-40 minutes after administration of the cIL-31 liquid formulation for 120 min.

The entire observation period of 120 min was split into 1-minute intervals (120 intervals). Categorical yes/no (“I/O”) decisions were made by the observers as following: All intervals, for which at least one pruritic behavior was displayed by the dog, were counted as “1”. All intervals without pruritic behavior were counted as “0”. The type of pruritic behavior was defined by the first behavior occurring in each interval. The cumulative count within each observation period of 120 min provided the pruritus score. Pruritus induction was considered successful if a dog showed pruritic behavior during more than 60 intervals.

Pruritus was observed in all dogs after administration of the cIL-31 liquid formulation. In accordance with the above-defined criteria, pruritus was successfully induced in 13 out of 16 dogs. Adverse events were not observed during this study. In summary, this study therefore provides proof that the expressed cIL-31 construct is biologically active.

### Example 5: Testing Whether the cIL-31 Polyprotein Comprising Three Segments of cIL-31 Also Elicits Pruritus in Dogs

To three dogs cIL-31 polyprotein purified as described above in Example 2 was administered subcutaneously at a single dose of 200 μg. To this end, cIL-31 polyprotein was prepared as liquid formulation in PBS with Polygen as adjuvant. The dose volume was 1 mL per dog.

To evaluate the local and systemic tolerance of dogs against the injected polyprotein, rectal temperature was measured, adspection and palpation of injection sites for clinical signs of swelling, pain, redness and increased heat were performed. If clinical symptoms were found, the aforedescribed local and systemic tolerance assessments were done once weekly until clinical findings disappeared or until the study ended.

None of the dogs showed any signs of pruritus. Mild clinical signs like redness and excoriation were observed at the injection sites and an increased body temperature was observed in all dogs after the first administration and in two dog after the second administration, indicative of an induced immune response against the polyprotein.

### Example 6a: Generation and Characterization of a Polyclonal Rabbit Antiserum Against cIL-31

Approximately 500 μg cIL-31 was used as an antigen for the following 63 day immunization regimen (Davids Biotechnology, Regensburg/FRG) in New Zealand White rabbits:
Day 1: collection of preimmune serum, first immunization
Day 14: second immunization
Day 28: third immunization
Day 35: Test serum for intermediate ELISA titer
Day 42: fourth immunization
Day 56: fifth immunization
Day 63: harvest of antiserum

### Example 6b: Generation and Characterization of a Polyclonal Rabbit Antiserum Against cIL-5

Approximately 500 μg cIL-5 was used as an antigen in the 63 day immunization regimen described above in Example 6a.

### Example 6c: Generation and Characterization of a Polyclonal Rabbit Antiserum Against cIL-13

Approximately 500 μg cIL-13 was used as an antigen in the 63 day immunization regimen described above in Example 6a.

### Example 6d: Generation and Characterization of a Polyclonal Rabbit Antiserum Against cIL-33-WT

Approximately 500 μg cIL-13-WT was used as an antigen in the 63 day immunization regimen described above in Example 6a.

### Example 6e: Generation and Characterization of a Polyclonal Rabbit Antiserum Against cIL-4

Approximately 500 μg cIL-4 was used as an antigen in the 63 day immunization regimen described above in Example 6a.

### Example 6f: Generation and Characterization of a Polyclonal Rabbit Antiserum Against cIL-13-cIL-4-Poly

Approximately 500 μg cIL-13-cIL-4-poly (SEQ ID NO: 203) was used as an antigen in the 63 day immunization regimen described above in Example 6a.

### Example 6g: Generation and Characterization of a Polyclonal Rabbit Antiserum Against cIL-31-cIL-13-cIL-4-poly

Approximately 500 μg cIL-31-cIL-13-cIL-4-poly (SEQ ID NO: 205) was used as an antigen in this immunization regimen.

### Example 6h: Generation and Characterization of a Polyclonal Rabbit Antiserum Against Bov-TNF-Alpha-Poly

Approximately 500 μg bov-TNF-alpha polyprotein (SEQ ID NO: 65) was used as an antigen in the 63 day immunization regimen described above in Example 6a.

### Example 7: Generation of a Chicken IgY Preparation Against cIL-31 from Egg Yolk

Approximately 500 μg cIL-31 were used as an antigen for the following 63 day immunization regimen (Davids Biotechnology, Regensburg/FRG) in chickens:
Day 1: collection of preimmune Egg Yolk IgG/IgY, approx. 90% pure
Day 1: first immunization
Day 14: second immunization
Day 28: third immunization
Day 35: fourth immunization
Day 45-55: collection of the eggs
Day 63: preparation of eggs by a proprietary (Davids Biotechnology, Regensburg; Prep I), IgY preparation yielding a purity and quality comparable to an antiserum from rabbits

### Example 8a: Setup of Anti-cIL-31 and Anti-cIL-31 Polyprotein, ELISA Formats for Rabbit Serum Antibody and Chicken Egg Yolk IgY Titer Determinations, Binding Studies to Native cIL-31 and to cIL-31 Polyprotein Comprising Three Segments of cIL-31

Polystyrene ELISA plates (384 well: Thermo Maxisorp, CatNo. 464718) were coated with 1 μg/ml of either cIL-31 or cIL-31 polyprotein (cIL-31:0.29 mg/ml, or cIL-31 polyprotein: 0.4 mg/ml) dissolved in coating buffer (50 mM NaHCO₃pH 9.6). Per well a volume of 10 μl coating solution was used. The Polystyrene ELISA plates were then incubated overnight (O/N) at 4° C. with a closed lid. After removal of the coating solution, three washes with PBS (ThermoFisher Phosphate-Buffered Saline, pH 7.2, CatNo. 20012-019), 0.05% (v/v) Tween 20 (50 μl per well) were performed. Subsequently, blocking of non-specific binding sites was performed with 50 μl per well of PBS, 0.05% (v/v) Tween 20, 3% (v/v) gelatin (from cold water fish skin, 40-50% in H₂O, Sigma C 7765). This blocking step was performed at room temperature (RT) for at least 1 h. After removal of the blocking solution, serial dilutions (from a non-adsorptive replica plate) of rabbit antiserum or chicken egg yolk preparations in PBS, 0.05% (v/v) Tween 20, 3% (v/v) gelatin were added (20 μl per well). Incubation with the serial dilutions was performed at RT for at least 1 h. Thereafter, the serial dilutions of rabbit antiserum or chicken egg yolk preparations were removed and three washes with PBS, 0.05% (v/v) Tween 20 (50 μl per well) were performed. Next, a 1:15,000 dilution of anti-Rabbit IgG (whole molecule), F (ab′)2 fragment-alkaline phosphatase (AP) antibody produced in goat (Sigma A3937, or equivalent), or a 1:5000 dilution of anti-Chicken IgY (IgG) (whole molecule)-AP antibody produced in rabbit (Sigma A9171, or equivalent) in PBS, 0.05% (v/v) Tween 20, 3% (v/v) gelatin (20 μl per well) were added and incubated at RT for at least 1 h. After removal of these antibody solutions, again three washes with PBS, 0.05% (v/v) Tween 20 (50 μl per well) were performed. Subsequently, the wells were washed once with AP buffer (50 mM NaHCO₃/Na2CO₃, 2 mM MgCl₂, pH 9.6, 50 μl per well). Finally, 5 mM 4-nitrophenyl phosphate disodium salt hexahydrate (pNPP, Applichem, via Sigma, A1442,0050, or equivalent) in AP buffer (90 μl per well) was added. The increase in optical density (OD) at 405 nm per minute (mOD/min) was recorded at RT in an ELISA reader, for example the Epoch Reader (150115E) or the Synergy H₁ Reder (180427C), and the curve slope was determined from a linear increase range.

The results of the ELISA using cIL-31 or cIL-31 polyprotein as antigen for the rabbit preimmune serum and antiserum are depicted in FIG. 5. This Figure shows that the rabbit antiserum recognized both the cIL-31 and the cIL-31 polyprotein construct, while the preimmune serum produced only a negligible signal in the applied ELISA format. The linear signal phase was observed for both antigens up to an antiserum dilution of 1:160, followed by a typical ELISA titration curve. The endpoint titers (last dilution with a signal higher than background signal plus two standard deviations) were >1:40,000 for cIL-31 and >1:80,000 for the cIL-31 polyprotein. These data suggest that the rabbit anti-cIL-31 serum generated in this study was of good quality.

The fact that cIL-31 polyprotein is equally well recognized as cIL-31 by an antiserum raised against the latter protein suggests that the copies of cIL-31 in the polyprotein are properly folded, despite the artificial repeat domain structure and the tetanus toxin spacer sequences. This result was surprising and could not be expected.

The results of the ELISA using cIL-31 or cIL-31 polyprotein as antigen for the chicken preimmune serum and egg yolk preparation are depicted in FIG. 6. As can be seen in this Figure, the chicken egg yolk preparation recognized cIL-31 and the cIL-31 polyprotein, while the chicken preimmune serum produced only a negligible signal in the applied ELISA format when diluted 1:250 (data not shown). The linear signal phase was observed for the cIL-31 polyprotein down to an egg yolk preparation dilution of 62.5 μg/ml, followed by a typical ELISA titration curve. For cIL-31, the linear phase ended at 125 μg/ml egg yolk protein. The endpoint titers of the egg yolk protein preparation (last dilution with a signal higher than background signal plus two standard deviations) were 122 ng/ml for cIL-31 and 61 ng/ml for cIL-31 polyprotein. These data suggest that the chicken egg yolk preparation generated in this study was of good quality.

The fact that cIL-31 polyprotein is roughly equally well recognized as cIL-31 by the chicken egg yolk antibodies which were raised against the latter protein again supports that the copies of cIL-31 in the polyprotein are properly folded, despite the artificial repeat domain structure and the tetanus toxin spacer sequences. This result was surprising and could not be expected.

### Example 8b: Setup of Anti-cIL-5 and Anti-cIL-5 Polyprotein ELISA Formats for Rabbit Serum Antibody Titer Determination, Binding Studies to Native cIL-5 and to cIL-5 Polyprotein Comprising Three Segments of cIL-5

An ELISA was set up to test antiserum generated against cIL-5 in the same manner as in Example 8a for cIL-31. The 384-well polystyrene ELISA plates were coated, however, with 1-5 μg/ml of cIL-5 or 0.5 μg/ml of cIL-31-poly, dissolved in coating buffer as described in Example 8a.

The results of the titer determination of a rabbit anti-cIL-5 antiserum compared to its corresponding preimmune serum are shown in FIG. 44. The rabbit antiserum recognizes cIL-5, while the preimmune serum produces only a negligible signal in the applied ELISA format. The linear signal phase is visible up to an antiserum dilution of ca. 1:500, followed by a typical ELISA titration curve. The endpoint titers (last dilution with a signal >than background+2 standard deviations) are close to 1:200,000. These data suggest that the rabbit anti-cIL-5 serum generated in this study is of good quality.

The results of the ELISA using cIL-5 or cIL-5 polyprotein as antigen for the rabbit preimmune serum and antiserum are depicted in FIG. 45. This Figure shows that the rabbit antiserum recognized both the cIL-5 and the cIL-5 polyprotein construct, while the preimmune serum produced only a negligible signal in the applied ELISA format. While the absolute signal strength is lower in the case of cIL-5-poly compared to cIL-5, the curve shape is very similar in FIG. 45. This suggests that at least a proportion of the cIL-5 polypeptides in cIL-5-poly is in a native-like conformation, despite the artificial repeat domain structure and the tetanus toxin spacer sequences. This result was surprising and could not be expected.

### Example 8c: Setup of Anti-cIL-13 and Anti-cIL-13 Polyprotein ELISA Formats for Rabbit Serum Antibody Titer Determination, Binding Studies to Native cIL-13 and to cIL-13 Polyprotein Comprising Three Segments of cIL-13

An ELISA was set up to test antiserum generated against cIL-13 in the same manner as in Example 8a for cIL-31. The 384-well polystyrene ELISA plates were coated, however, with 5 μg/ml of cIL-13 or 5 μg/ml of cIL-13-poly, dissolved in coating buffer as described in Example 8a.

The results of the titer determination of a rabbit anti-cIL-13 antiserum compared to its corresponding preimmune serum are shown in FIG. 53A. The rabbit antiserum recognizes cIL-13, while the preimmune serum produces only a negligible signal in the applied ELISA format. The linear signal phase is visible up to an antiserum dilution of ca. 1:5000, followed by a typical ELISA titration curve. The endpoint titers (last dilution with a signal >than background+2 standard deviations) exceed 1:500,000. These data suggest that the rabbit anti-cIL-13 serum generated in this study is of good quality.

In the case of cIl-13-poly as coating antigen, the endpoint titer was close to 1:100,000 (FIG. 53 B), which confirms the abundant presence of anti-cIL-13 antibody binding sites on the vaccine antigen construct.

### Example 8d: Setup of Anti-cIL-33-WT and Anti-cIL-33-CS Polyprotein ELISA Formats for Rabbit Serum Antibody Titer Determination, Binding Studies to Native cIL-33 and to cIL-33-CS Polyprotein Comprising Three Segments of cIL-33-CS

An ELISA was set up to test antiserum generated against cIL-33-WT in the same manner as in Example 8a for cIL-31. The 384-well polystyrene ELISA plates were coated, however, with 5 μg/ml of cIL-33-WT or cIL-33-CS-poly, dissolved in coating buffer as described in Example 8a.

The results of the titer determination of a rabbit anti-cIL-13-WT antiserum compared to its corresponding preimmune serum are shown in FIG. 59. The rabbit antiserum recognizes cIL-33-WT, while the preimmune serum produces only a negligible signal in the applied ELISA format. The endpoint titers (last dilution with a signal >than background+2 standard deviations) exceed 1:10,000. These data suggest that the rabbit anti-cIL-33-WT serum generated in this study is of fair quality.

The results of the titer determination of a rabbit anti-cIL-13-CS-poly antiserum compared to its corresponding preimmune serum are shown in FIG. 66. The rabbit antiserum raised against cIL-33-WT recognizes cIL-33-CS-poly, while the preimmune serum produces only a negligible signal in the applied ELISA format. The linear signal phase is visible up to an antiserum dilution of ca. 1:320, followed by a typical ELISA titration curve. The endpoint titers (last dilution with a signal >than background+2 standard deviations) exceed 1:100,000. These data suggest that the cIL-33-CS-poly is actually a better binding partner to the anti-cIL-33-WT serum than the parental antigen. It is likely that the polymeric nature of cIL-33-CS-poly contributes to this property. Also, the result suggests that the C→S mutations have not changed antigenicity of cIL-33-CS compared to cIL-33-WT to a large extent.

### Example 8e: Setup of Anti-cIL-4 and Anti-cIL-4 Polyprotein ELISA Formats for Rabbit Serum Antibody Titer Determination, Binding Studies to Native cIL-4, cIL-4 Polyprotein Comprising Three Segments of cIL-4, and to cIL-13

An ELISA was set up to test antiserum generated against cIL-4 in the same manner as in Example 8a for cIL-31. The 384-well polystyrene ELISA plates were coated, however, with 1 μg/ml of cIL-4 (FIG. 71A) or 2.5 μg/ml of cIL-4-poly (FIG. 71B), dissolved in coating buffer as described in Example 8a.

The results of the ELISA using cIL-4 or cIL-4 polyprotein as antigen for the rabbit preimmune serum and antiserum are depicted in FIG. 71A.

The rabbit antiserum recognizes cIL-4, while the preimmune serum produces only a negligible signal in the applied ELISA format. The linear signal phase is visible up to an antiserum dilution of ˜ 1:80, followed by a typical ELISA titration curve. The endpoint titers (last dilution with a signal >than background+2 standard deviations) exceed 1:150,000. These data suggest that the rabbit anti-cIL-4 serum generated in this study is of good quality.

In the case of cIL-4-poly as coating antigen, the endpoint titer was close to 1:50,000 (FIG. 71B), which confirms the abundant presence of anti-cIL-4 antibody binding sites on the vaccine antigen construct.

In addition, a rabbit serum raised against cIL-13 was tested. This serum produced only a minor signal at low dilutions, orders of magnitudes lower than that of the specific anti-cIL-4 antiserum (FIG. 71B). These data demonstrate that the cIL-4-poly construct contains antigenic determinants of canine IL-4.

### Example 8f: Setup of Anti-cIL-4, Anti-cIL-13, and Anti-cIL-13-cIL-4-Polyprotein ELISA Formats for Rabbit Serum Antibody Titer Determination, Binding Studies to cIL-13-cIL-4 Polyprotein According to the Invention

An ELISA was set up to test antiserum generated against cIL-4 (Example 8e) and cIL-13 (Example 8c) in the same manner as in Example 8a for cIL-31. To test how strongly rabbit antisera against cIL-4 and cIL-13 recognize the cIL-13-cIL-4-polyprotein, the 384-well polystyrene ELISA plates were coated with 2.5 μg/ml of cIL-13-cIL-4-poly, dissolved in coating buffer as described in Example 8a, and incubated with dilutions of antisera raised against cIL-4 and cIL-13. Results are depicted in FIG. 79.

Both rabbit antisera raised against cIL-4 (FIG. 79, triangles) and cIL-13 (FIG. 79, squares) strongly recognize the cIL-13-cIL-4-polyprotein, while preimmune cIL-4 serum (FIG. 79, circles) produces only a negligible signal in the applied ELISA format. The linear signal phase is visible up to an antiserum dilution of ˜ 1:80, followed by a typical ELISA titration curve. The endpoint titers (last dilution with a signal >than background +2 standard deviations) exceed 1:50,000. These data demonstrate that the cIL-13-cIL-4-poly construct contains antigenic determinants of both cytokines cIL-4 and cIL-13.

### Example 8g: Setup of Anti-cIL-13-cIL-4-Polyprotein ELISA Formats for Rabbit Serum Antibody Titer Determination

An ELISA was set up to test antiserum generated against cIL-13-cIL-4 polyprotein according to the invention in the same manner as in Example 8a for cIL-31. The 384-well polystyrene ELISA plates were coated, however, with 1 μg/ml of cIL-4 (FIG. 80A), 1 μg/ml of cIL-13 (FIG. 80B), or 1 μg/ml of cIL-13-cIL-4-polyprotein (FIG. 80C), dissolved in coating buffer as described in Example 8a. The results of the ELISA are depicted in FIG. 80.

The rabbit cIL-13-cIL-4-poly antiserum (triangles) strongly recognizes cIL-4 (FIG. 80A) and cIL-13 (FIG. 80B), with plateau phases up to ˜ 1:300, and endpoint titers reaching or exceeding 1:200,000, while the preimmune serum (circles) produces only a negligible signal in both applied ELISA format (FIGS. 80A,B). In the case of coating the ELISA plate with the immunogen cIL-13-cIL-4-poly (FIG. 80C), the plateau phase of recognition exceeds 1:2000, and the endpoint titer clearly exceeds 1:300,000. These data demonstrate the high immunogenicity of the cIL-13-cIL-4-poly construct in rabbits.

### Example 8h: Setup of Anti-cIL-31-cIL-13-cIL-4-Polyprotein ELISA Formats for Rabbit Serum Antibody Titer Determination

An ELISA was set up to test antiserum generated against cIL-31 (Example 8a), cIL-4

(Example 8e) and cIL-13 (Example 8c) in the same manner as in Example 8a for cIL-31. To test how strongly rabbit antisera against cIL-31, cIL-4, and cIL-13 recognize the cIL-31-cIL-13-cIL-4-polyprotein, the 384-well polystyrene ELISA plates were coated with 1 μg/ml of cIL-31-cIL-13-cIL-4-poly, dissolved in coating buffer as described in Example 8a, and incubated with dilutions of antisera raised against cIL-31, cIL-4, and cIL-13. Results are depicted in FIG. 86.

Rabbit antisera raised against cIL-31 (squares), against cIL-4 (closed circles), or against cIL-13 (triangles) strongly recognize the cIL-31-cIL-13-cIL-4-polyprotein, while preimmune cIL-4 serum (open circles) produces only a negligible signal in the applied ELISA format. These data demonstrate that the cIL-31-cIL-13-cIL-4-poly construct contains antigenic determinants of all cytokines, i.e. cIL-31, cIL-13, and cIL-4.

In addition, an ELISA was set up (results are depicted in FIG. 87) to test rabbit antiserum generated against cIL-31-cIL-13-cIL-4-polyprotein (Example 6f), also analogous to the set-up of Example 8a, except that the 384-well polystyrene ELISA plates were coated with 1 μg/ml of cIL-4 (FIG. 87, large circles), 1 μg/ml of cIL-13 (FIG. 87, large squares), 1 μg/ml of cIL-31 (FIG. 87, large diamonds), or 1 μg/ml of cIL-31-cIL-13-cIL-4 polyprotein (FIG. 87, large triangles), dissolved in coating buffer as described in Example 8a, and incubated with dilutions of antisera raised against the cIL-31-cIL-13-cIL-14-polyprotein. Dilutions of preimmune rabbit serum are also shown (all remain close to 0,0 mean OD405). The results show that immunization of a rabbit with cIL-31-cIL-13-cIL-4-polyprotein according to the invention resulted in an antiserum with high titers against the immunogen itself, as well as all three individual immunogen polypeptide components (cIL-31, cIL-4 and cIL-13). These results demonstrate that the protein construct cIL-31-cIL-13-cIL-4-polyprotein possesses immunogen properties for all three cytokines.

### Example 8i: Setup of Anti-Bov-TNF-Alpha Polyprotein ELISA Formats for Rabbit Serum Antibody Titer Determination

An ELISA was set up to test antiserum generated against bovine TNF-alpha-polyprotein comprising three segments of bovine TNF-alpha in the same manner as in Example 8a for cIL-31. The 384-well polystyrene ELISA plates were coated, however, with 1 μg/ml of bov-TNF-alpha (R&D Systems), dissolved in coating buffer as described in Example 8a.

The results of the titer determination of a rabbit anti-bov-TNF-alpha-poly antiserum compared to its corresponding preimmune serum are shown in FIG. 93. The rabbit antiserum recognizes bovine TNF-alpha, while the preimmune serum produces only a negligible signal in the applied ELISA format. Thus, immunization of a rabbit with bov-TNFa-poly leads to a high titer anti-bovine-TNF-α antiserum. The endpoint of this antiserum is beyond a 1:40,000 dilution, and saturation is seen up to 1:200 dilution. This demonstrates that the intended immunogenicity of bov-TNFa-poly has been achieved using the polyprotein construct comprising at least two segments of a TNF-alpha protein.

### Example 9: Setup of an ELISA Format for Binding of the cIL-31 Neutralizing Canine Monoclonal Antibody (mAb) Lokivetmab to Native cIL-31 and to cIL-31 Polyprotein Comprising Three Segments of cIL-31

Lokivetmab is a caninized monoclonal antibody directed against canine IL-31 (Michels et al., “A blinded, randomized, placebo-controlled, dose determination trial of lokivetmab (ZTS-00103289), a caninized, anti-canine IL-31 monoclonal antibody in client owned dogs with atopic dermatitis”, Veterinary dermatology 27.6 (2016): 478-e129) and forms the active of the veterinary drug Cytopoint®.

The following ELISA format to test the binding of lokivetmab to cIL-31 or cIL-31 polyprotein was developed:

Polystyrene ELISA plates (384 well: Thermo Maxisorp, CatNo. 464718) were coated with 1 μg/ml of either cIL-31 or cIL-31 polyprotein (cIL-31:0.29 mg/ml, or cIL-31 polyprotein: 0.4 mg/ml) dissolved in coating buffer (50 mM NaHCO₃pH 9.6). Per well a volume of 10 μl coating solution was used. The Polystyrene ELISA plates were then incubated overnight (O/N) at 4° C. with a closed lid. After removal of the coating solution, three washes with PBS (ThermoFisher Phosphate-Buffered Saline (PBS), pH 7.2, CatNo. 20012-019), 0.05% (v/v) Tween 20 (50 μl per well) were performed. Subsequently, blocking of non-specific binding sites was performed with 50 μl per well of PBS, 0.05% (v/v) Tween 20, 3% (v/v) gelatin (from cold water fish skin, 40-50% in H₂O, Sigma C 7765). This blocking step was conducted at RT for at least 1 h. After removal of the blocking solution, serial dilutions (from a non-adsorptive replica plate) of lokivetmab in PBS, 0.05% (v/v) Tween 20, 3% (v/v) gelatin were added (20 μl per well). Incubation with the serial dilutions of lokivetmab was performed at RT for at least 1 h. Thereafter, the serial dilutions of lokivetmab were removed and three washed with PBS, 0.05% (v/v) Tween 20 (50 μl per well) were performed. Next, a 1:5000 dilution of protein A-AP (P7488 Sigma) or a 1:2000 dilution of anti-dog-IgG-AP (Sigma Lot RI6082 0.61 mg/ml) in PBS, 0.05% (v/v) Tween 20, 3% (v/v) gelatin (20 μl per well) was added and incubated at RT for at least 1 h. After removal of these solutions, again three washes with PBS, 0.05% (v/v) Tween 20 (50 μl per well) were performed. Subsequently, the wells were washed once with AP buffer (50 mM NaHCO₃/Na₂CO₃, 2 mM MgCl₂, pH 9.6, 50 μl per well). Finally, 5 mM 4-nitrophenyl phosphate disodium salt hexahydrate (pNPP, Applichem, via Sigma, A1442,0050, or equivalent) in AP buffer (90 μl per well) was added. The increase in optical density (OD) at 405 nm per minute (mOD/min) was recorded at RT in an ELISA reader and the curve slope was determined from a linear increase range.

The results of the ELISA using cIL-31 as antigen for lokivetmab are depicted in FIG. 7. These results suggest that lokivetmab binds to cIL-31 and that it can be detected by anti-dog-IgG reagents. Lokivetmab bound to cIL-31, however, is only poorly recognized by protein A.

The results of the ELISA using cIL-31 and cIL-31 polyprotein as antigen for lokivetmab are depicted in FIG. 8. These results demonstrate that lokivetmab binds to cIL-31 and cIL-31 polyprotein. This suggest that copies of cIL-31 in the polyprotein have a native fold.

### Example 10: Generation and Characterization of Biotinylated Lokivetmab

For biotinylating lokivetmab, Zeba 0.5 ml Spin desalting columns (40K) were first equilibrated with 50 mM NaHCO₃, 150 mM NaCl, pH 8.5. Next, 100 μl lokivetmab (10 mg/ml) were passed twice through the eqilibated columns. Thereafter, 20 μl 10 mM EZ-Link™ sulfon-NHS-LC-LC-Biotin (Thermo Scientific 21338) was added to the lokivetmab-containing solution and incubated for 4 h at 37° C. After this incubation, 1 μl 1 M Tris pH 8.0 was added. After equilibration of further Zeba 0.5 ml Spin desalting columns with PBS pH 7.2, the reaction mixture containing lokivetmab was passed though the PBS-equilibrated columns and filled up with PBS to a final volume of 200 μl and a concentration of 5 mg/ml biotinylated lokivetmab was assumed.

Next, an ELISA format to test the cIL-31 binding of biotinylated lokivetmab was developed:

Polystyrene ELISA plates (384 well: Thermo Maxisorp, CatNo. 464718) were coated with 1 μg/ml of either cIL-31 or cIL-31 polyprotein (cIL-31:0.29 mg/ml, or cIL-31 polyprotein: 0.4 mg/ml) dissolved in coating buffer (50 mM NaHCO₃pH 9.6). Per well a volume of 10 μl coating solution was used. The Polystyrene ELISA plates were then incubated overnight (O/N) at 4° C. with a closed lid. After removal of the coating solution, three washes with PBS (ThermoFisher Phosphate-Buffered Saline (PBS), pH 7.2, CatNo. 20012-019), 0.05% (v/v) Tween 20 (50 μl per well) were performed. Subsequently, blocking of non-specific binding sites was performed with 50 μl per well of PBS, 0.05% (v/v) Tween 20, 3% (v/v) gelatin (from cold water fish skin, 40-50% in H₂O, Sigma C 7765). This blocking step was conducted at RT for at least 1 h. After removal of the blocking solution, serial dilutions (from a non-adsorptive replica plate) of lokivetmab in PBS, 0.05% (v/v) Tween 20, 3% (v/v) gelatin were added (20 μl per well). Incubation with the serial dilutions of biotinylated lokivetmab was performed at RT for at least 1 h. Thereafter, the serial dilutions of biotinylated lokivetmab were removed and three washed with PBS, 0.05% (v/v) Tween 20 (50 μl per well) were performed. Next, a 1:17.000 dilution of ExtrAvidin®-AP (Sigma E2636) in PBS, 0.05% (v/v) Tween 20, 3% (v/v) gelatin (20 μl per well) was added and incubated at RT for at least 1 h. After removal of this solution, again three washes with PBS, 0.05% (v/v) Tween 20 (50 μl per well) were performed. Subsequently, the wells were washed once with AP buffer (50 mM NaHCO₃/Na₂CO₃, 2 mM MgCl₂, pH 9.6, 50 μl per well). Finally, 5 mM 4-nitrophenyl phosphate disodium salt hexahydrate (pNPP, Applichem, via Sigma, A1442,0050, or equivalent) in AP buffer (90 μl per well) was added. The increase in optical density (OD) at 405 nm per minute (mOD/min) was recorded at RT in an ELISA reader and the curve slope was determined from a linear increase range.

The results of the ELISA using cIL-31 as antigen for biotinylated lokivetmab are depicted in FIG. 9. These results suggest that biotinylation of lokivetmab was successful since a strong titratable signal was observed in the employed ELISA setup.

### Example 11: Setup of a Biotinylated Lokivetmab Competition Assay Format

The rabbit preimmune serum, the rabbit anti-cIL-31 antiserum (see Example 6), cIL-31 polyprotein 0.4 mg/ml (Genscript), cIL-31 0.29 mg/ml (Genscript) and lokivetmab-biotin (see Example 10) were used to set up a lokivetmab competition assay.

The biotinylated lokivetmab competition assay format was designed as follows:

Polystyrene ELISA plates (384 well: Thermo Maxisorp, CatNo. 10192781) were coated with 1 μg/ml of either cIL-31 or cIL-31 polyprotein (stock solutions in PBS: cIL-31:0.29 mg/ml, or cIL-31 polyprotein: 0.4 mg/ml) dissolved in coating buffer (50 mM NaHCO₃pH 9.6). Per well a volume of 10 μl coating solution was used. The Polystyrene ELISA plates were then incubated overnight (O/N) at 4° C. with a closed lid. After removal of the coating solution, three washes with PBS (ThermoFisher Phosphate-Buffered Saline (PBS), pH 7.2, CatNo. 20012-019), 0.05% (v/v) Tween 20 (35 μl per well) were performed. Subsequently, blocking of non-specific binding sites was performed with 35 μl per well of PBS, 0.05% (v/v) Tween 20, 3% (v/v) gelatin (from cold water fish skin, 40-50% in H₂O, Sigma C 7765). This blocking step was conducted at RT for at least 1 h with a closed lid. Serial dilutions from a of rabbit preimmune, rabbit anti-cIL-31 immune serum or of chicken egg yolk IgY preparation in 100 ng/ml biotinylated lokivetmab in PBS, 0.05% (v/v) Tween 20, 3% (v/v) gelatin were prepared in a separate non-absorptive ELISA plate and incubated for about 1 h at RT. After removing the blocking solution from the cIL-31 or cIL-31 polyprotein coated ELISA plate, the preincubated serial antibody dilutions were added (20 μl per well). Incubation with the serial antibody dilustions was performed at RT for about 1 h. Thereafter, the serial antibody dilutions were removed and three washed with PBS, 0.05% (v/v) Tween 20 (35 μl per well) were performed. Next, a 1:17.000 dilution of ExtrAvidin®-AP (Sigma E2636) in PBS, 0.05% (v/v) Tween 20, 3% (v/v) gelatin (20 μl per well) was added and incubated at RT for at least 1 h with a closed lid. After removal of this solution, again two washes with PBS, 0.05% (v/v) Tween 20 (35 μl per well) were performed. Subsequently, the wells were washed once with AP buffer (50 mM NaHCO₃/Na₂CO₃, 2 mM MgCl₂, pH 9.6, 50 μl per well). Finally, 5 mM 4-nitrophenyl phosphate disodium salt hexahydrate (pNPP, Applichem, via Sigma, A1442,0050, or equivalent) in AP buffer (90 μl per well) was added. The increase in optical density (OD) at 405 nm per minute (mOD/min) was recorded at RT in an ELISA reader and the curve slope was determined from a linear increase range.

The ELISA set-up using a mixture of rabbit preimmune serum+biotinylated lokivetmab or a mixture of rabbit immune serum+biotinylated lokivetmab probes for the interaction between biotinylated lokivetmab as cIL-31 neutralizing antibody and cIL-31 or cIL-31 polyprotein immobilized on an ELISA plate. The results of FIGS. 10 and 11 show that rabbit preimmune serum does not interfere with the binding of lokivetmab to either cIL-31 or cIL-31 polyprotein. The anti-cIL-31 rabbit immunserum, however, exhibits inhibitory activity for this interaction starting at a dilution of 1:128 to 1:64 and leading to complete inhibition of lokivetmab binding at a dilution of 1:2.

The ELISA set-up using a mixture of chicken egg yolk IgY preparation+biotinylated lokivetmab probes for the interaction between biotinylated lokivetmab as cIL-31 neutralizing antibody and cIL-31 immobilized on an ELISA plate. The results of FIG. 12 show that the anti-cIL-31 chicken egg yolk IgY preparation exhibited inhibitory activity for this interaction starting between 122 and 244 μg/ml and leading to complete inhibition of biotinylated lokivetmab binding at 15.6 mg/ml.

### Example 12a: Generation of a Dog Monocyte Cell Line (DH82) Allowing to Evaluate the NFkB-Stimulating Potential of Oligodeoxynucleotides (CpG-ODNs)

The dog monocyte cell line DH82 (Wellman et al. 1988) was transfected with pcDNA3.1 (+)-bsd-NFkB-SEAP (plasmid map included as FIG. 13). This resulted in a blasticidin S-selected cell line that could be stimulated to secreted embryonic alkaline phosphatase (SEAP) secretion by a number of NFkB pathway activating ligands (such as LPS or TNF-«). Single cell cloning was performed to obtain a clonal cell line.

This clonal cell line (DH82-bsd-NFSEAP) was exposed to different phosphorothioate oligodeoxynucleotides (PTO-ODNs). A strong concentration-dependent SEAP signal was observed for three different PTO-ODNs with the order of potency 1668-PTO>>2006-PTO>2007-PTO (see FIG. 14).

The results of FIG. 14 suggest that DH82 expresses functional toll-like receptor 9 (TLR9) since it is the only known receptor recognizing PTO-ODNs and leading to NFkB signaling. Moreover, the results of FIG. 14 surprisingly show that in particular 1668-PTO (SEQ ID NO: A) is a potent activator of dog TLR9.

### Example 12b: Stimulating Potential of cIL-13 in HEK-Blue™ IL-13 Cells

HEK-Blue™ IL-4/IL-13 cells (Invivogen, hkb-il413) are stably transfected with the human STAT6 gene to obtain a fully active STAT6 pathway. Furthermore, the cells are transfected with a STAT6-inducible SEAP reporter gene. The receptor subunits IL4Ra and IL-13Ra1 as well as other genes of the signaling pathway are naturally expressed in sufficient amounts. These cells are responsive to human IL-4 and human Il-13 (https://www.invivogen.com/hek-blue-il4-il13, data not shown).

Testing of these cells with cIL-13 revealed that this cytokine is sensitively recognized by HEK-Blue™ IL-4/IL-13 cells and results in a SEAP reporter enzyme readout with ˜ 2 ng/ml (2000 μg/ml) EC50 (see FIG. 52).

### Example 12c: Stimulating Potential of cIL-33-WT and cIL-33-CS in HEK-Blue™ IL-33 Cells

HEK-Blue™ IL-33 cells (Invivogen, hkb-hiL33) were used to evaluate the stimulating potential of cIL-33-WT and cIL-33-CS proteins. The cells had been generated by stable transfection of human embryonic kidney HEK293-derived cells with the human IL1RL1 gene. In addition, the TNF-α and the IL-1B responses were blocked. Therefore, HEK-Blue™ IL-33 cells respond specifically to IL-33. These cells express an NF-κB/AP-1-inducible SEAP reporter gene. The binding of human IL-33 to the heterodimeric IL-1RL1/IL-1RAcP on the surface of these cells is known to trigger a signaling cascade leading to the activation of NF-κB and the subsequent production of SEAP.

These cells are responsive to human IL-33 (https://www.invivogen.com/hek-blue-il33). Testing of these cells with canine IL-33 revealed that cIL-33-WT from different sources is variably recognized (FIG. 62, “cIL-33-WT Batch1” with closed triangles, and “cIL-33-WT Batch2” with closed squares) and appears to lose stimulatory activity over time (inventors' own observation, data not shown), presumably due to oxidation of the thiol-containing cysteine residues.

However, cIL-33-CS is sensitively recognized by HEK-Blue™ IL-33 cells, and results in a SEAP reporter enzyme readout with ˜ 10 ng/ml EC50 (FIG. 62, closed circles) after the three cysteines in cIL-33-WT are mutated to serines (cIL-33-CS).

### Example 13a: Design of a cIL-31-Polyprotein Comprising Three Segments of cIL-31/PTO-ODN/Polygen Vaccine Formulation and Immunization Study

The cIL-31 polyprotein vaccine was defined to contain:
200 μg cIL-31-poly (SEQ ID NO: 4 and/or SEQ ID NO: 40)
50 μg 1668-PTO (SEQ ID NO: 5)
50 μg 2006-PTO (SEQ ID NO: 6)
in 900 μl PBS
+100 μl Polygen

The immunization of the dogs was performed by subcutaneous injections as follows:
Day 0: primary immunization with the cIL-31 polyprotein vaccine as defined above and collection of a blood sample (preimmune sample)
Day 7: collection of a blood sample
Day 14: collection of a blood sample
Day 21: collection of a blood sample
Day 28: secondary immunization with the cIL-31 polyprotein vaccine as defined
above and collection of a blood sample
Day 35: collection of a blood sample
Day 42: collection of a blood sample
Day 49: collection of a blood sample
Day 56: collection of a blood sample
Day 63: collection of a blood sample
Day 70: collection of a blood sample

### Example 13b: Design of a cIL-5-Polyprotein Comprising Three Segments of cIL-5/PTO-ODN/Polygen Vaccine Formulation and Immunization Study

The cIL-5 polyprotein vaccine was defined to contain:
23 μg cIL-5-poly (SEQ ID NO: 42)
50 μg 1668-PTO (SEQ ID NO: 5)
50 μg 2006-PTO (SEQ ID NO: 6)
in 1000 μl PBS
+110 μl Polygen

The immunization and blood sampling scheme of three dogs (Dog 1 (0368), Dog 2 (9641), Dog 3 (0852)) was performed as described for Example 13a, except that sampling did not include Day 70.

### Example 13c: Design of a cIL-13-Polyprotein Comprising Three Segments of cIL-13/PTO-ODN/Polygen Vaccine Formulation and Immunization Study

One injection dose of the cIL-5 polyprotein vaccine was defined to contain:
50 μg cIL-13-poly (SEQ ID NO: 47)
50 μg 1668-PTO (SEQ ID NO: 5)
50 μg 2006-PTO (SEQ ID NO: 6)
in 900 μl PBS
+100 μl Polygen

The immunization and blood sampling scheme of three dogs (Dog cIL-13-1 (0521), Dog cIL-13-2 (2579), Dog cIL-13-3 (6048)) was performed as described for Example 13a, except that sampling did not include Day 70.

### Example 13d: Design of a cIL-33-Polyprotein Comprising Three Segments of cIL-33/PTO-ODN/Polygen Vaccine Formulation and Immunization Study

The cIL-33-CS polyprotein vaccine was defined to contain:
100 μg cIL-33-CS-poly (SEQ ID NO: 54)
50 μg 1668-PTO (SEQ ID NO: 5)
50 μg 2006-PTO (SEQ ID NO: 6)
in 900 μl PBS
+100 μl Polygen

The immunization and blood sampling scheme of one dog (Dog 402) was performed as described for Example 13a, except that sampling did not include Day 70. The presence of specific anti-cIL-33 antibodies was assessed in a classical plate-bound antigen ELISA setup. The wild type form of cIL-33 was used in this analysis, to exclude any effect specific for the C→S mutations.

### Example 13e: Design of a cIL-4-Polyprotein Comprising Three Segments of cIL-4/PTO-ODN/Polygen Vaccine Formulation and Immunization Study

One injection dose of the cIL-4 polyprotein vaccine was defined to contain:
200 μg cIL-4-poly (SEQ ID NO: 57)
50 μg 1668-PTO (SEQ ID NO: 5)
50 μg 2006-PTO (SEQ ID NO: 6)
in 900 μl PBS
+100 μl Polygen

The immunization and blood sampling scheme of three dogs (Dog 5365, Dog 6523, Dog 7104) was performed as described for Example 13a, except that sampling did not include Day 70.

### Example 13f: Design of a fel-IL-31-polyprotein comprising three segments of fel-IL-31/PTO-ODN/Polygen vaccine formulation and immunization study

One injection dose of the fel-IL-31 polyprotein vaccine was defined to contain:
100 μg fel-IL-31-poly (SEQ ID NO: 61)
25 μg 1668-PTO (SEQ ID NO: 5)
25 μg 2006-PTO (SEQ ID NO: 6)
in 900 μl PBS
+100 μl Polygen

The immunization and blood sampling scheme of three cats (Cat 3132, Cat 0487, Cat 5674) was performed as described for Example 13a, except that the secondary immunization took place on Day 35 and blood samples were also drawn on Days 77, 84, 91, 98, 105, 112, 119, and 126.

### Example 13g: Design of Further Polyproteins Comprising Three Segments of a Single Self-Protein/PTO-ODN/Polygen Vaccine Formulations and Immunizations

Further polyprotein vaccines can be defined to contain for one dose:
200 μg of one of SEQ ID NOs: 68 to 201
50 μg 1668-PTO (SEQ ID NO: 5)
50 μg 2006-PTO (SEQ ID NO: 6)
in 900 μl PBS
+100 μl Polygen

The immunization and blood sampling can be performed as described for Example 13a.

### Example 13h: Design of a cIL-13-cIL-4-Polyprotein According to the Invention/PTO-ODN/Polygen Vaccine Formulations and Immunizations

One injection dose of the cIL-13-cIL-4-polyprotein vaccine was defined to contain:
200 μg cIL-13-cIL-4-polyprotein (SEQ ID NO: 203)
50 μg 1668-PTO (SEQ ID NO: 5)
50 μg 2006-PTO (SEQ ID NO: 6)
in 900 μl PBS
+100 μl Polygen

The immunization and blood sampling scheme of three dogs (Dog 8322, Dog 6504, Dog 6043) was performed as described for Example 13a, except that the secondary immunization took place on Day 35, sampling did not include Day 70, and blood was sampled at Day 27 instead of Day 28.

### Example 13i: Design of a cIL-31-cIL-13-cIL-4-Polyprotein According to the Invention/PTO-ODN/Polygen Vaccine Formulations and Immunizations

One injection dose of the cIL-13-cIL-4-polyprotein vaccine was defined to contain:
200 μg cIL-31-cIL-13-cIL-4-polyprotein (SEQ ID NO: 205)
50 μg 1668-PTO (SEQ ID NO: 5)
50 μg 2006-PTO (SEQ ID NO: 6)
in 900 μl PBS
+100 μl Polygen

The immunization and blood sampling scheme of three dogs (Dog 0720, Dog 6731, Dog 9214) was performed as described for Example 13a, except that sampling did not include Day 70.

### Example 14a: Determination of Anti-cIL-31 Titers in the Immunized Dogs

The dog sera obtained from the immunized dogs (see Example 13a) were tested for the presence anti-cIL-31 antibodies based on the following ELISA format:

Polystyrene ELISA plates (384 well: Thermo Maxisorp, CatNo. 464718) were coated with 1 μg/ml of either cIL-31 or cIL-31 polyprotein (cIL-31:0.29 mg/ml, or cIL-31 polyprotein: 0.4 mg/ml) dissolved in coating buffer (50 mM NaHCO₃pH 9.6). Per well a volume of 10 μl coating solution was used. The Polystyrene ELISA plates were then incubated overnight (O/N) at 4° C. with a closed lid. After removal of the coating solution, three washes with PBS (ThermoFisher Phosphate-Buffered Saline (PBS), pH 7.2, CatNo. 20012-019), 0.05% (v/v) Tween 20 (50 μl per well) were performed. Subsequently, blocking of non-specific binding sites was performed with 50 μl per well of PBS, 0.05% (v/v) Tween 20, 3% (v/v) gelatin (from cold water fish skin, 40-50% in H₂O, Sigma C 7765). This blocking step was conducted at RT for at least 1 h. After removal of the blocking solution, serial dilutions (from a non-adsorptive replica plate) of dog sera in PBS, 0.05% (v/v) Tween 20, 3% (v/v) gelatin were added (20 μl per well). Incubation with the serial dilutions of dog sera was performed at RT for at least 1 h.

Thereafter, the serial dilutions of dog sera were removed and thrice washed with PBS, 0.05% (v/v) Tween 20 (50 μl per well) were performed. Next, a 1:2,000 dilution of Rabbit IgG anti-Dog IgG (Fc)-Alk. Phos., MinX none (Dianova GmbH, SKU 304-055-008, or equivalent) in PBS, 0.05% (v/v) Tween 20, 3% (v/v) gelatin (20 μl per well) was added and incubated at RT for at least 1 h. After removal of this solution, again three washes with PBS, 0.05% (v/v) Tween 20 (50 μl per well) were performed. Subsequently, the wells were washed once with AP buffer (50 mM NaHCO₃/Na₂CO₃, 2 mM MgCl₂, pH 9.6, 50 μl per well). Finally, 5 mM 4-nitrophenyl phosphate disodium salt hexahydrate (pNPP, Applichem, via Sigma, A1442,0050, or equivalent) in AP buffer (90 μl per well) was added. The increase in optical density (OD) at 405 nm per minute (mOD/min) was recorded at RT in an ELISA reader and the curve slope was determined from a linear increase range.

FIGS. 15a-c to 17a-c depict the results of these ELISAs which show that the immunized dogs produced anti-cIL-31 antibodies in considerable amounts already 14 days after immunization. High anti-cIL-31 titers could also still be observed on day 112 after the primary immunization uniformly in all three dogs.

### Example 14b: Determination of Anti-cIL-5 Titers in the Immunized Dogs

The dog sera obtained from the immunized dogs (see Example 13b) were tested for the presence anti-cIL-5 antibodies based on the same ELISA format as described in Example 14a for anti-cIL-31 antibodies. The 384-well polystyrene ELISA plates were coated with 1-5 μg/ml of cIL-5 dissolved in coating buffer.

FIG. 46 (A-F) depicts the results of these ELISAs which show that two cIL-5-poly immunized dogs produced anti-cIL-5 antibodies in considerable amounts already 28 days after immunization (FIGS. 46A and 46 E). The booster immunization at day 28 led to a further massive anti-cIL-5 antibody titer increase in these two dogs, that peaked at day 42 (FIGS. 46 B and 46 F). Titers remained high at the last sampling point, day 63, and likely extend beyond. One dog showed an overall poor response to cIL-5-poly vaccination (FIG. 46 C-D), but an effect of the booster immunization at day 28 was visible (FIG. 46 D).

### Example 14c: Determination of Anti-cIL-13 Titers in the Immunized Dogs

The dog sera obtained from the immunized dogs (see Example 13c) were tested for the presence anti-cIL-13 antibodies based on the same ELISA format as described in Example 14a for anti-cIL-31 antibodies. The 384-well polystyrene ELISA plates were coated with 1-5 μg/ml of cIL-13 (stock solution: cIL-13:0.15 mg/mL) dissolved in the coating buffer.

FIG. 54 depicts the results of these ELISAs. The cIL-13-poly immunized dogs showed only minor anti-cIL-13 antibody development in the primary immunization up to day 28 (FIG. 54A-C). However, the booster immunization at day 28 led to a massive anti-cIL-13 antibody titer increase in all three dogs, that peaked around day 35-day 42, and lasted to day 63, and likely beyond (FIG. 54A-C).

### Example 14d: Determination of Anti-cIL-33-CS Titers in the Immunized Dogs

The dog sera obtained from the immunized dogs (see Example 13d) were tested for the presence anti-cIL-33 antibodies based on the same ELISA format as described in Example 14c for anti-cIL-33 antibodies. The 384-well polystyrene ELISA plates were coated with 1-5 μg/ml of cIL-33-WT (to exclude any C >S abnormalities) dissolved in the coating buffer.

FIG. 67 depicts the results of these ELISAs. The cIL-33-CS-poly immunized dog showed no anti-cIL-33-WT antibody development in the primary immunization up to day 28 (FIG. 67, open symbols). However, the booster immunization at day 28 led to an increase in anti-cIL-33-WT antibody titers, that peaked at day 42, and lasted to day 63, with little decrease over time (FIG. 67, closed symbols).

These results show that the designed cIL-33-CS-poly antigen leads to breakage of self-tolerance to cIL-33-WT.

### Example 14e: Determination of Anti-cIL-4 Titers in the Immunized Dogs

The dog sera obtained from the immunized dogs (see Example 13e) were tested for the presence anti-cIL-4 antibodies based on the same ELISA format as described in Example 14a for anti-cIL-31 antibodies. The only exception was that the 384-well polystyrene ELISA plates were coated with 1 μg/ml of cIL-4 (ex Genscript cIL-4, 0.84 mg/ml) dissolved in the coating buffer.

FIG. 72 depicts the results of these ELISAs. The cIL-4-poly immunized dogs showed only minor anti-cIL-4 antibody development in the primary immunization up to day 35 (FIG. 72 A-C). However, the booster immunization at day 35 led to a massive anti-cIL-4 antibody titer increase in one dog (Dog 5365; FIG. 72A) that peaked around day 42, and lasted through day 63, and likely beyond. Two dogs (Dog 6523, FIG. 72B and Dog 7104, FIG. 72C) showed essentially the same picture, but with weaker titers after boost than Dog 5365. This experiment shows that the designed cIL-4-poly antigen in the chosen formulation leads to breakage of self-tolerance to cIL-4.

### Example 14f: Determination of Anti-Fel-IL-31 Titers in the Immunized Cats

The feline sera obtained from the immunized cats (see Example 13f) were tested for the presence anti-cIL-31 antibodies based on the same ELISA format as described in Example 14a for anti-cIL-31 antibodies in dogs. The only exception was that the 384-well polystyrene ELISA plates were coated with 1 μg/ml of fel-IL-4 (ex Genscript U6344FL160-4, fel-IL-31, 0.54 mg/ml) dissolved in the coating buffer.

FIGS. 75 and 76 depict the results of these ELISAs. Two out of the three fel-IL-31-poly immunized cats (Cat 3132 and Cat 5674) showed only minor or no anti-fel-IL-31 antibody development in the primary immunization up to day 27 (FIG. 75A-C). However, the booster immunization at day 27 led to a massive anti-fel-IL-31 antibody titer increase in all three cats that remained high from day 35 to day 63 (FIG. 75A-C). Further ELISA analysis of blood samples until day 126 (FIG. 76) showed that specific antibody titers declined only very slowly, with significant titers still present 4 months after primary immunization and 3 months after secondary immunization (FIG. 76A-C).

This experiment shows that the designed fel-IL-31-poly antigen leads to breakage of self-tolerance to fel-IL-31 in cats, with long-lasting anti-fel-IL-31 antibody titers.

### Example 14g: Determination of Anti-cIL-4 Titers in the cIL-13-cIL-4-Polyprotein-Immunized Dogs According to the Invention

The dog sera obtained from three dogs immunized with cIL-13-cIL-4-polyprotein (see Example 13h) were tested for the presence anti-cIL-4 antibodies based on the same ELISA format as described in Example 14a for anti-cIL-31 antibodies. The only exception was that the 384-well polystyrene ELISA plates were coated with 1 μg/ml of cIL-4 (ex Genscript cIL-4) dissolved in the coating buffer.

FIG. 81 depicts the results of these ELISAs. One cIL-13-cIL-4-poly immunized dog (6504) showed only minor or no anti-cIL-4 antibody development in the primary immunization up to day 35 (FIG. 81B), while the two other dogs (8322, 6043) showed sizable titers already at days 14, 21, 27 and 35 (FIGS. 81A,C, respectively). However, the booster immunization at day 35 led to a massive anti-cIL-4 antibody titer increase in all dogs (FIGS. 81A-C), that peaked at days 42 and 49, and lasted to day 63, and likely beyond.

This experiment shows that the designed cIL-13-cIL-4-polyprotein antigen in the chosen formulation leads to breakage of self-tolerance to cIL-4.

### Example 14h: Determination of Anti-cIL-13 Titers in the cIL-13-cIL-4-Polyprotein-Immunized Dogs According to the Invention

The dog sera obtained from three dogs immunized with cIL-13-cIL-4-polyprotein (see Example 13h) were tested for the presence anti-cIL-13 antibodies based on the same ELISA format as described in Example 14a for anti-cIL-31 antibodies. The only exception was that the 384-well polystyrene ELISA plates were coated with 1 μg/ml of cIL-13 dissolved in the coating buffer.

FIG. 82 depicts the results of these ELISAs. One cIL-13-cIL-4-polyprotein-immunized dog (6504) showed only minor or no anti-cIL-13 antibody development in the primary immunization up to day 35 (FIG. 82B), while the two other dogs (8322, 6043) showed sizable titers already at days 14, 21, 27 and 35 (FIG. 82A,C, respectively). However, the booster immunization at day 35 led to a massive anti-cIL-13 antibody titer increase in all dogs, that peaked at days 42 and 49, and lasted to day 63, and likely beyond (FIG. 82A-C).

This experiment shows that the designed cIL-13-cIL-4-polyprotein antigen in the chosen formulation also leads to breakage of self-tolerance to cIL-13.

### Example 14i: Determination of Anti-cIL-4 and Anti-cIL-13 Titers in the cIL-31-cIL-13-cIL-4-Polyprotein-Immunized Dogs According to the Invention

The dog sera obtained from three dogs immunized with cIL-31-cIL-13-cIL-4-polyprotein (see Example 13i) were tested for the presence anti-cIL-13 antibodies based on the same ELISA format as described in Example 14a for anti-cIL-31 antibodies. The only exception was that the 384-well polystyrene ELISA plates were coated with 1 μg/ml cIL-4 (ex Genscript cIL-4, U1119GH110-3 0.84 mg/ml), or cIL-13 (ex Genscript U842WEG100-1, cIL-13, 0.15 mg/ml) or cIL-31-poly (ex Genscript U935DEG100-5, 0.29 mg/ml), dissolved in the coating buffer.

FIG. 88 depicts the results of the ELISAs using cIL-31 as a coating antigen. All three cIL-31-cIL-13-cIL-4-poly immunized dogs generated high antibody titers against the cIL-31 immunogen component. Titers were already apparent in the primary immunization phase (day SD-1-day SD28).

FIG. 89 depicts the results of the ELISAs using cIL-4 as a coating antigen. All three cIL-31-cIL-13-cIL-4-poly immunized dogs generated antibody titers against the cIL-4 immunogen component. Titers were only weakly present in the primary immunization phase (day SD-1-day SD 28), but became prominently apparent after the booster immunization (day SD 35-day SD 63).

FIG. 90 depicts the results of the ELISAs using cIL-13 as a coating antigen. All three cIL-31-cIL-13-cIL-4-poly immunized dogs generated antibody titers against the cIL-13 immunogen component. Titers were only weakly present in the primary immunization phase (day SD-1-day SD 28), but became prominently apparent after the booster immunization (day SD 35-day SD 63).

### Example 15: Test for Competition of the Polyclonal Dog Antibodies with Biotinylated Lokivetmab to Analyze the Presence of cIL-31 Neutralizing Antibodies in the Dog Sera

The ELISA assay was performed as described in Example 11, but using a mixture of dog sera in 100 ng/ml biotinylated lokivetmab in PBS.

FIGS. 18 to 23 depict the results of the ELISA using cIL-31 as antigen for the mixture comprising dog serum from day 42 after immunization and 100 ng/ml biotinylated lokivetmab. While in FIGS. 18, 20 and 22 the unit of the response is “mOD405/min” and thus the direct readout from the reporter response, FIGS. 19, 21 and 23 use as unit of the response “% lokivetmab binding” which was calculated based on the hightest readout of “mOD405/min” for the preimmune serum of day 0.

FIGS. 24 to 29 depict the results of the ELISA using cIL-31 polyprotein as antigen for the mixture comprising dog serum from day 42 after immunization and 100 ng/ml biotinylated lokivetmab. While in FIGS. 24, 26 and 28 the unit of the response is “mOD405/min” and thus the direct readout from the reporter response, FIGS. 25, 27 and 29 use as unit of the response “% lokivetmab binding” which was calculated based on the hightest readout of “mOD405/min” for the preimmune serum of day 0.

The preimmune serum of dogs showed some inhibitory activity against lokivetmab binding to cIL-31, but this effect was much weaker compared to the inhibitory activity of the dog serum obtained 42 days after immunization and was limited to 40-70% at a 1:2 dilution. It appears likely that this reflects the presence of cIL-31 cytokine autoantibodies in dogs, as described previously in humans for a variety of cytokines such as interferon alpha and gamma, tumor necrosis factor alpha, interleukins 1 beta and 10, and others (Bendtzen et al., “High-avidity autoantibodies to cytokines”, Immunology today 19.5 (1998): 209-211).

The neutralizing activity, however, was greatly increased by immunization with cIL-31 polyprotein. Serum samples from day 42 of all three dogs, immunized according to the regimen described in Example 13, contained antibodies that suppressed lokivetmab binding to cIL-31 by 95% when diluted 1:2 (see FIGS. 21 to 26). This inhibition became less pronounced when the serum samples were diluted further (titration), but at 1:16 dilutions a partial effect was still visible in all three dogs.

Similar results were obtained when cIL-31 poly was used as a lokivetmab binding agent (FIGS. 24 to 29).

Since lokivetmab is a cIL-31 neutralizing antibody, the successful induction of lokivetmab-competing autoantibodies with the described cIL-31 polyprotein immunization scheme indicates a clear potential that the autoantibodies can neutralize cIL-31 function in dogs.

As the therapeutic action of lokivetmab is well-established, it can be expected that the lokivetmab-competing autoantibodies induced in the host according to the invention will show similar therapeutic efficacy.

### Example 16: Second Immunization Study Using the Vaccine Formulation of Example 13

In a second immunization study dogs suffering from pruritus were immunized by subcutaneous injections with the vaccine formulation according to Example 13 as follows:
Day −7: challenge with cIL-31 (1.75 μg/kg bw intravenous injection for baseline evaluation)
Day 0: primary subcutaneous immunization with the cIL-31 polyprotein vaccine as defined in Example 13. Before the primary immunization, a preimmune blood sample was taken from all tested dogs.
Day 6: collection of a blood sample
Day 14: collection of a blood sample
Day 21: collection of a blood sample and challenge with cIL-31 (1.75 μg/kg intravenous injection cIL-31)
Day 28: first subcutaneous booster immunization with the cIL-31 polyprotein vaccine and collection of a blood sample
Day 35: collection of a blood sample
Day 42: challenge with cIL-31 (1.75 μg/kg intravenous injection cIL-31) and collection of a blood sample
Day 49: collection of a blood sample
Day 56: collection of a blood sample
Day 63: challenge with cIL-31 (0.85 μg/kg intravenous injection cIL-31) and collection of a blood sample
Day 70: collection of a bood sample
Day 77: collection of blood sample
Day 84: second subcutaneous booster immunization with the cIL-31 polyprotein vaccine and collection of blood sample
Day 91: collection of blood sample
Day 98: challenge with cIL-31 (0.85 μg/kg intravenous injection cIL-31) and collection of blood sample
Day 105: collection of blood sample
Day 112: collection of blood sample
Day 119: collection of blood sample
Day 126: collection of blood sample

During the booster immunizations on days 28 and 84, the tested dogs received again the vaccine formulation according to Example 13.

The serum samples were analyzed with an ELISA assay as described in Example 14 using cIL-31 as coating antigen. Serial dilutions of the dog sera from 1:20 to 1:20480 were used. FIGS. 30a-b to 37a-b depict the results of these ELISAs and show that the immunized dogs produced anti-cIL-31 antibodies in considerable amounts. The booster immunizations at day 28 led to an increase in anti-cIL-31 antibody titers in the tested dogs. High anti-cIL-31 antibody titers were still observed beyond day 42. Only a partial loss of anti-cIL-31 antibody titers occurred in the tested dogs up to the second booster immunization. Taken together, these results show the successful breakage of self-tolerance against cIL-31 in the tested dogs.

The dogs subjected to the above-described immunization scheme were analyzed for the presence of any pruritic behavior as described in Example 4 at different time points of the immunization scheme: before the begin of immunization study (day-7), three weeks after the first immunization (day 21), two weeks after the first boost (day 42), five weeks after the first boost (day 63) and two weeks after the second boost (day 98). Exemplary results of pruritic behavior analysis from three tested dogs are depicted in FIG. 38. The results of FIG. 38 demonstrate that the pruritus scores can be decreased by the applied immunization scheme and thus provide proof-of-concept for a successful prophylactic/therapeutic vaccine treatment using a vaccine composition according to the invention.

### Example 17: Vaccine Formulation Using mRNA to Encode the Polyprotein According to the Invention

In recent years vaccination with mRNA has gained increasing attention due to the fact that the production of mRNA vaccines is rather simple. For such vaccines, the steps of expressing and purifying the protein antigen are no longer necessary, which are often bottlenecks in terms of cost and speed of vaccine production (see Zhang C, Maruggi G, Shan H, Li J. Advances in mRNA Vaccines for Infectious Diseases. Front Immunol. 2019 Mar. 27; 10:594. doi: 10.3389/fimmu.2019.00594. eCollection 2019.). mRNA vaccines rely on the production of the antigen by the host's own cells based on the mRNA introduced.

It is conceivable that the vaccination of dogs against its own IL-31 can also be achieved with a vaccine containing an mRNA encoding for a polyprotein comprising at least two segments of a cIL-31. This could be achieved as follows:

The cIL-31-poly construct described in Example 1 is transferred into an in vitro transcription vector. To this aim, the insert encoding cIL-31-poly is cloned from pcDNA3.4 into pcDNA3.1 (+) using the restriction enzymes EcoRI and HindIII. pcDNA3.1 (+) possesses a T7 RNA polymerase promoter upstream the EcoRI cloning site. To make the production of capped run-off RNA transcripts from this vector possible, the pcDNA3.1 (+)-cIL-31-poly vector is linearized 3′ of the insert. In vitro transcription is then performed using T7-RNA polymerase in the presence of cap nucleotides, the four canonical dNTP and/or noncanonical dNTPs to modify transcript properties. In the next step, the obtained run-off transcripts are polyadenylated by using a Poly-A polymerase. Alternatively, it is also possible to include a poly-dT tail in the cIL-31-poly construct 3′ of the stop codon.

The capped and polyadenylated mRNA can then be used for vaccination. Dogs could receive mRNA amounts of 1 μg to 1 mg, more preferably 10 μg to 300 μg, e.g., by injecting naked mRNA together with the remaining components of the claimed vaccine composition intramuscularly, subcutaneously or interadermally or with a gene gun. It is also possible to inject the mRNA encoding for the polyprotein together with the remaining components of the claimed vaccine composition in form of a liposomal formulation, in form of a formulation comprising complexes with cationic proteins, cationic polymers or cationic cell penetrating peptides or in other forms which enhance halflife, cellular upatake and translatability of the introduced mRNA.

The mRNA injection is then repeated in 2-6 week intervals. The presence of anti-cIL-31 antibodies can be assessed as described in Example 14.

Generally, it is also possible to encode the cIL-31-poly construct by a self-replicating mRNA, e.g., by an alpha virus derived self-replicating mRNA. The use of self-replicating mRNA could have the advantage that a longer protein production is achieved from the RNA construct upon administration to the host so that also higher anti-cIL-31 antibodies titers could be obtained.

The same can be achieved for the cIL-13-cIL-4-poly and cIL-31-cIL-13-cIL-4-poly constructs according to the invention in the same manner.

### Example 18: Vaccine Formulation Using DNA to Encode Polyproteins

DNA vaccines contain DNA, usually plasmid DNA. Plasmid DNA is often administered to the host in naked form via injection or gene gun. It is however, also possible to deliver the plasmid DNA to the host as, e.g., lipoplex with cationic lipids, as liposomal formulation or as complex with cationic polymers. Sometimes the DNA is also encapsulated in a protein shell akin to a virus, which ensures efficient uptake by cells. (for review: Ghaffarifar F. Plasmid DNA vaccines: where are we now? Drugs Today (Barc). 2018 May; 54 (5): 315-333. doi: 10.1358/dot.2018.54.5.2807864).

It is conceivable that the vaccination of dogs against one of its own self-proteins, e.g. against a cytokine, in particular an interleukin preferably derived from IL-31 can also be achieved with a vaccine containing DNA encoding for a polyprotein comprising at least two segments of a cIL-31.

As an example, it is conceivable that the vaccination of dogs against its own IL-31 can also be achieved with a vaccine containing DNA encoding for a polyprotein comprising at least two segments of a cIL-31. This could be achieved as follows:

The cIL-31-poly construct described in Example 1a is transferred into an mammalian expression vector. To this aim, the insert encoding cIL-31-poly is cloned from pcDNA3.4 into pcDNA3.1 (+) using the restriction enzymes EcoRI and HindIII. pcDNA3.1 (+) possesses all elements necessary for expression of cIL-31-poly in the host cells: a strong mammalian promoter in form of a human cytomegalovirus immediate-early (CMV) promoter and a strong polyadenylation/termination signal in form of the bovine growth hormone BGH gene.

Dogs could receive highly purified pcDNA3.1 (+)-cIL-31-poly (LPS-free) in amounts of 10 μg to 3 mg, more preferably 50 μg to 1000 μg, e.g., by injecting naked plasmid DNA together with the remaining components of the claimed vaccine composition intramuscularly, subcutaneously or interadermally or with a gene gun. It is also possible to inject the plasmid DNA encoding for the polyprotein together with the remaining components of the claimed vaccine composition in form of a liposomal formulation, in form of a formulation comprising complexes with cationic proteins, cationic polymers or cationic cell penetrating peptides or in other forms which enhance halflife, cellular upatake and translatability of the introduced mRNA.

The plasmid DNA injection is then repeated in 2-6 week intervals. The presence of anti-cIL-31 antibodies can be assessed as described in Example 14a.

Analgous protocols are envisioned for the other cytokines, in particular for those exemplified herein (e.g. IL-4, IL-5, IL-13, IL-33-CS, and TNF-alpha), in particular when comprised in the polyprotein constructs according to the invention. These protocols are specifically envisioned for the cIL-13-cIL-4-poly and cIL-31-cIL-13-cIL-4-poly constructs according to the invention.

### Example 19a: Neutralization Assay of cIL-13 Signal Transduction Using a Specific Rabbit Anti-cIL-13 Serum

HEKBlue IL4/IL13 (Invivogen, hek-il413) were grown in DMEM (Thermo Fisher, 616965-026), 10% iFCS, at 37° C., 5% CO₂. For selection purposes, culture medium was supplemented with 10 μg/ml blasticidin (Invivogen, ant-bl-5b) and 100 μg/ml zeocin (Invivogen, ant-zn-5).

Dilution of rabbit anti-cIL-13 serum (Example 6a) was performed in 40 μl full growth medium in a 384 well cell culture plate supplemented with 10 ng/ml cIL-13 and incubated for 1 h. HEKBlue IL4/IL13 cells were harvested, adjusted to 2.2×10⁵ cells/mL in full growth medium, and 40 μl cell suspension were added to each of the sera dilutions. The cells were incubated at 37º° C., 5% (v/v) CO₂ for 96 h. Cell culture supernatant samples were used for measurement of alkaline phosphatase (SEAP) activity by adding them to 5 mM para-nitrophenyl phosphate (pNPP) in AP buffer (50 mM NaHCO₃/Na₂CO₃, 2 mM MgCl₂, pH 9.6), 90 μl/well for 384 well plates. Measurement of the optical density (OD) at 405 nm at RT in an ELISA reader in a kinetic mode (mOD/min) by determining the curve slope in a linear area (=mOD405 nm/min), or in an endpoint mode (OD) by determining the optical density at a fixed time point.

The ELISA results of the rabbit cIL-13 antiserum's neutralization effect on HEKBlue IL4/IL13 cells are depicted in FIG. 55. The rabbit cIL-13 antiserum inhibited the cIL-13 stimulation of HEKBlue IL-4/IL-13 cells, already starting at a dilution of 1:320.

Complete inhibition was achieved at a dilution of 1:20. This experiment established an antibody neutralization assay of cIL-13's biological effect.

### Example 19b: Neutralization Assay of cIL-13 Signal Transduction Using Preimmune and Anti-cIL-13 Dog Sera

The same neutralization assay of Example 19a was performed on sera from three dogs (Dogs 0521, 2579, and 6048) collected on Day 0 (preimmune, SD-1), and on Day 42 of the cIL-13-poly vaccination study of Example 14c. The ELISA results are depicted in FIG. 56A-C.

While dilutions of preimmune sera (“SD-1”) did not lead to a decrease in cIL-13 signal strength in HEKBlue IL-4/IL-13 cells, Day 42 cIL-13-poly antisera from all three dogs led to a complete suppression of the signal at 1:320, 1:20 and 1:40 dilutions, respectively, and titration curves of inhibition following these dilutions (see FIGS. 56A-C).

### Example 19c: Neutralization Assay of cIL-4 Signal Transduction Using Preimmune and Anti-cIL-4 Dog Sera

IL-4 is known to induce expression of thymus1- and activation-regulated chemokine (TARC or CCL17) at the mRNA and protein level and in dogs an upregulation of TARC in atopic dermatitis has been documented. Therefore, TARC was used as a positive marker (strong mRNA upregulation) of canine blood exposure to IL-4.

The inhibition of TARC mRNA upregulation was assessed to probe for the presence of neutralizing antibodies following the cIL-4-poly vaccination as follows: EDTA-stabilized blood samples were taken from the IL-4-poly-immunized animals (Dog 5365, Dog 6523 and Dog 7104) at day 49 (2 weeks into the booster immunization) and blood samples were pooled from control animals. 500 μl of blood was supplemented with cIL-4 (R&D systems, 754-CL-025/CF) to 1 ng/ml, and the blood was then incubated for 6 h at 35° C., 5% CO₂ and 96% relative humidity. Blood lysis and RNA stabilization was done with RNAprotect Animal Blood Tubes 500 μl (Qiagen 76554) and an incubation for 2 h at room temperature.

RNA was then isolated using the RNeasy Protect Animal Blood Kit (Qiagen 73224). Isolated RNA was analysed and quantified using an Implen NanoPhotometer®, type NP80. Quantitative RT-PCR (qPCR) was performed with the TaqMan® Assay Cf02622128_m1 (Thermo) with probe and primers for canine CCL-17 (TARC) and the QuantiNova Probe RT-PCR Kit (Qiagen 208354), with the primers, reaction mixtures and conditions outlined by the manufacturers. Homemade canine B-actin TaqMan probe and PCR primers were used as a housekeeping gene control. Typically 25 ng total RNA was used as template. The qPCR was performed using a CFX96 Real-Time System (BioRad).

Results of the experiments are depicted in FIG. 73.

The pooled blood of control dogs was highly responsive to the incubation with 1 ng/ml cIL-4 with respect to TARC/CCL-17 mRNA induction (see ΔΔCq in FIG. 73A). By contrast, all three dogs vaccinated with cIL-4-poly had a strongly suppressed TARC/CCL-17 mRNA induction in cIL-4 incubation, an inhibition apparently complete for all three dogs in a linear scale graph (FIG. 73A). In a log scale graph, inhibition of cIL-4-induced TARC mRNA production exceeds 99.99% for dogs 5365 and 7104, and 99% for dog 6523 (FIG. 73B). Some minor background mRNA expression of TARC in the absence of cIL-4 stimulation was detected in all dogs (FIG. 73B, condition “w/o”).

Taken together, the data demonstrate that self tolerance to cIL-4 was broken in all three dogs immunized with cIL-4-poly vaccine, and antibody titers against native cIL-4 were present. These antibodies possess neutralizing potential in cell culture assay systems for cIL-4 action, and are neutralizing to cIL-4 added in an ex vivo blood assay of the immunized dogs.

### Example 19d: Neutralization Assay of cIL-4 and cIL-13 Signal Transduction Using Preimmune and Anti-cIL-13-cIL-4-Polyprotein Dog Sera According to the Invention

The inhibition of TARC mRNA upregulation was assessed to probe for the presence of neutralizing antibodies against either cIL-4 (1 ng/mL) or cIL-13 (1 ng/mL) following the cIL-13-cIL-4-polyprotein vaccination, in the same manner as described for cIL-4-poly-vaccinated dogs and cIL-4 in Example 19c.

Results of the experiments are depicted in FIG. 83. FIG. 83A depicts the results on a linear scale, FIG. 83B depicts the same results on a logarithmic scale for better visualization. The figures depict the ΔΔCq values for TARC/CCL-17 mRNA induction, where the x-axis defines the sample's (i.e. dog's) identity (e.g. 6504, 8322, or 6043, or pooled naïve blood) and the stimulating protein (“+IL4″=stimulated with cIL-4; “+IL13” =stimulated with cIL-13; “w/o”=indicates blood samples incubated and processed in the same way, but having not received cIL-4 or cIL-13, control value).

The pooled blood of control dogs was highly responsive to the incubation with either cIL-4 or cIL-13 with respect to TARC/CCL-17 mRNA induction (see ΔΔCq in FIG. 83A, “+IL4” and “+IL33”). By contrast, all three dogs vaccinated with cIL-13-cIL-4-polyprotein had a strongly suppressed TARC/CCL-17 mRNA induction in cIL-4 and cIL-13 incubation, an inhibition apparently complete for all three dogs in a linear scale graph (FIG. 83A, 6504, 8322, and 6043 in conditions “+IL4” and “+IL13”). In a log scale graph (FIG. 83B), inhibition of cIL-4- or cIL-13-induced TARC mRNA production exceeds 99.9% for dogs 8022 and 6043 for both cytokines, and for dog 6504 99.9% for cIL-4 and >99% for cIL-13. Some minor background mRNA expression of TARC in the absence of cIL-4 and cIL-13 was detected in all dogs (FIG. 83B, “w/o”).

Taken together, the data demonstrate that using the vaccine construct cIL-13-cIL-4-polyprotein, self tolerance to both cIL-4 and cIL-13 was broken in all three dogs immunized in this study, and high antibody titers against native cIL-4 and native cIL-13 were present. These canine self-antibodies have also been observed to possess neutralizing potential in a dog monocyte cell culture assay systems against both cIL-4 and cIL-13 action (data not shown), and here are shown to mediate neutralization of both added cIL-4 and added cIL-13 in an ex vivo blood assay with blood samples of the immunized dogs.

### Example 19e: Neutralization Assay of cIL-4 and cIL-13 Signal Transduction Using Preimmune and Anti-cIL-31-cIL-13-cIL4Polyprotein Dog Sera According to the Invention

The inhibition of TARC mRNA upregulation was assessed to probe for the presence of neutralizing antibodies against either cIL-4 (1 ng/mL) or cIL-13 (1 ng/mL) following the cIL-31-cIL-13-cIL-4-polyprotein vaccination, in the same manner as described for cIL-4-poly-vaccinated dogs and cIL-4 in Example 19c.

Results of the experiments are depicted in FIG. 91. FIG. 91A depicts the results on a linear scale, FIG. 91B depicts the same results on a logarithmic (log 10) scale for better visualization. The figures depict the ΔΔCq values for TARC/CCL-17 mRNA induction, where the x-axis defines the sample's (i.e. dog's) identity (e.g. 0720, 6731, or 9214, or pooled naïve blood) and the stimulating protein (“+IL4″=stimulated with cIL-4; “+IL13” =stimulated with cIL-13; “w/o”=indicates blood samples incubated and processed in the same way, but having not received cIL-4 or cIL-13, control value).

The pooled blood of control dogs was highly responsive to the incubation with either cIL-4 or cIL-13 with respect to TARC/CCL-17 mRNA induction. By contrast, all three dogs vaccinated with cIL-31-cIL-13-cIL-4-poly had a strongly suppressed TARC/CCL-17 mRNA induction in cIL-4 and in cIL-13 incubations, an inhibition apparently complete for all three dogs in a linear scale graph (FIG. 91A). In a log scale graph, inhibition of cIL-4- or cIL-13-induced TARC mRNA production exceeds 99% for all dogs for both cytokines (FIG. 91B). Some minor background mRNA expression of TARC in the absence of cIL-4 and cIL-13 was detected in all dogs (FIG. 91B, samples “w/o”).

Taken together, the data demonstrate that using the vaccine construct cIl-31-cIL-14-cIL-4-poly, self tolerance to canine IL-31, canine IL-4 and canine IL-13 was broken in all three dogs immunized in this study, and high antibody titers against native cIL-31, native cIL-4 and native cIL-13 were present. These canine self-antibodies possess neutralizing potential in a dog monocyte cell culture assay systems against both cIL-4 and cIL-13 action (data not shown), and mediate neutralization of both added cIL-4 and added cIL-13 also in an ex vivo blood assay with blood samples of the immunized dogs.

### BRIEF DESCRIPTION OF THE DRAWINGS

FIG. 1 depicts an embodiment of the polyprotein according to the invention. The N-terminus of the polyprotein begins with an artificial signal sequence for ER-import to allow expression in HEK 293 cells. This is followed by a first copy of mature canine IL-31 (SEQ ID NO: 3). After this first copy of mature canine IL-31, the Tetanus toxin p2 T-cell epitope (amino acids 1273-1284 of the tetanus toxin, SEQ ID NO: 1) is included, followed by a second copy of mature canine IL-31. After this second copy of mature canine IL-31, the Tetanus toxin p30 T-cell epitope (amino acids 947-968 of the tetanus toxin, SEQ ID NO: 2) is attached, followed by a third copy of mature canine IL-31. Therafter, two Tetanus toxin T-cell epitopes (p30 and p2) are included. These two Tetanus toxin T-cell epitopes are followed by a His-tag for protein purification.

FIG. 2 depicts a plasmid map of the vector pcDNA3.4-cIL31-poly which encodes the cIL-31 polyprotein according to FIG. 1.

FIG. 3 depicts the results of the SDS-PAGE analysis with Coomassie Blue staining of cIL-31-poly, which are further explained in Example 2. Lane M1 depcits the protein marker (TaKaRa, Cat. No. 3452). Lane 1 depicts the size of the cIL-31 polyprotein under reducing conditions and lane 2 under non-reducing conditions.

FIG. 4 depicts the results of the SDS-PAGE analysis with Coomassie Blue staining of cIL-31, which are further explained in Example 3. Lane M1 depcits the protein marker (TaKaRa, Cat. No. 3452). Lane 1 depicts the size of the cIL-31 protein under reducing conditions and lane 2 under non-reducing conditions.

FIG. 5 depicts the results of the ELISA using cIL-31 or cIL-31 polyprotein as ELISA plate coating antigen for the rabbit preimmune serum and antiserum raised against cIL-31. These results are further explained in Example 8.

FIG. 6 depicts the results of the ELISA using cIL-31 or cIL-31 polyprotein as ELISA plate coating antigen for the chicken egg yolk preparation raised against cIL-31. These results are further explained in Example 8.

FIG. 7 depicts the results of the ELISA using cIL-31 as ELISA plate coating antigen for lokivetmab which are further explained in Example 9.

FIG. 8 depicts the results of the ELISA using cIL-31 and cIL-31 polyprotein as ELISA plate coating antigens for lokivetmab. These results are further explained in Example 9.

FIG. 9 depicts the results of the ELISA using cIL-31 as ELISA plate coating antigen for biotinylated lokivetmab. These results are further explained in Example 10.

FIG. 10 depicts the results of the ELISA using cIL-31 as ELISA plate coating antigen for the mixture of rabbit preimmune serum+biotinylated lokivetmab or the mixture of rabbit immune serum+biotinylated lokivetmab to probe for the interaction between biotinylated lokivetmab as cIL-31 neutralizing antibody and cIL-31 immobilized on an ELISA plate. These results are further explained in Example 11.

FIG. 11 depicts the results of the ELISA using cIL-31 polyprotein as ELISA plate coating antigen for the mixture of rabbit preimmune serum+biotinylated lokivetmab or the mixture of rabbit immune serum+biotinylated lokivetmab to probe for the interaction between biotinylated lokivetmab as cIL-31 neutralizing antibody and cIL-31 polyprotein immobilized on an ELISA plate. These results are further explained in Example 11.

FIG. 12 depicts the results of the ELISA using cIL-31 as ELISA plate coating antigen for the mixture of chicken egg yolk IgY preparation+biotinylated lokivetmab to probe for the interaction between lokivetmab as cIL-31 neutralizing antibody and cIL-31 immobilized on an ELISA plate. These results are further explained in Example 11.

FIG. 13 depicts a plasmid map of the construct pcDNA3.1 (+)-bsd-NFkB-SEAP in which
181 bp-448 bp encode for five NFkB binding sites followed by a minimal ELAM promoter (NFkB-5-ELAM)
454 bp-2022 bp encode for the SEAP gene as NFkB reporter gene
3214 bp-3613 bp encode for the Blasticidin resistance gene (bsd-R) to allow for eukaryotic cell selection
5961 bp-5100 bp encode for the Ampicillin resistance gene (amp-R) to allow for E. coli selection

FIG. 14 depicts the results of NFkB signal activation upon treatment of the clonal cell line DH82-bsd-NFSEAP with PTO-ODNs. These results are further described in Example 12.

FIGS. 15a-c depict the results of the ELISA using cIL-31 as ELISA plate coating antigen for dog sera of animal 4315 at different sampling time points. These results are further explained in Example 14.

FIGS. 16a-c depict the results of the ELISA using cIL-31 as ELISA plate coating antigen for dog sera of animal 6962 at different sampling time points. These results are further explained in Example 14.

FIGS. 17a-c depict the results of the ELISA using cIL-31 as ELISA plate coating antigen for dog sera of animal 8523 at different sampling time points. These results are further explained in Example 14.

FIG. 18 depicts the results of the ELISA using cIL-31 as ELISA plate coating antigen for the mixture comprising serum from day 42 after immunization of dog 4315 and 100 ng/ml biotinylated lokivetmab. The the unit of the response is indicated as “mOD405/min” and thus the direct readout from the reporter response. These results are further explained in Example 15.

FIG. 19 depicts the results of the ELISA using cIL-31 as ELISA plate coating antigen for the mixture comprising serum from day 42 after immunization of dog 4315 and 100 ng/ml biotinylated lokivetmab. The the unit of the response is indicated as “% lokivetmab binding” which was calculated based on the hightest readout of “mOD405/min” for the preiummune serum of day 0. These results are further explained in Example 15.

FIG. 20 depicts the results of the ELISA using cIL-31 as ELISA plate coating antigen for the mixture comprising serum from day 42 after immunization of dog 6962 and 100 ng/ml biotinylated lokivetmab. The the unit of the response is indicated as “mOD405/min” and thus the direct readout from the reporter response. These results are further explained in Example 15.

FIG. 21 depicts the results of the ELISA using cIL-31 as ELISA plate coating antigen for the mixture comprising serum from day 42 after immunization of dog 6962 and 100 ng/ml biotinylated lokivetmab. The the unit of the response is indicated as “% lokivetmab binding” which was calculated based on the hightest readout of “mOD405/min” for the preimmune serum of day 0. These results are further explained in Example 15.

FIG. 22 depicts the results of the ELISA using cIL-31 as ELISA plate coating antigen for the mixture comprising serum from day 42 after immunization of dog 8523 and 100 ng/ml biotinylated lokivetmab. The the unit of the response is indicated as “mOD405/min” and thus the direct readout from the reporter response. These results are further explained in Example 15.

FIG. 23 depicts the results of the ELISA using cIL-31 as ELISA plate coating antigen for the mixture comprising serum from day 42 after immunization of dog 8523 and 100 ng/ml biotinylated lokivetmab. The the unit of the response is indicated as “% lokivetmab binding” which was calculated based on the hightest readout of “mOD405/min” for the preiummune serum of day 0. These results are further explained in Example 15.

FIG. 24 depicts the results of the ELISA using cIL-31 polyprotein as ELISA plate coating antigen for the mixture comprising serum from day 42 after immunization of dog 4315 and 100 ng/ml biotinylated lokivetmab. The the unit of the response is indicated as “mOD405/min” and thus the direct readout from the reporter response. These results are further explained in Example 15.

FIG. 25 depicts the results of the ELISA using cIL-31 polyprotein as ELISA plate coating antigen for the mixture comprising serum from day 42 after immunization of dog 4315 and 100 ng/ml biotinylated lokivetmab. The the unit of the response is indicated as “% lokivetmab binding” which was calculated based on the hightest readout of “mOD405/min” for the preimmune serum of day 0. These results are further explained in Example 15.

FIG. 26 depicts the results of the ELISA using cIL-31 polyprotein as ELISA plate coating antigen for the mixture comprising serum from day 42 after immunization of dog 6962 and 100 ng/ml biotinylated lokivetmab. The the unit of the response is indicated as “mOD405/min” and thus the direct readout from the reporter response. These results are further explained in Example 15.

FIG. 27 depicts the results of the ELISA using cIL-31 polyprotein as ELISA plate coating antigen for the mixture comprising serum from day 42 after immunization of dog 6962 and 100 ng/ml biotinylated lokivetmab. The the unit of the response is indicated as “% lokivetmab binding” which was calculated based on the hightest readout of “mOD405/min” for the preimmune serum of day 0. These results are further explained in Example 15.

FIG. 28 depicts the results of the ELISA using cIL-31 polyprotein as ELISA plate coating antigen for the mixture comprising serum from day 42 after immunization of dog 8523 and 100 ng/ml biotinylated lokivetmab. The the unit of the response is indicated as “mOD405/min” and thus the direct readout from the reporter response. These results are further explained in Example 15.

FIG. 29 depicts the results of the ELISA using cIL-31 polyprotein as ELISA plate coating antigen for the mixture comprising serum from day 42 after immunization of dog 8523 and 100 ng/ml biotinylated lokivetmab. The the unit of the response is indicated as “% lokivetmab binding” which was calculated based on the hightest readout of “mOD405/min” for the preimmune serum of day 0. These results are further explained in Example 15.

FIGS. 30a-b depict the results of the ELISA using cIL-31 as ELISA plate coating antigen for dog sera of animal 1672 at different sampling time points. These results are further explained in Example 16.

FIGS. 31a-b depict the results of the ELISA using cIL-31 as ELISA plate coating antigen for dog sera of animal 5096 at different sampling time points. These results are further explained in Example 16.

FIGS. 32a-b depict the results of the ELISA using cIL-31 as ELISA plate coating antigen for dog sera of animal 5583 at different sampling time points. These results are further explained in Example 16.

FIGS. 33a-b depict the results of the ELISA using cIL-31 as ELISA plate coating antigen for dog sera of animal 7918 at different sampling time points. These results are further explained in Example 16.

FIGS. 34a-b depict the results of the ELISA using cIL-31 as ELISA plate coating antigen for dog sera of animal 9351 at different sampling time points. These results are further explained in Example 16.

FIGS. 35a-b depict the results of the ELISA using cIL-31 as ELISA plate coating antigen for dog sera of animal 8779 at different sampling time points. These results are further explained in Example 16.

FIGS. 36a-b depict the results of the ELISA using cIL-31 as ELISA plate coating antigen for dog sera of animal 1368 at different sampling time points. These results are further explained in Example 16.

FIGS. 37a-b depict the results of the ELISA using cIL-31 as ELISA plate coating antigen for dog sera of animal 3432 at different sampling time points. These results are further explained in Example 16.

FIG. 38 depicts exemplary results of a pruritic behavior analysis of three tested dogs which were subjected to the immunization scheme described in Example 16. The results are further explained in Example 16.

FIG. 39 depicts an embodiment of the polyprotein according to the invention. The N-terminus of the polyprotein begins with an artificial signal sequence for ER-import to allow expression in HEK 293 cells. This is followed by a first copy of mature canine IL-5 (SEQ ID NO: 41). After this first copy of mature canine IL-5, the Tetanus toxin p2 T-cell epitope (amino acids 1273-1284 of the tetanus toxin, SEQ ID NO: 1) is included, followed by a second copy of mature canine IL-5. After this second copy of mature canine IL-5, the Tetanus toxin p30 T-cell epitope (amino acids 947-968 of the tetanus toxin, SEQ ID NO:

2) is attached, followed by a third copy of mature canine IL-5. Therafter, two Tetanus toxin T-cell epitopes (p30 and p2) are included. These two Tetanus toxin T-cell epitopes are followed by a His₆-tag for protein purification.

FIG. 40 depicts a plasmid map of the vector pcDNA3.4-cIL-5-poly, which encodes the cIL-5 polyprotein according to FIG. 39.

FIG. 41 depicts the results of the SDS-PAGE analysis with Coomassie Blue staining of cIL-5-poly, which are further explained in Example 2b. Lane “M2” depicts the protein marker (GenScript, Cat. No. M00521). Lane “R” depicts the size of the cIL-5 polyprotein under reducing conditions and lane “NR” under non-reducing conditions. Lane “P” depicts the multiple-tag protein (GenScript, Cat.No. M0101) as a positive control. The primary antibody was mouse-anti-His₆ mAb (GenScript, Cat.No. A00186).

FIG. 42 depicts a plasmid map of the vector pcDNA3.4-cIL-5, which encodes the cIL-5 protein.

FIG. 43 depicts the results of the SDS-PAGE analysis with Coomassie Blue staining of cIL-5, which are further explained in Example 3b. Lane M1 depicts the protein marker (TaKaRa, Cat. No. 3452). Lane 1 depicts the size of the cIL-5 protein under reducing conditions and lane 2 under non-reducing conditions.

FIG. 44 depicts the titer determination results of the ELISA using cIL-5 as ELISA plate coating antigen for the rabbit preimmune serum and antiserum raised against cIL-5. These results are further explained in Example 8b.

FIG. 45 depicts the results of the ELISA using cIL-5 or cIL-5 polyprotein as ELISA plate coating antigen for the rabbit preimmune serum and antiserum raised against cIL-5. These results are further explained in Example 8b.

FIGS. 46A-F depict the results of the ELISA at different sampling time points using cIL-5 as ELISA plate coating antigen for dog sera of animal “Dog 1” in FIGS. 46A and 46B, of animal “Dog 2” in FIGS. 46C and 46D, and of animal “Dog 3” in FIGS. 46E and 46F. These results are further explained in Example 14b.

FIG. 47 depicts an embodiment of the polyprotein according to the invention. The N-terminus of the polyprotein begins with an artificial signal sequence for ER-import to allow expression in HEK 293 cells. This is followed by a first copy of mature canine IL-13 (SEQ ID NO: 46). After this first copy of mature canine IL-13, the Tetanus toxin p2 T-cell epitope (amino acids 1273-1284 of the tetanus toxin, SEQ ID NO: 1) is included, followed by a second copy of mature canine IL-13. After this second copy of mature canine IL-13, the Tetanus toxin p30 T-cell epitope (amino acids 947-968 of the tetanus toxin, SEQ ID NO: 2) is attached, followed by a third copy of mature canine IL-13. Therafter, two Tetanus toxin T-cell epitopes (p30 and p2) are included. These two Tetanus toxin T-cell epitopes are followed by a His₆-tag for protein purification.

FIG. 48 depicts a plasmid map of the vector pcDNA3.4-cIL-13, which encodes the cIL-13 protein.

FIG. 49 depicts the results of the SDS-PAGE analysis with Coomassie Blue staining of cIL-13-poly, which are further explained in Example 2b. Lane M1 depicts the protein marker (TaKaRa, Cat. No. 3452). Lane 1 depicts the size of the cIL-13 polyprotein under reducing conditions and lane 2 under non-reducing conditions.

FIG. 50 depicts a plasmid map of the vector pET30a-cIL-13, which encodes the cIL-13 protein.

FIG. 51 depicts the results of the SDS-PAGE analysis with Coomassie Blue staining of cIL-13, which are further explained in Example 3c. Lane M1 depicts the protein marker

(TaKaRa, Cat. No. 3452). Lane 1 depicts the size of bovine serum albumin (BSA, 2 μg). Lane 2 depicts the size of the cIL-13 protein (1.86 μg).

FIG. 52 depicts the results of HEKBlue IL-4/IL-13 cell stimulation by cIL-13, based on SEAP reporter gene readout. These results are further described in Example 12b.

FIG. 53 depicts the results of the ELISA using cIL-13 protein (A) or cIL-13 polyprotein (B) as ELISA plate coating antigen for the rabbit preimmune serum (open symbols) and antiserum (closed symbols) raised against cIL-13. These results are further explained in Example 8c.

FIG. 54A-C depict the results of the ELISA at different sampling time points using cIL-13 as ELISA plate coating antigen for dog sera of animal “Dog 0521” (A), animal “Dog 2579” (B), and of animal “Dog 6048” (C). These results are further explained in Example 14c.

FIG. 55 depicts the results cIL-13 stimulation of HEKBlue IL-4/IL-13 cells treated with rabbit anti-cIL-13 serum. These results are further described in Example 19a.

FIG. 56A-C depicts the results cIL-13 stimulation of HEKBlue IL-4/IL-13 cells treated with dog serum collected from Dog 0521 (A), Dog 3579 (B), and Dog 6048 (C) either at Day 0 (preimmune, open symbols, “SD-1”) or Day 42 (closed symbols, “SD42”) following immunization with cIL-13-poly. These results are further described in Example 19b.

FIG. 57 depicts a plasmid map of the vector pET30a(+)-canIL33-WT, which encodes the cIL-33-WT protein.

FIG. 58 depicts the results of the SDS-PAGE analysis with Coomassie Blue staining of cIL-33-WT, which are further explained in Example 3d. Lane M1 depicts the protein marker (TaKaRa, Cat. No. 3452). Lane 1 depicts the size of bovine serum albumin (BSA). Lane 2 depicts the size of the cIL-33-WT protein under reducing conditions.

FIG. 59 depicts the results of the ELISA using cIL-33-WT protein as ELISA plate coating antigen for the rabbit preimmune serum (open symbols) and antiserum (closed symbols) raised against cIL-33-WT. These results are further explained in Example 8d.

FIG. 60 depicts a plasmid map of the vector pET30a-canIL33-CS, which encodes the cIL-33-CS protein.

FIG. 61 depicts the results of the SDS-PAGE analysis with Coomassie Blue staining of cIL-33-CS, which are further explained in Example 3e. Lane M1 depicts the protein marker (TaKaRa, Cat. No. 3452). Lane 1 depicts the size of bovine serum albumin (BSA). Lane 2 depicts the size of the cIL-33-CS protein under reducing conditions.

FIG. 62 depicts the results of HEKBlue IL-4/IL-13 cell stimulation by different forms of cIL-33, based on SEAP reporter gene readout. The different forms used are IL-33-WT-NovoPro (https://novoprolabs.com/p/human-il33-c9orf26-il1f11-nfhev-519146.html; open circles), cIL-33-WT Batch1 (closed triangles), cIL-33-WT Batch2 (closed squares), and cIL-33-CS (closed circles). These results are further described in Example 12c.

FIG. 63 depicts an embodiment of the polyprotein according to the invention. The N-terminus of the polyprotein begins with an artificial signal sequence for ER-import to allow expression in HEK 293 cells. This is followed by a first copy of mature canine IL-33 (meaning cIL-33-CS; SEQ ID NO: 51). After this first copy of mature canine IL-33-CS, the Tetanus toxin p2 T-cell epitope (amino acids 1273-1284 of the tetanus toxin, SEQ ID NO: 1) is included, followed by a second copy of mature canine IL-33-CS. After this second copy of mature canine IL-33-CS, the Tetanus toxin p30 T-cell epitope (amino acids 947-968 of the tetanus toxin, SEQ ID NO: 2) is attached, followed by a third copy of mature canine IL-33-CS. Therafter, two Tetanus toxin T-cell epitopes (p30 and p2) are included. These two Tetanus toxin T-cell epitopes are followed by a His₆-tag for protein purification.

FIG. 64 depicts a plasmid map of the vector pET30a-cIL33-poly, which encodes the cIL-33-CS protein.

FIG. 65 depicts the results of the SDS-PAGE analysis with Coomassie Blue staining of cIL-33-CS-poly (also referred to here as cIL-33-poly), which are further explained in Example 2d. Lane M1 depicts the protein marker (TaKaRa, Cat. No. 3452). Lane 1 depicts the size of the cIL-33-CS polyprotein under reducing conditions and lane 2 under non-reducing conditions.

FIG. 66 depicts the results of the ELISA using cIL-33-CS polyprotein as ELISA plate coating antigen for the rabbit preimmune serum (open symbols) and antiserum (closed symbols) raised against cIL-33-WT. These results are further explained in Example 8d.

FIG. 67 depicts the results of the ELISA at different sampling time points using cIL-33-WT as ELISA plate coating antigen for dog sera of animal “Dog 402”, which was immunized using cIL-33-CS polyprotein. Time points are Preimmune serum (open circles), Day 28 post immunization (open squares), Day 35 post immunization (closed circles), Day 42 post immunization (closed squares); Day 49 post immunization (closed triangles); Day 56 post immunization (closed diamonds); Day 63 post immunization (open triangles). These results are further explained in Example 14d.

FIG. 68 depicts an embodiment of the polyprotein according to the invention (SEQ ID NO: 57). The N-terminus of the polyprotein begins with an artificial signal sequence for ER-import to allow expression in HEK 293 cells. This is followed by a first copy of mature canine IL-4 (SEQ ID NO: 56). After this first copy of mature canine IL-4, the Tetanus toxin p2 T-cell epitope (amino acids 1273-1284 of the tetanus toxin, SEQ ID NO: 1) is included, followed by a second copy of mature canine IL-4. After this second copy of mature canine IL-4, the Tetanus toxin p30 T-cell epitope (amino acids 947-968 of the tetanus toxin, SEQ ID NO: 2) is attached, followed by a third copy of mature canine IL-4. Therafter, two Tetanus toxin T-cell epitopes (p30 and p2) are included. These two Tetanus toxin T-cell epitopes are followed by a His₆-tag for protein purification.

FIG. 69 depicts a plasmid map of the vector pcDNA3.4-cIL-4, which encodes the cIL-4 protein.

FIG. 70 depicts a plasmid map of the vector pET30a-cIL-4, which encodes the cIL-4 protein.

FIG. 71 depicts the results of the ELISA using cIL-4 protein (A) or cIL-4 polyprotein (B) as ELISA plate coating antigen for the rabbit preimmune serum (circles) and antiserum (triangles) raised against cIL-4. The results using cIL-4-polyprotein as the coating antigen for rabbit antiserum raised against cIL-13 (squares) are also shown. These results are further explained in Example 8e.

FIG. 72A-C depict the results of the ELISA at different sampling time points using cIL-4 as ELISA plate coating antigen for dog sera of animal “Dog 5365” (A), animal “Dog 6523” (B), and of animal “Dog 7104” (C). These results are further explained in Example 14e.

FIG. 73 depicts the qPCR results for TARC mRNA expression following cIL-4 stimulation (“+IL4”) of EDTA-stabilized blood taken from the three IL-4-poly-immunized dogs (Dog 5365, Dog 6523 and Dog 7104) at day 49, or of a pooled blood sample from control dogs with no vaccine exposure (“Pooled naïve blood”). ΔΔCq values are given on a linear (A) or log₁₀ scale (B) for better visualization of low values. “w/o” indicates blood samples incubated and processed in the same way, but having not received cIL-4. The results are further described in Example 19c.

FIG. 74 depicts an embodiment of the polyprotein according to the invention. The N-terminus of the polyprotein begins with an artificial signal sequence for ER-import to allow expression in HEK 293 cells. This is followed by a first copy of mature feline IL-31 (meaning fel-IL-31; SEQ ID NO: 61). After this first copy of mature feline IL-31 (SEQ ID NO: 60), the Tetanus toxin p2 T-cell epitope (amino acids 1273-1284 of the tetanus toxin, SEQ ID NO: 1) is included, followed by a second copy of mature feline IL-31. After this second copy of mature feline IL-31, the Tetanus toxin p30 T-cell epitope (amino acids 947-968 of the tetanus toxin, SEQ ID NO: 2) is attached, followed by a third copy of mature feline IL-31. Thereafter, two Tetanus toxin T-cell epitopes (p30 and p2) are included. These two Tetanus toxin T-cell epitopes are followed by a His₆-tag for protein purification.

FIG. 75A-C depicts the results of the ELISA at different sampling time points using fel-IL-31 as ELISA plate coating antigen for cat sera of animals “Cat 3132,” “Cat 0487,” and “Cat 5674”, which were immunized using fel-IL-31 polyprotein. Time points are Day 2 serum (open circles), Day 7 post immunization (open diamonds), Day 14 post immunization (open triangles), Day 21 post immunization (“x”), Day 27 post immunization (star), Day 35 post immunization (closed circles); Day 42 post immunization (closed diamonds), Day 49 post-immunization (closed triangles), Day 56 post-immunization (bold “x”), and Day 63 post-immunization (closed square). These results are further explained in Example 14f.

FIG. 76A-C depicts the results of the ELISA at different sampling time points using fel-IL-31 as ELISA plate coating antigen for cat sera of animals “Cat 3132,” “Cat 0487,” and “Cat 5674”, which were immunized using fel-IL-31 polyprotein. Time points are Days 63, 70, 77, 84, 91, 98, 105, 112, 119, and 126 post-immunization serum (see legend for symbols). These results are further explained in Example 14f.

FIG. 77 depicts an embodiment of the polyprotein double construct cIL-13-cIL-4-poly according to the invention. The N-terminus of the polyprotein begins with an artificial signal sequence for ER-import to allow expression in HEK 293 cells. This is followed by a first copy of mature cIL 13, the tetanus toxin T cell epitope p2, a first copy of mature cIL-4, followed by the tetanus toxin T cell epitope p30, a second copy of mature cIL 13, a second copy of mature cIL-4, followed by two copies of the tetanus toxin T cell epitope p30 and one tetanus toxin T cell epitope p2. The second copy of cIL 13 and the second copy of mature cIL-4 were fused, only separated by a tetraglycine spacer. All the other individual elements were separated by G/S/A-containing tetrapeptide bridges. C-terminally a tag (His₆) for straightforward purification was added.

FIG. 78 depicts a plasmid map of the vector pcDNA3.4-cIL-13-cIL-4-poly which encodes the cIL-13-cIL-4 polyprotein.

FIG. 79 depicts the results of the ELISA using cIL-4-IL-13-polyprotein as the ELISA plate coating antigen for the rabbit preimmune serum (circles) and antiserum raised against cIL-4 (triangles) or against cIL-13 (squares). These results are further explained in Example 8f.

FIG. 80 depicts the results of the ELISA using rabbit anti-cIL-13-cIL-4-polyprotein antiserum (triangles) compared to its corresponding preimmune serum (circles) with (A) cIL-4, (B) cIL-13, or (C) cIL-13-cIL-4-polyprotein as the ELISA plate coating antigen. The results are further explained in Example 8g.

FIG. 81 depicts the results of the ELISA at different sampling time points using cIL4 as ELISA plate coating antigen for dog sera of animals (A) “Dog 8322,” (B) “Dog 6504,” and (C) “Dog 6403”, which were immunized using cIL-13-cIL-4-polyprotein on study days 1 and 35. “SD” represents the study day on which the samples were collected, wherein SD-1 is preimmune serum. These results are further explained in Example 14g.

FIG. 82 depicts the results of the ELISA at different sampling time points using cIL13 as ELISA plate coating antigen for dog sera of animals (A) “Dog 8322,” (B) “Dog 6504,” and (C) “Dog 6403”, which were immunized using cIL-13-cIL-4-polyprotein on study days 1 and 35. “SD” represents the study day on which the samples were collected, wherein SD-1 is preimmune serum. These results are further explained in Example 14h.

FIG. 83 depicts the qPCR results for TARC mRNA expression following cIL-4 (“+IL4”) or cIL-13 (“+IL13”) stimulation of EDTA-stabilized blood taken from the three cIL-13-cIL-4-poly-immunized dogs (Dog 6504, Dog 8322 and Dog 6403) at day 49, or of a pooled blood sample from control dogs with no vaccine exposure (“Pooled naïve blood”). ΔΔCq values are given on a linear (A) or log₁₀ scale (B) for better visualization of low values. “w/o” indicates blood samples incubated and processed in the same way, but having not received cIL-4 or cIL-13. The results are further described in Example 19d.

FIG. 84 depicts an embodiment of the polyprotein triple construct cIL-31-cIL-13-cIL-4-poly according to the invention. The N-terminus of the polyprotein begins with an artificial signal sequence for ER-import to allow expression in HEK 293 cells. This is followed by a first copy of mature cIL-31, followed by a tetanus toxin T cell epitope p30, followed by a first copy of mature cIL13, followed by a tetanus toxin T cell epitope p2, followed by a first copy of mature cIL-4, followed by a tetanus toxin T cell epitope p30, followed by a second copy of mature cIL-31, followed by a tetanus toxin T cell epitope p2, followed by a second copy of mature cIL 13, followed by a tetanus toxin T cell epitope p30, followed by a second copy of mature cIL-4, followed by a tetanus toxin T cell epitope p30 and a tetanus toxin T cell epitope p2. All the individual elements are separated by G/S/A-containing tetrapeptide bridges. C-terminally a tag (His₆) for straightforward purification is added.

FIG. 85 depicts a plasmid map of the vector pcDNA3.4-cIL-31-cIL-13-cIL-4-poly (mislabeled in figure), which encodes the cIL-31-cIL-13-cIL4 polyprotein.

FIG. 86 depicts the results of the ELISA using cIL-31-cIL-13-IL-4-polyprotein (mislabeled in the figure text) as the ELISA plate coating antigen for the rabbit preimmune serum (open circles) and antiserum raised against cIL-4 (closed circles), against cIL-13 (triangles), or against cIL-31 (squares). These results are further explained in Example 8h.

FIG. 87 depicts the results of the ELISA using rabbit anti-cIL-31-cIL-13-cIL-4-polyprotein antiserum with cIL-4 (large circles), cIL-13 (large squares), cIL-31 (diamonds), or cIL-31-cIL-13-cIL-4 polyprotein (large triangles), as the ELISA plate coating antigen, compared to its corresponding preimmune serum with the same coating antigens (all small circles). The results are further explained in Example 8h.

FIG. 88 depicts the results of the ELISA at different sampling time points using cIL-31 as ELISA plate coating antigen for dog sera of animals (A) “Dog 0720,” (B) “Dog 6731,” and (C) “Dog 9214”, which were immunized using cIL-31-cIL-13-cIL-4-polyprotein on study days 1 and 28. “SD” represents the study day on which the samples were collected, wherein SD-1 is preimmune serum. These results are further explained in Example 14i.

FIG. 89 depicts the results of the ELISA at different sampling time points using cIL-4 as ELISA plate coating antigen for dog sera of animals (A) “Dog 0720,” (B) “Dog 6731,” and (C) “Dog 9214”, which were immunized using cIL-31-cIL-13-cIL-4-polyprotein on study days 1 and 28. “SD” represents the study day on which the samples were collected, wherein SD-1 is preimmune serum. These results are further explained in Example 14i.

FIG. 90 depicts the results of the ELISA at different sampling time points using cIL-13 as ELISA plate coating antigen for dog sera of animals (A) “Dog 0720,” (B) “Dog 6731,” and (C) “Dog 9214”, which were immunized using cIL-31-cIL-13-cIL-4-polyprotein on study days 1 and 28. “SD” represents the study day on which the samples were collected, wherein SD-1 is preimmune serum. These results are further explained in Example 14i.

FIG. 91 depicts the qPCR results for TARC mRNA expression following cIL-4 (“+IL4”) or cIL-13 (“+IL13”) stimulation of EDTA-stabilized blood taken from the three cIL-31-cIL-13-cIL-4-poly-immunized dogs (Dog 0720, Dog 6731 and Dog 9214) at day 49, or of a pooled blood sample from control dogs with no vaccine exposure (“Pooled naïve blood”). ΔΔCq values are given on a linear (A) or log₁₀ scale (B) for better visualization of low values. “w/o” indicates blood samples incubated and processed in the same way, but having not received cIL-4 or cIL-13. The results are further described in Example 19e.

FIG. 92 depicts an embodiment of the polyprotein according to the invention. The N-terminus of the polyprotein begins with a start methionine and the His tag to allow expression in E. coli cells. This is followed by a first copy of mature bovine TNF-alpha (meaning SEQ ID NO: 64). After this first copy of mature bovine TNF-alpha, the Tetanus toxin p30 T-cell epitope (amino acids 947-968 of the tetanus toxin, SEQ ID NO: 2) is included, followed by a second copy of mature bovine TNF-alpha. After this second copy of mature bovine TNF-alpha, the Tetanus toxin p2 T-cell epitope (amino acids 1273-1284 of the tetanus toxin, SEQ ID NO: 1) is attached, followed by a third copy of mature bovine TNF-alpha. Therafter, two Tetanus toxin T-cell epitopes (p30 and p2) are included.

FIG. 93 depicts the results of the ELISA using bovine-TNF-alpha as ELISA plate coating antigen for rabbit preimmune serum (“x”) and antiserum (circles) raised against bovine-TNF-alpha-polyprotein. The results are further explained in Example 8i.

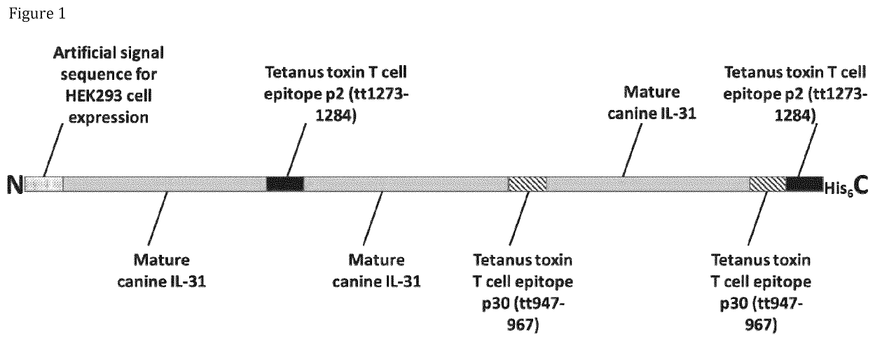

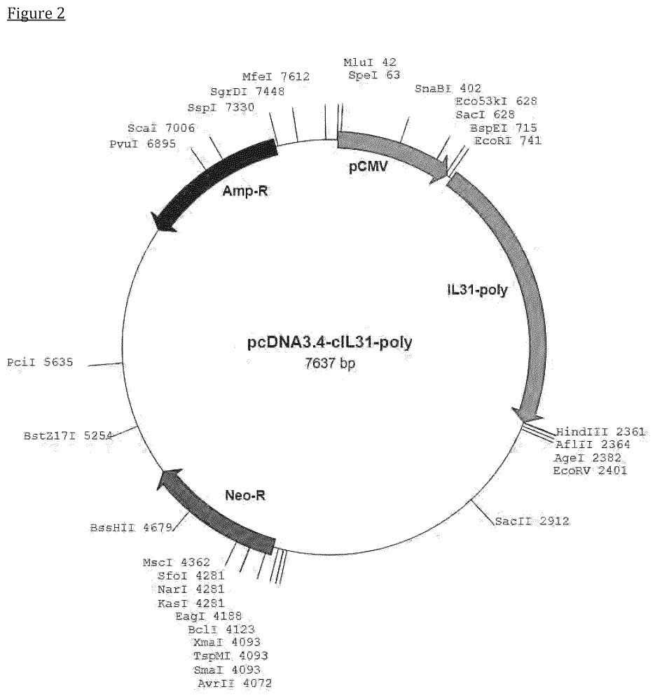

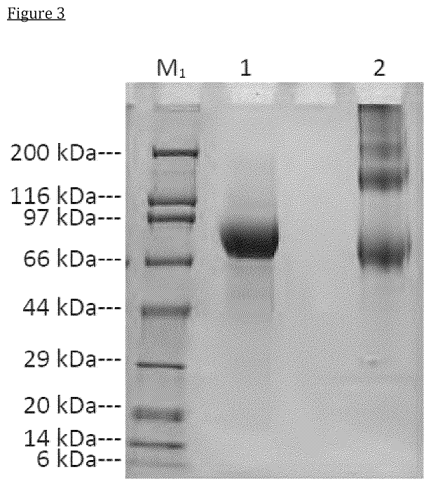

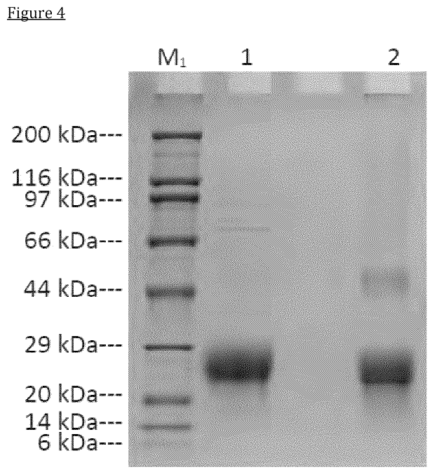

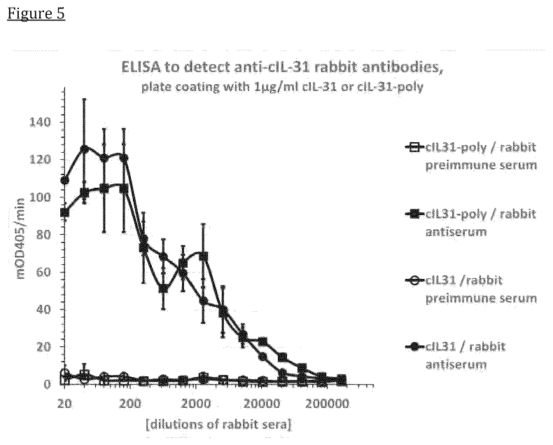

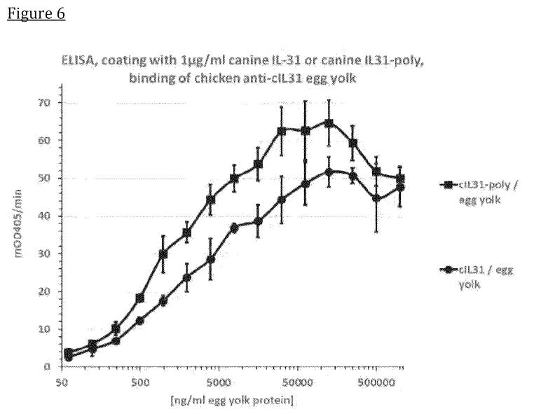

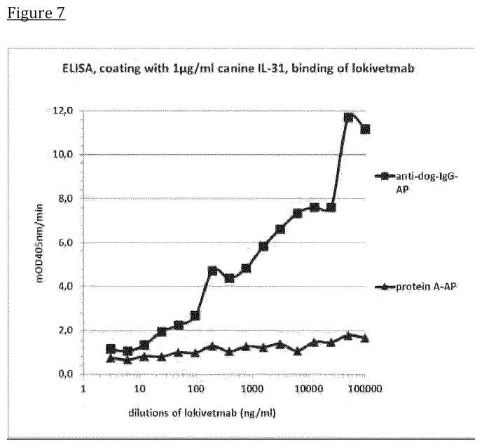

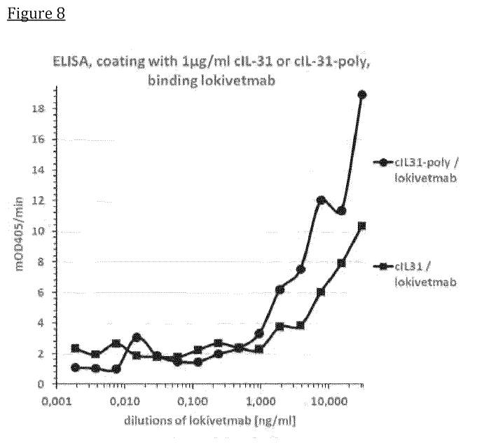

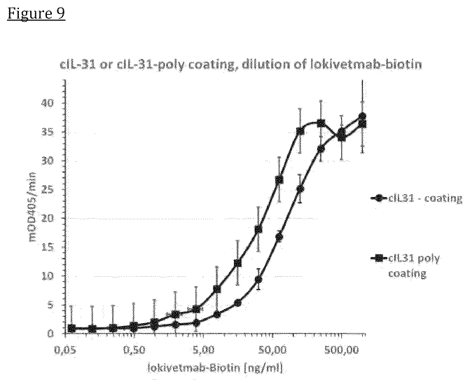

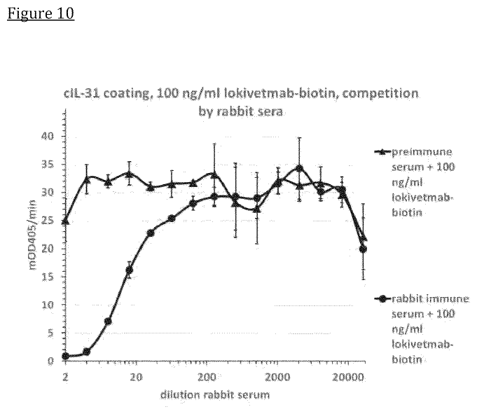

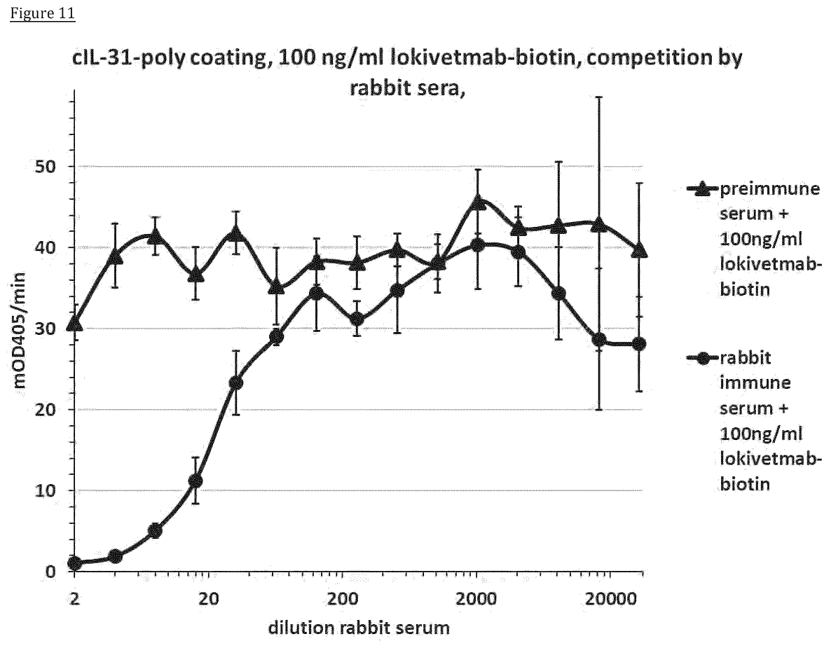

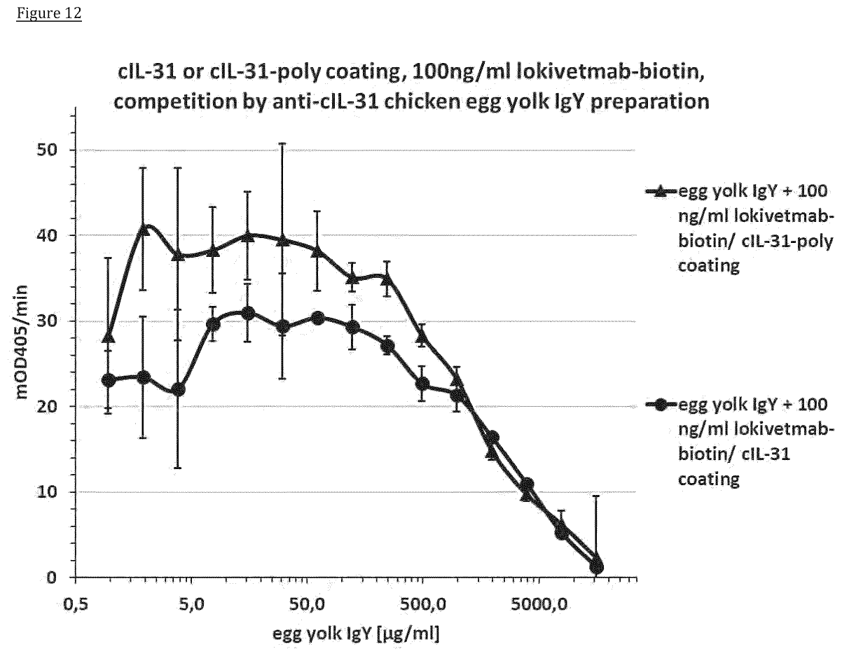

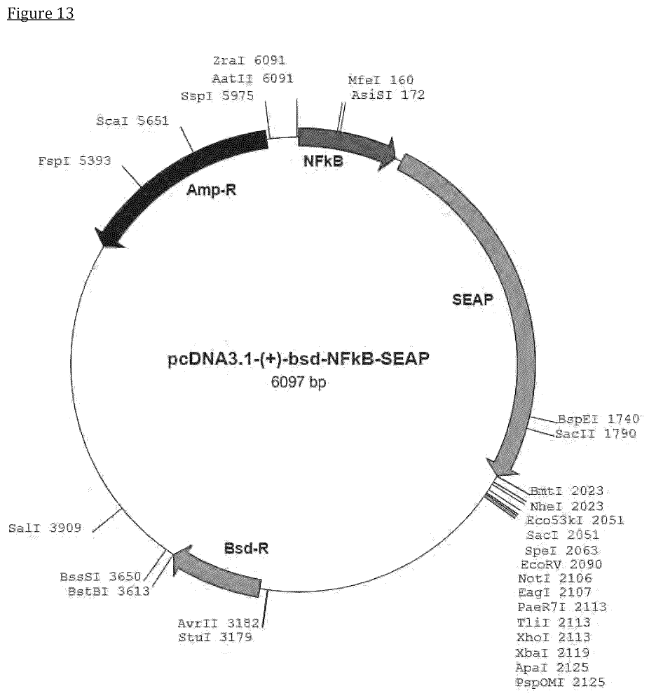

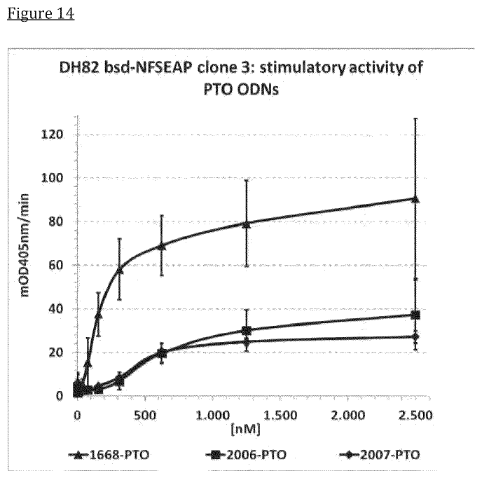

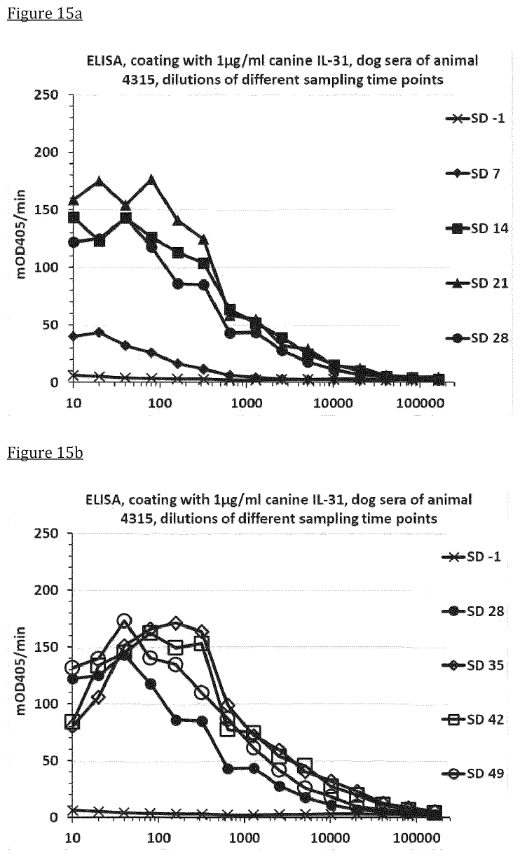

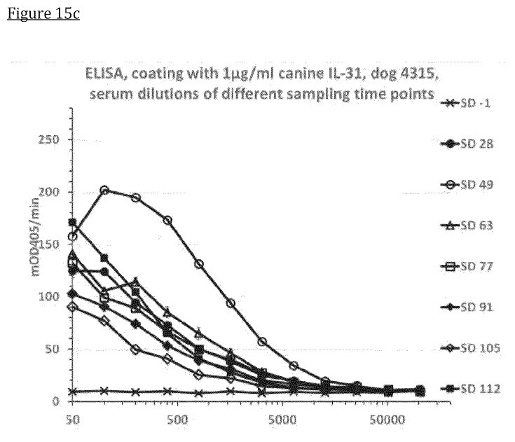

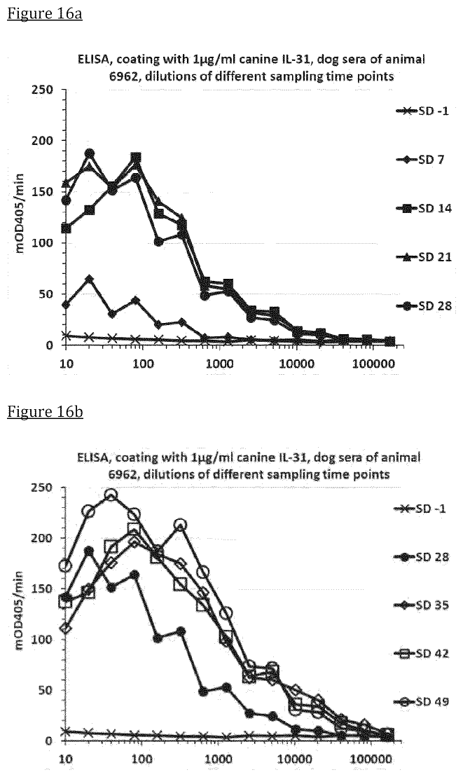

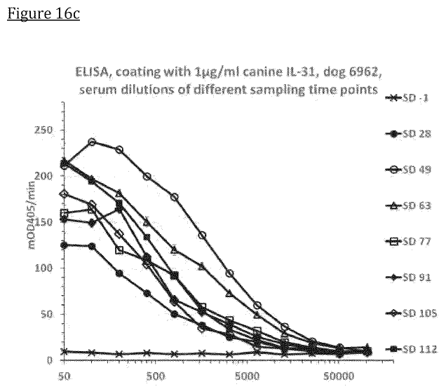

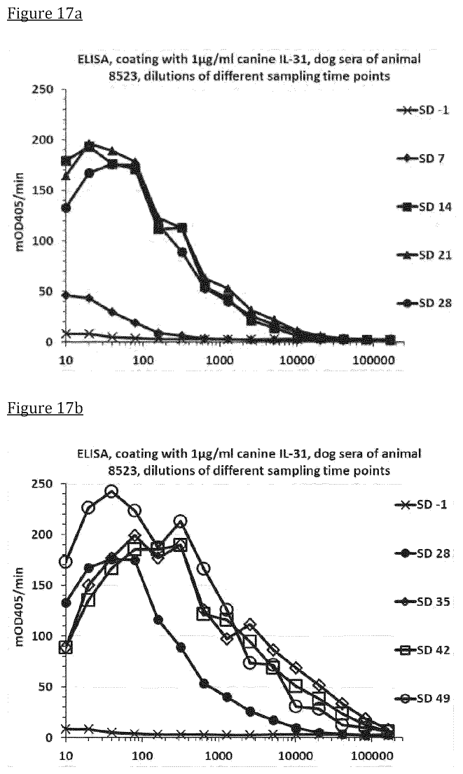

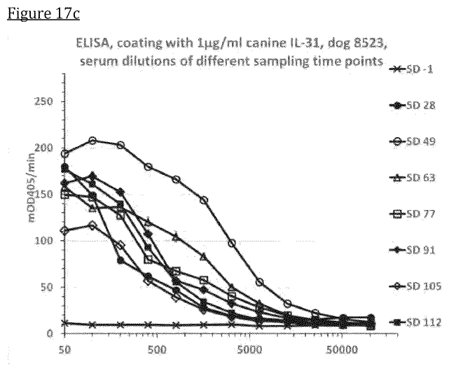

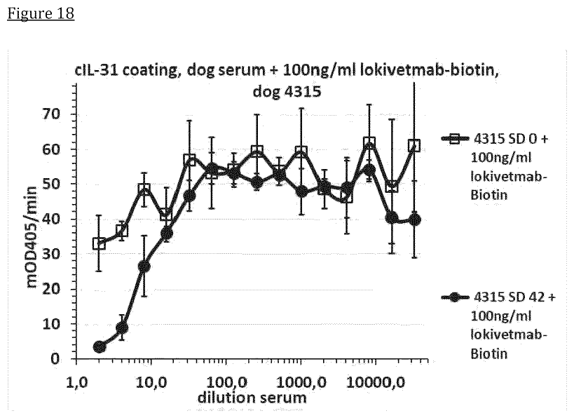

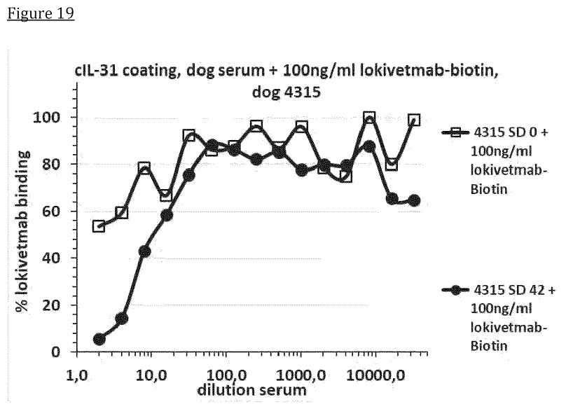

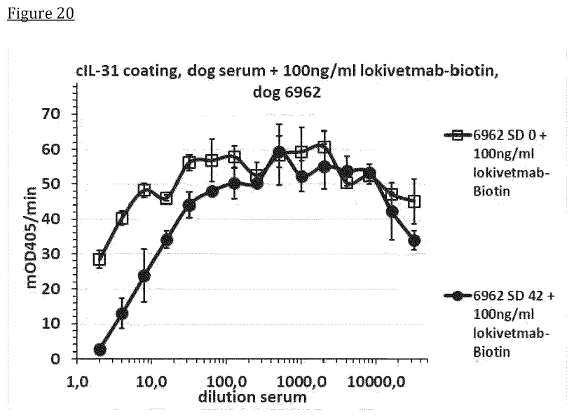

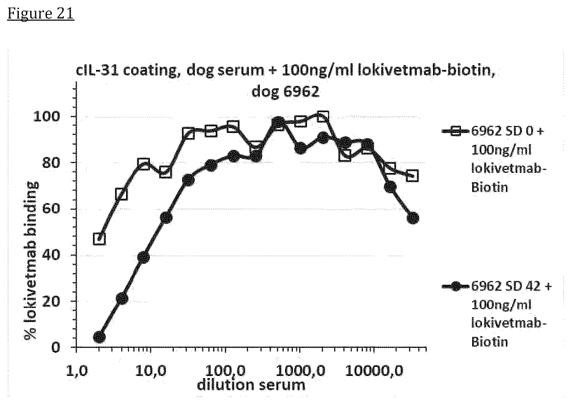

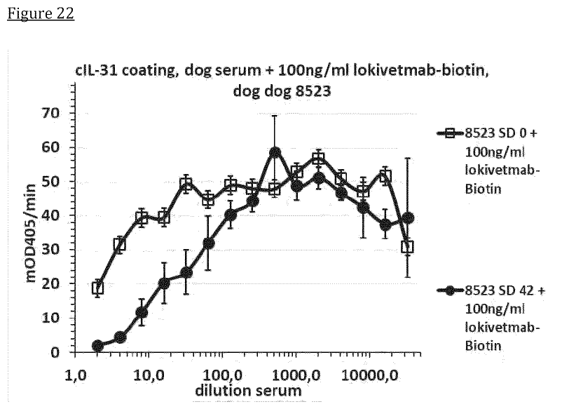

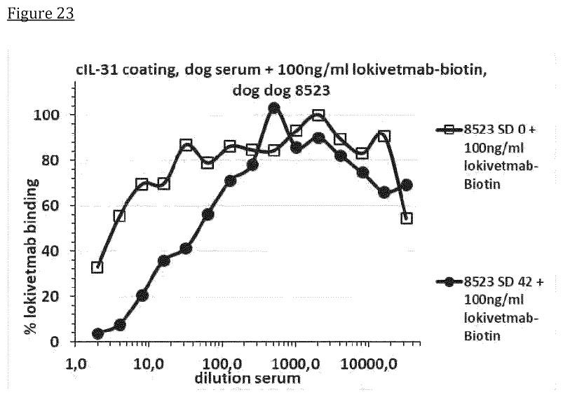

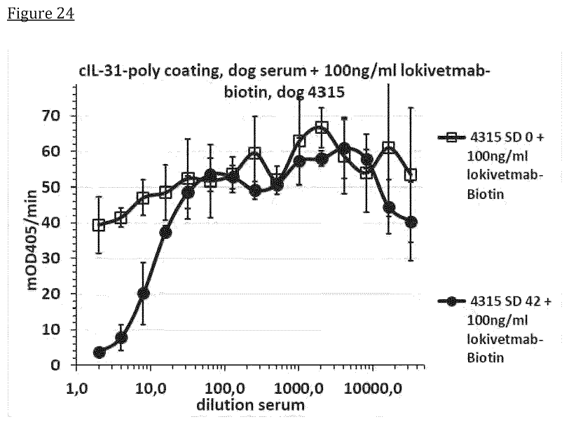

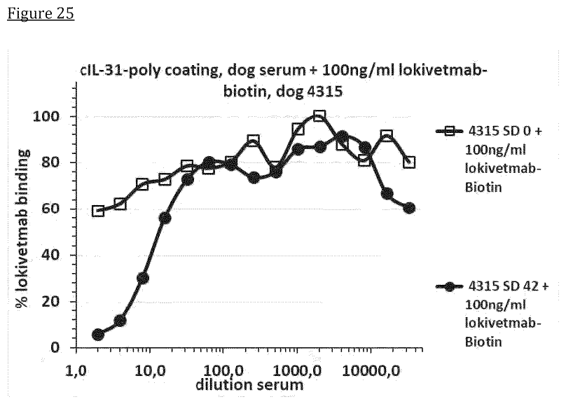

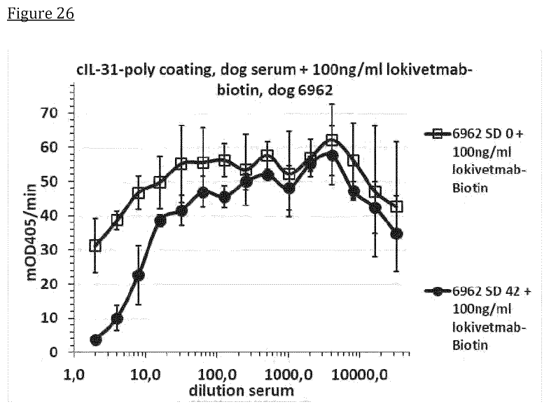

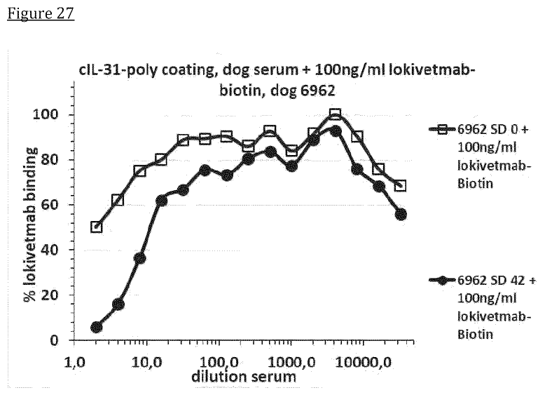

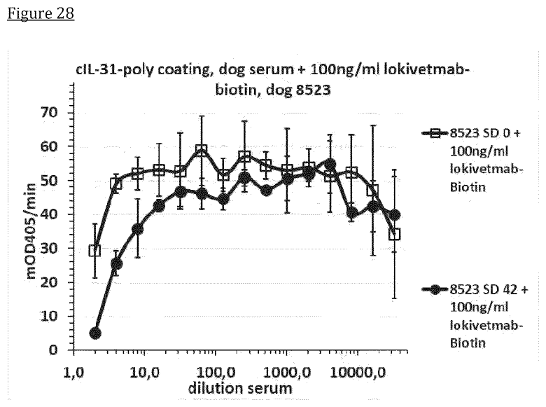

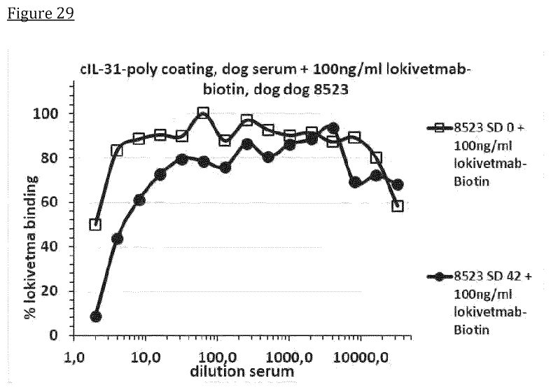

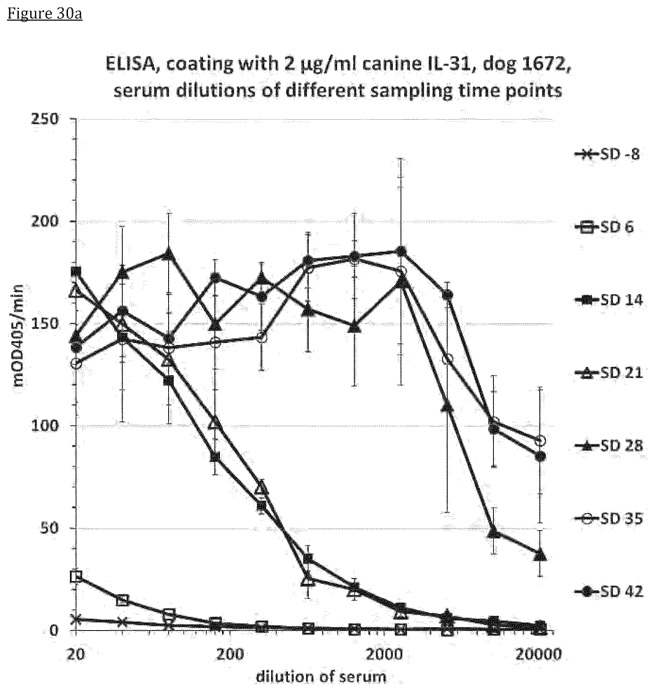

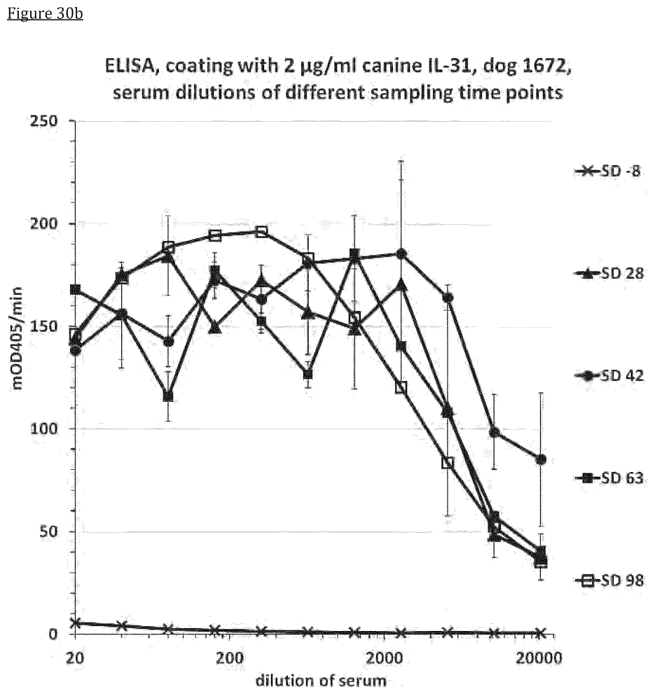

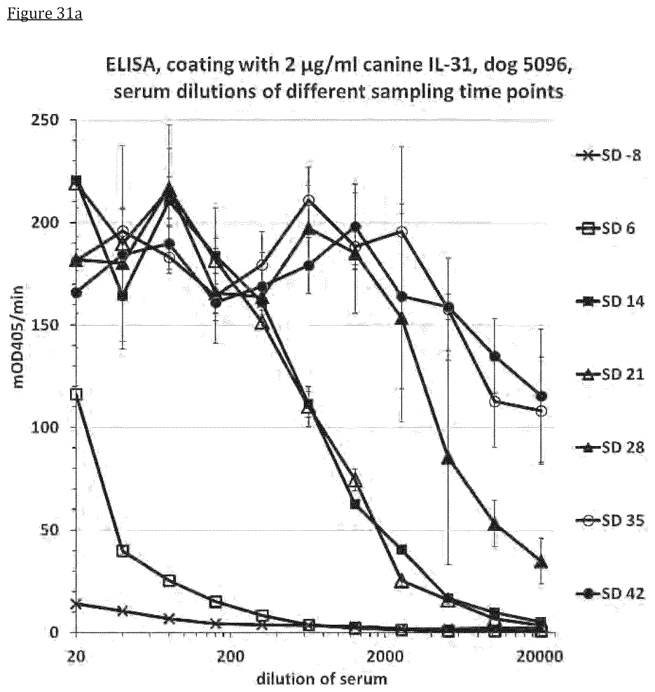

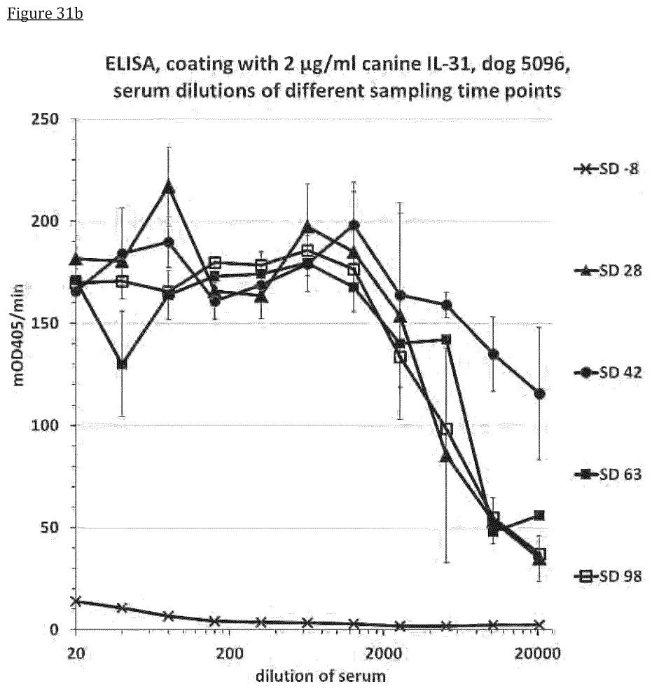

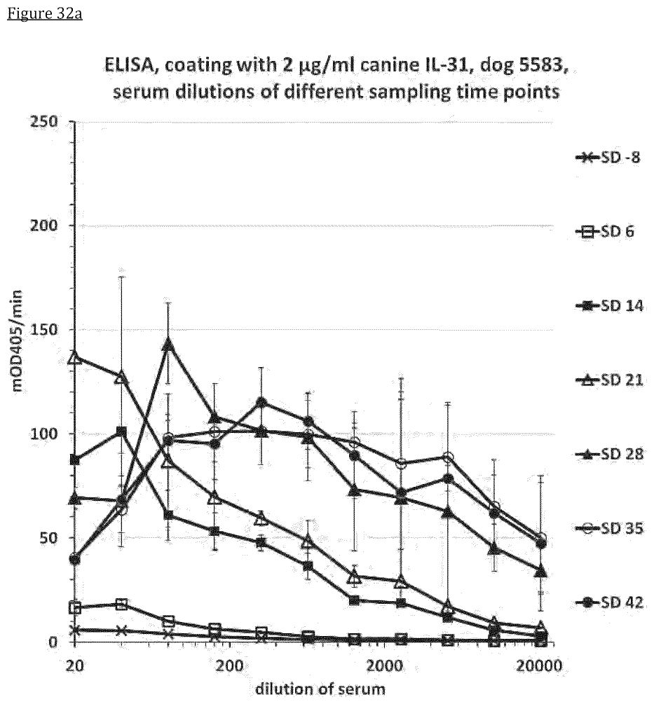

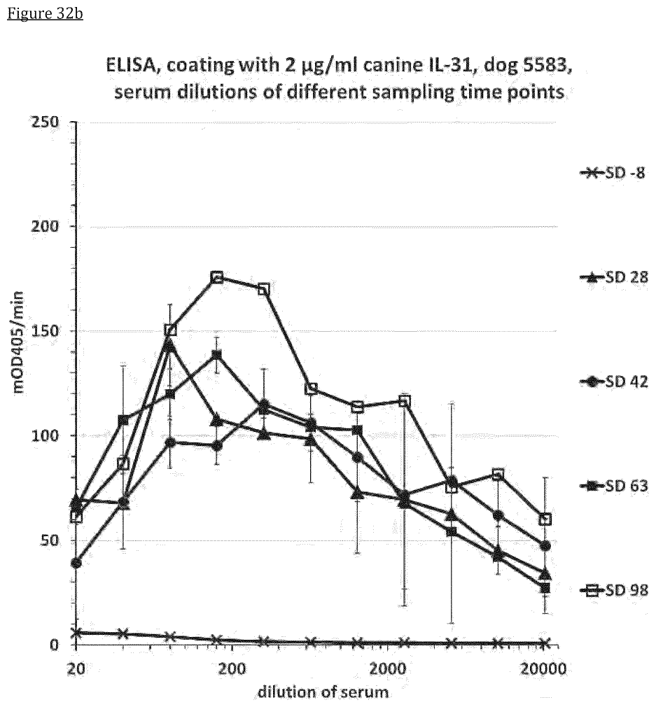

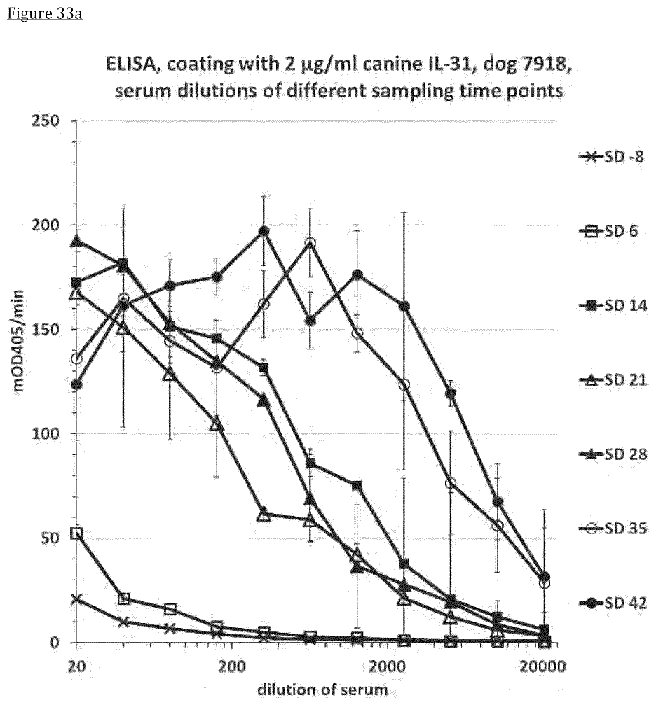

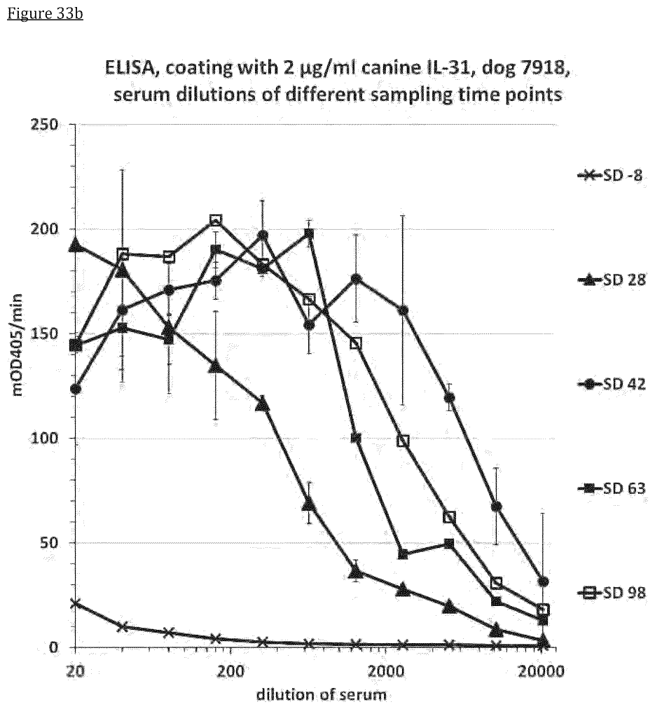

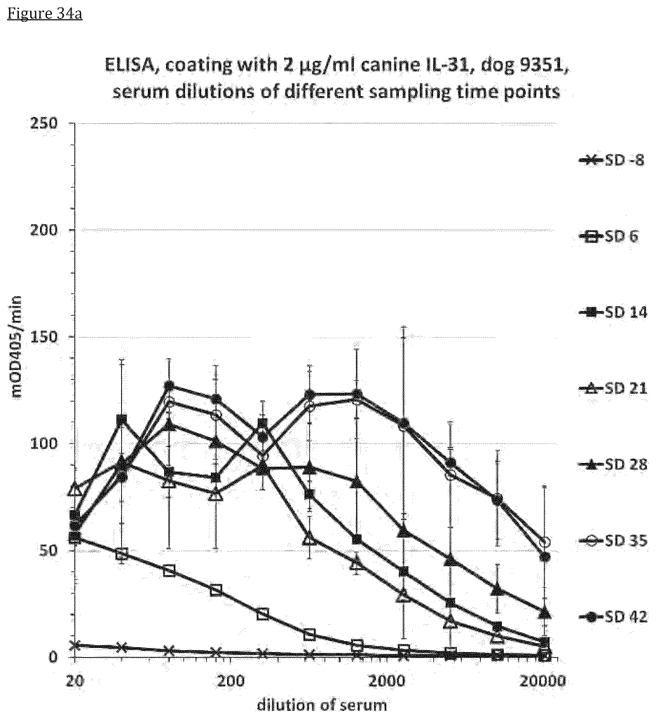

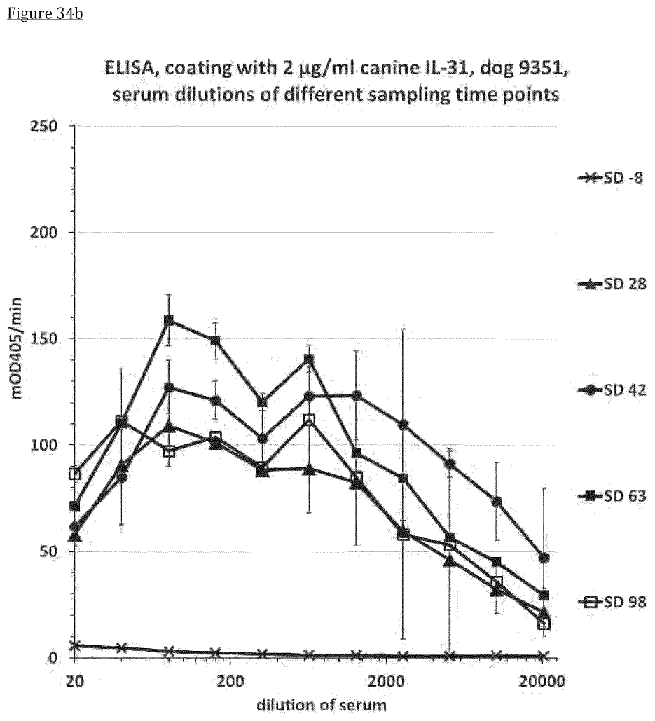

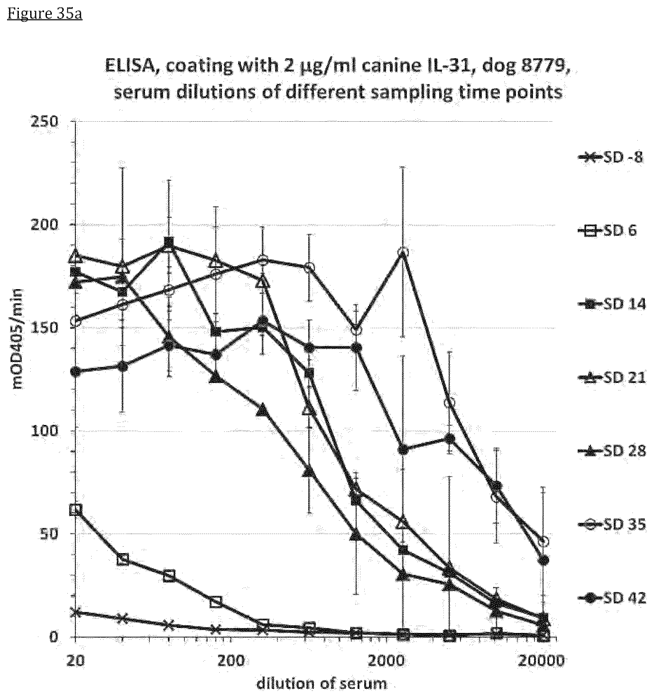

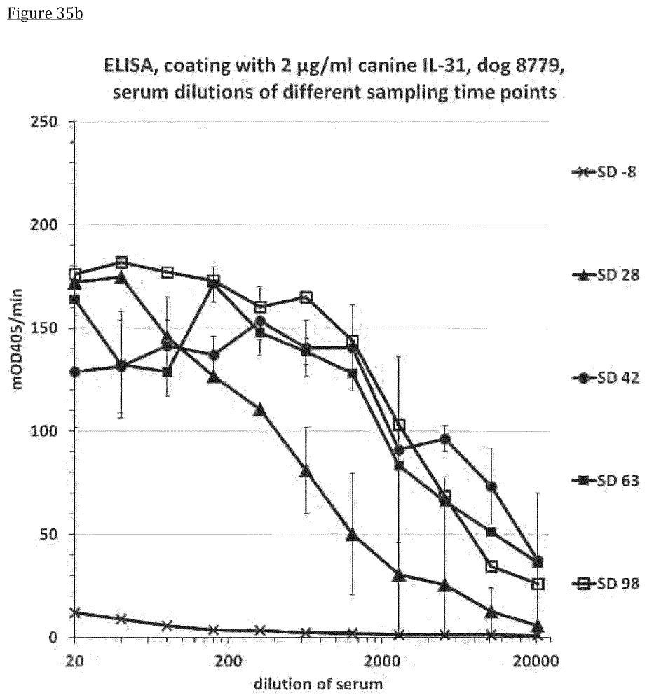

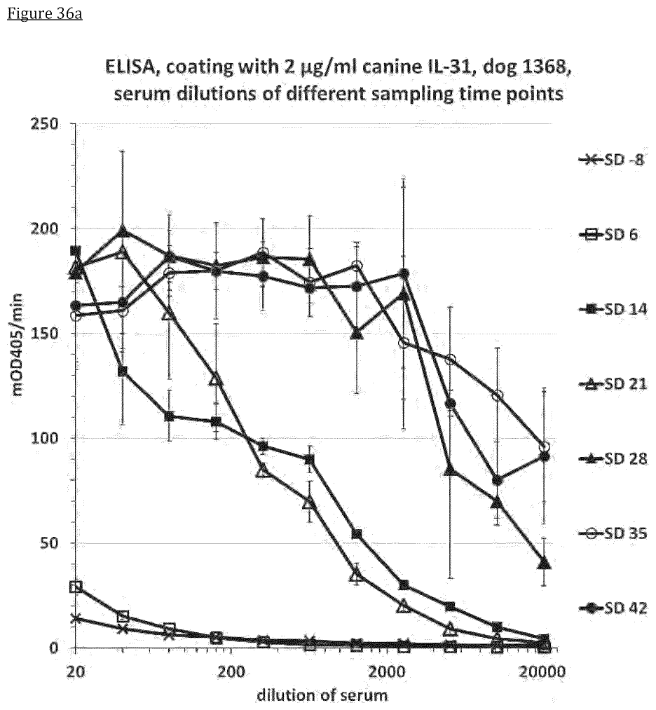

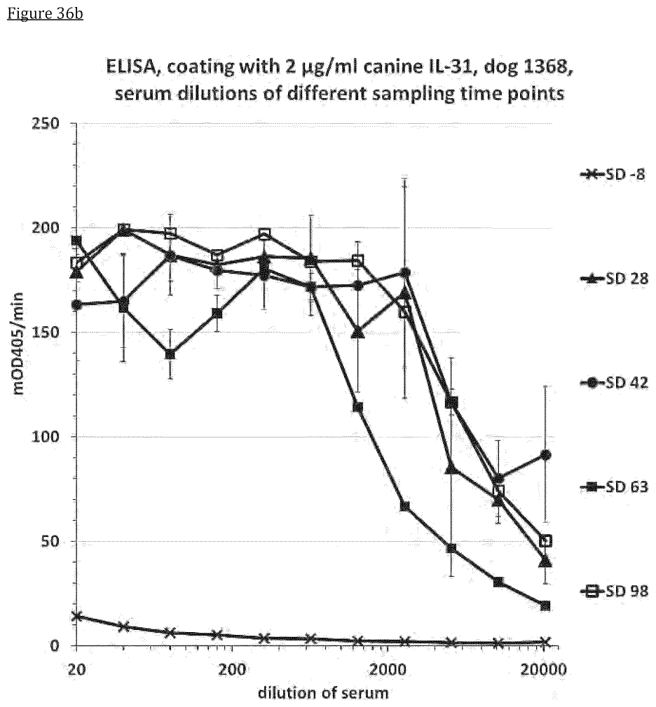

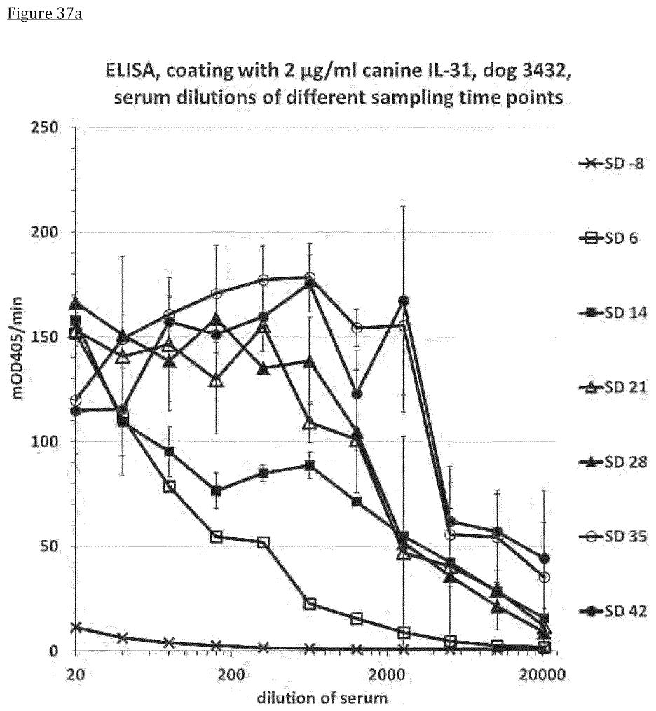

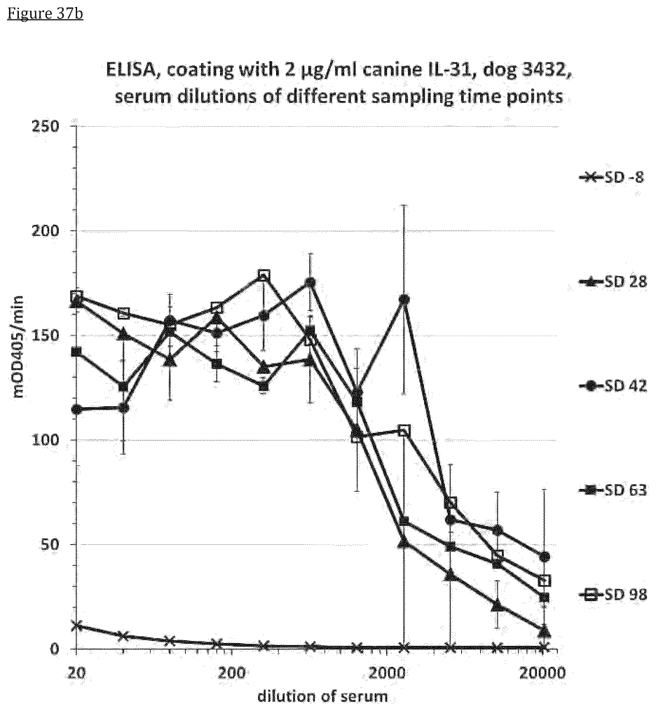

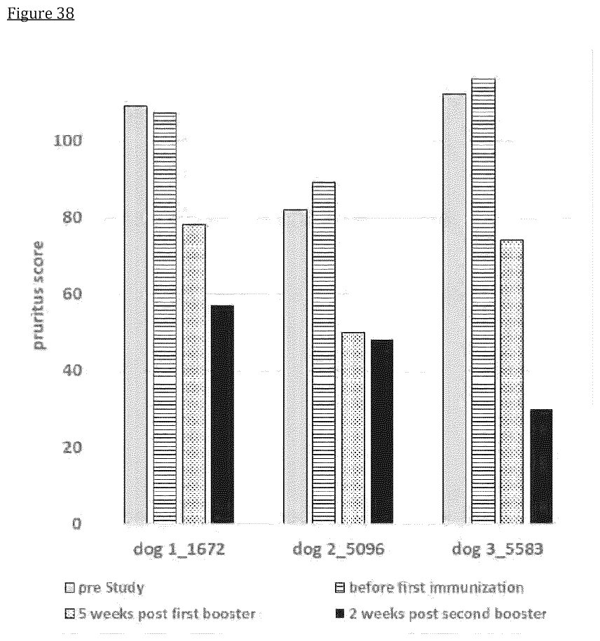

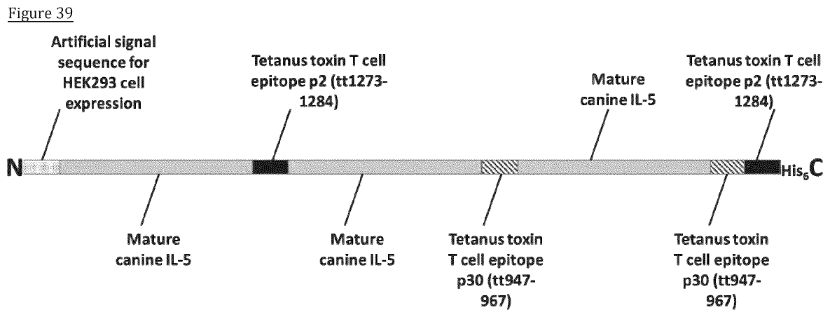

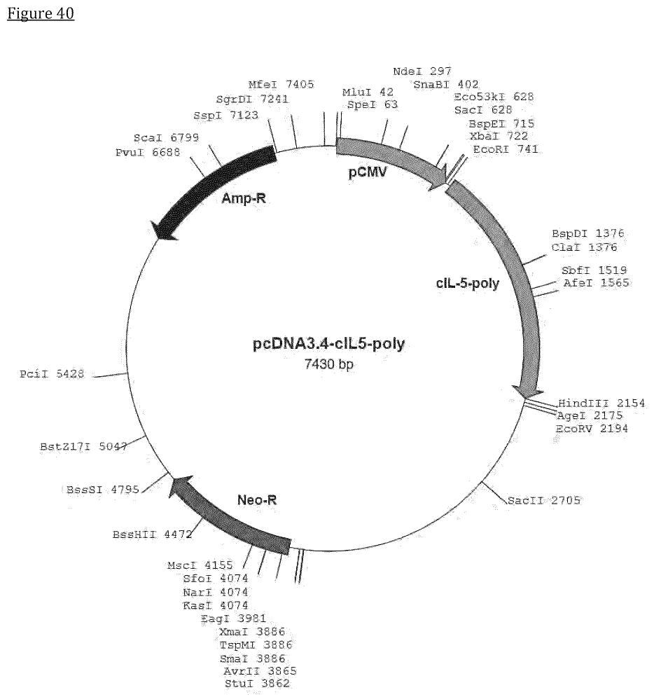

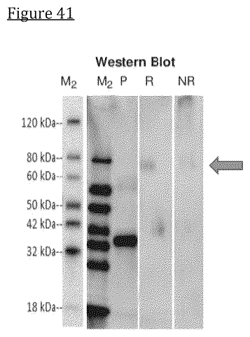

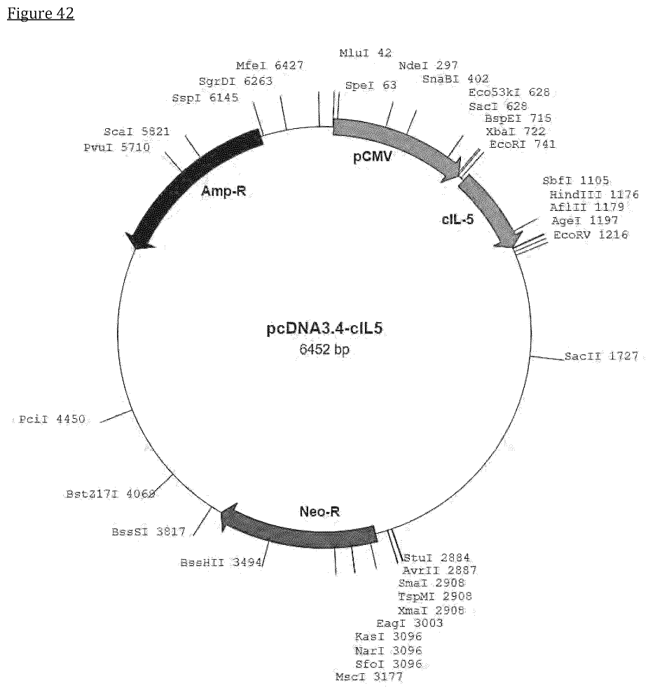

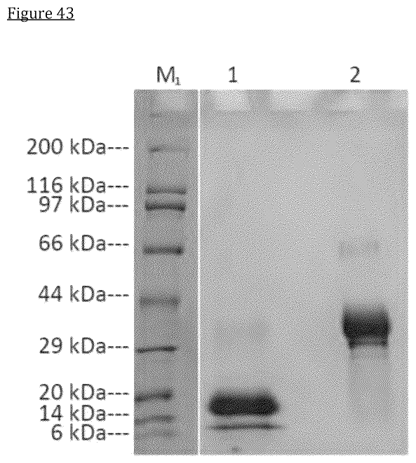

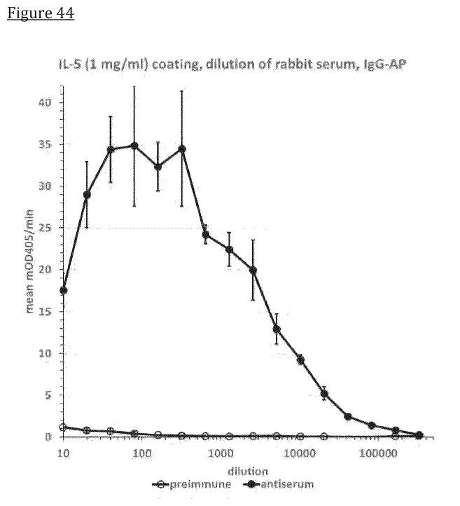

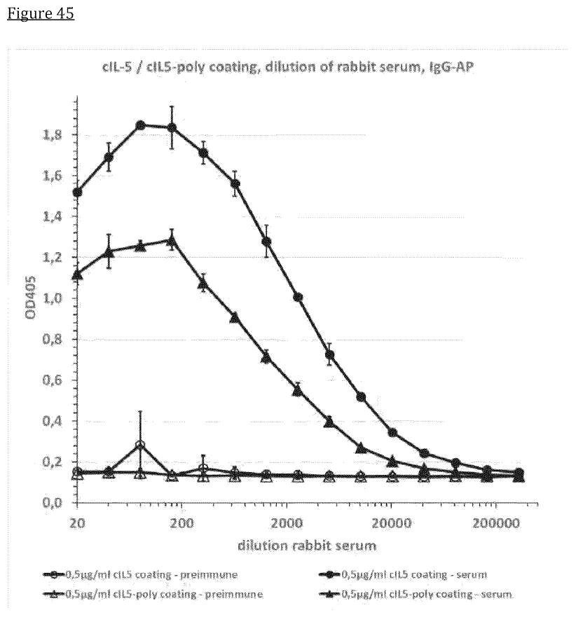

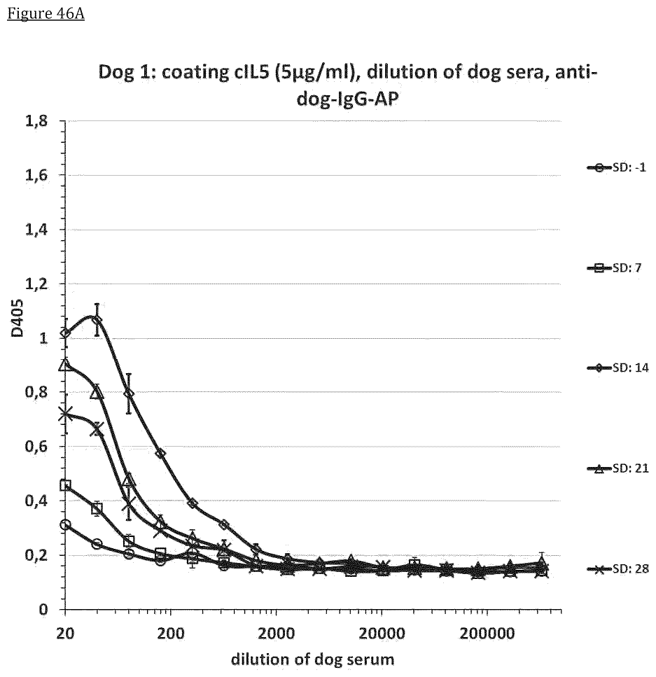

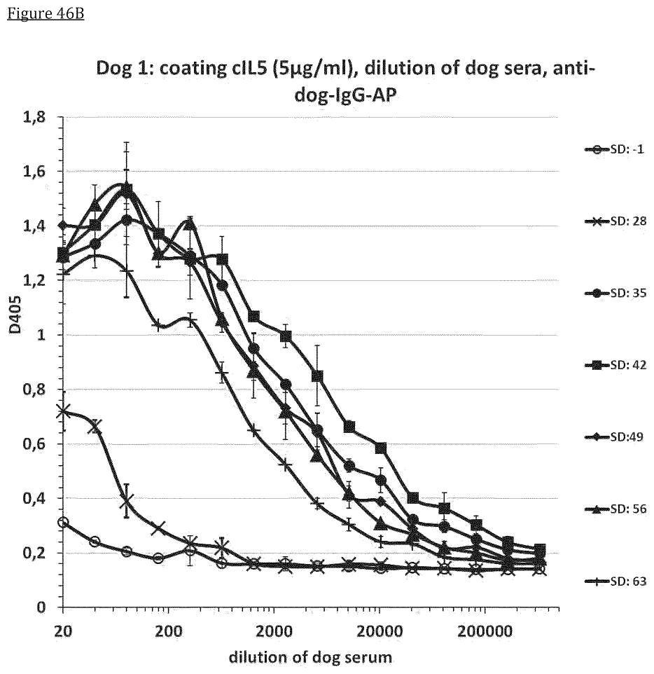

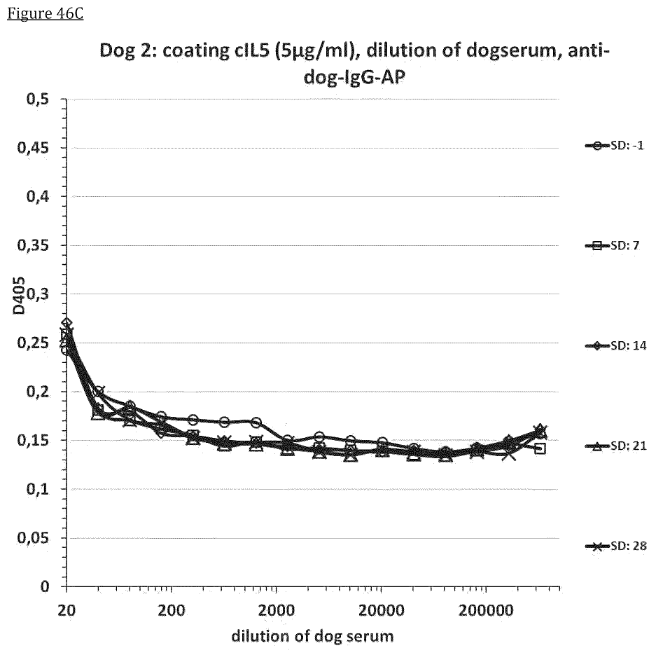

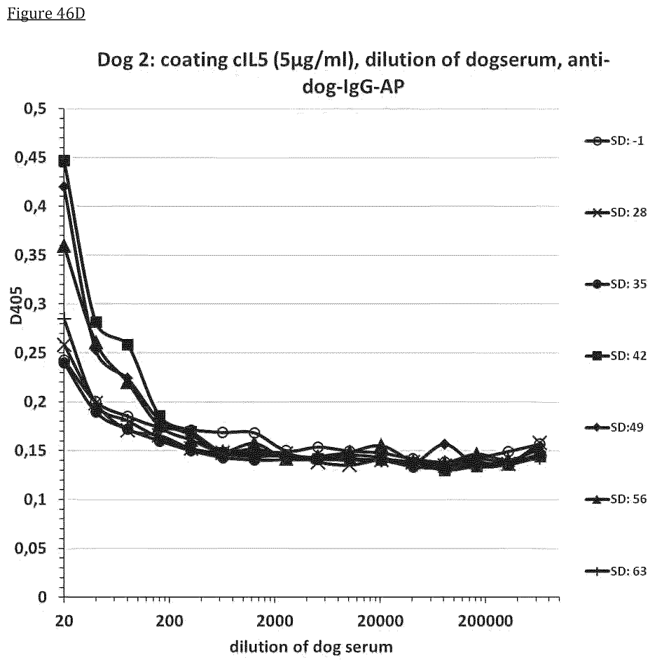

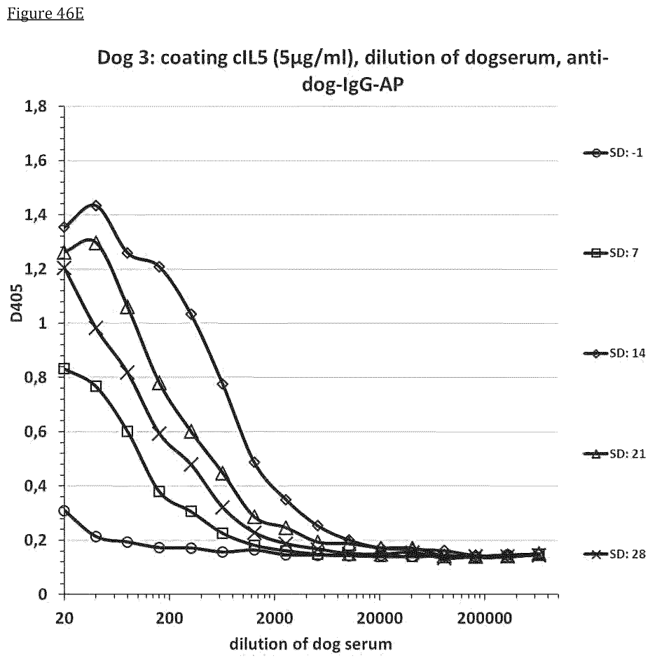

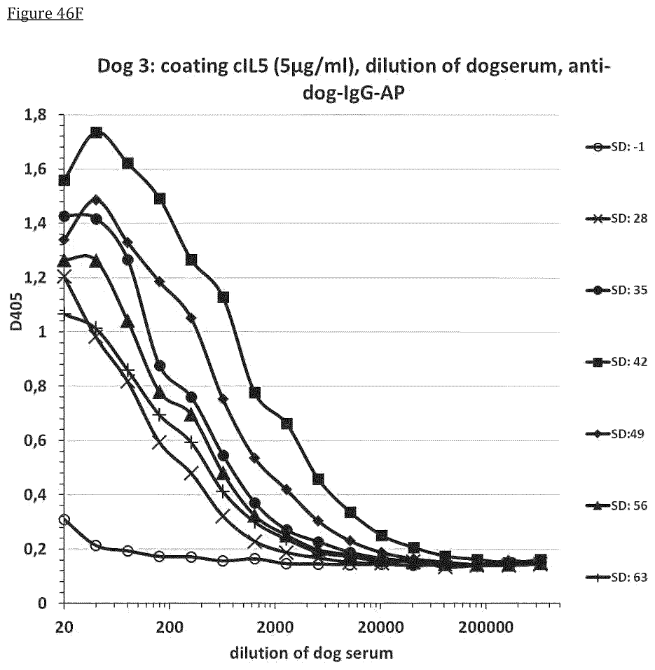

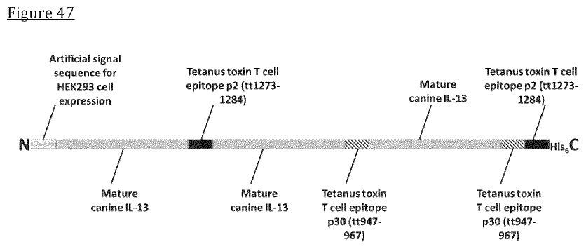

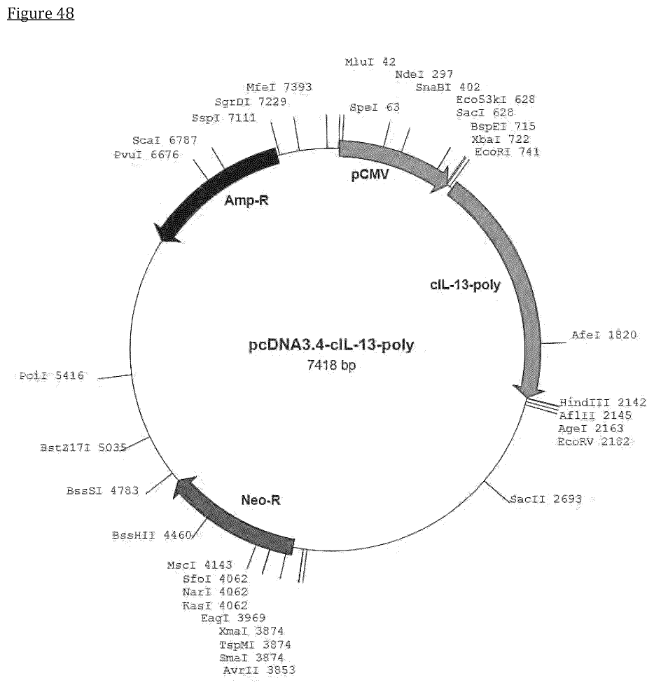

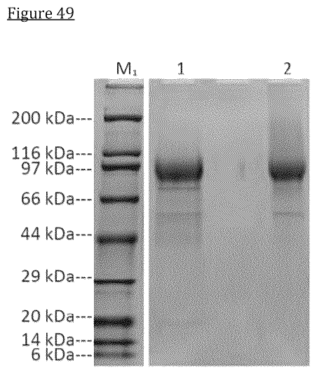

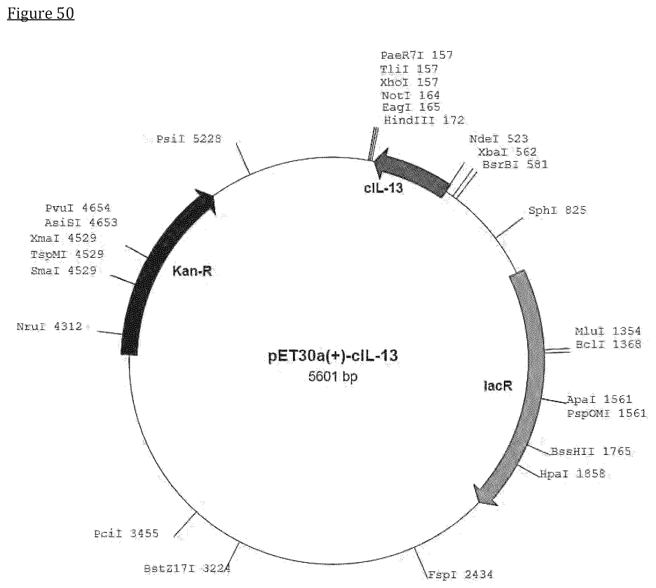

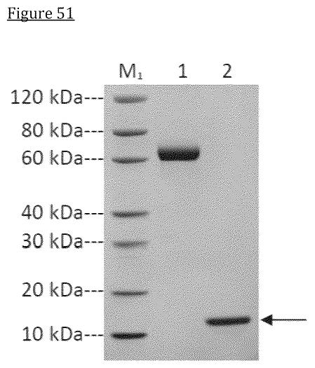

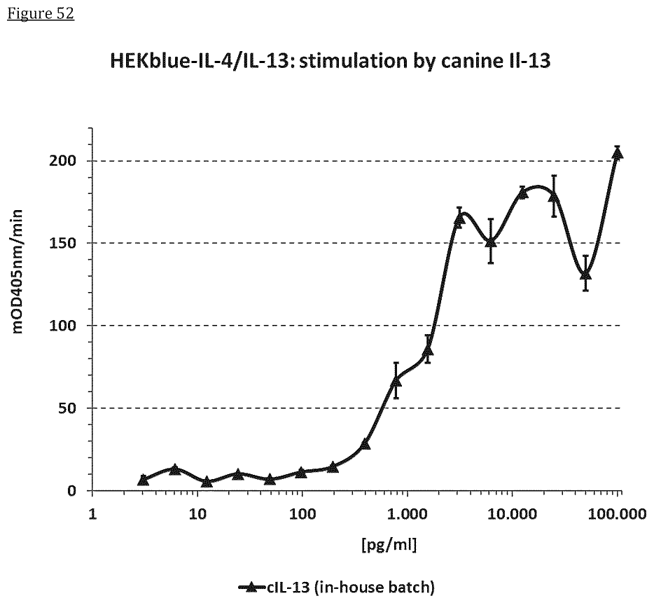

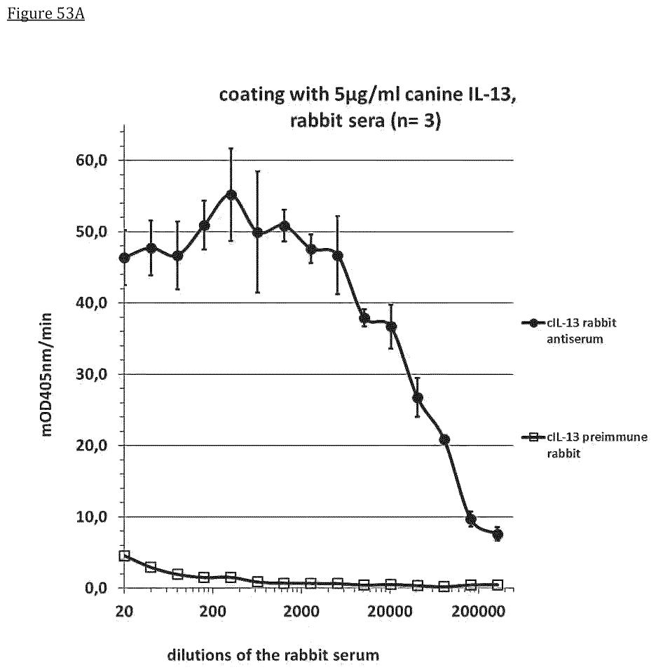

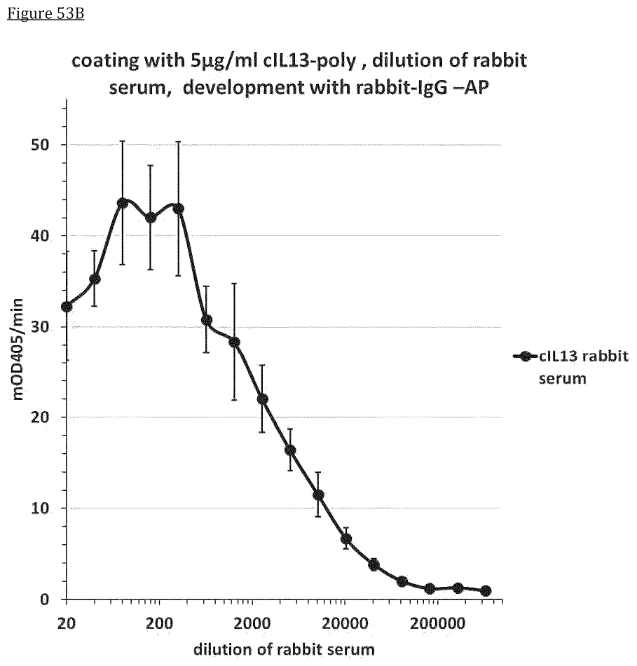

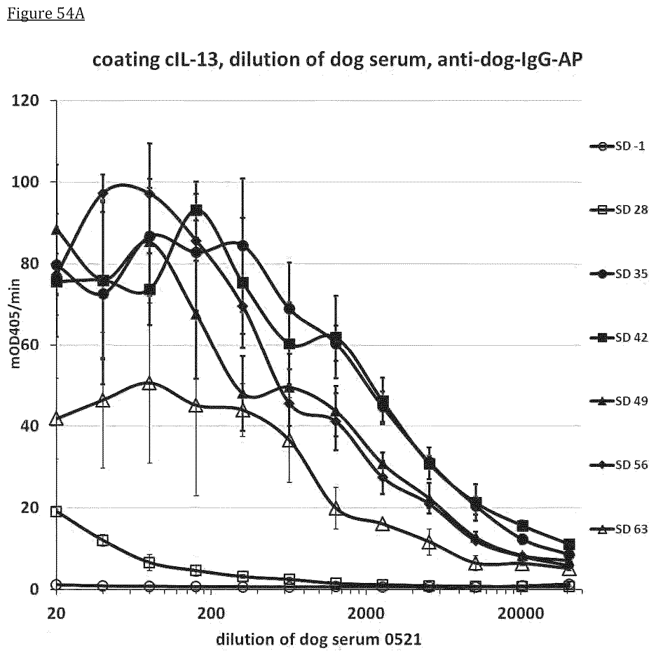

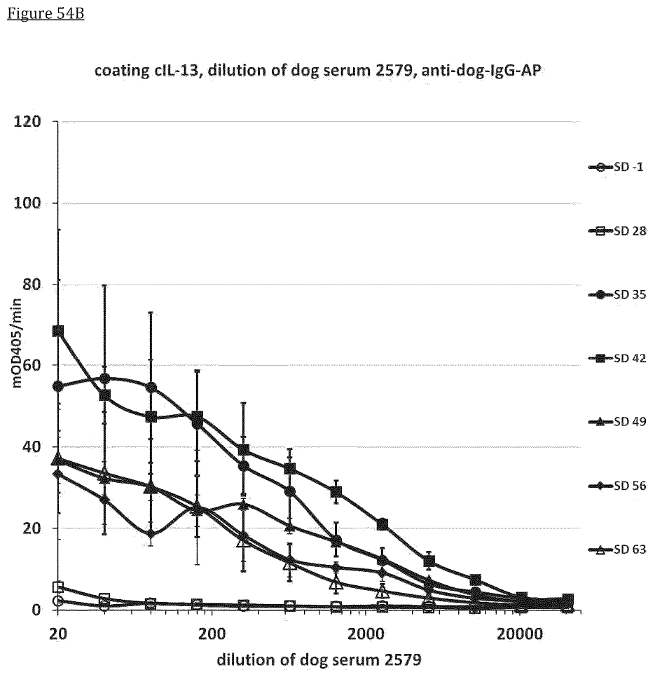

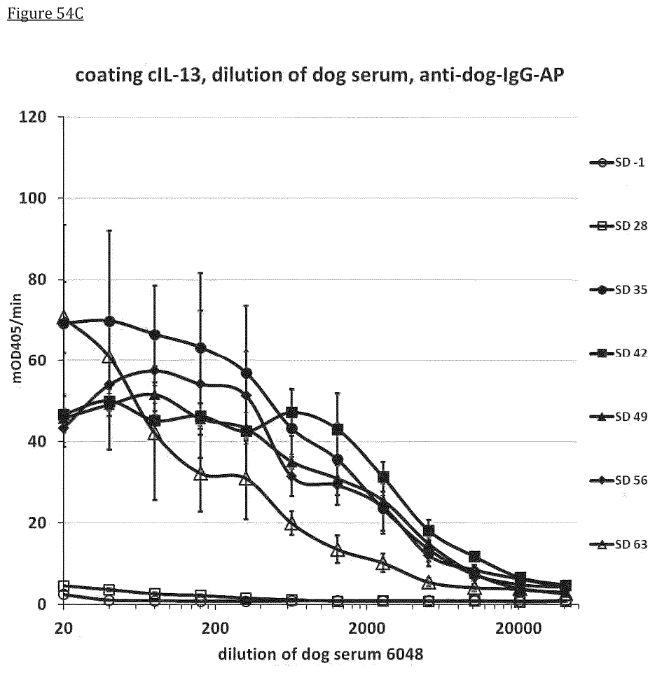

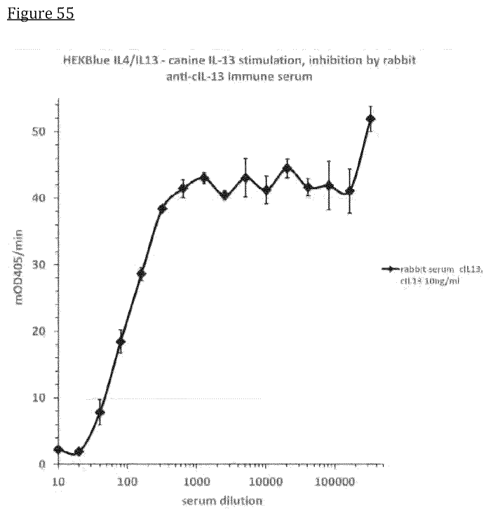

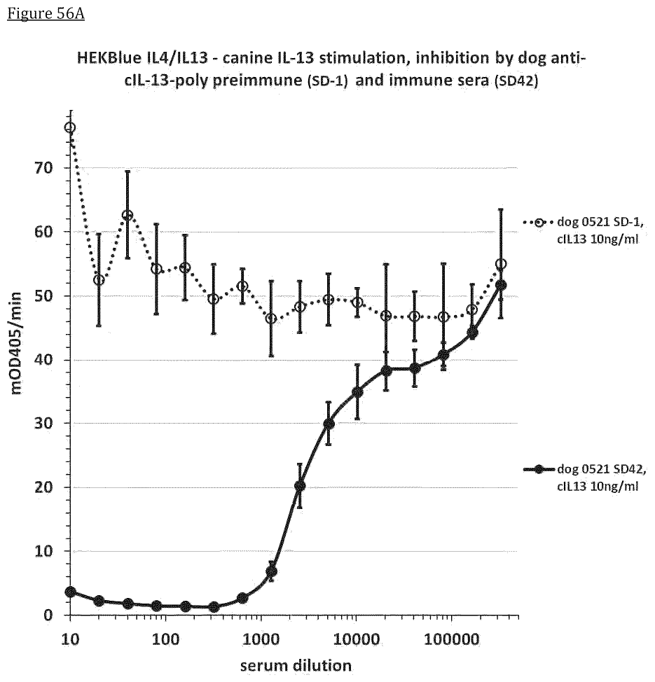

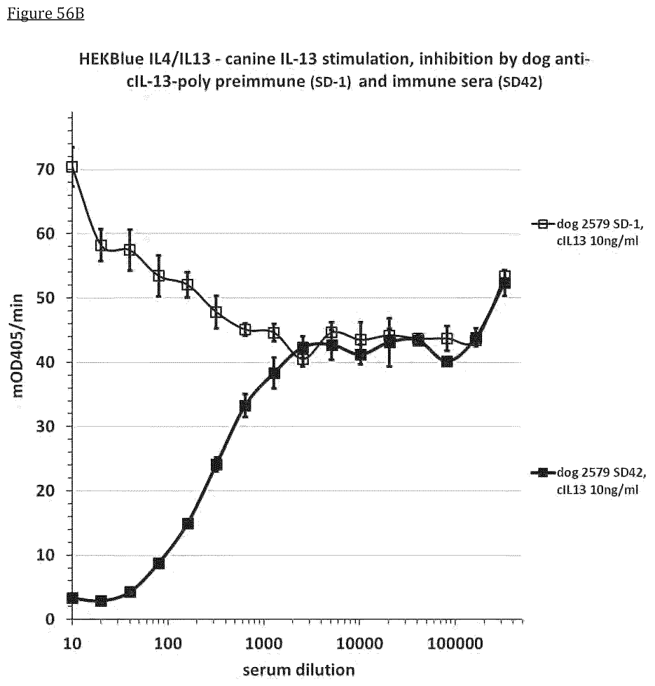

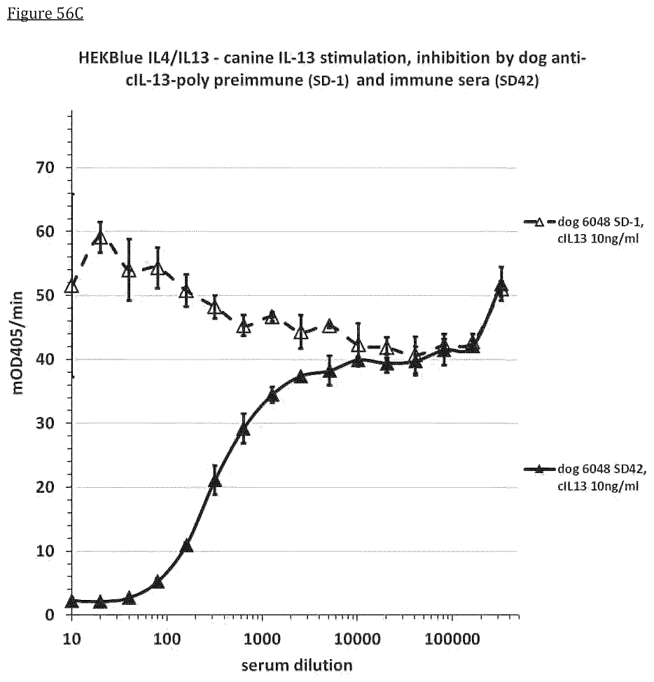

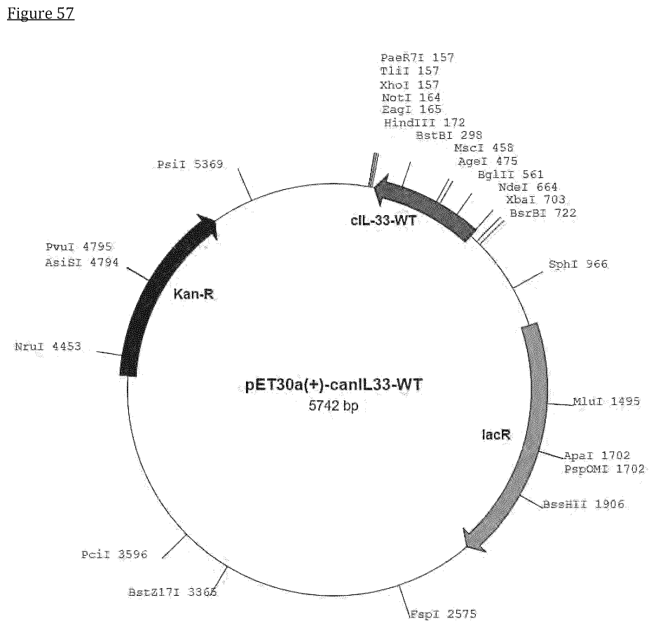

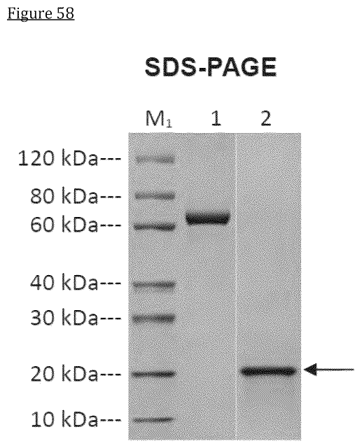

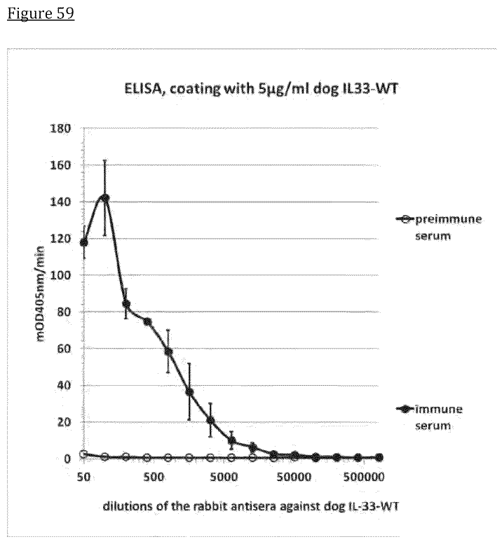

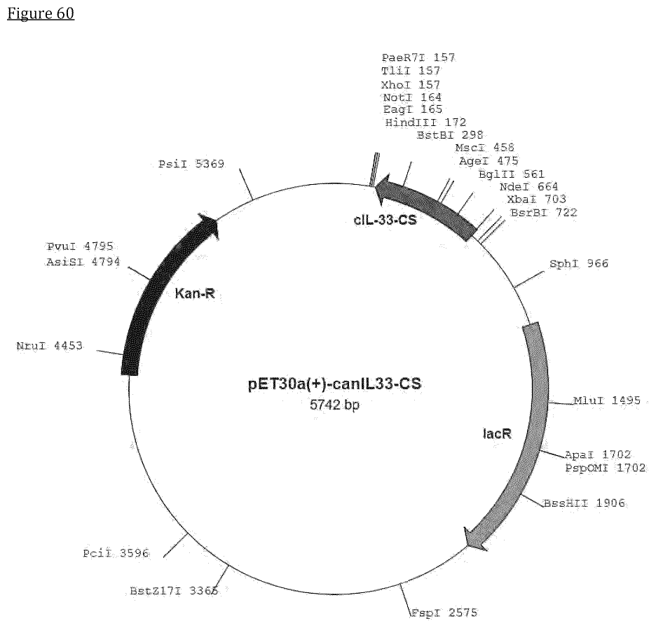

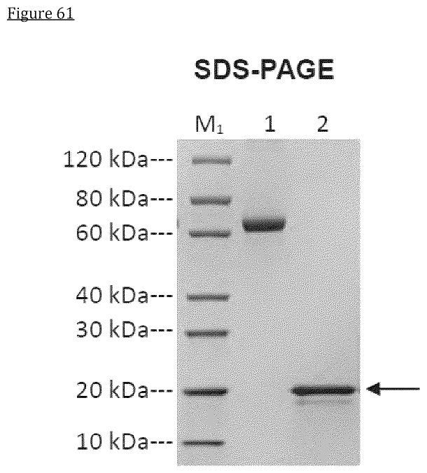

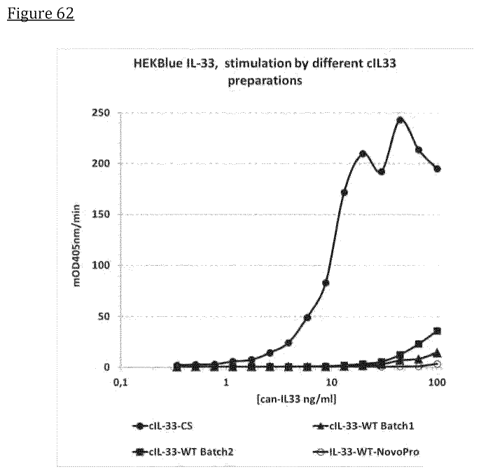

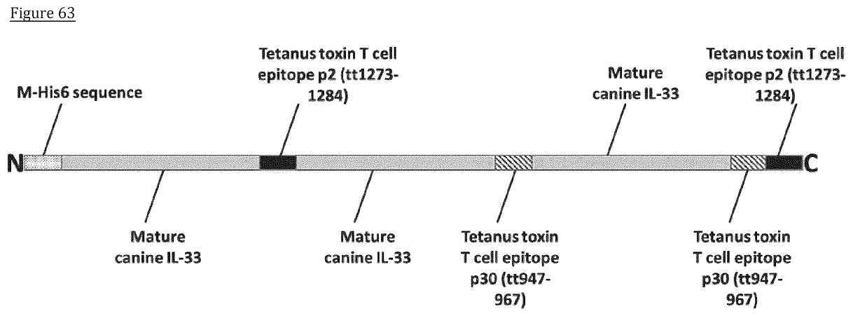

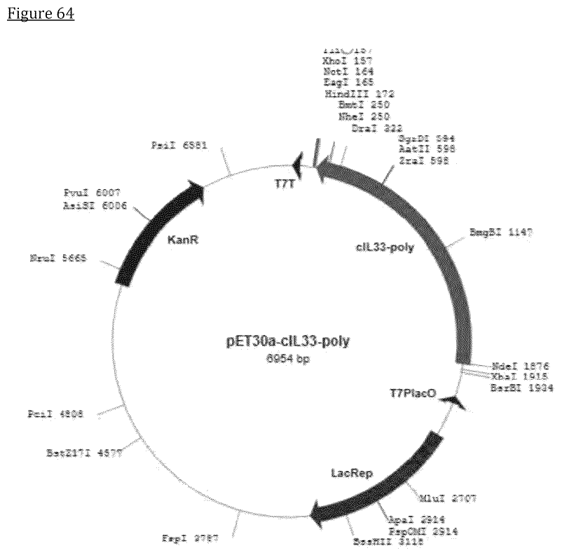

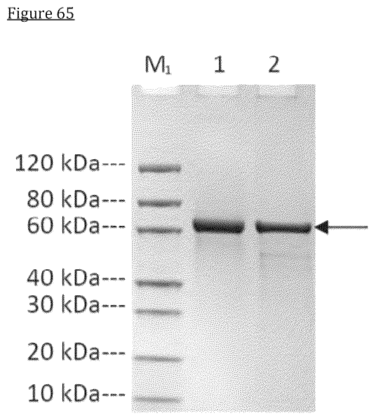

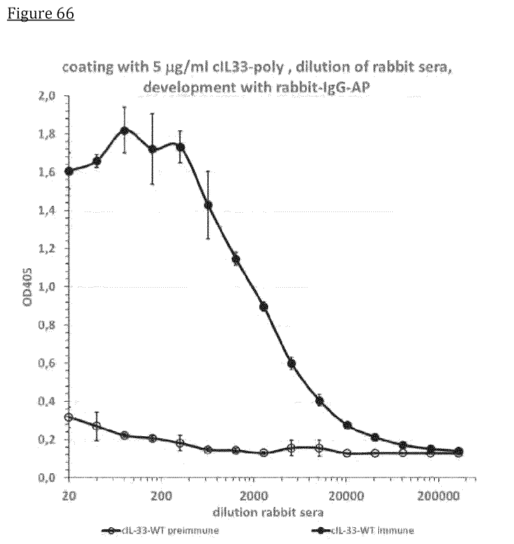

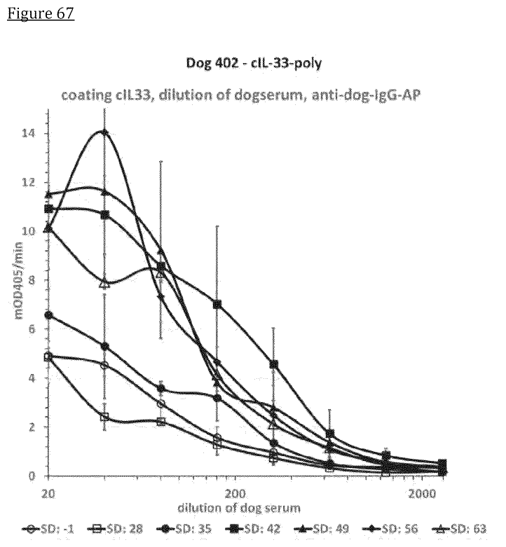

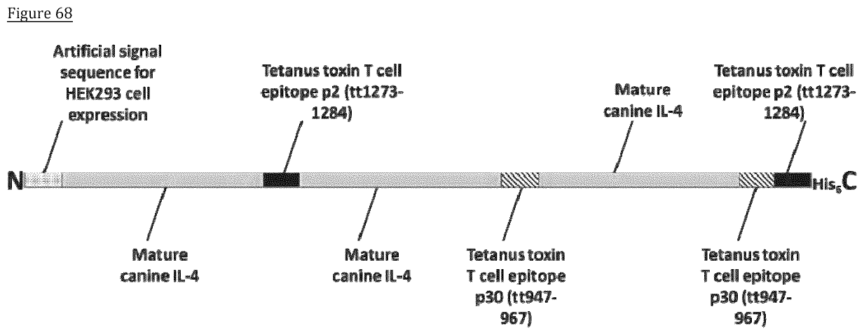

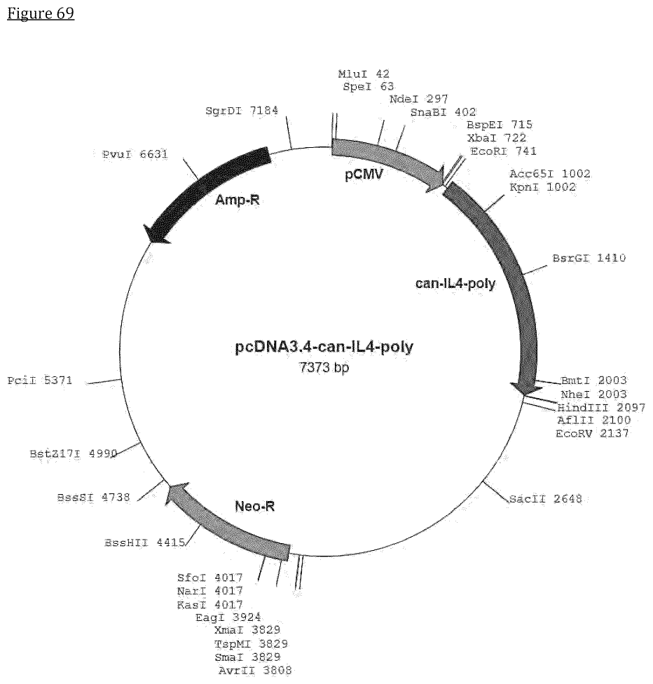

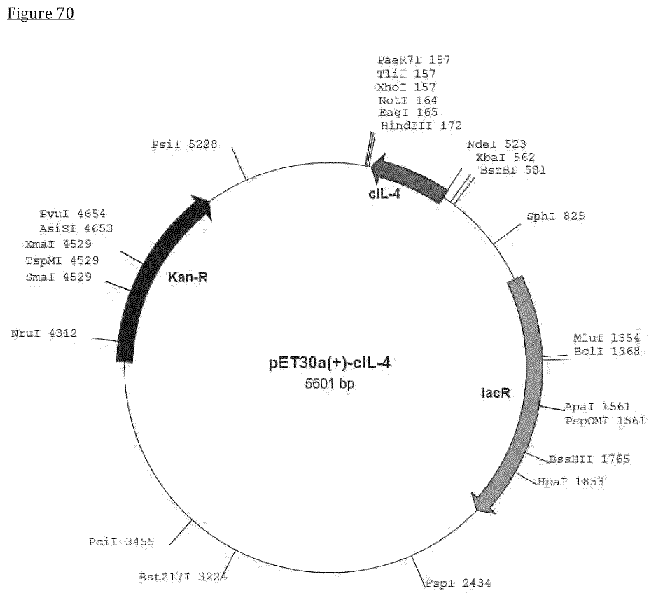

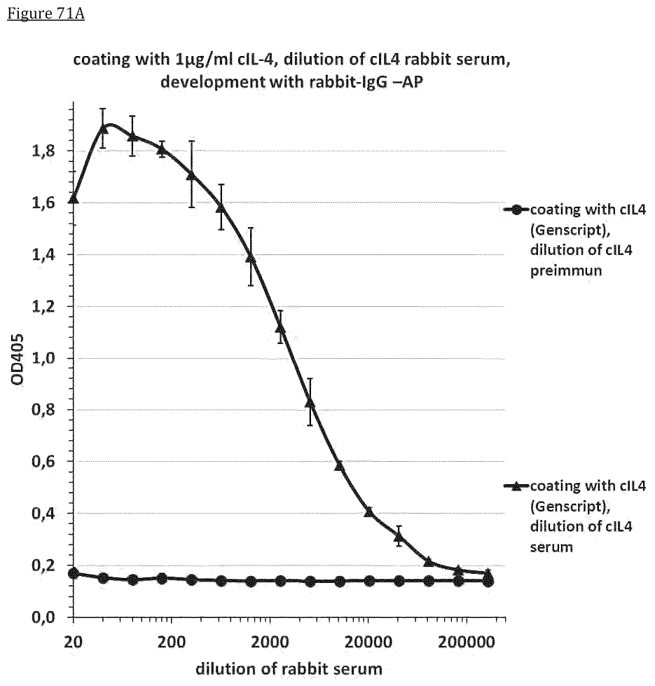

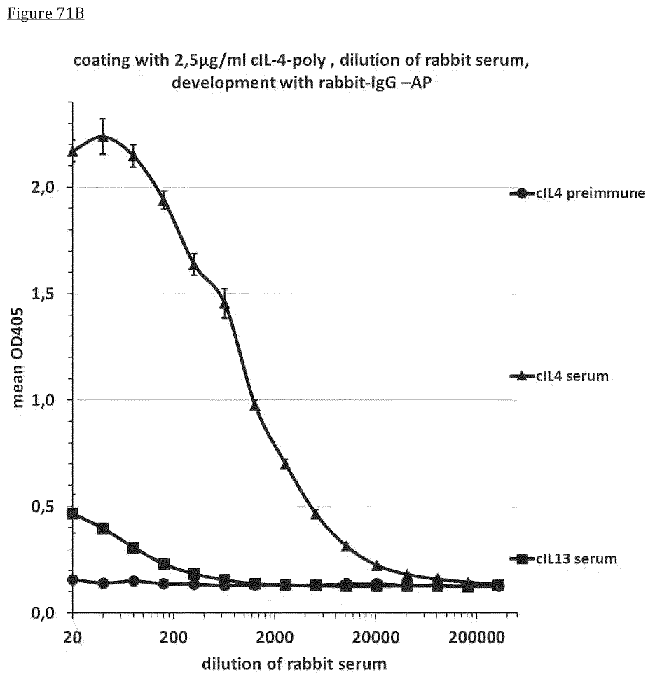

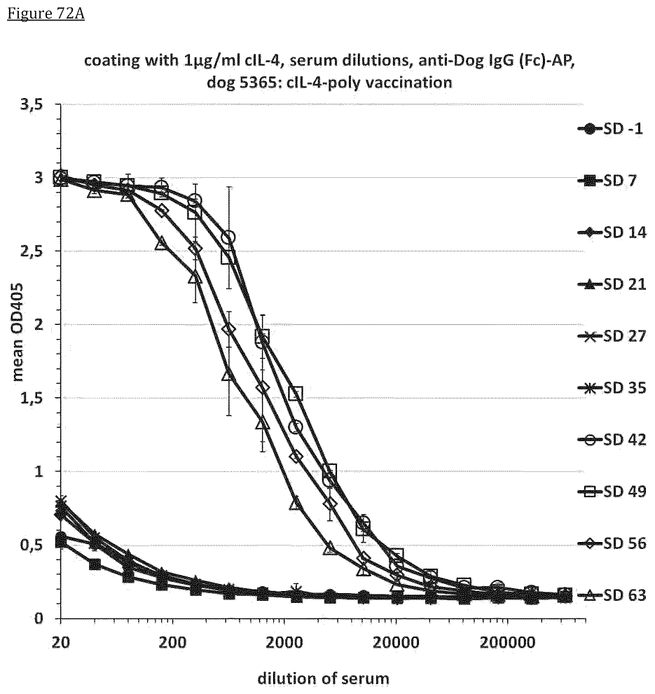

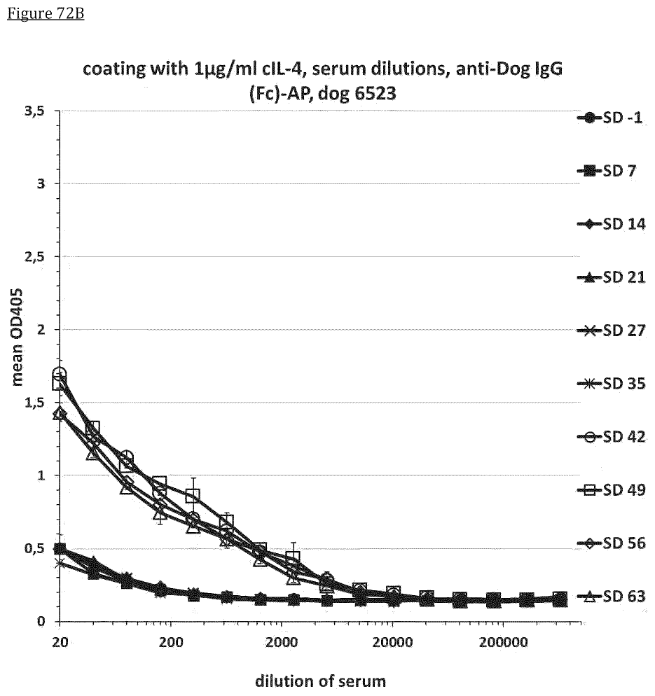

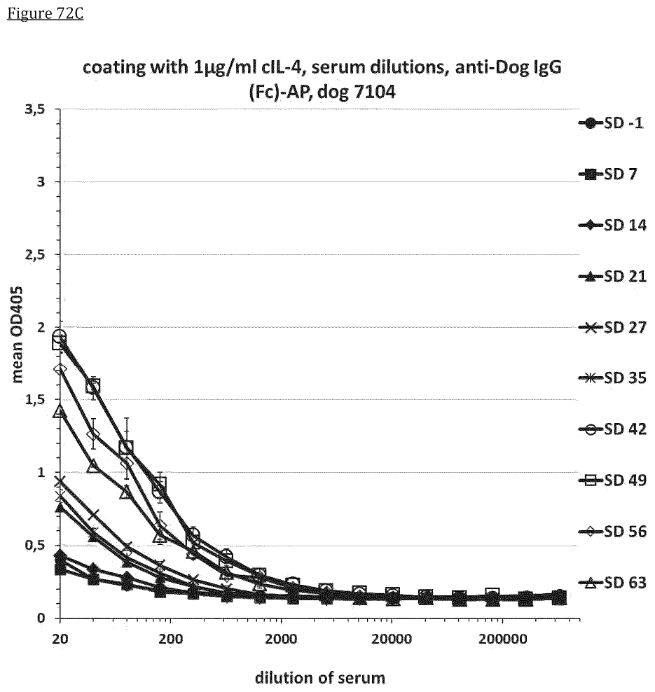

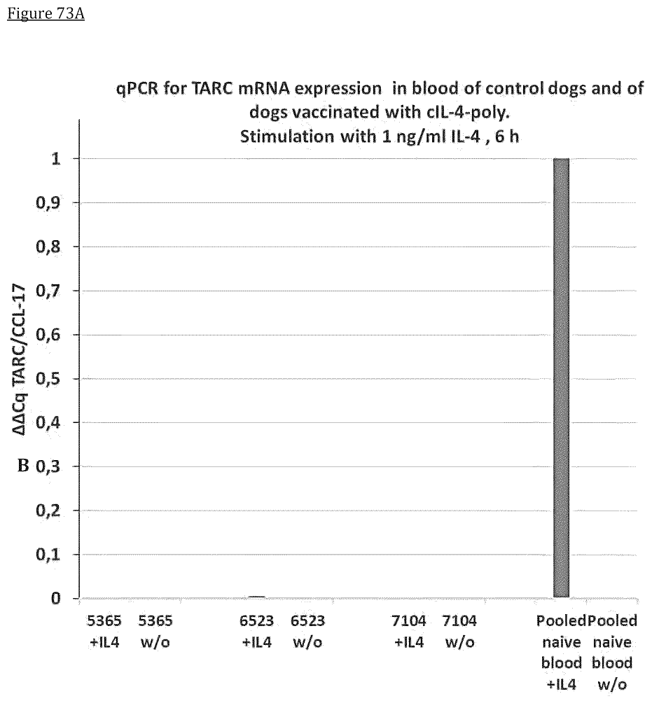

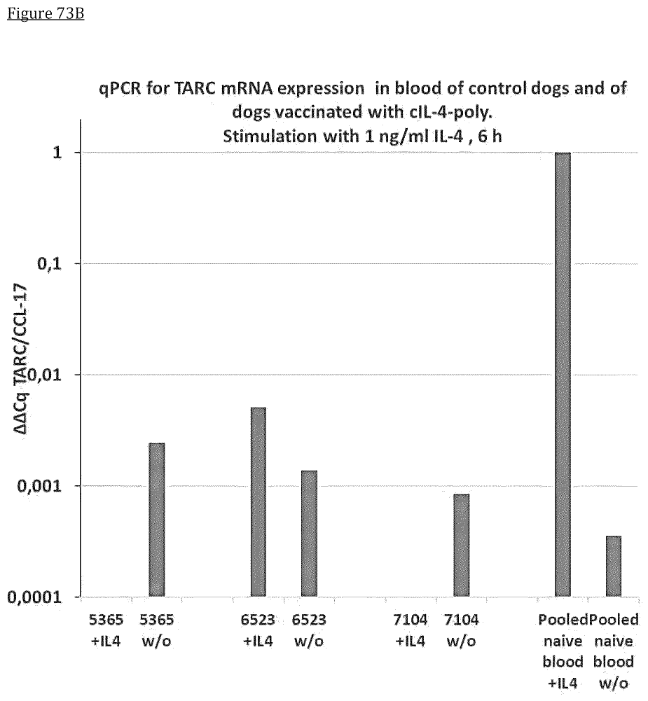

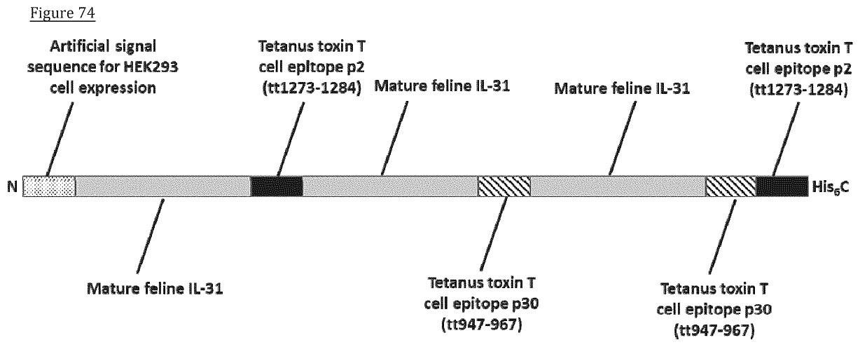

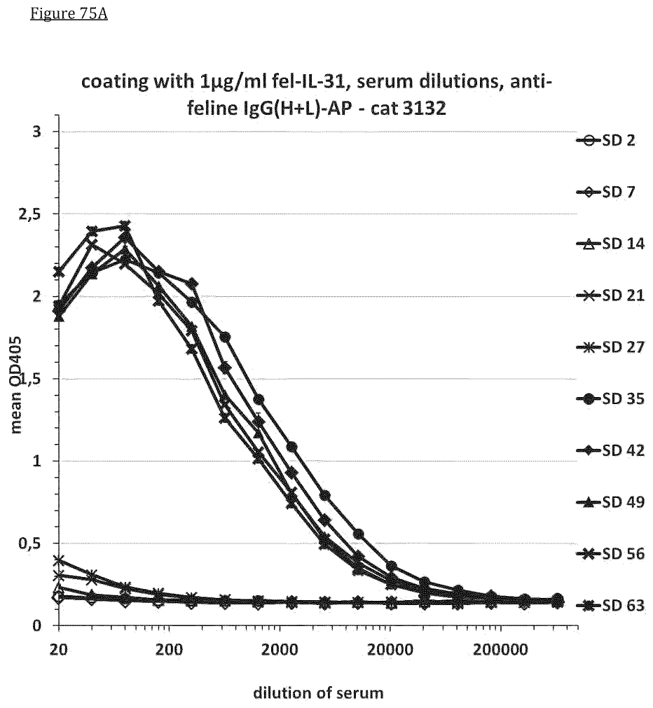

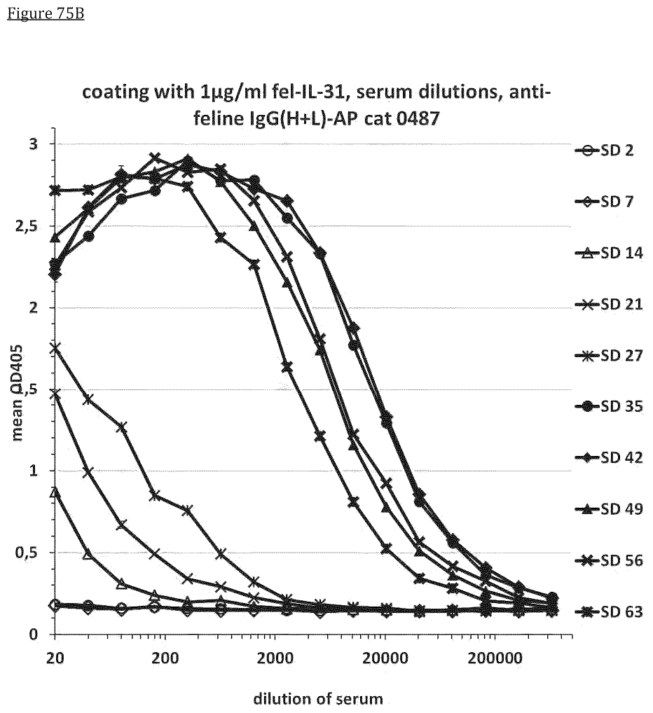

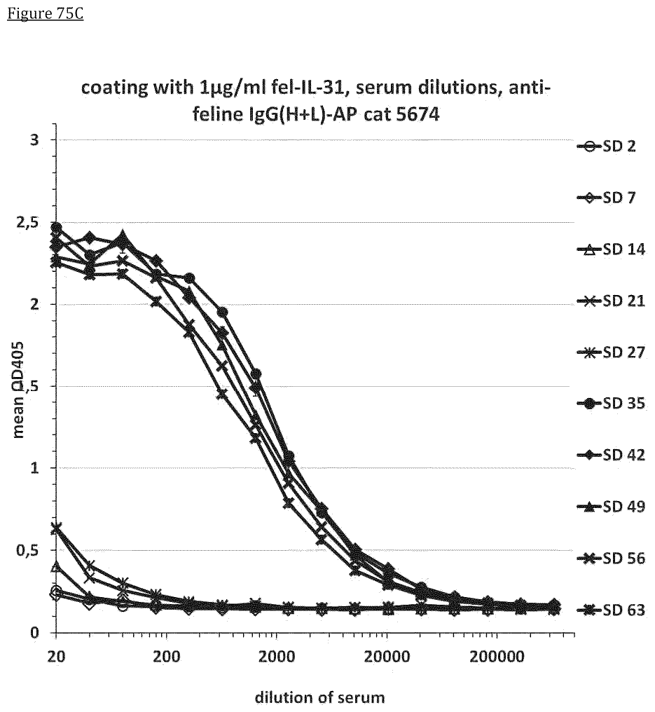

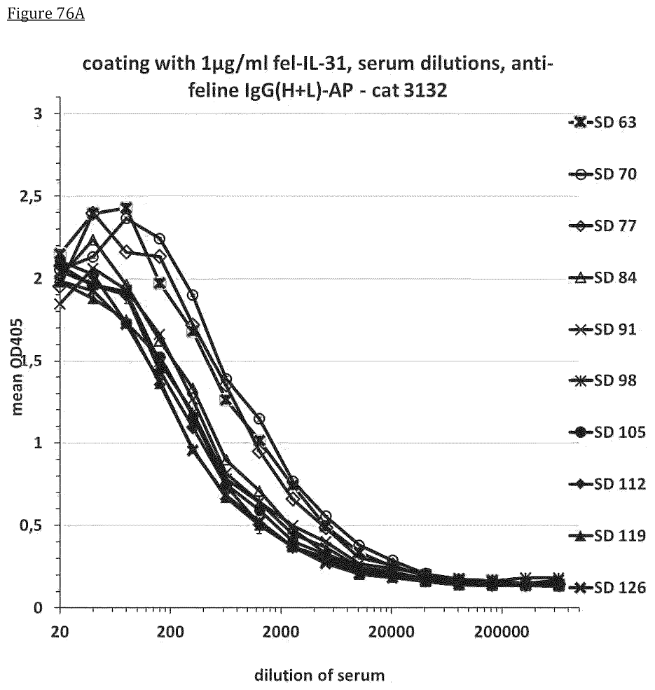

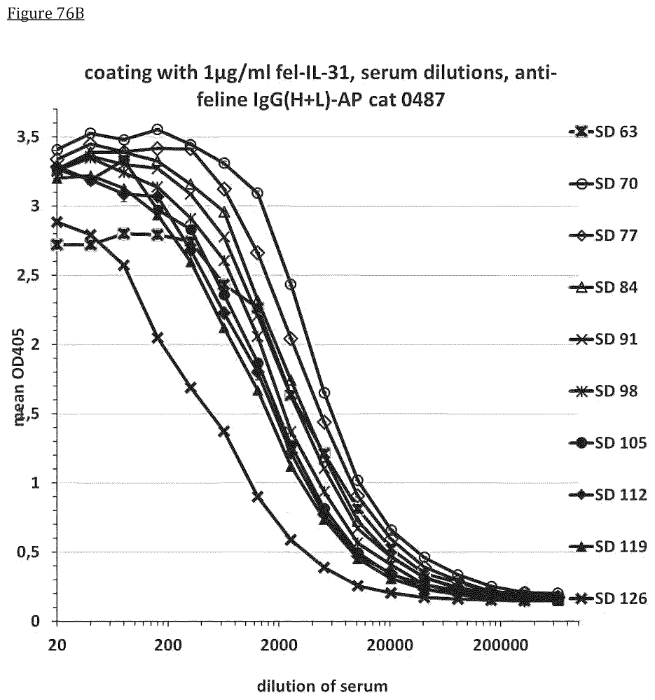

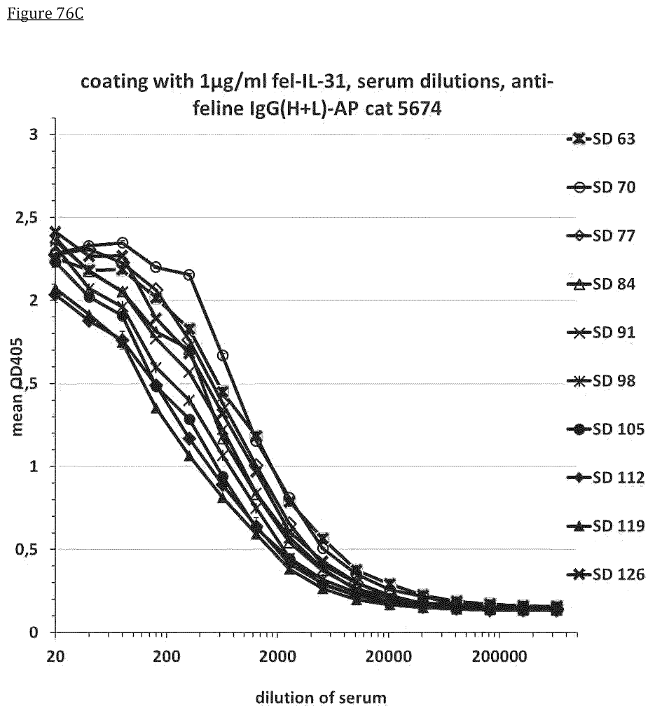

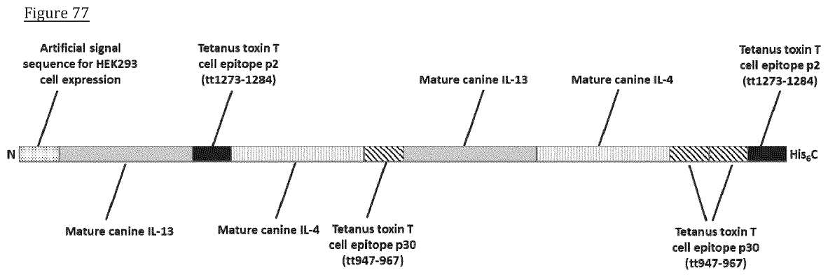

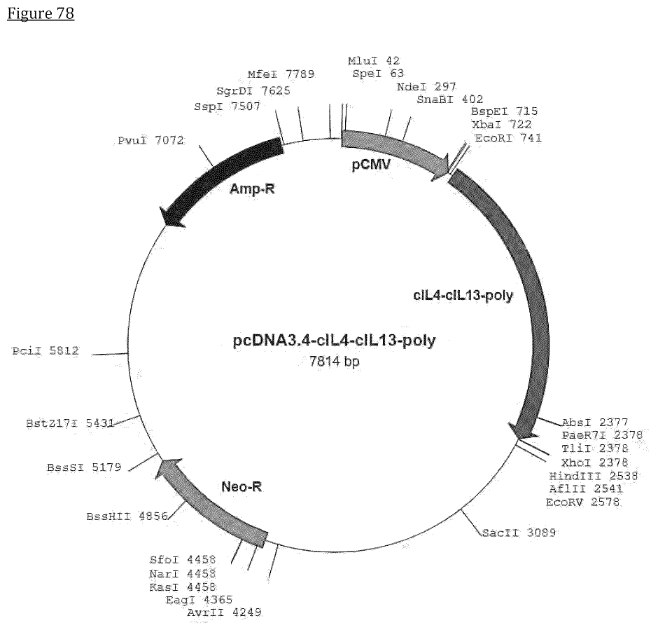

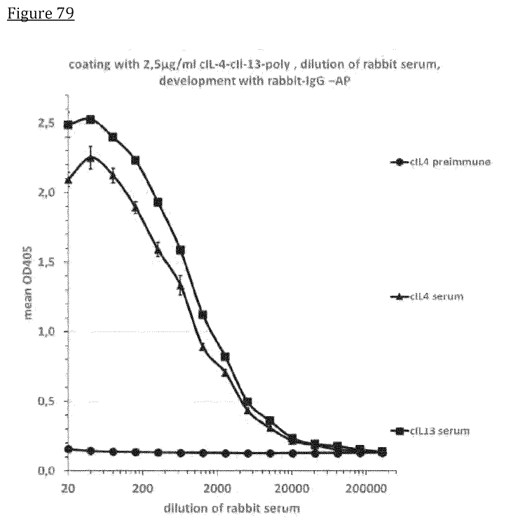

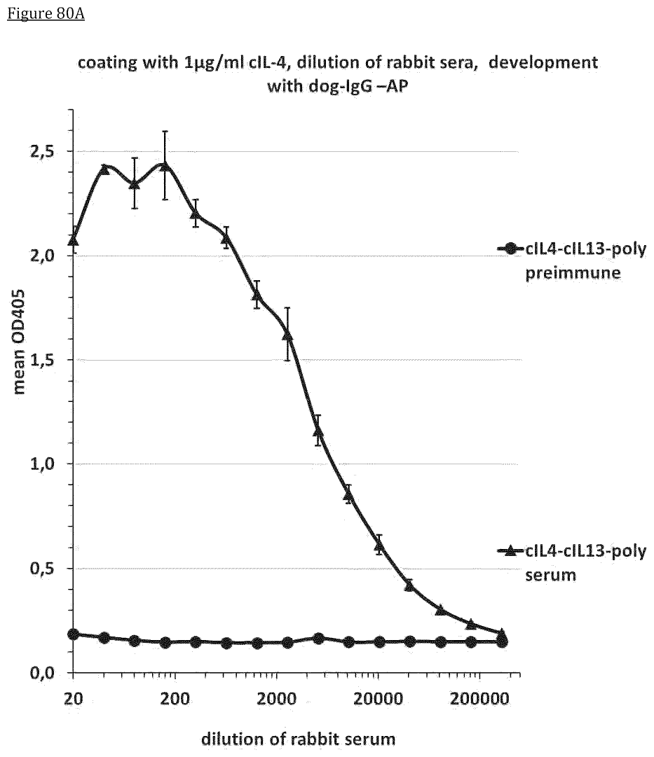

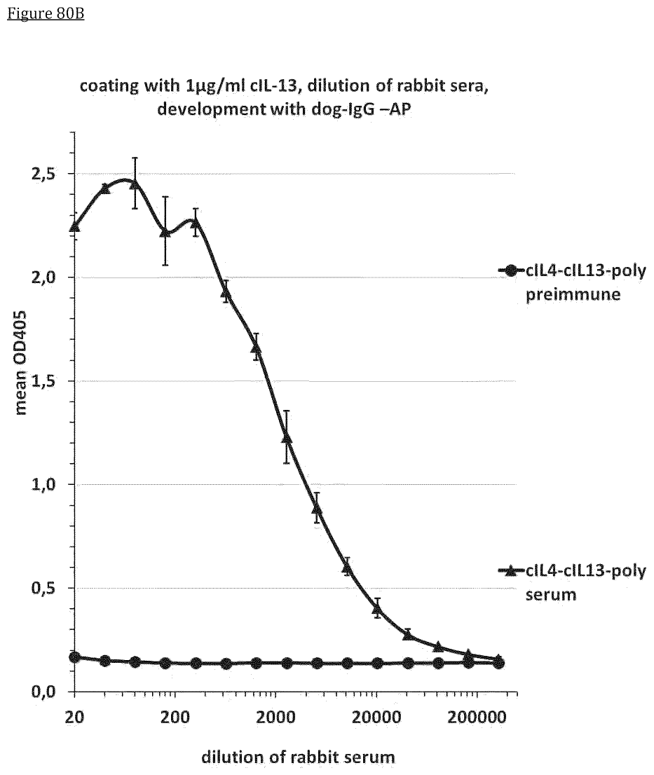

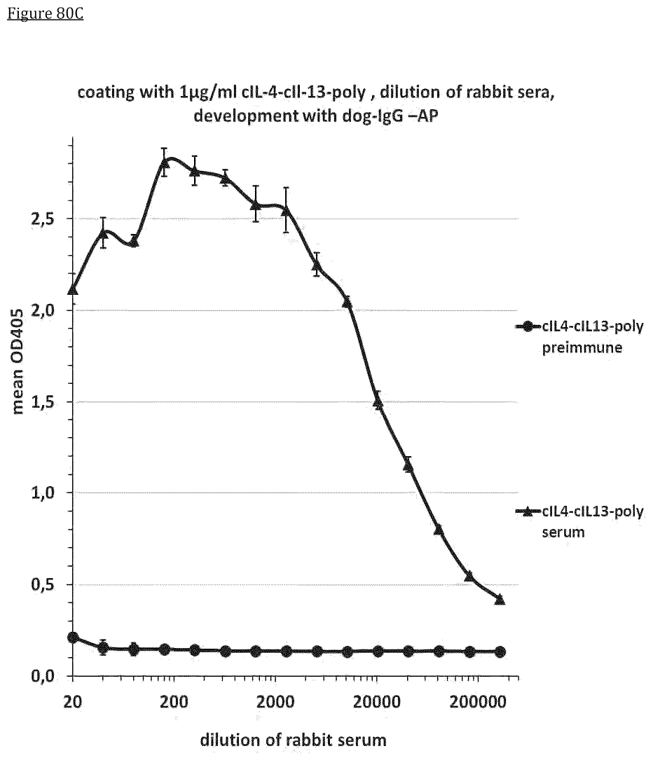

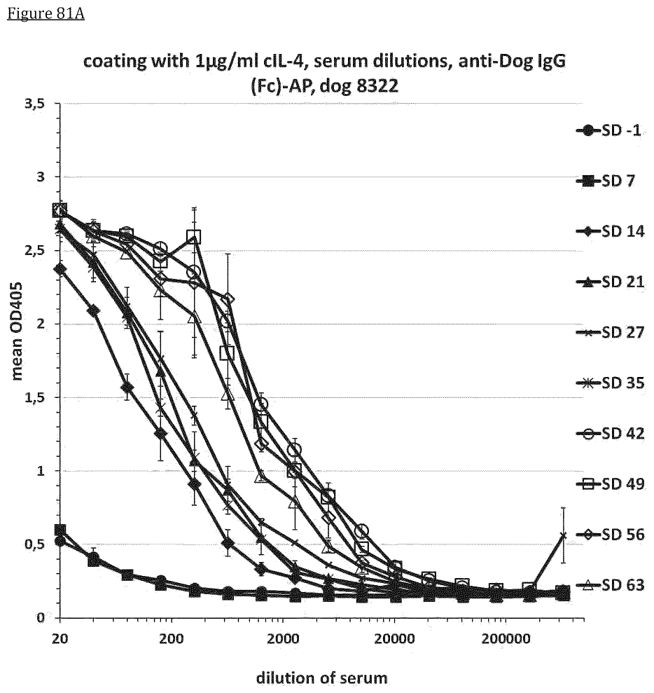

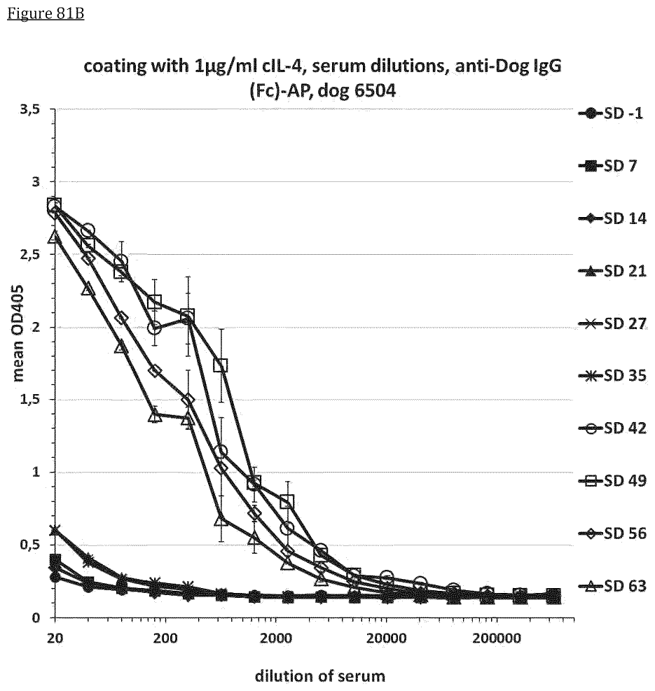

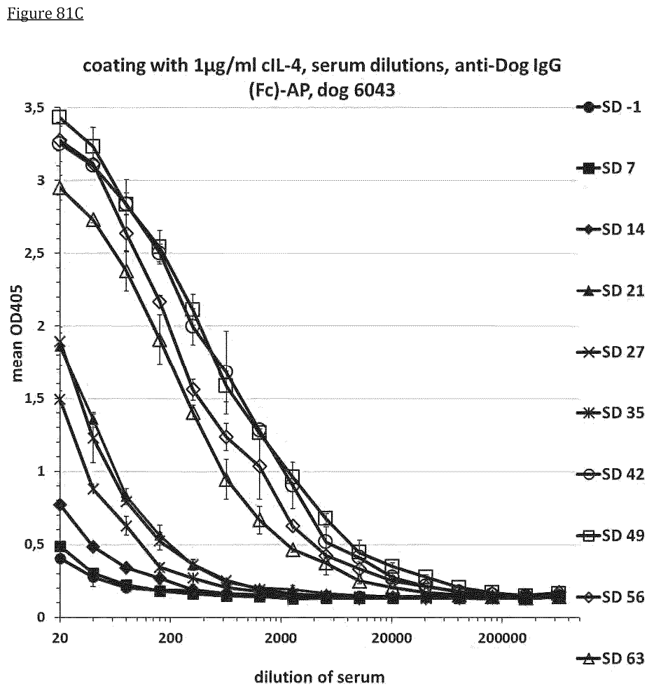

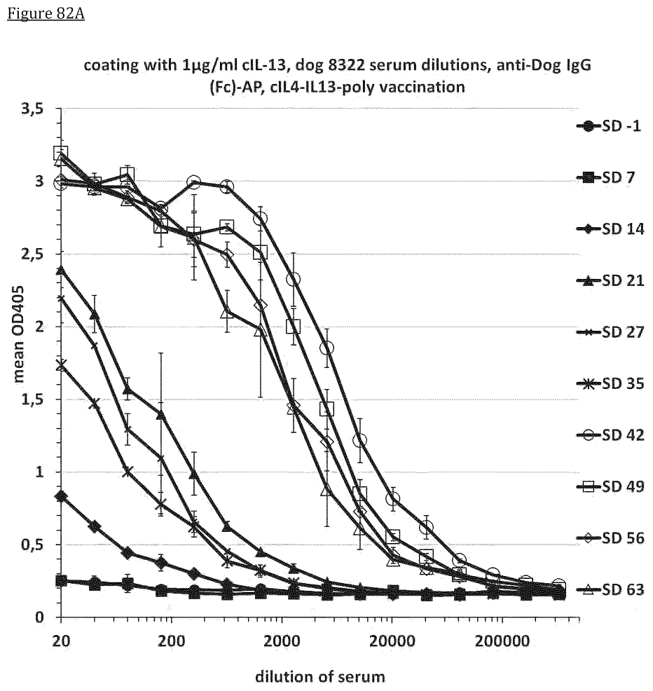

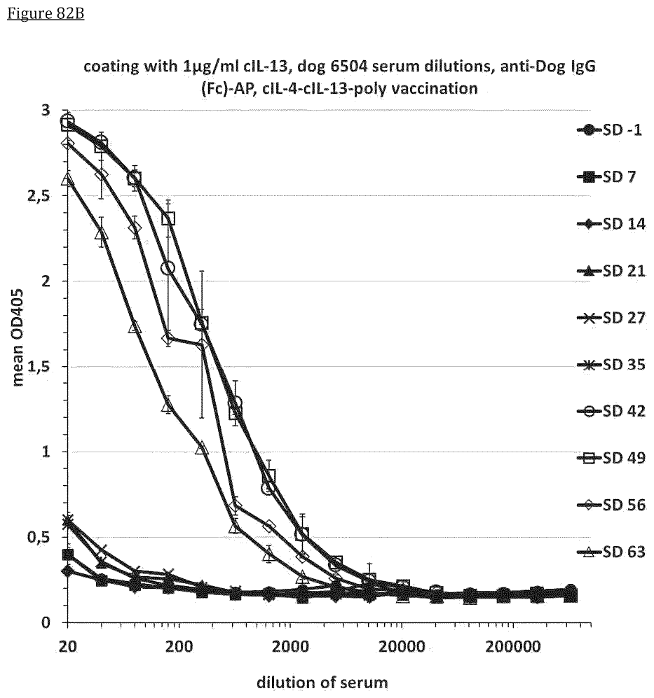

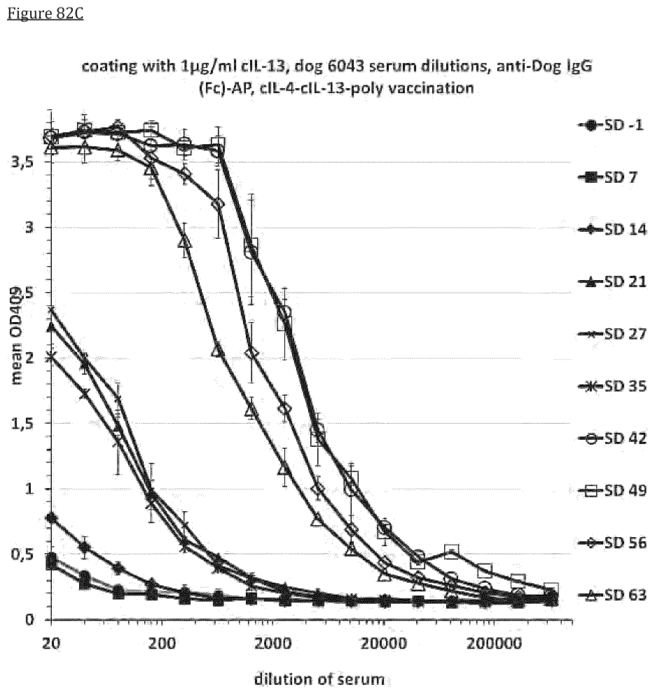

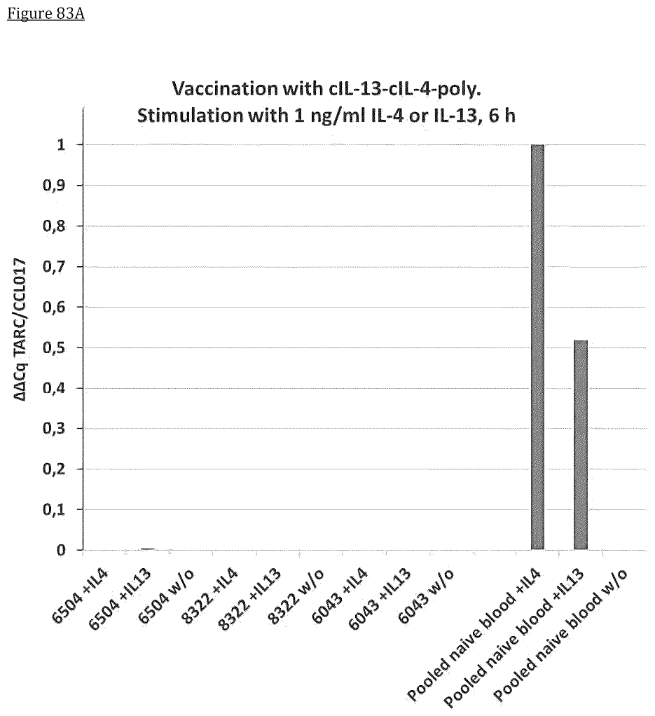

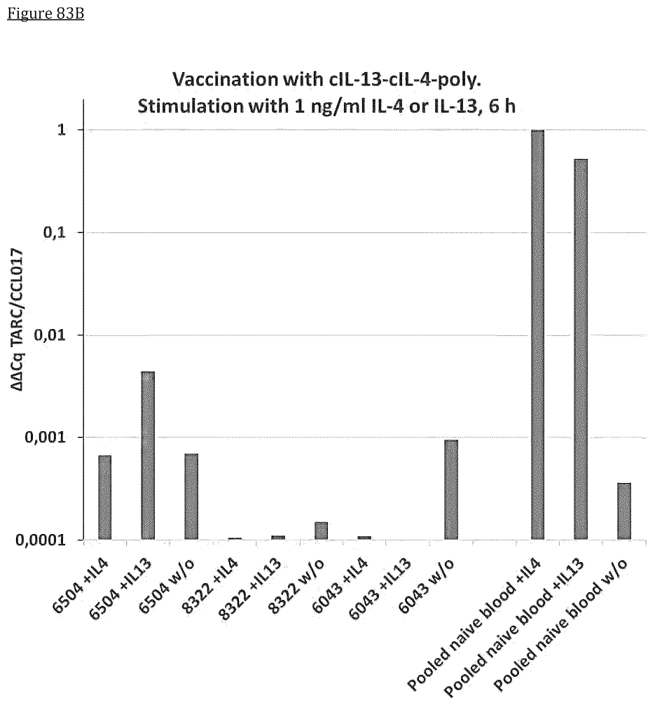

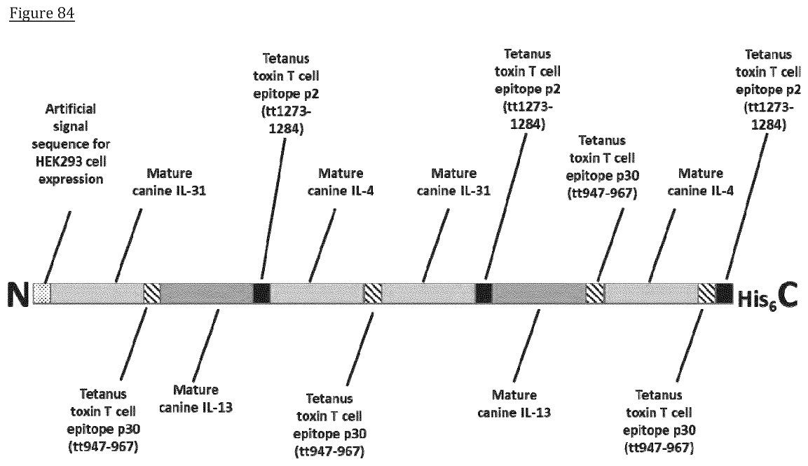

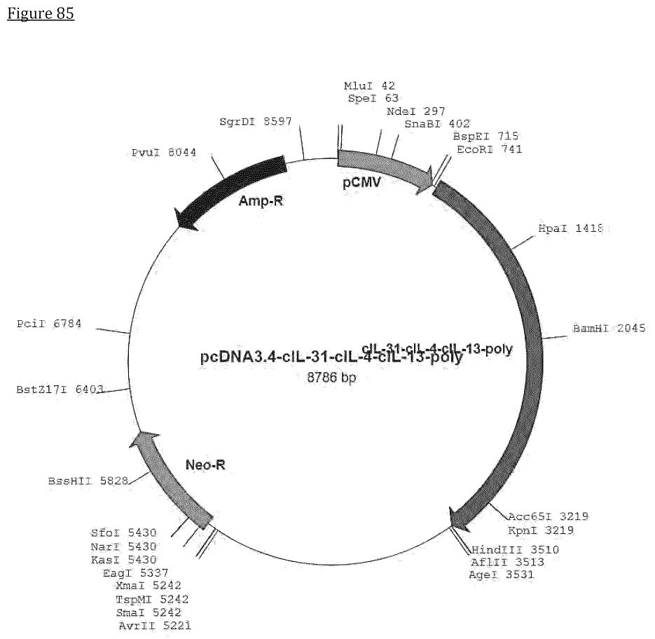

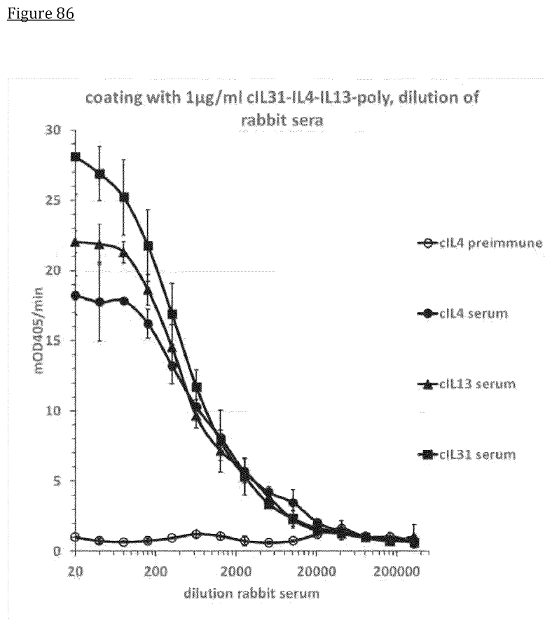

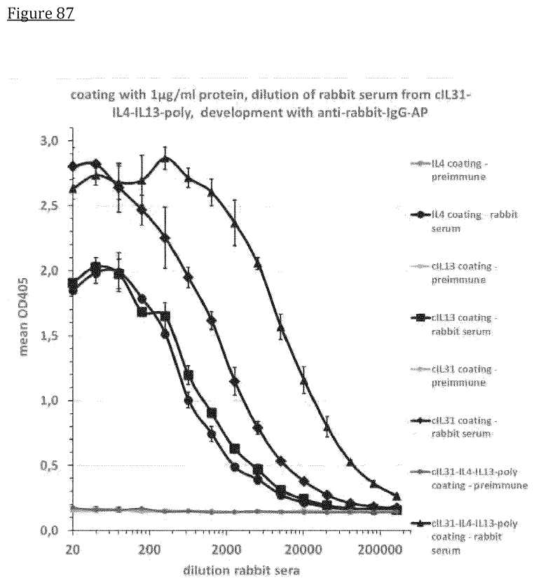

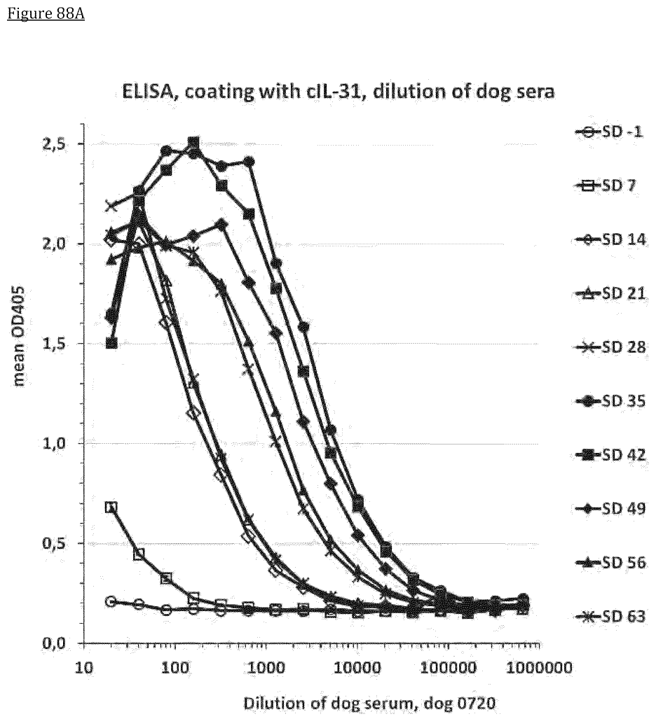

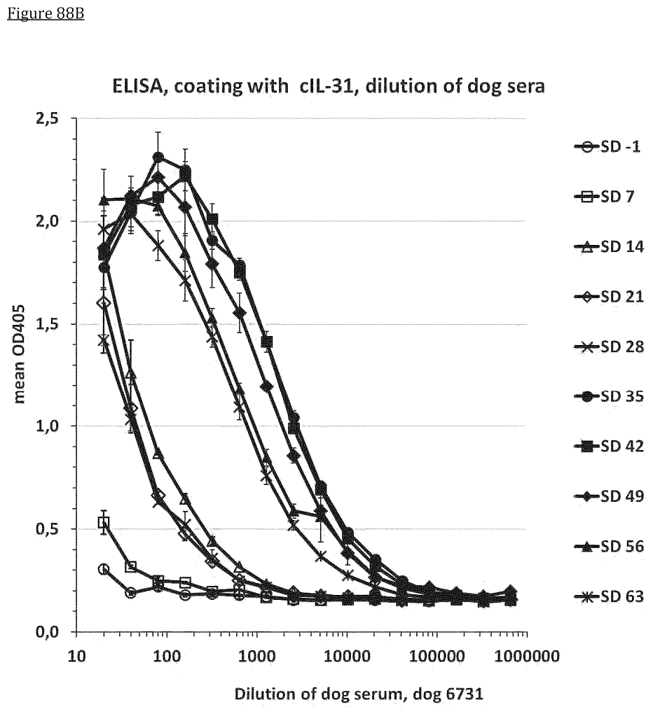

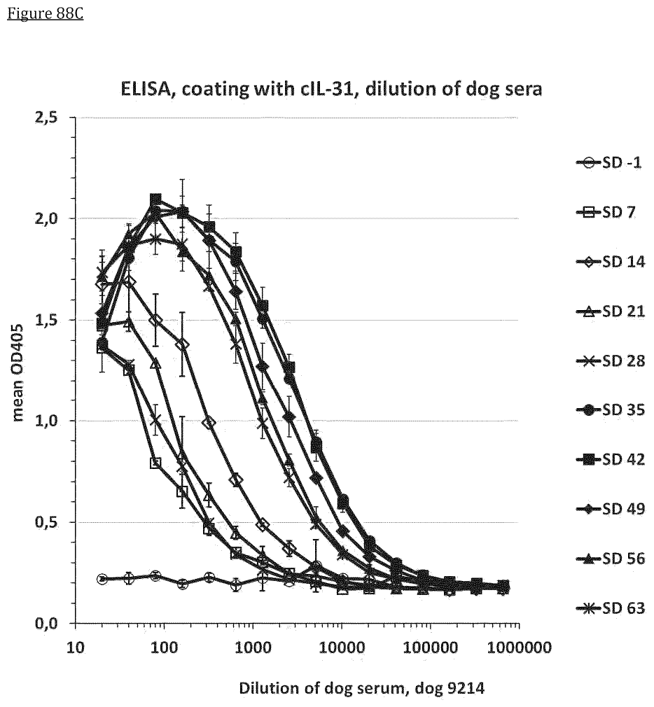

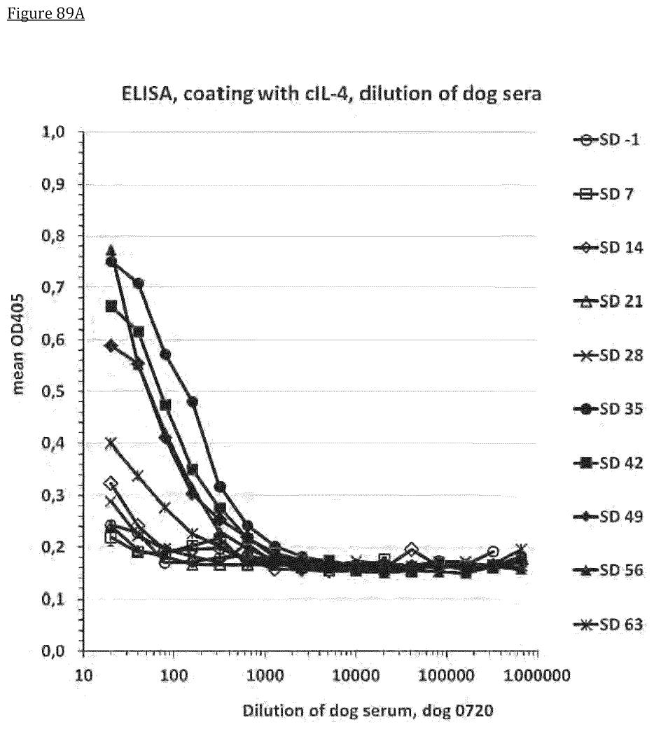

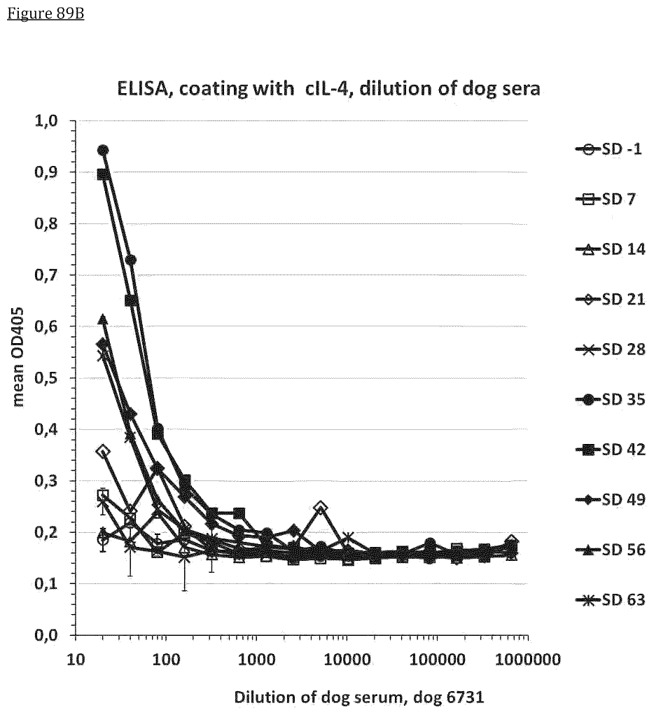

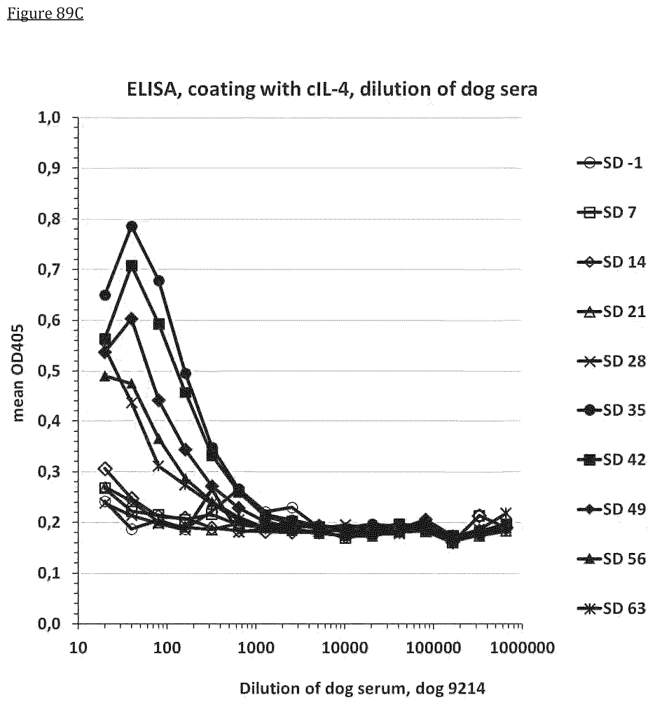

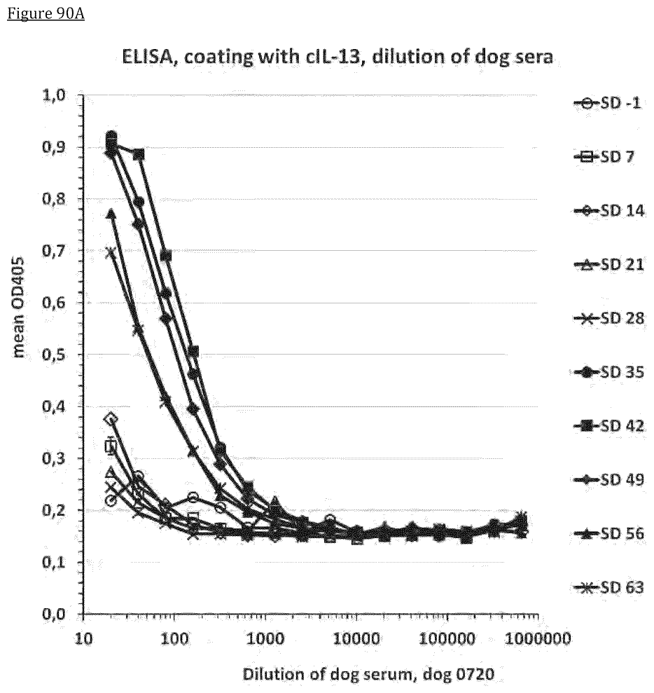

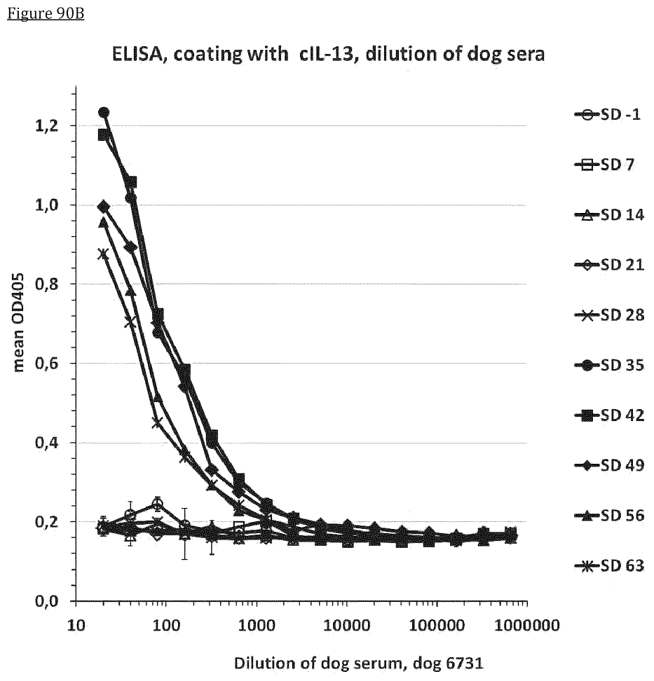

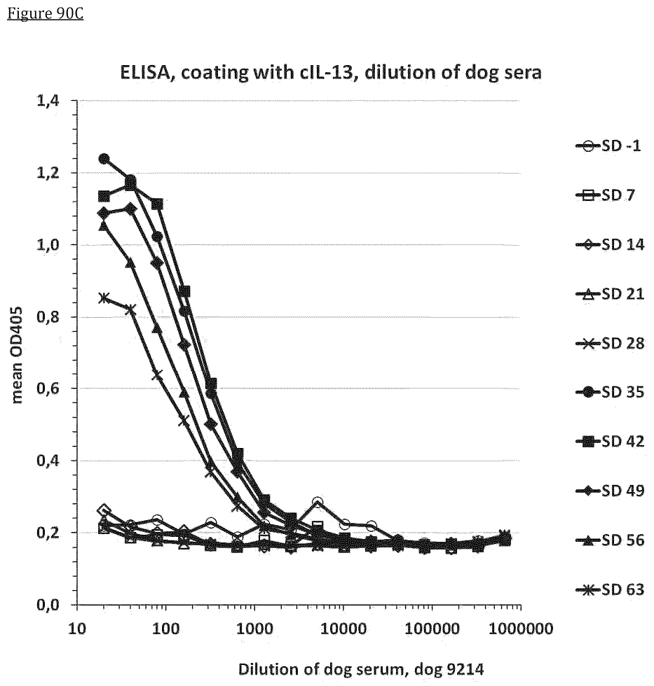

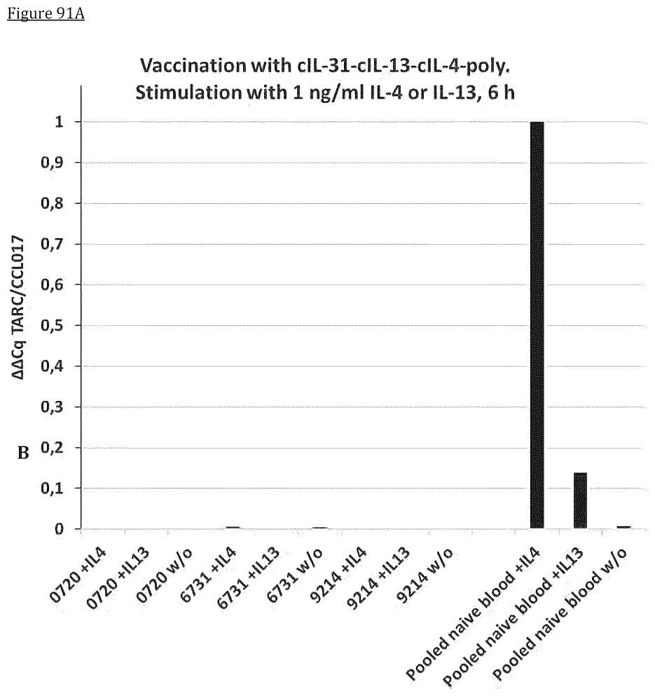

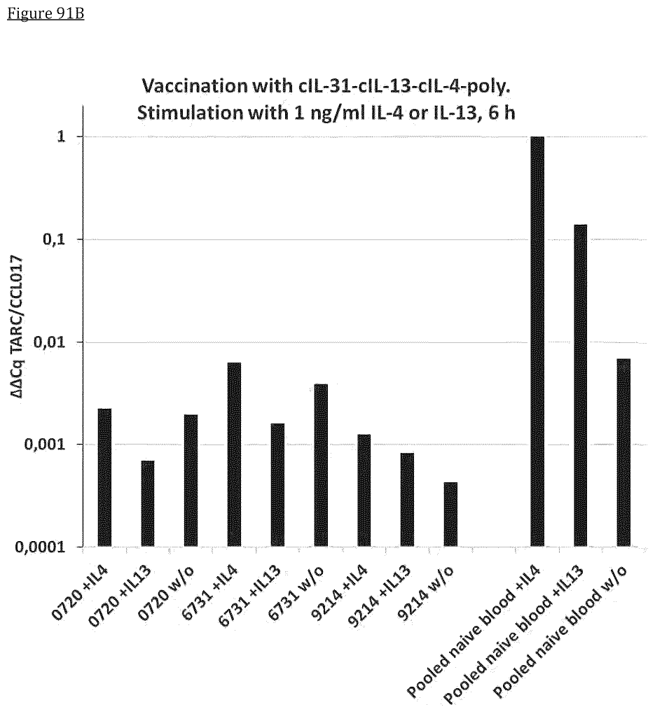

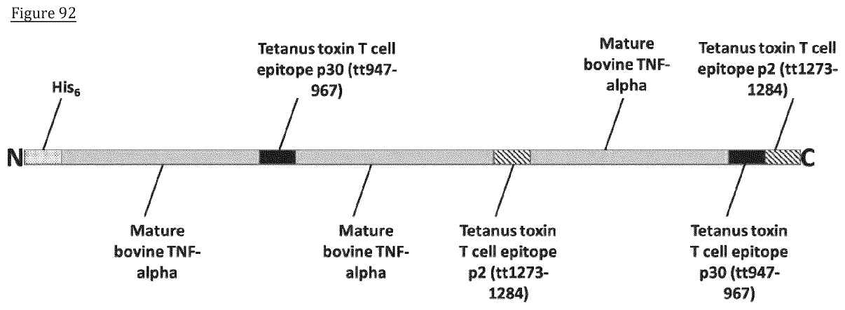

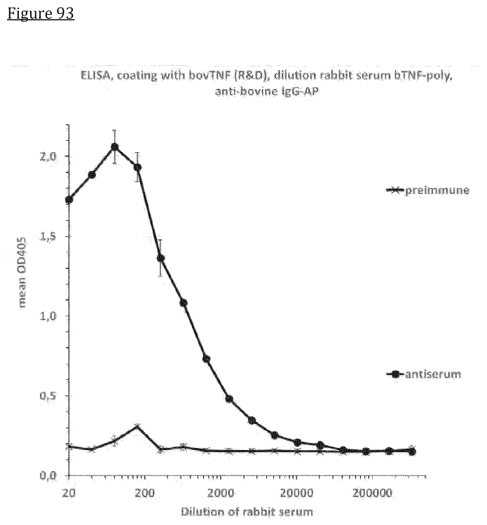

1. A vaccine composition for breaking self-tolerance against a self-protein of a host, wherein the vaccine composition is capable of raising autoantibodies against said self-protein when the vaccine composition is administered to the host, and wherein the vaccine composition comprises:
a) a polyprotein, a DNA encoding for the polyprotein and/or an RNA encoding for the polyprotein, wherein the polyprotein comprises
at least two self-protein segments derived from a first self-protein of the host;
at least two self-protein segments derived from a second self-protein of the host;
optionally at least two self-protein segments derived from a third self-protein of the host; and
one or more T-cell epitopes of non-host origin in between and/or adjacent to the self-protein segments;
and
b) one or more immunostimulatory oligonucleotides.

2. The vaccine composition according to claim 1, wherein the one or more T-cell epitopes are selected from the group consisting of an artificial T-cell epitope peptide sequence and a T-cell epitope peptide sequence derived from a non-self protein, optionally from a pathogenic protein.

3. The vaccine composition according to claim 1, wherein the one or more T-cell epitopes are Tetanus toxin T-cell epitopes, optionally a Tetanus toxin T-cell epitope
(i) comprising at least 95% sequence identity with SEQ ID NO: 1, SEQ ID NO: 39 or SEQ ID NO: 2
or
(ii) selected from the group consisting of SEQ ID NO: 1, SEQ ID NO: 39 and SEQ ID NO: 2

4. The vaccine composition according to claim 1, wherein the polyprotein comprises two or three self-protein segments derived from the first, second, and optionally third, self-protein.

5. The vaccine composition according to claim 1, wherein the self-protein segment is
(i) a full-length self-protein; or
(ii) a truncated self-protein containing a B-cell epitope; or
(iii) a derivative of a self-protein which has at least 80% sequence identity, optionally at least 90% sequence identity and optionally at least 95% sequence identity to the full-length self-protein.

6. The vaccine composition according to claim 1, wherein the first and/or second, and/or optionally third, self-protein is/are derived from a cytokine; optionally a cytokine selected from the group consisting of an IL-31, IL-4, IL-5, IL-13, IL-33, and TNF-alpha protein.

7. The vaccine composition according to claim 1, wherein the first or second, or optionally third self-protein is a IL-31 protein, optionally is canine IL-31 (SEQ ID NO: 3), feline IL-31 (SEQ ID NO: 60), pig IL-31 (SEQ ID NO: 68), bovine IL-31, or human IL-31 (SEQ ID NO: 69).

8. The vaccine composition according to claim 1, wherein the first or second, or optionally third self-protein is an IL-5 protein, optionally canine IL-5 (SEQ ID NO: 41), feline IL-5 (SEQ ID NO: 76), pig IL-5 (SEQ ID NO: 77), chicken IL-5 (SEQ ID NO: 78 or 79), bovine IL-5 (SEQ ID NO: 80) or human IL-5 (SEQ ID NO: 81).

9. The vaccine composition according to claim 1, wherein the first or second, or optionally third self-protein is an IL-4 protein, optionally canine IL-4 (SEQ ID NO: 56), feline IL-4 (SEQ ID NO: 70), pig IL-4 (SEQ ID NO: 71), chicken IL-4 (SEQ ID NO: 72), bovine IL-4 (SEQ ID NO: 73) or human IL-4 (SEQ ID NO: 74 or 75).

10. The vaccine composition according to claim 1, wherein the first or second, or optionally third self-protein is derived from an optionally canine IL-13 (SEQ ID NO: 46), feline IL-13 (SEQ ID NO: 82), pig IL-13 (SEQ ID NO: 83), chicken IL-13 (SEQ ID NO: 84), bovine IL-13 (SEQ ID NO: 85) or human IL-13 (SEQ ID NO: 86).

11. The vaccine composition according to claim 1, wherein the first or second, or optionally third self-protein is an IL-33 protein, optionally canine IL-33 (SEQ ID NO: 50 or 51), feline IL-33 (SEQ ID NO: 87, 88, 89, or 90), pig IL-33 (SEQ ID NO: 91, 92, 93, or 94), bovine IL-33 (SEQ ID NO: 95 or 96) or human IL-33 (SEQ ID NO: 97, 98, 99, or 100).

12. The vaccine composition according to claim 1, wherein the polyprotein has
(i) at least 85% sequence identity with SEQ ID NO: 203 or 205;
or
(ii) the sequence of SEQ ID NO: 203 or 205.

13. The vaccine composition according claim 1, wherein the one or more immunostimulatory oligonucleotides are selected from the group consisting of A-class, B-class, and C-class immunostimulatory oligonucleotides, and mixtures thereof, and wherein optionally the one or more immunostimulatory oligonucleotides are selected from the group consisting of B-class immunostimulatory oligonucleotides.

14. The vaccine composition according to claim 1, wherein at least one or each of the one or more immunostimulatory oligonucleotides
(i) comprise at least 75% sequence identity with SEQ ID NO: 5 or SEQ ID NO: 6; or
(ii) are selected from the group consisting of SEQ ID NO: 5 and SEQ ID NO: 6.

15. The vaccine composition according to claim 1, wherein at least some phosphodiester moieties in the one or more immunostimulatory oligonucleotides have been chemically modified to increase nuclease resistance, optionally have been replaced by phosphorothioate moieties.

16. The vaccine composition according to claim 1, wherein the vaccine composition further comprises an adjuvant conferring a depot effect.

17. A Polyprotein, a DNA encoding for the polyprotein and/or an RNA encoding for the polyprotein for use in a vaccine composition to break self-tolerance against a self-protein of a host, wherein the polyprotein comprises at least two self-protein segments derived from a first self-protein of the host; at least two self-protein segments derived from a second self-protein of the host;
optionally at least two self-protein segments derived from a third self-protein of the host; and one or more T-cell epitopes of non-host origin in between and/or adjacent to the self-protein segments.

18. A product comprising-Use of a polyprotein to break self-tolerance against a self-protein of a host, wherein the self-tolerance is broken by the production of autoantibodies when the polyprotein is capable of being administered to the host, and wherein the polyprotein comprises at least two self-protein segments derived from a first self-protein of the host; at least two self-protein segments derived from a second self-protein of the host; optionally at least two self-protein segments derived from a third self-protein of the host; and
one or more T-cell epitopes of non-host origin in between and/or adjacent to the self-protein segments.

19. The vaccine composition according to claim 1 or polyprotein comprising at least two self-protein segments derived from a first self-protein of the host; at least two self-protein segments derived from a second self-protein of the host; optionally at least two self-protein segments derived from a third self-protein of the host; and one or more T-cell epitopes of non-host origin in between and/or adjacent to the self-protein segments for a method of preventing or treating a disease in a subject, wherein the method comprises administering the vaccine composition or the polyprotein to the subject.

20. The vaccine composition or polyprotein according to claim 19 wherein the subject is a mammal, said mammal comprising a human and/or non-human animal.

21. The vaccine composition or polyprotein according to claim 20 wherein the subject is an animal selected from the group consisting of cattle, poultry, swine, and companion animals such as cats and dogs.

22. The vaccine composition or polyprotein according to claim 19, wherein the disease is
a chronic diseases selected from the group consisting of an autoimmune disease, AIDS and cancer; or
a pruritic condition, optionally selected from the group consisting of atopic dermatitis, eczema, psoriasis, scleroderma and pruritis; or
an allergic condition, optionally selected from the group consisting of allergic dermatitis, summer eczema, urticaria, heaves, inflammatory airway disease, recurrent airway obstruction, airway hyper-responsivness, chronic obstruction pulmonary disease and inflammatory process resulting from autoimmunity,
wherein optionally the disease is a pruritic condition or an allergic condition, optionally atopic dermatitis.

23. An enzyme-linked immunosorbent assay method for detecting autoantibodies, optionally obtained against the polyprotein contained in the vaccine composition of claim 1, wherein the method comprises
c) Adsorbing an antigen onto a test surface;
d) Blocking free binding sites on the test surface;
e) Incubating the antigen-coated and blocked test surface with a mixture comprising a labeled antibody against the antigen and a to-be-tested autoantibody against the antigen; and
f) Detecting the binding of the labeled antibody.

24. The enzyme-linked immunosorbent assay method according to claim 23, wherein the antigen comprises or is a polyprotein comprising at least two self-protein segments derived from a first self-protein of the host; at least two self-protein segments derived from a second self-protein of the host; optionally at least two self-protein segments derived from a third self-protein of the host; and one or more T-cell epitopes of non-host origin in between and/or adjacent to the self-protein segments or a single protein segment or epitope-carrying peptide thereof.


SEQUENCE LISTING (205 sequences)

SEQ ID NO: 1 - protein, 15 aa, Clostridium tetani
  MISC_FEATURE (1)..(15): SEQ ID NO: 1 is the amino acid sequence of the Tetanus Toxin T-cell epitope p2
QYIKANSKFIGITEL

SEQ ID NO: 2 - protein, 21 aa, Clostridium tetani
  MISC_FEATURE (1)..(21): SEQ ID NO: 2 is the amino acid sequence of the Tetanus Toxin T-cell epitope p30
FNNFTVSFWLRVPKVSASHLE

SEQ ID NO: 3 - protein, 136 aa, Canis lupus
  MISC_FEATURE (1)..(136): SEQ ID NO: 3 is the amino acid sequence of canine IL-31
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 4 - protein, 507 aa, Artificial Sequence
  SEQ ID NO: 4 is one version of the amino acid sequence of the cIL-31 polyprotein used for the vaccine composition of Example 13
APTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQGSGGQYIKANSKFIGITELAGSGSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQAGSGFNNFTVSFWLRVPKVSASHLESGGSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQAGSGFNNFTVSFWLRVPKVSASHLEGSGGQYIKANSKFIGITELHHHHHH

SEQ ID NO: 5 - DNA, 20 bp, Artificial Sequence
  SEQ ID NO: 5 is the nucleic acid sequence of the immunostimulatory oligonucleotide 1668-PTO
tccatgacgttcctgatgct

SEQ ID NO: 6 - DNA, 24 bp, Artificial Sequence
  SEQ ID NO: 6 is the nucleic acid sequence of the immunostimulatory oligonucleotide 2006-PTO
tcgtcgttttgtcgttttgtcgtt

SEQ ID NO: 7 - DNA, 7637 bp, Artificial Sequence
  SEQ ID NO: 7 is the nucleic acid sequence of the plasmid pcDNA3.4-cIL31-poly encoding the cIL-31 plyprotein construct of Example 1
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgctgtctcacacaggcccttctagattcgccctgttcctgctgtgcagcatggaaaccctgctgagcagccacatggcccctacacatcagctgcctccatccgacgtgcggaagatcatcctggaactgcagcctctgagcagaggcctgctggaagattaccagaaaaaagaaaccggcgtgcccgagagcaacaggaccctgctgctgtgtctgaccagcgatagccagcctcctagactgaacagcagcgccatcctgccttacttcagagccatcagacccctgagcgacaagaacatcatcgacaagatcatcgagcagctggacaagctgaagttccagcacgagcccgagacagagattagcgtgcccgccgataccttcgagtgcaagagcttcatcctgaccatcctgcagcagttcagcgcctgcctggaaagcgtgttcaagagcctgaacagcggccctcaaggatctggcggccagtacatcaaggccaacagcaagttcatcggcatcaccgagctggccggctctggatctcatatggctccaactcaccagctgccacctagtgatgtgcgcaagattattctcgagctgcagcccctgtctaggggcctgcttgaggactatcaaaagaaagagacaggcgtccccgagtccaatcgcacactgctgctttgcctgacatccgacagccagccacctcggctgaatagctctgccattctgccctactttcgggccattaggcccctgtccgataagaatatcattgataagattattgaacagctcgataagctcaaatttcagcacgaaccggaaaccgagatctccgtgcctgccgacacatttgaatgcaagtcttttatcctcacgattctccagcagttttccgcctgtctggaatccgtgtttaagtccctgaatagcggaccccaggccggcagcggcttcaacaatttcaccgtgtccttttggctgcgggtgcccaaagtgtctgccagccatcttgagtctggcggcagctcacacatggcacccactcatcaactcccaccttcagatgtgcggaaaatcatacttgagctgcaaccactgagccggggactgttggaggattaccaaaagaaagaaactggggtcccagaaagcaatcggactctgctcctgtgcctcacctccgactctcagcctccaaggctcaactcctccgctatcctgccatatttccgggccatccggcctctgtctgacaaaaacattattgacaaaattatcgaacaacttgacaaactcaagtttcaacatgagcccgaaaccgaaatttctgtgcccgctgacacttttgagtgtaaatccttcattctcacgatcctgcaacaattctctgcctgcctcgagtctgtgttcaagtctctgaactcaggccctcaggccggctccgggtttaacaactttacagtgtctttctggctcagagtccccaaggtgtccgcctctcaccttgaaggttccggcggacagtatattaaggctaactctaagtttatcgggattacagagctgcaccaccaccatcaccactgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 8 - protein, 5 aa, Artificial Sequence
  Sequence related to the labeled neutralizing antibody suitable for the assay of the invention (cf. WO2013/011407 A1)
YYDIN

SEQ ID NO: 9 - protein, 5 aa, Artificial Sequence
  Sequence related to the labeled neutralizing antibody suitable for the assay of the invention (cf. WO2013/011407 A1)
SYDMS

SEQ ID NO: 10 - protein, 5 aa, Artificial Sequence
  Sequence related to the labeled neutralizing antibody suitable for the assay of the invention (cf. WO2013/011407 A1)
NYGMS

SEQ ID NO: 11 - protein, 17 aa, Artificial Sequence
  Sequence related to the labeled neutralizing antibody suitable for the assay of the invention (cf. WO2013/011407 A1)
WIFPGDGGTKYNETFKG

SEQ ID NO: 12 - protein, 17 aa, Artificial Sequence
  Sequence related to the labeled neutralizing antibody suitable for the assay of the invention (cf. WO2013/011407 A1)
TITSGGGYTYSADSVKG

SEQ ID NO: 13 - protein, 17 aa, Artificial Sequence
  Sequence related to the labeled neutralizing antibody suitable for the assay of the invention (cf. WO2013/011407 A1)
TISYGGSYTYYPDNIKG

SEQ ID NO: 14 - protein, 14 aa, Artificial Sequence
  Sequence related to the labeled neutralizing antibody suitable for the assay of the invention (cf. WO2013/011407 A1)
ARGGTSVIRDAMDY

SEQ ID NO: 15 - protein, 11 aa, Artificial Sequence
  Sequence related to the labeled neutralizing antibody suitable for the assay of the invention (cf. WO2013/011407 A1)
ARQNWVVGLAY

SEQ ID NO: 16 - protein, 11 aa, Artificial Sequence
  Sequence related to the labeled neutralizing antibody suitable for the assay of the invention (cf. WO2013/011407 A1)
VRGYGYDTMDY

SEQ ID NO: 17 - protein, 15 aa, Artificial Sequence
  Sequence related to the labeled neutralizing antibody suitable for the assay of the invention (cf. WO2013/011407 A1)
RASESVDNYGISFMH

SEQ ID NO: 18 - protein, 17 aa, Artificial Sequence
  Sequence related to the labeled neutralizing antibody suitable for the assay of the invention (cf. WO2013/011407 A1)
KSSQSLLNSGNQKNYLA

SEQ ID NO: 19 - protein, 15 aa, Artificial Sequence
  Sequence related to the labeled neutralizing antibody suitable for the assay of the invention (cf. WO2013/011407 A1)
KASQSVSFAGTGLMH

SEQ ID NO: 20 - protein, 7 aa, Artificial Sequence
  Sequence related to the labeled neutralizing antibody suitable for the assay of the invention (cf. WO2013/011407 A1)
RASNLES

SEQ ID NO: 21 - protein, 7 aa, Artificial Sequence
  Sequence related to the labeled neutralizing antibody suitable for the assay of the invention (cf. WO2013/011407 A1)
GASTRES

SEQ ID NO: 22 - protein, 7 aa, Artificial Sequence
  Sequence related to the labeled neutralizing antibody suitable for the assay of the invention (cf. WO2013/011407 A1)
RASNLEA

SEQ ID NO: 23 - protein, 9 aa, Artificial Sequence
  Sequence related to the labeled neutralizing antibody suitable for the assay of the invention (cf. WO2013/011407 A1)
QQSNKDPLT

SEQ ID NO: 24 - protein, 9 aa, Artificial Sequence
  Sequence related to the labeled neutralizing antibody suitable for the assay of the invention (cf. WO2013/011407 A1)
QNDYSYPYT

SEQ ID NO: 25 - protein, 9 aa, Artificial Sequence
  Sequence related to the labeled neutralizing antibody suitable for the assay of the invention (cf. WO2013/011407 A1)
QQSREYPWT

SEQ ID NO: 26 - protein, 109 aa, Artificial Sequence
  Sequence related to the labeled neutralizing antibody suitable for the assay of the invention (cf. WO2013/011407 A1)
DIVLTQSPASLAVSLGQRATISCRASESVDNYGISFMHWYQQKPGQPPKWYRASNLESGIPARFSGSGSRTDFTLTINPVETDDVATYYCQQSNKDPLTFGAGTKLELK

SEQ ID NO: 27 - protein, 109 aa, Artificial Sequence
  Sequence related to the labeled neutralizing antibody suitable for the assay of the invention (cf. WO2013/011407 A1)
DIVMTQTPLSLSVSPGEPASISCRASESVDNYGISFMHWYQQKPGQPPKWYRASNLESGVPDRFSGSGSGTDFTLRISRVEADDAGVYYCQQSNKDPLTFGAGTKLEIK

SEQ ID NO: 28 - protein, 111 aa, Artificial Sequence
  Sequence related to the labeled neutralizing antibody suitable for the assay of the invention (cf. WO2013/011407 A1)
DIVMTQTPLSLSVSPGEPASISCRASESVDNYGISFMHWFQQKPGQSPQLLIYRASNLESGVPDRFSGSGSGTDFTLRISRVEADDAGVYYCQQSNKDPLTFGAGTKLEIK

SEQ ID NO: 29 - protein, 113 aa, Artificial Sequence
  Sequence related to the labeled neutralizing antibody suitable for the assay of the invention (cf. WO2013/011407 A1)
DIVMSQSPSSLSVSAGDKVTMSCKSSQSLLNSGNQKNYLAWYQQKPWQPPKLLIYGASTRESGVPDRFTGSGSGTDFTLTISSVQAEDLAVYYCQNDYSYPYTFGGGTKLEIK

SEQ ID NO: 30 - protein, 113 aa, Artificial Sequence
  Sequence related to the labeled neutralizing antibody suitable for the assay of the invention (cf. WO2013/011407 A1)
EIVMTQSPASLSLSQEEKVTITCKSSQSLLNSGNQKNYLAWYQQKPGQAPKLLIYGASTRESGVPSRFSGSGSGTDFSFTISSLEPEDVAVYYCQNDYSYPYTFGQGTKLEIK

SEQ ID NO: 31 - protein, 111 aa, Artificial Sequence
  Sequence related to the labeled neutralizing antibody suitable for the assay of the invention (cf. WO2013/011407 A1)
DILLTQSPASLAVSLGQRAIISCKASQSVSFAGTGLMHWYQQKPGQQPKLLIYRASNLEAGVPTRFSGSGSRTDFTLNIHPVEEEDAATYFCQQSREYPWTFGGGTKLEIK

SEQ ID NO: 32 - protein, 109 aa, Artificial Sequence
  Sequence related to the labeled neutralizing antibody suitable for the assay of the invention (cf. WO2013/011407 A1)
EIVMTQSPASLSLSQEEKVTITCKASQSVSFAGTGLMHWYQQKPGQAPKWYRASNLEAGVPSRFSGSGSGTDFSFTISSLEPEDVAVYYCQQSREYPWTFGQGTKLEIK

SEQ ID NO: 33 - protein, 121 aa, Artificial Sequence
  Sequence related to the labeled neutralizing antibody suitable for the assay of the invention (cf. WO2013/011407 A1)
QVQLQQSGAELVKPGASVKLSCKASGYTFKYYDINWVRQRPEQGLEWIGWIFPGDGGTKYNETFKGKATLTTDKSSSTAYMQLSRLTSEDSAVYFCARGGTSVIRDAMDYWGQGTSVTVSS

SEQ ID NO: 34 - protein, 121 aa, Artificial Sequence
  Sequence related to the labeled neutralizing antibody suitable for the assay of the invention (cf. WO2013/011407 A1)
EVQLVQSGAEVKKPGASVKVSCKTSGYTFKYYDINWVRQAPGAGLDWMGWIFPGDGGTKYNETFKGRVTLTADTSTSTAYMELSSLRAGDIAVYYCARGGTSVIRDAMDYWGQGTLVTVSS

SEQ ID NO: 35 - protein, 118 aa, Artificial Sequence
  Sequence related to the labeled neutralizing antibody suitable for the assay of the invention (cf. WO2013/011407 A1)
EVKLVESGGGLVKPGGSLKLSCAASGFAFSSYDMSWVRQIPEKRLEWVATITSGGGYTYSADSVKGRFTISRDNARNTLYLQMSSLRSEDTAVYYCARQNWVVGLAYWGQGTLVTVSA

SEQ ID NO: 36 - protein, 118 aa, Artificial Sequence
  Sequence related to the labeled neutralizing antibody suitable for the assay of the invention (cf. WO2013/011407 A1)
EVQLVESGGDLVKPGGSLRLSCVASGFTFSSYDMSWVRQAPGKGLQWVATITSGGGYTYSADSVKGRFTISRDNARNTLYLQMNSLRSEDTAVYYCARQNWVVGLAYWGQGTLVTVSS

SEQ ID NO: 37 - protein, 118 aa, Artificial Sequence
  Sequence related to the labeled neutralizing antibody suitable for the assay of the invention (cf. WO2013/011407 A1)
EVQLVESGGDLVKPGGSLKLSCAASGFSFSNYGMSWVRQTPDKRLEWVATISYGGSYTYYPDNIKGRFTISRDNAKNTLYLQMSSLKSEDTAMYYCVRGYGYDTMDYWGQGTSVTVSS

SEQ ID NO: 38 - protein, 119 aa, Artificial Sequence
  Sequence related to the labeled neutralizing antibody suitable for the assay of the invention (cf. WO2013/011407 A1)
EVQLVESGGDLVKPGGSLRLSCVASGFTFSNYGMSWVRQAPGKGLQWVATISYGGSYTYYPDNIKGRFTISRDNAKNTLYLQMNSLRAEDTAMYYCVRGYGYDTMDYVVGQGTLVTVSS

SEQ ID NO: 39 - protein, 12 aa, Clostridium tetani
  MISC_FEATURE (1)..(12): SEQ ID NO: 39 is the amino acid sequence of the Tetanus Toxin T-cell epitope p4
GQIGNDPNRDIL

SEQ ID NO: 40 - protein, 510 aa, Artificial Sequence
  SEQ ID NO: 40 is another version of the amino acid sequence of the cIL-31 polyprotein used for the vaccine of Example 13
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQGSGGQYIKANSKFIGITELAGSGSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQAGSGFNNFTVSFWLRVPKVSASHLESGGSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQAGSGFNNFTVSFWLRVPKVSASHLEGSGGQYIKANSKFIGITELHHHHHH

SEQ ID NO: 41 - protein, 115 aa, Canis lupus
  MISC_FEATURE (1)..(115): SEQ ID NO: 41 is the amino acid sequence of canine IL-5 protein.
FAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPES

SEQ ID NO: 42 - protein, 445 aa, Artificial sequence
  SEQ ID NO: 42 is the amino acid sequence of the cIL-5 polyprotein used for the vaccine construct of Example 13b.
VENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPESGSGGQYIKANSKFIGITELAGSGFAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPESAGSGFNNFTVSFWLRVPKVSASHLESGGSFAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPESAGSGFNNFTVSFWLRVPKVSASHLEGSGGQYIKANSKFIGITELHHHHHH

SEQ ID NO: 43 - protein, 447 aa, Artificial sequence
  SEQ ID NO: 43 is an alternative amino acid sequence of the cIL-5 polyprotein that can be used for the vaccine construct of Example 13b.
FAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPESGSGGQYIKANSKFIGITELAGSGFAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPESAGSGFNNFTVSFWLRVPKVSASHLESGGSFAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPESAGSGFNNFTVSFWLRVPKVSASHLEGSGGQYIKANSKFIGITELHHHHHH

SEQ ID NO: 44 - DNA, 1419 bp, Canis lupus
  MISC_FEATURE (1)..(1419): SEQ ID NO: 44 is the nucleic acid sequence of the plasmid pcDNA3.4-cIL-5-poly encoding the cIL-5 polyprotein construct of Example 1b.
gaattcccgccgccaccatgggatggagctgcatcatcctgttcctggtcgctacagccaccggcgtgcacagcgtggaaaaccccatgaatagactggtggccgaaaccttgacactcctgagcacccacagaacatggctgatcggcgacggaaacctgatgattcctacccctgagaacaagaaccaccagctgtgtatcaaggaggtgttccagggcatagacacactgaagaatcagaccgctcacggcgaggccgtggataagctgttccaaaatttaagcctgatcaaggagcacatcgagagacagaagaaaagatgcgccggcgagagatggagagtgaccaagttcctggactacctgcaagtgttcctgggagttatcaataccgagtggacccctgaatccggcagcggcggccagtacatcaaggccaatagcaagttcatcggcattacagaactggccggcagcggcttcgccgtcgagaaccctatgaaccgcctggtggctgaaaccctgaccctgctctctacccacagaacctggctgatcggcgacggcaacctgatgatcccaacaccagagaacaagaaccaccagctgtgcattaaagaggtgttccagggtatcgataccctgaaaaaccagacagcccatggagaagccgtggacaagctgttccaaaacctgtccctgatcaaagaacacatcgagcggcagaaaaaaagatgtgccggagagaggtggcgggtcaccaagttcctggattacctgcaggtgtttctgggcgtgatcaacacggagtggacacctgagagcgctggcagcggctttaacaacttcaccgtgtccttttggctgagagttcctaaggtgtccgccagccacctggaaagcggcggatcttttgccgtggaaaatcccatgaaccggctggtggccgagaccctgacactgctgtctacacaccggacctggctgatcggcgatggcaatctgatgatccccacacctgagaacaagaaccaccagctgtgcatcaaggaagtgttccagggcatcgacacccttaagaaccagaccgcccacggcgaggctgtggacaagctgttccagaacctgtctctgatcaaggaacacatcgagcggcagaagaagcggtgcgccggcgaaagatggagagtgacaaagttcctggactatctgcaggtgttcctgggcgttatcaacaccgagtggacccctgagagcgccggcagcggtttcaacaacttcaccgtgagcttctggcttagagtgcccaaggtgtctgcatctcacctggaaggcagcggaggccagtacatcaaagccaacagcaagtttatcggcatcaccgagctgcaccatcatcaccaccactgataagctt

SEQ ID NO: 45 - DNA, 441 bp, Canis lupus
  MISC_FEATURE (1)..(441): SEQ ID NO: 45 is the nucleic acid sequence of the plasmid pcDNA3.4-cIL-5 encoding the cIL-5 protein construct of Example 3b.
gaattcccgccgccaccatgggctggtcctgcatcatcctgttcctggtcgccaccgccacaggcgtgcacagcgtggaaaatcctatgaacagactggtggctgaaaccctcaccctgctgagcacacacagaacctggctgatcggcgacggcaacctgatgatccccacccctgagaacaagaatcaccagctgtgtatcaaagaggtgttccaaggcatcgacaccctgaagaaccagacagcccacggcgaggccgtggacaagctgtttcagaacctgtctctgattaaggaacacatcgagagacagaaaaagcggtgcgccggagaaagatggcgggtgacaaagttcctggattacctgcaggtgttcctgggagtgatcaacaccgagtggaccccagagagccaccaccaccaccatcactgataagctt

SEQ ID NO: 46 - protein, 113 aa, Canis lupus
  MISC_FEATURE (1)..(113): SEQ ID NO: 46 is the amino acid sequence of canine IL-13.
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 47 - protein, 441 aa, Artificial sequence
  SEQ ID NO: 47 is the amino acid sequence of the cIL-13 polyprotein used for the vaccine construct of Example 13c.
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRGSGGQYIKANSKFIGITELAGSGSPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRAGSGFNNFTVSFWLRVPKVSASHLESGGSSPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRAGSGFNNFTVSFWLRVPKVSASHLEGSGGQYIKANSKFIGITELHHHHHH

SEQ ID NO: 48 - DNA, 1407 bp, Artificial sequence
  SEQ ID NO: 48 is the nucleic acid sequence of the plasmid pcDNA3.4-cIL-13-poly encoding the cIL-13 polyprotein construct of Example 1c.
gaattcccgccgccaccatgggctggtcctgcatcatcctgttcctggtcgccacagccaccggcgtgcacagcagcccatctcctgtgacccctagccctaccctgaaggagctgatcgaggaactggtgaatatcacccagaaccaggcctctctgtgcaacggcagcatggtgtggtccgtgaacctgacagccggaatgtactgcgccgccctggaaagcctgatcaacgtgtcagattgcagcgccatccaaagaacacagagaatgctgaaagccctgtgctctcagaagcctgctgccgggcagatcagctctgagagaagccgggacacaaaaatcgaggtgatccagctggtgaagaacctgctgacctacgttagaggcgtgtacagacacggcaacttccggggctctggaggacagtacatcaaggcaaatagcaagttcatcggcatcacagagctggccggcagcggctctccttcacccgtgacccctagccctactctgaaggaactcattgaggaactggtgaatatcacacaaaatcaagccagcctgtgtaacggctccatggtctggagtgtgaacctgaccgctggcatgtactgcgccgctctggaatctctgatcaacgtgtccgactgcagcgccatccagcgaacccagagaatgctgaaggctctgtgttcccagaaacctgccgccggccagatcagcagcgagagaagcagagatacaaagatcgaggtgatccagctggtgaagaacctgctgacctacgtgcggggcgtgtacagacacggaaactttcgggccggcagcggatttaacaacttcaccgtgtccttctggctgagagtgcctaaggtgtctgcttctcacctggaaagcggaggctccagcccatctcctgtgacacccagccccaccctgaaagagctgatcgaagagctggtgaacatcacccagaatcaggccagcctgtgcaacggcagcatggtgtggagcgtgaacctgacagccggaatgtattgtgccgctctggagagcctgattaacgtgagcgactgcagcgctatccagagaacccagcggatgctgaaagccctgtgttctcagaagcccgccgccggccagatcagcagcgagcggagccgcgacaccaagatcgaggtgatccaactggtcaagaatctcctgacctacgtgaggggcgtttaccggcacggcaacttcagagccggcagcggcttcaacaacttcaccgtgtctttttggctcagagtgcccaaggtgtccgccagccacctggaaggcagcggcggccagtacatcaaggccaacagcaagttcatcggcatcaccgagctgcaccaccatcaccaccactgataagctt

SEQ ID NO: 49 - DNA, 375 bp, Artificial sequence
  SEQ ID NO: 49 is the nucleic acid sequence of the bacterial expression plasmid pET30a-cIL-13 encoding the cIL-13 protein construct of Example 3c.
catatgtcacccagtccagtaacaccctcaccaactctaaaggaactaatagaagaactggtgaacattacccaaaaccaagcgagcctgtgcaacggtagcatggtgtggagcgttaacctgaccgcgggcatgtactgcgcggcgctggagagcctgatcaacgtgagcgactgcagcgcgattcagcgtacccaacgtatgctgaaggcgctgtgcagccagaaaccggcggcgggtcaaatcagcagcgagcgtagccgtgataccaagatcgaagtgattcagctggttaaaaacctgctgacctatgttcgcggcgtttatcgccacggcaactttcgtcaccaccatcatcaccattaatgaaagctt

SEQ ID NO: 50 - protein, 154 aa, Canis lupus
  MISC_FEATURE (1)..(154): SEQ ID NO: 50 is the amino acid sequence of amino acids 110-263 of the full length dog IL-33 protein (Uniprot O97863) canine IL-33-WT.
SIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 51 - protein, 154 aa, Artificial sequence
  SEQ ID NO: 51 is the altered amino acid sequence of canine IL-33-CS , which is amino acids 110-263 of the full length dog IL-33 protein (Uniprot O97863), where the 3 cysteine residues are replaced by serine (IL-33-CS) to improve stability of the gene
SIQEYSASLSTYNDQSITFVFEDGSYEIYVEDLRKGQEKDKVLFRYYDSQSPSHETGDDVDGQTLLVNLSPTKDKDFLLHANNEEHSVELQKSENQLPDQAFFLLHRKSSESVSFESKNNPGVFIGVKDNHLALIKVGDQTKDSYIEKTIFKLS

SEQ ID NO: 52 - DNA, 498 bp, Artificial sequence
  SEQ ID NO: 52 is the nucleic acid sequence of the plasmid pET30a(+)-canIL33-WT encoding the cIL-33_WT protein construct of Example 3d.
catatgcaccaccatcatcatcacagcatccaagaatacagcgcgagcctgagcacctacaacgaccagagcatcaccttcgtgtttgaagatggtagctacgagatctatgtggaagacctgcgtaaaggccaggagaaggataaagttctgttccgttactatgacagccaaagcccgagccacgaaaccggtgacgatgtggacggccagaccctgctggttaacctgagcccgaccaaggacaaagattttctgctgcacgcgaacaacgaggaacacagcgtggagctgcaaaagtgcgaaaaccagctgccggatcaagcgttctttctgctgcaccgtaagagcagcgagtgcgttagcttcgaatgcaaaaacaacccgggtgtgtttattggcgttaaggacaaccacctggcgctgatcaaagtgggcgaccaaaccaaggacagctacatcgagaagaccattttcaaactgagctaatgaaagctt

SEQ ID NO: 53 - DNA, 498 bp, Artificial sequence
  SEQ ID NO: 53 is the nucleic acid sequence of the plasmid pET30a(+)-canIL33-CS encoding the cIL-33_CS protein construct construct of Example 3e.
catatgcaccaccaccaccaccactcaatacaagaatactcagctagtctatcaacctataacgaccagagcattacctttgtttttgaagatggtagctacgagatctatgtggaagacctgcgtaaaggccaggagaaggataaagttctgttccgttactatgacagccaaagcccgagccacgaaaccggtgacgatgtggatggccagaccctgctggttaacctgagcccgaccaaggacaaagattttctgctgcacgcgaacaacgaggaacacagcgtggagctgcaaaagagcgaaaaccagctgccggatcaagcgttctttctgctgcaccgtaagagcagcgagagcgttagcttcgaaagcaaaaacaacccgggtgtgtttattggcgttaaggacaaccacctggcgctgatcaaggttggcgaccagaccaaagatagctacatcgagaagaccattttcaaactgagctaatgaaagctt

SEQ ID NO: 54 - protein, 564 aa, Artificial sequence
  SEQ ID NO: 54 is the amino acid sequence of the cIL-33-CS polyprotein used for the vaccine construct of Example 13d.
SIQEYSASLSTYNDQSITFVFEDGSYEIYVEDLRKGQEKDKVLFRYYDSQSPSHETGDDVDGQTLLVNLSPTKDKDFLLHANNEEHSVELQKSENQLPDQAFFLLHRKSSESVSFESKNNPGVFIGVKDNHLALIKVGDQTKDSYIEKTIFKLSGSGGQYIKANSKFIGITELAGSGSIQEYSASLSTYNDQSITFVFEDGSYEIYVEDLRKGQEKDKVLFRYYDSQSPSHETGDDVDGQTLLVNLSPTKDKDFLLHANNEEHSVELQKSENQLPDQAFFLLHRKSSESVSFESKNNPGVFIGVKDNHLALIKVGDQTKDSYIEKTIFKLSAGSGFNNFTVSFWLRVPKVSASHLESGGSSIQEYSASLSTYNDQSITFVFEDGSYEIYVEDLRKGQEKDKVLFRYYDSQSPSHETGDDVDGQTLLVNLSPTKDKDFLLHANNEEHSVELQKSENQLPDQAFFLLHRKSSESVSFESKNNPGVFIGVKDNHLALIKVGDQTKDSYIEKTIFKLSAGSGFNNFTVSFWLRVPKVSASHLEGSGGQYIKANSKFIGITELHHHHHH

SEQ ID NO: 55 - DNA, 1710 bp, Artificial sequence
  SEQ ID NO: 55 is the nucleic acid sequence of the plasmid pET30a-cIL33-(CS-)poly encoding the cIL-33-CS polyprotein construct of Example 1d.
catatgcaccaccatcatcaccactccatccaagaatactccgcttccctgagcacctataacgaccaatccattacctttgtgtttgaagatggcagttatgaaatttacgtcgaagatctgcgtaagggtcaggaaaaagacaaagtgctgtttcgctattacgatagtcaatccccgtcacatgaaaccggcgatgacgttgatggtcagacgctgctggtcaacctgagcccgaccaaagataaagacttcctgctgcatgcgaacaatgaagaacactcggtggaactgcagaaaagcgaaaatcagctgccggaccaagcctttttcctgctgcatcgtaaaagctctgaatcggttagctttgaaagcaaaaacaatccgggcgttttcattggtgtcaaagataaccacctggcactgatcaaagtcggcgatcagacgaaagactcgtacatcgaaaaaaccatcttcaaactgtctggcagtggcggtcaatacattaaagcgaactcaaaattcattggtatcaccgaactggcgggctccggttcaatccaggaatattcggccagcctgtctacctacaatgaccaatcgattacgtttgttttcgaagatggcagctatgaaatctacgtcgaagacctgcgtaaaggtcaagaaaaagataaagttctgttccgctattacgacagtcagtccccgtcacacgaaacgggcgatgacgtggatggtcaaaccctgctggttaacctgagcccgacgaaagataaagactttctgctgcatgcaaacaatgaagaacactctgttgaactgcagaaaagtgaaaatcagctgccggatcaggcgtttttcctgctgcaccgcaaaagttccgaatcggtgagctttgaatctaaaaataatccgggcgtgttcattggtgttaaagataaccacctggcgctgatcaaagttggtgatcagaccaaagactcttacatcgaaaaaacgatcttcaaactgtctgccggcagtggttttaacaatttcaccgtgtccttctggctgcgcgtgccgaaagtttctgcaagtcatctggaatcaggcggttcatcgattcaggaatattccgcttcactgtcgacctacaatgaccaatctattacgtttgtgttcgaagatggcagttatgaaatctacgttgaagatctgcgtaagggccaagaaaaagataaagtcctgttccgctattacgatagccaatctccgagtcacgaaaccggcgatgacgtcgacggccagacgctgctggtgaacctgtctccgacgaaagacaaagactttctgctgcacgccaataatgaagaacactccgttgaactgcagaaatcagaaaaccaactgccggatcaagcctttttcctgttacaccgcaaaagctctgaatccgtctcatttgaatctaaaaataacccgggcgtcttcattggtgtgaaagataatcacctggccctgatcaaagttggcgaccaaacgaaagactcctatattgaaaaaacgatttttaaactgtccgcaggctcaggcttcaacaatttcaccgtgagtttctggctgcgcgtcccgaaagtgtcggctagccatctggaaggctcaggcggtcaatacatcaaagcgaacagcaaattcatcggcatcacggaactgtaatgaaagctt

SEQ ID NO: 56 - protein, 108 aa, Canis lupus
  MISC_FEATURE (1)..(108): SEQ ID NO: 56 is the amino acid sequence of canine IL-4.
HNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRH

SEQ ID NO: 57 - protein, 426 aa, Artificial sequence
  SEQ ID NO: 57 is the amino acid sequence of the cIL-4 polyprotein used for the vaccine construct of Example 13e.
HNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHGSGGQYIKANSKFIGITELAGSGHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHAGSGFNNFTVSFWLRVPKVSASHLESGGSHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHAGSGFNNFTVSFWLRVPKVSASHLEGSGGQYIKANSKFIGITELHHHHHH

SEQ ID NO: 58 - DNA, 1362 bp, Artificial sequence
  SEQ ID NO: 58 is the nucleic acid sequence of the plasmid pcDNA3.4-cIL-5-poly encoding the cIL-4 polyprotein construct of Example 1e.
gaattcccgccgccaccatgggttggagttgcatcatcctatttctagtggccacagccaccggcgtgcactcgcacaacttcaacataacaatcaaagagatcatcaagatgctgaatattctgaccgccagaaacgattcctgcatggaactgacagtcaaggacgtgtttaccgctccaaagaacacctctgacaaggagatcttctgcagagctgctaccgtgctgcggcaaatctacacccacaactgcagcaaccggtacctgcggggcctgtaccggaacctgtcctctatggccaacaagacctgcagcatgaacgagatcaagaagtccaccctgaaggactttctggagcggctgaaggtcatcatgcagaagaaatattacaggcacggcagtggcggccagtacatcaaggccaattctaagttcatcggcatcaccgagctggccggctctggccacaacttcaacatcaccatcaaagagatcatcaagatgctgaacatcctgaccgcacggaacgactcttgcatggaactgaccgtgaaggacgtgttcactgctcctaagaacacctctgataaagaaatcttctgtagagccgctaccgtgctgagacagatctacacacataactgctccaacagatacctgagaggcctgtacagaaatctgtcctctatggccaacaagacctgttccatgaacgaaatcaaaaagtccaccctaaaggatttcctggaacggctgaaggtgatcatgcagaaaaagtactaccggcacgctggcagcggctttaacaactttacagtgtccttctggctgagagtgcctaaggtgtccgcctcccatttggagtccggcggatctcacaacttcaacatcaccatcaaggagatcatcaagatgctgaacatcctgaccgctcggaacgactcctgtatggaactcacagtgaaggacgtgttcaccgcccccaagaatacctccgacaaggagatcttctgcagagccgccacagttctgcggcagatctatacccacaattgctccaacagatacctcagaggcctgtaccgcaacctgagctccatggctaacaaaacctgctccatgaacgagatcaagaagagcaccctgaaagacttcctggagagactgaaggtgatcatgcagaagaagtactacagacacgccggatctggcttcaataacttcaccgtgtctttctggctgagagtgcctaaggtgtctgctagccacctggaaggttctggaggccagtacatcaaggccaactccaagttcatcggcattaccgagctgcaccatcaccaccaccactgataagctt

SEQ ID NO: 59 - DNA, 357 bp, Artificial sequence
  SEQ ID NO: 59 is the nucleic acid sequence of the bacterial expression plasmid pET30a-cIL-4 encoding the cIL-4 protein construct of Example 3f.
catatgcaccaccaccaccaccacaacttcaacataacaataaaggaaataatcaaaatgctgaacatcctgaccgcgcgtaacgatagctgcatggagctgaccgtgaaggacgttttcaccgcgccgaagaacaccagcgataaagaaatcttttgccgtgcggcgaccgtgctgcgtcagatttacacccacaactgcagcaaccgttacctgcgtggtctgtatcgtaacctgagcagcatggcgaacaaaacctgcagcatgaacgagatcaagaaaagcaccctgaaggatttcctggagcgcctgaaggttattatgcagaagaagtattaccgccactaatgaaagctt

SEQ ID NO: 60 - protein, 133 aa, Felis catus
  MISC_FEATURE (1)..(133): SEQ ID NO: 60 is the amino acid sequence of feline IL-31.
APAHRLQPSDVRKIILELRPMSKGLLQDYVSKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 61 - protein, 501 aa, Artificial Sequence
  SEQ ID NO: 61 is the amino acid sequence of the feline IL-31 polyprotein used for the vaccine construct of Example 13f.
APAHRLQPSDVRKIILELRPMSKGLLQDYVSKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQGSGGQYIKANSKFIGITELAGSGAPAHRLQPSDVRKIILELRPMSKGLLQDYVSKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQAGSGFNNFTVSFWLRVPKVSASHLESGGSAPAHRLQPSDVRKIILELRPMSKGLLQDYVSKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQAGSGFNNFTVSFWLRVPKVSASHLEGSGGQYIKANSKFIGITELHHHHHH

SEQ ID NO: 62 - DNA, 7597 bp, Artificial sequence
  SEQ ID NO: 62 is the nucleic acid sequence of the plasmid pcDNA3.4-felIL31-poly encoding the feline IL-31 polyprotein construct of Example 1g.
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggatggtcctgcatcattctgttcctggtggccaccgccacaggcgtgcacagcgcccccgcccacagactgcaacctagcgacgtcaggaaaatcatcttggagctgagacctatgagcaaaggcctgctgcaggactacgtgtccaaagagattggcctgccagaatctaatcactctagcctgccttgcctgtctagcgacagccagctgccccacatcaacggctccgccatcctgccctactttagagccatcagacctctgtccgacaaaaacaccatcgacaagatcatcgagcagctggacaagctgaagttccagcgggaacccgaagccaaggtttccatgcctgctgataatttcgagagaaagaacttcatcctggctgtgctgcagcagttcagcgcttgcctggagcacgtgctgcagagcctgaatagcggacctcagggcagcggcggccagtacatcaaagccaattctaaattcatcggcatcaccgagctggccggcagcggcgcccctgctcacagactgcagcctagcgatgtgcggaagatcatcctggagctgagacctatgagcaagggcctgctgcaggactacgtgtccaaggaaatcggcctgcccgagagcaaccacagtagcctgccatgtctgagcagcgacagccagctgccacatatcaacggcagcgccatcctgccttacttcagagccatccggcctctgagcgacaagaacaccatcgacaagatcatcgagcagctggataagctgaaattccagagagagcctgaggccaaggtcagcatgcctgccgacaactttgagcggaagaattttatcctggccgtgctgcagcagttcagcgcctgcctggaacacgtgctgcaatctctgaacagcggcccccaggccggatctggcttcaacaacttcacagtgtctttttggctgcgggtgcccaaggtgtccgccagccacctggaaagcggcggatctgctccagctcacagactccagccttctgatgtgcggaagatcattctggaactcaggcccatgagcaagggcctgctgcaggattacgtgagcaaggaaatcggactgcctgagagcaaccacagcagcctgccttgtctgtccagcgatagccagctgccacacatcaacggctctgccattctgccttatttccgggccattagacccctgagcgacaagaacacaatcgataagatcatcgaacagctggacaagctgaagttccaaagagagcccgaggccaaggtgtccatgcccgccgacaacttcgagcgcaagaacttcatcctggctgtgctccagcaatttagcgcctgcctggaacacgtgctgcagagcctgaacagcggacctcaagccggctctggttttaacaacttcaccgtgtccttctggctgagagtgcctaaggtctctgcatctcatctggaaggcagcggcggccagtacatcaaggccaacagcaagttcatcggcatcaccgagctgcaccaccaccaccaccactgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 63 - DNA, 6511 bp, Felis catus
  MISC_FEATURE (1)..(6511): SEQ ID NO: 63 is the nucleic acid sequence of the plasmid pcDNA3.4-fel-IL31 encoding the feline IL-31 protein construct of Example 3g.
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggagctgcatcatcctgttcctggtggccaccgccacaggcgtgcacagcgcccctgcccacagactgcagcctagcgacgtgcgcaagatcattctggagctcagacctatgagcaaaggactgctgcaggactacgtgtccaaggaaatcggcctgcctgagagcaaccactcttctctgccttgtctgagcagcgacagccagctgccccacatcaacggctccgctatccttccctactttagagccatccggccactgtctgataagaacaccatcgacaaaatcatcgagcagctggataagctgaagttccagcgggaacctgaggccaaggtcagcatgccagccgacaacttcgagagaaagaatttcatcctggccgtgctgcaacagttcagcgcttgcctggaacacgtgctgcagagcctgaactccggcccccagcaccaccaccaccatcactgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 64 - protein, 157 aa, Bos taurus
  MISC_FEATURE (1)..(157): SEQ ID NO: 64 is the amino acid sequence of bovine TNF-alpha.
LRSSSQASSNKPVAHVVADINSPGQLRWWDSYANALMANGVKLEDNQLVVPADGLYLIYSQVLFRGQGCPSTPLFLTHTISRIAVSYQTKVNILSAIKSPCHRETPEWAEAKPWYEPIYQGGVFQLEKGDRLSAEINLPDYLDYAESGQVYFGIIAL

SEQ ID NO: 65 - protein, 586 aa, Artificial Sequence
  SEQ ID NO: 65 is the amino acid sequence of the bovine TNF-alpha polyprotein used for the immunization of Example 6f.
MHHHHHHLRSSSQASSNKPVAHVVADINSPGQLRWWDSYANALMANGVKLEDNQLVVPADGLYLIYSQVLFRGQGCPSTPLFLTHTISRIAVSYQTKVNILSAIKSPCHRETPEWAEAKPWYEPIYQGGVFQLEKGDRLSAEINLPDYLDYAESGQVYFGIIALGGSGGSFNNFTVSFWLRVPKVSASHLEGSGGSGLRSSSQASSNKPVAHVVADINSPGQLRWWDSYANALMANGVKLEDNQLVVPADGLYLIYSQVLFRGQGCPSTPLFLTHTISRIAVSYQTKVNILSAIKSPCHRETPEWAEAKPWYEPIYQGGVFQLEKGDRLSAEINLPDYLDYAESGQVYFGIIALGGSGGGQYIKANSKFIGITELGGGSGGLRSSSQASSNKPVAHVVADINSPGQLRWWDSYANALMANGVKLEDNQLVVPADGLYLIYSQVLFRGQGCPSTPLFLTHTISRIAVSYQTKVNILSAIKSPCHRETPEWAEAKPWYEPIYQGGVFQLEKGDRLSAEINLPDYLDYAESGQVYFGIIALGGSGGSFNNFTVSFWLRVPKVSASHLEGGSGGGQYIKANSKFIGITEL

SEQ ID NO: 66 - DNA, 7017 bp, Artificial sequence
  SEQ ID NO: 66 is the nucleic acid sequence of the bacterial expression plasmid pET30a-bov-TNF-alpha-poly encoding the bovine TNF-alpha polyprotein construct of Example 1h.
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagctttcattacagctcggtgatgccgataaacttgctattcgccttgatgtattggccgccgccgctaccaccctccaggtggcttgcgctaaccttcggcacacgtaaccaaaagctaacggtgaaattattgaagctgccaccgctgccacccagagctataataccaaaataaacttggccgctttccgcgtagtccaggtaatccggcaggttaatttccgcagataaacgatcacccttctccagctggaaaacgccaccctgataaatcggctcgtaccacggtttcgcttcagcccattccggggtttcgcgatgacaaggagatttgatcgcgctcaggatattcactttagtctggtagctcaccgcaatgcggctaatggtatgcgtcagaaacagcggggtactaggacatccctggccgcgaaacagaacttggctgtaaatcagatacaggccgtctgccggcacaaccagctggttatcctccagtttcacaccgttcgccattaaagcattcgcgtagctgtcccaccaacgcagttggcccgggctgttaatgtccgcaacaacatgcgccaccggtttgttagagctcgcctgagaactagagcgcaggccaccgctgccaccgcccagttcggtaataccgataaatttgctgttcgccttgatatattgaccgccaccgctgccacccagagcgataataccaaaataaacctgaccgctctccgcgtagtccagataatccggcaggttgatttccgcgctcagacggtcacctttttccagttgaaacacgccaccctggtaaatcggctcataccacggtttcgcttccgcccactccggggtttcgcgatggcacgggctcttgatcgcgctcagaatattcactttggtctggtagctcaccgcgatgcggctaatggtatgagtcagaaacagcggggtagaaggacagccctggccgcgaaacagcacttggctgtaaatcaggtacaggccatcagccggcacaaccagctgattatcctccagtttcacgccattcgccatcagcgcattcgcgtagctatcccaccaacgcagctggcccgggctgttgatgtccgcaaccacgtgcgcaaccggtttgttgctgctcgcctggctagaagagcgcaggccgctgccaccgctaccttccagatggctcgcgctaaccttcggcacacgcagccaaaagctaacggtaaagttgttgaagctgccaccgctgccacccagcgcaatgatgccgaagtacacctgaccgctctccgcataatccaggtagtccggcaggttaatctccgcgctcagacgatcacctttttccagttggaacacgccaccctgatagatcggttcgtaccacggcttcgcctccgcccattccggggtttcacggtggcacgggcttttaatcgcgctcaggatgttaaccttggtttgatagctcaccgcaatacggctgatggtgtgggtcagaaacagcggggtgctcgggcaaccttgaccacgaaacagaacctggctatagatcaggtacaggccatccgccggcacaaccagctggttgtcttccagcttcacaccgttcgccatcagcgcgttcgcgtagctgtcccaccaacgcagttgacccgggctattgatgtccgccactacgtgagctacgggtttatttgaactagcttgtgaacttgaccttaggtggtgatggtgatgatgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 67 - protein, 19 aa, Artificial sequence
  SEQ ID NO: 67 is the amino acid sequence of the artificial ER import signal used for mammalian expression in the given examples.
MGWSCIILFLVATATGVHS

SEQ ID NO: 68 - protein, 138 aa, Sus scrofa
  MISC_FEATURE (1)..(138): This is the amino acid sequence of Pig IL-31 protein.
MAPTSVPSPEEQKGIIKELQASVKKVLKDYEEKEKGVQEQERFQIPCLTFNPQSPHNIESSAILPYLRAIKRSFDNDDDIIDIINHLHKLKFQQGPETTVPVPTNSFEGKSFTLSILKAFSKCLTSDFNSHDPRRGQS

SEQ ID NO: 69 - protein, 141 aa, Homo sapiens [sequence omitted]

SEQ ID NO: 70 - protein, 109 aa, Felis catus
  MISC_FEATURE (1)..(109): This is the amino acid sequence of Cat IL-4 protein.
QNFNNTLKEIIKTLNILTARNDSCMELTVMDVLAAPKNTSDKEIFCRATTVLRQIYTHHNCSTKFLKGLDRNLSSMANRTCSVNEVKKCTLKDFLERLKAIMQKKYSKH

SEQ ID NO: 71 - protein, 109 aa, Sus scrofa
  MISC_FEATURE (1)..(109): This is the amino acid sequence of Pig IL-4 protein.
HKCDITLQEIIKTLNILTARKNSCMELPVTDVFAAPENTTEKETFCRASTVLRHIYRHHTCMKSLLSGLDRNLSSMANMTCSVHEAKKSTLKDFLERLKTIMKEKYSKC

SEQ ID NO: 72 - protein, 116 aa, Gallus gallus
  MISC_FEATURE (1)..(116): This is the amino acid sequence of Chicken IL-4 protein.
VPMLCLQLSVPLMESIRIVNDIQGEVSCVKMNVTDIFADNKTNNKTELLCKASTIVWESQHCHKNLQGLFLNMRQLLNASSTSLKAPCPTAAGNTTSMEKFLADLRTFFHQLAKNK

SEQ ID NO: 73 - protein, 111 aa, Bos taurus
  MISC_FEATURE (1)..(111): This is the amino acid sequence of Bovine IL-4 protein.
HKCDITLAEIIKTLNILTTRKNSCMELPVADVFAAPKNTTEKETFCRVGIELRRIYRSHTCLNKFLGGLDRNLNSLASKTCSVNEAKTSTSTLKDLLERLKTIMKEKYSKC

SEQ ID NO: 74 - protein, 129 aa, Homo sapiens [sequence omitted]

SEQ ID NO: 75 - protein, 113 aa, Homo sapiens
  MISC_FEATURE (1)..(113): This is the amino acid sequence of Human IL-4 Isoform 2 protein.
HKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 76 - protein, 115 aa, Felis catus
  MISC_FEATURE (1)..(115): This is the amino acid sequence of Cat IL-5 protein.
IAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 77 - protein, 113 aa, Sus scrofa
  MISC_FEATURE (1)..(113): This is the amino acid sequence of Pig IL-5 protein.
VENTMNRLVAETLTLLSIHRTLLIGDGNLMISTPVHTNHQLCIEEVFQGIDTLKNQTARGDAVEKLFQNLSLIKEYIDRQKKNCGGERWRVTQFLDYLQVFLGVINTEWTMES

SEQ ID NO: 78 - protein, 110 aa, Gallus gallus
  MISC_FEATURE (1)..(110): This is the amino acid sequence of Chicken IL-5, 126 aa protein.
TPLRSNLAELQTWLQQIYQSVDMLNLRIETPVSADDENCIKTLFEGTALLKNNPEMRRFGTFFQSFDKLRPSLTAQLTGEGECDTERKNVKKFIEKLRTFIRKLSRDARV

SEQ ID NO: 79 - protein, 120 aa, Gallus gallus
  MISC_FEATURE (1)..(120): This is the amino acid sequence of Chicken IL-5, 136 aa protein.
TPLRSNLAELQTWLQQIYQSVDMLVSSIFYFCSMNLRIETPVSADDENCIKTLFEGTALLKNNPEMRRFGTFFQSFDKLRPSLTAQLTGEGECDTERKNVKKFIEKLRTFIRKLSRDARV

SEQ ID NO: 80 - protein, 113 aa, Bos taurus
  MISC_FEATURE (1)..(113): This is the amino acid sequence of Bovine IL-5 protein.
VESTMNRLVAETLTLLSSHRTLLIGDGNLMIPTPQHTNHQLCIEEVFQGIDTLKNQTAQGDAVKKIFQNLSLIKEYIDLQKRKCGGERWRVKQFLDYLQVFLGVINTEWTMES

SEQ ID NO: 81 - protein, 115 aa, Homo sapiens
  MISC_FEATURE (1)..(115): This is the amino acid sequence of Human IL-5 protein.
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 82 - protein, 112 aa, Felis catus
  MISC_FEATURE (1)..(112): This is the amino acid sequence of Cat IL-13 protein.
SPGPHSRRELKELIEELVNITQNQVSLCNGSMVWSVNLTTGMYCAALESLINVSDCTAIQRTQRMLKALCTQKPSAGQTASERSRDTKIEVIQLVKNLLNHLRRNFRHGNFK

SEQ ID NO: 83 - protein, 113 aa, Sus scrofa
  MISC_FEATURE (1)..(113): This is the amino acid sequence of Pig IL-13 protein.
SPGPVPPHSTALKELIEELVNITQNQKTPLCNGSMVWSVNLTTSMQYCAALESLINISDCSAIQKTQRMLSALCSHKPPSEQVPGKHIRDTKIEVAQFVKDLLKHLRMIFRHG

SEQ ID NO: 84 - protein, 101 aa, Gallus gallus
  MISC_FEATURE (1)..(101): This is the amino acid sequence of Chicken IL-13 protein.
TPLAMNLSKLKLSDITQGIQKLNRGVQLSEQELLCQAATVLDNMTDCKKDYEPLITSLKSLHGMTNCPPSTDNEIYLRNFLPALGNYTQALYRRISATAAN

SEQ ID NO: 85 - protein, 114 aa, Bos taurus
  MISC_FEATURE (1)..(114): This is the amino acid sequence of Bovine IL-13 protein.
SPSPVPSATALKELIEELVNITQNQKVPLCNGSMVWSLNLTSSMYCAALDSLISISNCSVIQRTKRMLNALCPHKPSAKQVSSEYVRDTKIEVAQFLKDLLRHSRIVFRNERFN

SEQ ID NO: 86 - protein, 122 aa, Homo sapiens
  MISC_FEATURE (1)..(122): This is the amino acid sequence of Human IL-13 protein.
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 87 - protein, 246 aa, Felis catus
  MISC_FEATURE (1)..(246): This is the amino acid sequence of Cat IL-33 canonical isoform protein.
MKTKMEYSTTKISPAKMNSSAGKALVKSPKLGKSQQKAEEVCQMYFMQLRSGLIKKTACYFKKETTKRHSTRRAGKYKEHLVFTARHQQLERPVEGLAFGVPMVQKCFGTTDVPSIQEYSASLSTYNDQSITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECRPAPPFPPDLRILGR

SEQ ID NO: 88 - protein, 117 aa, Felis catus
  MISC_FEATURE (1)..(117): This is the amino acid sequence of alternative form of Cat IL-33 canonical isoform protein.
SITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECRPAPPFPPDLRILGR

SEQ ID NO: 89 - protein, 268 aa, Felis catus
  MISC_FEATURE (1)..(268): This is the amino acid sequence of Cat IL-33 Isoform 268 aa protein.
MKTKMEYSTTKISPAKMNSSAGKALVKSPKLGKSQQKAEEVCQMYFMQLRSGLIKKTACYFKKETTKRHSTRRAGKYKEHLVFTARHQQLERPVEGLAFGVPMVQKCFGTTDVPSIQEYSASLSTYNDQSITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECKNDPGVFIGVKDNHLALIKVEDQTEYFSTENIIFKLS

SEQ ID NO: 90 - protein, 139 aa, Felis catus
  MISC_FEATURE (1)..(139): This is the amino acid sequence of alternative fomr of Cat IL-33 Isoform 268 aa protein.
SITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECKNDPGVFIGVKDNHLALIKVEDQTEYFSTENIIFKLS

SEQ ID NO: 91 - protein, 276 aa, Sus scrofa
  MISC_FEATURE (1)..(276): This is the amino acid sequence of Pig IL-33 (K7GS29) protein.
MKPEVKYSTTKISTAKMNSSAGKVSIKSPKLRKSQQKAKEVCQMYFMQLRSGLIIEKKACYFGKETTKRHSPTKGKKCKKQPLVLAACQQQLGHLERSVEGFTFTNTMVQKYTTATNLLSIKEHSASLSTYNDQYITFAFEDGSYEIYVEDLRKDQEKDKVLLRYYDSQIPSSETDGGGDHRKLMVNLSPTKDKDFLLHANSKEHSVELQKCENPLPEQAFFVLHEQPSKCVSFECKSHPGVFLGVKNNQLALIKLGEHPEDSNRENTTFKLSNLM

SEQ ID NO: 92 - protein, 142 aa, Sus scrofa
  MISC_FEATURE (1)..(142): This is the amino acid sequence of Pig IL-33 thought to be the probable mature sequence of K7GS29.
YITFAFEDGSYEIYVEDLRKDQEKDKVLLRYYDSQIPSSETDGGGDHRKLMVNLSPTKDKDFLLHANSKEHSVELQKCENPLPEQAFFVLHEQPSKCVSFECKSHPGVFLGVKNNQLALIKLGEHPEDSNRENTTFKLSNLM

SEQ ID NO: 93 - protein, 259 aa, Sus scrofa
  MISC_FEATURE (1)..(259): This is another amino acid sequence of Pig IL-33 (K7GRC7).
MKPEVKYSTTKISTAKMNSSAGKVSIKSPKLRKSQQKAKEVCQMYFMQLRSGLIIEKKACYFGKETTKRHSPTKGKKCKKQPLVLAACQQQLGHLERSVEGFTFTNTMVQKYTTATNLLSIKEHSASLSTYNDQYITFAFEDGSYEIYVEDLRKDQEKDGGGDHRKLMVNLSPTKDKDFLLHANSKEHSVELQKCENPLPEQAFFVLHEQPSKCVSFECKSHPGVFLGVKNNQLALIKLGEHPEDSNRENTTFKLSNLM

SEQ ID NO: 94 - protein, 125 aa, Sus scrofa
  MISC_FEATURE (1)..(125): This is another amino acid sequence of Pig IL-33 thought to be the probable mature sequence of K7GRC7.
YITFAFEDGSYEIYVEDLRKDQEKDGGGDHRKLMVNLSPTKDKDFLLHANSKEHSVELQKCENPLPEQAFFVLHEQPSKCVSFECKSHPGVFLGVKNNQLALIKLGEHPEDSNRENTTFKLSNLM

SEQ ID NO: 95 - protein, 273 aa, Bos taurus
  MISC_FEATURE (1)..(273): This is the amino acid sequence of Bovine IL-33.
MKPKMKYSTTKISPAKMKCSAGKALVKSPKLRKSQQKAEEVCQIYYMQLRSGLKIEKKVCYFRKETTKRHSLTAEKYKEHLALVACEQLNHLQQSVEQGFTLGKTMVPYTTATGLPSIKEHSASLSTYNDQFITFVLEDGSYEIYVEDLIDNQEKDKVLLRYYDSQFPSSETDDGGSHRKLMVNLSPTKDKDFLLHANSKEHSVELQKCENQLPEQTFFVLHETSSQCVSFECKSNPGVFLGVKDNQLALIKRGEHPEDSNSQNITFKLSNLM

SEQ ID NO: 96 - protein, 142 aa, Bos taurus
  MISC_FEATURE (1)..(142): This is the amino acid sequence of the probable mature sequence of Bovine IL-33.
FITFVLEDGSYEIYVEDLIDNQEKDKVLLRYYDSQFPSSETDDGGSHRKLMVNLSPTKDKDFLLHANSKEHSVELQKCENQLPEQTFFVLHETSSQCVSFECKSNPGVFLGVKDNQLALIKRGEHPEDSNSQNITFKLSNLM

SEQ ID NO: 97 - protein, 270 aa, Homo sapiens
  MISC_FEATURE (1)..(270): This is the amino acid sequence of Human IL- 33 - Full length (1-270).
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 98 - protein, 176 aa, Homo sapiens
  MISC_FEATURE (1)..(176): This is the amino acid sequence of Human IL-33 - Chain 1(95-270).
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 99 - protein, 172 aa, Homo sapiens
  MISC_FEATURE (1)..(172): This is the amino acid sequence of Human IL-33 - Chain 2(99-270).
SGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 100 - protein, 162 aa, Homo sapiens
  MISC_FEATURE (1)..(162): This is the amino acid sequence of Human IL- 33 - Chain 3 (109-270).
HDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 101 - protein, 157 aa, Canis lupus
  MISC_FEATURE (1)..(157): This is the amino acid sequence of Canine TNF-alpha protein.
VKSSSRTPSDKPVAHVVANPEAEGQLQWLSRRANALLANGVELTDNQLIVPSDGLYLIYSQVLFKGQGCPSTHVLLTHTISRFAVSYQTKVNLLSAIKSPCQRETPEGTEAKPWYEPIYLGGVFQLEKGDRLSAEINLPNYLDFAESGQVYFGIIAL

SEQ ID NO: 102 - protein, 157 aa, Felis catus
  MISC_FEATURE (1)..(157): This is the amino acid sequence of Feline TNF-alpha protein.
LRSSSRTPSDKPVAHVVANPEAEGQLQWLSRRANALLANGVELTDNQLKVPSDGLYLIYSQVLFTGQGCPSTHVLLTHTISRFAVSYQTKVNLLSAIKSPCQRETPEGAEAKPWYEPIYLGGVFQLEKGDRLSTEINLPAYLDFAESGQVYFGIIAL

SEQ ID NO: 103 - protein, 153 aa, Bos taurus
  MISC_FEATURE (1)..(153): This is the amino acid sequence of Bovine IL1-beta protein.
APVQSIKCKLQDREQKSLVLASPCVLKALHLLSQEMNREVVFCMSFVQGEERDNKIPVALGIKDKNLYLSCVKKGDTPTLQLEEVDPKVYPKRNMEKRFVFYKTEIKNTVEFESVLYPNWYISTSQIEERPVFLGHFRGGQDITDFRMETLSP

SEQ ID NO: 104 - protein, 152 aa, Canis lupus
  MISC_FEATURE (1)..(152): This is the amino acid sequence of Canine IL1-beta protein.
AAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 105 - protein, 152 aa, Felis catus
  MISC_FEATURE (1)..(152): This is the amino acid sequence of Feline IL1-beta protein.
AAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESAS

SEQ ID NO: 106 - protein, 135 aa, Canis lupus
  MISC_FEATURE (1)..(135): This is the amino acid sequence of Dog IL-2 protein.
APITSSSTKETEQQMEQLLLDLQLLLNGVNNYENPQLSRMLTFKFYTPKKATEFTHLQCLAEELKNLEEVLGLPQSKNVHLTDTKELISNMNVTLLKLKGSETSYNCEYDDETATITEFLNKWITFCQSIFSTLT

SEQ ID NO: 107 - protein, 134 aa, Felis catus
  MISC_FEATURE (1)..(134): This is the amino acid sequence of Cat IL-2 protein.
APASSSTKETQQQLEQLLLDLRLLLNGVNNPENPKLSRMLTFKFYVPKKATELTHLQCLVEELKPLEEVLYLAQSKNFHLNHIKELMSNINVTVLKLKGSETRFTCNYDDETATIVEFLNKWITFCQSIFSTLT

SEQ ID NO: 108 - protein, 133 aa, Homo sapiens [sequence omitted]

SEQ ID NO: 109 - protein, 120 aa, Canis lupus
  MISC_FEATURE (1)..(120): This is the amino acid sequence of Dog IL-3 protein.
RPFSTDLPKQYFTMINEIMEMLNKSPSPSEEPLDSNEKETLLEDTLLRPNLDVFLNASSKFHKNGLLIWNNLKEFLPLLPTPTPRGEPISIMENNWGDFQRKLKKYLEALDNFLNFKNKP

SEQ ID NO: 110 - protein, 133 aa, Homo sapiens
  MISC_FEATURE (1)..(133): This is the amino acid sequence of Human IL-3 protein.
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 111 - protein, 187 aa, Canis lupus
  MISC_FEATURE (1)..(187): This is the amino acid sequence of Dog IL-6 protein.
FPTPGPLAGDSKDDATSNSLPLTSANKVEELIKYILGKISALRKEMCDKFNKCEDSKEALAENNLHLPKLEGKDGCFQSGFNQETCLTRITTGLVEFQLHLNILQNNYEGDKENVKSVHMSTKILVQMLKSKVKNQDEVTTPDPTTDASLQAILQSQDECVKHTTIHLILRSLEDFLQFSLRAVRIM

SEQ ID NO: 112 - protein, 181 aa, Felis catus
  MISC_FEATURE (1)..(181): This is the amino acid sequence of Cat IL-6 protein.
TPGPLGGDATSNRLPLTSADKMEELIKYILGKISALKKEMCDNYNKCEDSKEALAENNLNLPKLAEKDGCFQSGFNQETCLTRITTGLQEFQIYLKFLQDKYEGDEENAKSVYTSTNVLLQMLKRKGKNQDEVTIPVPTVEVGLQAKLQSQEEWLRHTTIHLTLRRLEDFLQFSLRAVRIM

SEQ ID NO: 113 - protein, 179 aa, Bos taurus
  MISC_FEATURE (1)..(179): This is the amino acid sequence of Bovine IL-6 protein.
GPLGEDFKNDTTPGRLLLTTPEKTEALIKRMVDKISAMRKEICEKNDECESSKETLAENKLNLPKMEEKDGCFQSGFNQAICLIRTTAGLLEYQIYLDYLQNEYEGNQENVRDLRKNIRTLIQILKQKIADLITTPATNTDLLEKMQSSNEWVKNAKIILILRNLENFLQFSLRAIRMK

SEQ ID NO: 114 - protein, 183 aa, Homo sapiens
  MISC_FEATURE (1)..(183): This is the amino acid sequence of Human IL-6 protein.
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 115 - protein, 152 aa, Homo sapiens
  MISC_FEATURE (1)..(152): This is the amino acid sequence of Human IL-7 protein.
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 116 - protein, 79 aa, Canis lupus
  MISC_FEATURE (1)..(79): This is the amino acid sequence of Dog IL-8 protein.
AVLSRVSSELRCQCIKTHSTPFHPKYIKELRVIDSGPHCENSEIIVKLFNGNEVCLDPKEKWVQKVVQIFLKKAEKQDP

SEQ ID NO: 117 - protein, 79 aa, Felis catus
  MISC_FEATURE (1)..(79): This is the amino acid sequence of Cat IL-8 protein.
AVLSRISSELRCQCIKTHSTPFNPKLIKELTVIDSGPHCENSEIIVKLVNGKEVCLDPKQKWVQKVVEIFLKKAEKQNA

SEQ ID NO: 118 - protein, 79 aa, Bos taurus
  MISC_FEATURE (1)..(79): This is the amino acid sequence of Bovine IL-8 protein.
AVLSRMSTELRCQCIKTHSTPFHPKFIKELRVIESGPHCENSEIIVKLTNGNEVCLNPKEKWVQKVVQVFVKRAEKQDP

SEQ ID NO: 119 - protein, 79 aa, Homo sapiens
  MISC_FEATURE (1)..(79): This is the amino acid sequence of Human IL- 8 - aa 21-99 protein.
EGAVLPRSAKELRCQCIKTYSKPFHPKFIKELRVIESGPHCANTEIIVKLSDGRELCLDPKENWVQRVVEKFLKRAENS

SEQ ID NO: 120 - protein, 77 aa, Homo sapiens
  MISC_FEATURE (1)..(77): This is the amino acid sequence of Human IL- 8 - aa 23-99 protein.
AVLPRSAKELRCQCIKTYSKPFHPKFIKELRVIESGPHCANTEIIVKLSDGRELCLDPKENWVQRVVEKFLKRAENS

SEQ ID NO: 121 - protein, 73 aa, Homo sapiens
  MISC_FEATURE (1)..(73): This is the amino acid sequence of Human IL- 8 - aa 27-99 protein.
RSAKELRCQCIKTYSKPFHPKFIKELRVIESGPHCANTEIIVKLSDGRELCLDPKENWVQRVVEKFLKRAENS

SEQ ID NO: 122 - protein, 72 aa, Homo sapiens
  MISC_FEATURE (1)..(72): This is the amino acid sequence of Human IL- 8 - aa 28-99 protein.
SAKELRCQCIKTYSKPFHPKFIKELRVIESGPHCANTEIIVKLSDGRELCLDPKENWVQRVVEKFLKRAENS

SEQ ID NO: 123 - protein, 71 aa, Homo sapiens
  MISC_FEATURE (1)..(71): This is the amino acid sequence of Human IL- 8 - aa 29-99 protein.
AKELRCQCIKTYSKPFHPKFIKELRVIESGPHCANTEIIVKLSDGRELCLDPKENWVQRVVEKFLKRAENS

SEQ ID NO: 124 - protein, 70 aa, Homo sapiens [sequence omitted]

SEQ ID NO: 125 - protein, 69 aa, Homo sapiens
  MISC_FEATURE (1)..(69): This is the amino acid sequence of Human IL-8 - aa 31-99 protein.
ELRCQCIKTYSKPFHPKFIKELRVIESGPHCANTEIIVKLSDGRELCLDPKENWVQRVVEKFLKRAENS

SEQ ID NO: 126 - protein, 126 aa, Homo sapiens
  MISC_FEATURE (1)..(126): This is the amino acid sequence of Human IL-9 protein.
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 127 - protein, 162 aa, Canis lupus
  MISC_FEATURE (1)..(162): This is the amino acid sequence of Dog IL-10 protein.
SRHQSTLLEDDCTHFPASLPHMLRELRAAFGRVKIFFQMKDKLDNILLTGSLLEDFKSYLGCQALSEMIQFYLEEVMPRAENHDPDIKNHVNSLGEKLKTLRLRLRLRRCHRFLPCENKSKAVEQVKSAFSKLQEKGVYKAMSEFDIFINYIETYMTMRMKI

SEQ ID NO: 128 - protein, 160 aa, Felis catus
  MISC_FEATURE (1)..(160): This is the amino acid sequence of Cat IL-10 protein.
SRHQSTLSEDNCTHFSVSLPHMLRELRAAFGKVKTFFQTKDELHSILLTRSLLEDFKGYLGCQALSEMIQFYLEEVMPQAENEDPDIKQHVNSLGEKLKTLRLRLRRCHRFLPCENKSKVVEQVKSTFSKLQEKGVYKAMGEFDIFINYIEAYMTMKMKI

SEQ ID NO: 129 - protein, 160 aa, Homo sapiens
  MISC_FEATURE (1)..(160): This is the amino acid sequence of Human IL-10 protein.
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 130 - protein, 178 aa, Homo sapiens
  MISC_FEATURE (1)..(178): This is the amino acid sequence of Human IL-11 protein.
PGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 131 - protein, 546 aa, Homo sapiens
  MISC_FEATURE (1)..(546): This is the amino acid sequence of Human IL-14 protein.
MKNQDKKNGAAKQSNPKSSPGQPEAGPEGAQERPSQAAPAVEAEGPGSSQAPRKPEGAQARTAQSGALRDVSEELSRQLEDILSTYCVDNNQGGPGEDGAQGEPAEPEDAEKSRTYVARNGEPEPTPVVNGEKEPSKGDPNTEEIRQSDEVGDRDHRRPQEKKKAKGLGKEITLLMQTLNTLSTPEEKLAALCKKYAELLEEHRNSQKQMKLLQKKQSQLVQEKDHLRGEHSKAVLARSKLESLCRELQRHNRSLKEEGVQRAREEEEKRKEVTSHFQVTLNDIQLQMEQHNERNSKLRQENMELAERLKKLIEQYELREEHIDKVFKHKDLQQQLVDAKLQQAQEMLKEAEERHQREKDFLLKEAVESQRMCELMKQQETHLKQQLALYTEKFEEFQNTLSKSSEVFTTFKQEMEKMTKKIKKLEKETTMYRSRWESSNKALLEMAEEKTVRDKELEGLQVKIQRLEKLCRALQTERNDLNKRVQDLSAGGQGSLTDSGPERRPEGPGAQAPSSPRVTEAPCYPGAPSTEASGQTGPQEPTSARA

SEQ ID NO: 132 - protein, 114 aa, Felis catus
  MISC_FEATURE (1)..(114): This is the amino acid sequence of Cat IL-15 protein.
NWQDVISDLKIIDKIIQSLHIDATLYTESDVHPNCKVTAMKCFLLELHVISLESKNETIHQTVENIIILANSGLSSNRNITETGCKECEELEEKNIKEFLQSFVHIVQMFINTS

SEQ ID NO: 133 - protein, 114 aa, Homo sapiens
  MISC_FEATURE (1)..(114): This is the amino acid sequence of Human IL-15 protein.
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 134 - protein, 1332 aa, Homo sapiens
  MISC_FEATURE (1)..(1332): This is the amino acid sequence of Human IL- 16 - 1-1332 aa protein.
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEILVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 135 - protein, 121 aa, Homo sapiens
  MISC_FEATURE (1)..(121): This is the amino acid sequence of Human IL- 16 - 1212-1332 aa protein.
SAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 136 - protein, 127 aa, Canis lupus
  MISC_FEATURE (1)..(127): This is the amino acid sequence of Dog IL-17A protein.
FPQNPGCRNTEDKNFPQHVKVNLNILNRNTNSRRPSDYYNRSTSPWNLHRNEDPERYPSVIWEAKCRHLGCVNNEGNINYHMNSVPIQQEILVLRRESQHCPHSFRLEKMLVAVGCTCVTPIVRHVA

SEQ ID NO: 137 - protein, 127 aa, Felis catus
  MISC_FEATURE (1)..(127): This is the amino acid sequence of Cat IL-17A protein.
FPQNPGCPTTEDKNFPQHVKVNVNILNGNKSSRRPLDYYRRSTSPWSLHRNEDPERYPSVIWEAKCLHWGCVNTEGKEDHHMNSVPIQQEILVLRRESRHCPHSFRLEKMLVTVGCTCVTPIVRHVV

SEQ ID NO: 138 - protein, 158 aa, Canis lupus
  MISC_FEATURE (1)..(158): This is the amino acid sequence of Dog IL-17B protein.
RNPKGKRKGPGRPGTLAPGPHQVPLDLVSQAKPYARMEEYERNLSEMVAQLRNSSEPARRKCEVNLQLWLSNKRSLSPWGYSINHDPSRVPADLPEARCLCLGCVNPFTMQEDRSMVSVPVFSQVPVRRRLCPLPPRTGPCRQRAVMETIAVGCTCIF

SEQ ID NO: 139 - protein, 158 aa, Felis catus
  MISC_FEATURE (1)..(158): This is the amino acid sequence of Cat IL-17B protein.
RNPKGKRKGPGRPGTLASGPHQVPLDLVSQAKPYARMEEYERNLGEMVAQLRNSSEPARRKCEVNLQLWLSNKRSLSPWGYSINHDPSRIPADLPEARCLCLGCVNPFTMQEDRSMVSVPVFSQVPVRRRLCPLPPRTGPCRQRAVMETIAVGCTCIF

SEQ ID NO: 140 - protein, 160 aa, Homo sapiens
  MISC_FEATURE (1)..(160): This is the amino acid sequence of Human IL-17B protein.
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 141 - protein, 179 aa, Canis lupus
  MISC_FEATURE (1)..(179): This is the amino acid sequence of Dog IL-17C protein.
RHGPQLWRGLHTHGTPRCYSAEELPRGQAPPHLLARAAKWEQALPVALVSSLEAGGRGREQDGPPAGAQCPVLQPEEVLEADIHQRSISPWRYRVDTDESRYPQKLAFAECLCRGCISARTGRETAALNSVPLLQSLLVLRRRPCSRDTTGLPTPGAFSFHAEFIRVPIGCTCVLPRST

SEQ ID NO: 142 - protein, 180 aa, Felis catus
  MISC_FEATURE (1)..(180): This is the amino acid sequence of Cat IL-17C protein.
RHGPQLWGGPHAHGTPRCYSAEELPLGQPPPHLLARAAKWEQALPVALVSSLEAGGRRRRHDGPPAGTQCPVLRPEDVLEAGIHQRSISPWRYRVDTDESRYPQKLAFAQCLCRGCVSARTGRETAALNSVPLLQSLLVLRRQPCSREATGLPTPGAFSFHAEFIRVPVGCTCVLPRSAR

SEQ ID NO: 143 - protein, 179 aa, Homo sapiens
  MISC_FEATURE (1)..(179): This is the amino acid sequence of Human IL-17C protein.
HHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 144 - protein, 185 aa, Felis catus
  MISC_FEATURE (1)..(185): This is the amino acid sequence of Cat IL-17D protein.
ALRASRRPARPRGCAERPEELLEQLYGRLAAGVLGAFHHTLQLGPREQARNASCPAGGRPGDRRFRPPTNLRSVSPWAYRISYDPARYPKYLPEAYCLCRGCLTGLFGEEDLRFRSAPVYMPTVILRRTSACAGGRWVYTEEYVTVPVGCTCVPEQEKEADAVNSSMDKQGARLLLGPGDKPGRP

SEQ ID NO: 145 - protein, 187 aa, Homo sapiens
  MISC_FEATURE (1)..(187): This is the amino acid sequence of Human IL-17D protein.
AGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 146 - protein, 129 aa, Canis lupus
  MISC_FEATURE (1)..(129): This is the amino acid sequence of Dog IL-17F protein.
RKHLKAGETALCPPLEDNSVRVDIRILRQNRGISISNDFQNRSSSPWDYNITRDPHRFPSEIAEAQCRHSGCINAEGQEDSSMNSVPIQQEFLVLRREPQGCSRSFRLEKVLVTVGCTCVTPIVRYVRA

SEQ ID NO: 147 - protein, 130 aa, Felis catus
  MISC_FEATURE (1)..(130): This is the amino acid sequence of Cat IL-17F protein.
RKNPKAGDTALCPPLEDNSVRVDIRILRQNQGGISISNDFQNRSSSPWDYNVTRDPNRFPSEIVEAQCRHSGCINAEGQEDSSMNSVPIQQEFLVLRREPQGCSRTFRLEKVRVTVGCTCVTPIVRYVRA

SEQ ID NO: 148 - protein, 133 aa, Homo sapiens [sequence omitted]

SEQ ID NO: 149 - protein, 157 aa, Canis lupus
  MISC_FEATURE (1)..(157): This is the amino acid sequence of Dog IL-18 protein.
YFGKLEPKLSIIRNLNDQVLFVNEGNQPVFEDMPDSDCTDNAPHTIFIIYMYKDSLTRGLAVTISVKYKTMSTLSCKNKTISFQKMSPPDSINDEGNDIIFFQRSVPGHDDKIQFESSLYKGHFLACKKENDLFKLILKDKDENGDKSIMFTVQNKS

SEQ ID NO: 150 - protein, 157 aa, Felis catus
  MISC_FEATURE (1)..(157): This is the amino acid sequence of Cat IL-18 protein.
YFGKLEHKLSILRNLNDQVLFINQGDQPVFEDMPDSDCTDNAPRTEFIIYMYKDSLTRGLAVTISVNYKTMSTLSCENKIISFKEMSPPESINDEGNDIIFFQRSVPGHDDKIQFESSLYKGYFLACEKEKDLFKLILKKKDENGDKSIMFTVQNKN

SEQ ID NO: 151 - protein, 157 aa, Homo sapiens
  MISC_FEATURE (1)..(157): This is the amino acid sequence of Human IL-18 protein.
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 152 - protein, 153 aa, Homo sapiens
  MISC_FEATURE (1)..(153): This is the amino acid sequence of Human IL-19 protein.
LRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 153 - protein, 152 aa, Homo sapiens
  MISC_FEATURE (1)..(152): This is the amino acid sequence of Human IL-20 protein.
LKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 154 - protein, 129 aa, Canis lupus familiaris
  MISC_FEATURE (1)..(129): This is the amino acid sequence of Dog IL-21 protein.
HKSSFQEQDLLLIRMRQLIDIVDQLKNYVNDLDPESLPAPEDVKRHCERSAFSCFQKVQLKAANTGGNEQIINVLTKQLKRKLPPTNAGRRQKHRPACPSCDSYEKAPPKEFLERLKSLIQKMIHQHLS

SEQ ID NO: 155 - protein, 138 aa, Homo sapiens
  MISC_FEATURE (1)..(138): This is the amino acid sequence of Human IL-21 protein.
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 156 - protein, 146 aa, Canis lupus
  MISC_FEATURE (1)..(146): This is the amino acid sequence of Dog IL-22 protein.
LPISSHCRLDKSNFQQPYITNRTFMLAKEASLADNNTDVRLIGEKLFHGVNMGERCYLMKEVLNFTLEEVLLPQSDRFQPYMQEVVPFLARLSNKLSQCHIENDDQHIQRNVQKLKDTVQKLGENGEIKAIGELDLLFMALRNACV

SEQ ID NO: 157 - protein, 146 aa, Felis catus
  MISC_FEATURE (1)..(146): This is the amino acid sequence of Cat IL-22 protein.
VPISSHCRLDGSNFQQPYITNRTFMLAKEASLADNNTDVRLIGEKLFYEVNMGERCYLMKQVLNFTLEEVLLPQSDRFQPYMQEVVPFLARLSNKLSQCHIDSDDQHIQRNVQNLKDTVKKLGENGEIKVIGELDLLFMALRNACN

SEQ ID NO: 158 - protein, 146 aa, Homo sapiens
  MISC_FEATURE (1)..(146): This is the amino acid sequence of Human IL-22 protein.
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 159 - protein, 102 aa, Canis lupus
  MISC_FEATURE (1)..(102): This is the amino acid sequence of Dog IL-24 protein.
QEFRFGPCRVQGVALRELREAFWTVKDTVQAKDNITSVRLLRKEVLQDVSQEDEMFSISESARRRFLLFQRAFKQLDIQAAQTKAFGEVDILLTWMEKFYEF

SEQ ID NO: 160 - protein, 155 aa, Canis lupus
  MISC_FEATURE (1)..(155): This is the amino acid sequence of Dog IL-24 Family Protein.
QEFRFGPCRVQGVALRELREAFWTVKDTVQAKDNITSVRLLRKEVLQDVSDAESCYLIRALLKFYLNTVFKNYLDEAADVRIRRSFSTLANNFFVIASKLQPSQEDEMFSISESARRRFLLFQRAFKQLDIQAAQTKAFGEVDILLTWMEKFYEF

SEQ ID NO: 161 - protein, 102 aa, Felis catus
  MISC_FEATURE (1)..(102): This is the amino acid sequence of Cat IL-24 protein.
QEFQFGPCRVEGVVLQELWEAFWAMKDIVQAKDNITNVRLLRKEVLQNVSQENEMFSVSDSARRRFLLFQRAFKQLDIEAAQTKAFGEVDILLTWMEKFYQL

SEQ ID NO: 162 - protein, 155 aa, Felis catus
  MISC_FEATURE (1)..(155): This is the amino acid sequence of Cat IL-24 Family Protein.
QEFQFGPCRVEGVVLQELWEAFWAMKDIVQAKDNITNVRLLRKEVLQNVSNAESCYLIRALLKFYLNTVFKNYQDKAADFRVRKSFSTLANNFVVIVSKLQPSQENEMFSVSDSARRRFLLFQRAFKQLDIEAAQTKAFGEVDILLTWMEKFYQL

SEQ ID NO: 163 - protein, 155 aa, Homo sapiens
  MISC_FEATURE (1)..(155): This is the amino acid sequence of Human IL-24 protein.
QEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 164 - protein, 145 aa, Canis lupus
  MISC_FEATURE (1)..(145): This is the amino acid sequence of Dog IL-25 protein.
CTHWPNCCPSKRQDPTHEWLKRDTVLKFPGETTSLTHHPESCKASEDGPLNSRSISPWKYELDRDLNRLPQDLYHARCLCQHCVSLQTGSHMDPLGNSELLYHNQTVFYRRPCPGEQGAPDGYCLEQRLYRVSLACVCVRPRVMA

SEQ ID NO: 165 - protein, 145 aa, Felis catus
  MISC_FEATURE (1)..(145): This is the amino acid sequence of Cat IL-25 protein.
LSHWSSCCPSKGQNPTHEWLTENTVLMTPPESASLIHSLESCRASEDGPLNSRSISPWRYELDRDLNRLPQDLYHARCLCPHCVSLQTGSHMDPLGNSELLYHNQTVFYRRPCPGEQGTRDGYCLEQRLYRVSLACVCVRPRVMA

SEQ ID NO: 166 - protein, 145 aa, Homo sapiens
  MISC_FEATURE (1)..(145): This is the amino acid sequence of Human IL-25 protein.
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 167 - protein, 150 aa, Canis lupus
  MISC_FEATURE (1)..(150): This is the amino acid sequence of Dog IL-26 family protein.
KHKQSSSAKGCYPRGTLSQAVDTLYVKAAWLKATIPEDRIKNIRLLRKKTKNLFMKNCRFQEQLLSFFMEDVFGQLQLKICREIRFVEELHSLRQQLSRCISCASSAREMKTITRMKSTFYGLGNKGIYKAISELNILLSWIKQFLESIK

SEQ ID NO: 168 - protein, 127 aa, Felis catus
  MISC_FEATURE (1)..(127): This is the amino acid sequence of Cat IL-26 protein.
KHKQSSSAKGCYPRGTLSQAVDRLYVKAAWLKATIPEDRIKNIRLLKKKTKKLFMKNCRFQEQLLSFFMDDVFGQLQLQVCKERHFVEEFHSLRQQLSRCVLSWVRKTSLIKSPVSGACTLLTGREM

SEQ ID NO: 169 - protein, 150 aa, Felis catus
  MISC_FEATURE (1)..(150): This is the amino acid sequence of Cat IL-26 family protein.
KHKQSSSAKGCYPRGTLSQAVDRLYVKAAWLKATIPEDRIKNIRLLKKKTKKLFMKNCRFQEQLLSFFMDDVFGQLQLQVCKERHFVEEFHSLRQQLSRCISCASSAREMKTITRMKRTFYGIGNKGIYKAVSELDILLSWIKQFLESIK

SEQ ID NO: 170 - protein, 150 aa, Homo sapiens
  MISC_FEATURE (1)..(150): This is the amino acid sequence of Human IL-26 protein.
KHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 171 - protein, 204 aa, Canis lupus
  MISC_FEATURE (1)..(204): This is the amino acid sequence of Dog IL-27 protein.
FPRPPGRSPLSLQELRREFKVSLQLAKKLFSEVRIQAHHFAESQLPGVSLDLLPLGDQLPNVSLPFQAWHSLSDPERLCFLSMMLHPFHALLESLGSQGGWTSSEKMHLWTMRLDLRDLQRHLRFQVEYPPTCSTPRDQHTKLKLIGSLLKTSPAQRLGKFGGRCSRSSVCVCVCVCVCVCVCPRPRLCSLNLCLPMESWSEQL

SEQ ID NO: 172 - protein, 208 aa, Felis catus
  MISC_FEATURE (1)..(208): This is the amino acid sequence of Cat IL-27 protein.
FPRPPGRPPLSLQELRREFKVSLHLARKLFSEVRTQAHRFAESHLPGVSLDLLPLGDRLPNVSLTFQAWHSLSDPERLCFLFMTLRPFHALLGSLGSQGGWTSSEKIDLWTMRLDLRDLQRHLHFQVLAAGLNLPEEEDDEERKEPLPGAPSGLSRVSGQPSWPQLLYTYQLLHSLELVLARAVRDLLLLSQAGNPAPASGSSFGSWP

SEQ ID NO: 173 - protein, 171 aa, Canis lupus
  MISC_FEATURE (1)..(171): This is the amino acid sequence of Dog IL-29 protein.
GPVPTSKPTTTRRGCHMDRFQSLSPRELEAFKKTKDALEESLSWKNWSCSSRLFPRSRDLRLLQAWERPVALEAELDLTLKVLENMTDSSLGVTLDQPLRTLHHIHSELQACVPAQPTADPRPHGRLHHWLHRLQKAPKESQGCLEASITFNLFRLLTRDLKCVASRDLCV

SEQ ID NO: 174 - protein, 170 aa, Canis lupus
  MISC_FEATURE (1)..(170): This is the amino acid sequence of Dog IL-29 protein.
GPVPTSKPTMAWRGCDIGRFKSLSPRELEAFKKAKDALEYSLKNWSCNSRLFPRNRDLRQLQVWERPVALEAELALTLKVLETMADRSLGDILDQPLHTLRHIHSQLQACVSAQPPAGPQPRGRLHPWLHRLHEASKKESQGCLEASVLFNLFRLLKKDLECVAVGDLCV

SEQ ID NO: 175 - protein, 171 aa, Felis catus
  MISC_FEATURE (1)..(171): This is the amino acid sequence of Cat IL-29 protein.
GPVPTSRPPPTRRGCHVGKFPSLSPRELEAFKKARDALEESLPLKNWSCGSRLFPRTRDLIRLQAWERPAALEAELDLTLKVLGNMTDSSLGVVLDQPLRVVRHIHSELQACVRAQPTAGPRPRGRLHHWLHRLQRPPEESQGCLEASVTFNLFRLLTRDLKCVTGGDLCV

SEQ ID NO: 176 - protein, 171 aa, Felis catus
  MISC_FEATURE (1)..(171): This is the amino acid sequence of Cat IL-29, IFN-lambda1 protein.
GPVPTSRPPPTRRGCHVGKFPSLSPRELEAFKKARDALEESLPLKNWSCGSRLFPRTRDLIRLQAWERPAALEAELDLTLKVLGNMTDSSLGVVLDQPLRVLRHIHSELQACVRAQPTAGPRPRGRLHHWLHRLQRAPEESQGCLEASVTFNLFRLLTRDLKCVTGGDLCV

SEQ ID NO: 177 - protein, 181 aa, Homo sapiens
  MISC_FEATURE (1)..(181): This is the amino acid sequence of Human IL-29, IFN-lambda1 protein.
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 178 - protein, 160 aa, Felis catus
  MISC_FEATURE (1)..(160): This is the amino acid sequence of Cat IL-32 protein.
MRHQMHQLVDIFCDRTQHQSRGAPQQRLRLGEVEDGFNEIMLEAVDLHHHQNDDEESSPLLPEVRQELRSRVLRSSVLDLEEKEHPVVQKTEESFCDRALRLFRRLLYQLQLKWQAALAWLREKVAAGFQAFCDAVEAICSAFNSFCTSVAQVFRSAVQA

SEQ ID NO: 179 - protein, 214 aa, Bos taurus
  MISC_FEATURE (1)..(214): This is the amino acid sequence of Bovine IL-34 protein.
NEGLEPWPLTRSDECAITGFLRDKLQYRNRLQYMKHYFPINYRVSVPYEGVLRTANVTRLQRAQVSQQELRYLWVLVSLSATEWVQEVLLEGHPSWKYLEEVHTLLLDVKQGLGGVEVSPQVEAVLNLLSAPGSLKLVRPKALLDNCFRVMQLLYCPCCKESSVLNWQDCEAPQPQPRSPASAQCEAAQLYPLPQPPSTSLPRVLGPSAGPPTQ

SEQ ID NO: 180 - protein, 153 aa, Homo sapiens
  MISC_FEATURE (1)..(153): This is the amino acid sequence of Human IL-36-alpha protein.
KIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 181 - protein, 160 aa, Homo sapiens
  MISC_FEATURE (1)..(160): This is the amino acid sequence of Human IL-36-beta protein.
REAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 182 - protein, 152 aa, Homo sapiens
  MISC_FEATURE (1)..(152): This is the amino acid sequence of Human IL-36-gamma protein.
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 183 - protein, 193 aa, Canis lupus
  MISC_FEATURE (1)..(193): This is the amino acid sequence of Canis IL-37 protein.
MSFLEDSGVKMESEDWERDEPHGCSEGPKVKVSYPEKFSIHDRDHKVLVLDCDTLRAVPFKTYIRPEMFFVLASSLSSASKEKGSPILLAVCKGELCLCCEKDRRQSQPSLQLKKKKLTNLAAQKESARQPFIFYRTKVGSQNRLESAAHPGWFICTSRNSGEPVGVTNVLGKRKHTEFSFHRAETSPSEVSG

SEQ ID NO: 184 - protein, 152 aa, Homo sapiens
  MISC_FEATURE (1)..(152): This is the amino acid sequence of Human IL-38 protein.
MCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 185 - protein, 245 aa, Homo sapiens
  MISC_FEATURE (1)..(245): This is the amino acid sequence of Human IL-40 protein.
REEEITPVVSIAYKVLEVFPKGRWVLITCCAPQPPPPITYSLCGTKNIKVAKKVVKTHEPASFNLNVTLKSSPDLLTYFCWASSTSGAHVDSARLQMHWELWSKPVSELRANFTLQDRGAGPRVEMICQASSGSPPITNSLIGKDGQVHLQQRPCHRQPANFSFLPSQTSDWFWCQAANNANVQHSALTVVPPGGDQKMEDWQGPLESPILALPLYRSTRRLSEEEFGGFRIGNGEVRGRKAAAM

SEQ ID NO: 186 - protein, 247 aa, Canis lupus
  MISC_FEATURE (1)..(247): This is the amino acid sequence of Canis IL-40 isoform X2 protein.
MRLLLLLCLALLATCSFSLEQEVEVTPETFIAYKVREVFPSSRRVVITCHSPRAPPPITYSLWGSQGVEVAKKVVKTGDPASFSINITLKSRPELLTYSCRAASLRGEHSASTKLQMYWELWAKPMAQPRANFVLLERGSGPRVEISCQVSSGSPPITYSLVRKDGYVHRQQRPTYGQPANFSFPLTRTSNWLRCQAANDISVQSSPFRLVPPGYLPQGPVLVLAGSLTSIAAVTSWVLGPALWTRL

SEQ ID NO: 187 - protein, 266 aa, Homo sapiens
  MISC_FEATURE (1)..(266): This is the amino acid sequence of Human IL-41, Meteorin-like protein.
QYSSDRCSWKGSGLTHEAHRKEVEQVYLRCAAGAVEWMYPTGALIVNLRPNTFSPARHLTVCIRSFTDSSGANIYLEKTGELRLLVPDGDGRPGRVQCFGLEQGGLFVEATPQQDIGRRTTGFQYELVRRHRASDLHELSAPCRPCSDTEVLLAVCTSDFAVRGSIQQVTHEPERQDSAIHLRVSRLYRQKSRVFEPVPEGDGHWQGRVRTLLECGVRPGHGDFLFTGHMHFGEARLGCAPRFKDFQRMYRDAQERGLNPCEVGTD

SEQ ID NO: 188 - protein, 296 aa, Canis lupus
  MISC_FEATURE (1)..(296): This is the amino acid sequence of Canine IL- 41, Meteorin-like protein.
MPPSVMSQMCTFLHRQQFCTNRSFFGFAVALEHRSERFDQIFCGLTHEAHRKEVEQVYLRCSAGSVEWMYPTGALIVNVRPNTFPPSRHLTLCIKPLKDSSGANIYLEKTGELKLLVRDGDRGPGQVRCFGFEQGGLFVEAAPQQDISRRTTGFQYELTSRHAESDLHTLSAPCRPCSDAEVLLAVCTSDFVVRGSIQNVTHVPEQQESAIHLRVSRLYRQKSRVFRPAPEGGGWRGRVATLLECGVRPGRGEFLFTGHMHFGEARLGCAPRFKDFQRMYQDAEERGLNPCEMGKD

SEQ ID NO: 189 - protein, 266 aa, Felis catus
  MISC_FEATURE (1)..(266): This is the amino acid sequence of Feline IL- 41, Meteorin-like protein.
QYSSDRCSWKGSGLTHEAHRKEVEQVYLRCSAGSVEWMYPTGALIVNVRPNTFPPSRRHLTLCIKPLRDSSGANIYLEKTGELQLLVRDGDRGPRQVHCFGFEQRGLFVEATPQQDISRRTTGFQYELTSRHAESDLRAQSAPCRPCSDTEVLLAVCTSDFVVRGSIQDVTHEPEQQESAIHLHVNRLYRQKSTVFRPAPEGGGWRGHVTTLLECGVRPGRGEFLFTGPIHFGEAHLGCAPRFKDFQRMYKDAEEKGLNPCEMGTE

SEQ ID NO: 190 - protein, 143 aa, Bos taurus
  MISC_FEATURE (1)..(143): This is the amino acid sequence of Bovine interferon-gamma protein.
QGQFFREIENLKEYFNASSPDVAKGGPLFSEILKNWKDESDKKIIQSQIVSFYFKLFENLKDNQVIQRSMDIIKQDMFQKFLNGSSEKLEDFKKLIQIPVDDLQIQRKAINELIKVMNDLSPKSNLRKRKRSQNLFRGRRASM

SEQ ID NO: 191 - protein, 143 aa, Canis lupus
  MISC_FEATURE (1)..(143): This is the amino acid sequence of Canis interferon-gamma protein.
QAMFFKEIENLKEYFNASNPDVSDGGSLFVDILKKWREESDKTIIQSQIVSFYLKLFDNFKDNQIIQRSMDTIKEDMLGKFLNSSTSKREDFLKLIQIPVNDLQVQRKAINELIKVMNDLSPRSNLRKRKRSQNLFRGRRASK

SEQ ID NO: 192 - protein, 144 aa, Felis catus
  MISC_FEATURE (1)..(144): This is the amino acid sequence of Felis interferon-gamma protein.
QAMFFKEIEELKGYFNASNPDVADGGSLFVDILKNWKEESDKTIIQSQIVSFYLKMFENLKDDDQRIQRSMDTIKEDMLDKLLNTSSSKRDDFLKLIQIPVNDLQVQRKAINELFKVMNDLSPRSNLRKRKRSQNLFRGRRASK

SEQ ID NO: 193 - protein, 166 aa, Bos taurus
  MISC_FEATURE (1)..(166): This is the amino acid sequence of Bovine interferon-alpha A protein.
CHLPHTHSLANRRVLMLLQQLRRVSPSSCLQDRNDFEFLQEALGGSQLQKAQAISVLHEVTQHTFQLFSTEGSPATWDKSLLDKLRAALDQQLTDLQACLTQEEGLRGAPLLKEDSSLAVRKYFHRLTLYLQEKRHSPCAWEVVRAEVMRAFSSSTNLQESFRRKD

SEQ ID NO: 194 - protein, 164 aa, Canis lupus
  MISC_FEATURE (1)..(164): This is the amino acid sequence of Canine interferon-alpha 1 of 2 protein.
CHLPDTHGLRNWRVLTLLGQMRRLSAGSCDHYTNDFAFPKELFDGQRLQEAQALSVVHVMTQKVFHLFCPDTSSAPWNMTLLEELCSGLSEQLDDLEACPLQEAGLAETPLMHEDSTLRTYFQRISLYLQDRNHSPCAWEMVRAEIGRSFFSSTILQERIRRRK

SEQ ID NO: 195 - protein, 171 aa, Felis catus
  MISC_FEATURE (1)..(171): This is the amino acid sequence of Feline interferon-alpha protein.
CDLPQTHGLLNRRALTLLGQMRRLPASSCQKDRNDFAFPQDVFGGDQSHKAQALSVVHVTNQKIFHFFCTEASSSAAWNTTLLEEFCTGLDRQLTRLEACVLQEVEEGEAPLTNEDIHPEDSILRNYFQRLSLYLQEKKYSPCAWEIVRAEIMRSLYYSSTALQKRLRSEK

SEQ ID NO: 196 - protein, 126 aa, Bos taurus
  MISC_FEATURE (1)..(126): This is the amino acid sequence of Bovine Granulocyte-macrophage colony-stimulating factor protein.
APTRPPNTATRPWQHVDAIKEALSLLNHSSDTDAVMNDTEVVSEKFDSQEPTCLQTRLKLYKNGLQGSLTSLMGSLTMMATHYEKHCPPTPETSCGTQFISFKNFKEDLKEFLFIIPFDCWEPAQK

SEQ ID NO: 197 - protein, 127 aa, Canis lupus
  MISC_FEATURE (1)..(127): This is the amino acid sequence of Canine Granulocyte-macrophage colony-stimulating factor protein.
APTRSPTLVTRPSQHVDAIQEALSLLNNSNDVTAVMNKAVKVVSEVFDPEGPTCLETRLQLYKEGLQGSLTSLKNPLTMMANHYKQHCPPTPESPCATQNINFKSFKENLKDFLFNIPFDCWKPVKK

SEQ ID NO: 198 - protein, 127 aa, Felis catus
  MISC_FEATURE (1)..(127): This is the amino acid sequence of Feline GM-CSF protein.
APTSSPSSVTRPWQHVDAIKEALSLLNNSSEITAVMNEAVEVVSEMFDPEEPKCMQTHLKLYEQGLRGSLISLKEPLRMMANHYKQHCPLTPETPCETQTITFKNFKENLKDFLFNIPFDCWEPDQK

SEQ ID NO: 199 - protein, 174 aa, Bos taurus
  MISC_FEATURE (1)..(174): This is the amino acid sequence of Bovine Granulocyte colony-stimulating factor protein.
TPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 200 - protein, 175 aa, Canis lupus
  MISC_FEATURE (1)..(175): This is the amino acid sequence of Canine Granulocyte colony-stimulating factor protein.
MAPLGPTGPLPQSFLLKCLEQMRKVQADGTALQETLCATHQLCHPEELVLLGHALGIPQPPLSSCSSQALQLMGCLRQLHSGLFLYQGLLQALAGISPELAPTLDTLQLDTTDFAINIWQQMEDLGMAPAVPPTQGTMPAFTSAFQRRAGGVLVASNLQSFLELAYRALRHFAKP

SEQ ID NO: 201 - protein, 174 aa, Felis catus
  MISC_FEATURE (1)..(174): This is the amino acid sequence of Feline Granulocyte colony-stimulating factor protein.
TPLGPTSSLPQSFLLKCLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPTQGTMPTFTSAFQRRAGGTLVASNLQSFLEVAYRALRHFTKP

SEQ ID NO: 202 - DNA, 1803 bp, Artificial sequence
  SEQ ID NO: 202 is the nucleic acid sequence of the plasmid pcDNA3.4-cIL-4-cIL-13-poly encoding the cIL-4 cIL-13 polyprotein construct of Example 1h.
gaattcccgccgccaccatgggttggagttgcatcatcctatttctagtggccacagctaccggcgtgcactccagcccctctcctgtgaccccatcccctacactgaaggagctgatcgaggaactcgtgaacatcacccagaaccaggcctctctgtgtaacggctccatggtctggtccgtgaatctgaccgccggcatgtactgtgccgctctggaatccctcatcaacgtgtctgactgttccgccatccaaagaacccagcggatgctgaaagctctgtgctcccagaagcctgctgccggccagatcagctccgagagatccagagataccaagatcgaggtgattcagctggtgaagaacctgctgacctacgtgcggggagtgtatagacatggcaatttcagaggctctggcggacagtacatcaaggccaactccaagttcatcggcatcaccgagctggcgggctctggccacaacttcaatatcacaatcaaggagatcatcaagatgctgaacatcctgaccgcccggaacgactcttgcatggaactgactgtgaaggacgtgttcaccgctcccaagaacacatctgacaaggaaatcttctgccgggccgccaccgtgctgagacagatctacacccacaactgctccaacagatacctgaggggcctgtaccgtaacctgtcttccatggccaacaaaacctgctccatgaacgagatcaagaagtctaccctgaaggacttcctggaacggctgaaggtgatcatgcaaaagaagtactaccggcacgccggttctgggtttaacaacttcacagtgtctttttggctgagagtgcccaaagtctccgcttcgcacctggagtccggcggctcgtctccttctcctgtgacccctagccctaccctgaaagagctcatcgaggaactggtcaacatcacccagaatcaggcttccctgtgcaacggctccatggtctggagcgtaaacctgaccgctggaatgtactgtgccgccctggaatctctgattaacgtgtccgactgctccgctatccagagaacacaaagaatgctgaaggccctgtgctctcagaagcctgccgctggccagatctcctctgagaggtcccgggacaccaagatcgaagtgatccagctggtgaagaacctgctgacctacgtgcggggcgtgtacagacacggcaacttccggggcggcggcggccataacttcaacatcaccatcaaagagatcatcaagatgctgaacatcctgaccgccagaaacgattcttgcatggaactgactgtgaaggacgtgttcaccgcccctaagaacacctccgataaggagatcttctgcagagctgctaccgtgctgcggcagatctacacccacaactgctccaaccggtatctgagaggcctgtacagaaacctgagctctatggccaacaagacctgtagcatgaacgagatcaagaagtccaccctgaaggatttcttggagagactgaaggtgatcatgcagaaaaagtactacagacacgctggctctggcttcaacaactttacagtgtccttctggctgcgggtgccaaaagtgtccgcctcccacctcgaggccggcagcggcttcaacaattttaccgtcagcttctggctgcgcgtccctaaggtgtccgcttctcacctggagggcagcggaggacagtacatcaaggccaattctaagttcatcggcatcaccgagctgcaccaccaccatcatcactgataagctt

SEQ ID NO: 203 - protein, 573 aa, Artificial sequence
  SEQ ID NO: 203 is the amino acid sequence of the cIL-13-cIL-4 polyprotein used for the vaccine construct of Example 13h
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRGSGGQYIKANSKFIGITELAGSGHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHAGSGFNNFTVSFWLRVPKVSASHLESGGSSPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRGGGGHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHAGSGFNNFTVSFWLRVPKVSASHLEAGSGFNNFTVSFWLRVPKVSASHLEGSGGQYIKANSKFIGITELHHHHHH

SEQ ID NO: 204 - DNA, 2775 bp, Artificial sequence
  SEQ ID NO: 204 is the nucleic acid sequence of the plasmid pcDNA3.4-cIL31-cIL-13-cIL-4-poly encoding the cIL-31-cIL-13-cIL-4-poly-His6 construct of Example 1i.
gaattcccgccgccaccatgggatggagctgcatcatcctgttcctggtggccacagccaccggcgtgcactccagccacatggcccctacccaccagctgccaccttctgatgtgcggaagatcatcctggagctgcagcctctgagcagaggcctgctggaagattaccagaagaaagaaaccggcgtgcccgagagcaatagaaccctcctgctgtgtctgaccagcgattcccagccccctagactgaacagctccgcaatcctgccttacttcagagccatccggcccctgtccgacaagaacatcatagacaagatcatcgagcagctggacaagctgaagttccagcacgagcctgagactgagatctccgtgcctgctgataccttcgagtgtaaatccttcatcctgaccatcttgcagcagttcagcgcctgtctggaatcagtgttcaagagcctgaactctggccctcagggcggcggcggattcaacaacttcacagtgtccttctggctgagggtgcccaaggtgtctgcctctcatctggagagcggaggctctagccctagcccagtgacccctagccccaccctgaaggagttgatcgaggaactggtgaatatcacgcagaaccaggccagcctgtgcaacggctccatggtttggagcgttaacctgacggctgggatgtactgcgctgctctggagtccctgatcaacgtgagcgattgcagcgctatccagagaactcagcggatgctgaaggccctgtgcagccagaagcctgccgccggtcagatcagctctgagagaagcagagacaccaagattgaagtgatccagctggtgaagaacctcctgacctacgtgcggggcgtgtacagacacggcaatttccgcggctctggcggccagtacatcaaggccaactccaagttcattggaatcacagagctggccggctcgggtcacaacttcaatataacaatcaaagagatcatcaagatgctaaatatcctgaccgccagaaacgactcctgtatggaactcacagtcaaagacgtctttacagccccaaagaacacctcggacaaggagattttctgcagagctgcaaccgtgctgcggcagatctatacccacaactgcagcaacagatatctgcgcggcctgtacagaaacctgagcagcatggccaataagacctgcagcatgaacgagatcaagaaaagtaccctgaaagacttcctggaaagactgaaagtgatcatgcagaagaagtactacaggcatgccggatccggttttaacaacttcacagttagcttctggctgcgggtgcccaaggtgagcgccagccacctggagagcggcggcagtagccacatggcaccaacccaccaactgcctccttccgacgtgcggaagatcatcctcgaactgcagcctctgagcagaggactgctggaggactaccagaagaaggaaaccggagttcctgaaagcaacagaaccctgctgctgtgcctgacatctgacagccagcctcctcggctgaacagcagcgctatccttccatactttcgggccatcagacccctgagcgacaagaacatcatcgacaagatcatcgaacagctcgacaagctgaaatttcagcatgagcctgaaacagaaatcagcgtgcccgccgatacattcgagtgcaagagcttcattctgaccatcctgcaacagttcagcgcttgcctggagagcgtcttcaagtctctgaacagcggcccccagggcggcggcggccaatacatcaaggccaacagcaagttcatcggaattacagagctggccggaagcggctctcccagccctgtgacaccttctcctacactgaaggaactgatcgaggaactcgtcaacatcacccagaatcaggcctccctgtgtaacggcagcatggtgtggtcggtgaacctgactgccggcatgtactgtgccgccctggagagcctgatcaacgtgtctgactgcagcgccatccagagaacccagagaatgctcaaggccctttgttctcagaaacctgccgccggacaaatcagcagcgagagatctagagataccaagatcgaggtgatccaactggtgaagaacctgctgacatacgtgcggggcgtgtaccggcacggaaattttcggggcggaggcggcttcaacaacttcaccgtgagcttctggctgagagtgcctaaggtgagcgccagccacctggaaggcagcggcggccacaacttcaacatcaccatcaaggagatcatcaagatgctgaacatcctgacagccagaaacgacagctgcatggaactgaccgtgaaggacgtgttcaccgcccctaagaacaccagcgataaagaaatcttttgcagagccgcaacagtgctgagacagatctacacccacaattgctccaacaggtaccttagaggcctgtaccggaacctgagctctatggccaacaagacatgctcaatgaatgagatcaagaagagtaccctgaaggatttcctggagcggctgaaggtgatcatgcagaaaaaatattacagacacgccggcagcggctttaacaatttcaccgtgagcttctggctgagagtgcctaaggtgagcgccagccacctggagggcagcggcggccaatacatcaaagctaattctaagtttatcggcatcaccgagctgcaccaccatcaccaccactgataagctt

SEQ ID NO: 205 - protein, 897 aa, Artificial sequence
  SEQ ID NO: 205 is the amino acid sequence of the cIL-31-cIL-13-cIL-4 polyprotein used for the vaccine construct of Example 13i.
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQGGGGFNNFTVSFWLRVPKVSASHLESGGSSPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRGSGGQYIKANSKFIGITELAGSGHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHAGSGFNNFTVSFWLRVPKVSASHLESGGSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQGGGGQYIKANSKFIGITELAGSGSPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRGGGGFNNFTVSFWLRVPKVSASHLEGSGGHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHAGSGFNNFTVSFWLRVPKVSASHLEGSGGQYIKANSKFIGITELHHHHHH